(19) United States
(12) Patent Application Publication
(10) Pub. No.: US 2025/0042970 A1
(43) Pub. Date: Feb. 6, 2025

(54) B CELL ACTIVATING FACTOR (BAFF)-A PROLIFERATION INDUCING LIGAND (APRIL) DUAL INHIBITORS

(71) Applicant: AURINIA PHARMACEUTICALS INC., Edmonton (CA)

(72) Inventors: Jennifer CROSS, Edmonton (CA); Robert B. HUIZINGA, Edmonton (CA); James William LARRICK, Sunnyvale, CA (US); Bo YU, Mountain View, CA (US); Stephen PARMLEY, San Diego, CA (US)

(73) Assignee: AURINIA PHARMACEUTICALS INC., Edmonton (CA)

(21) Appl. No.: 18/663,461

(22) Filed: May 14, 2024

Related U.S. Application Data
(63) Continuation of application No. PCT/IB2022/061038, filed on Nov. 16, 2022.
(60) Provisional application No. 63/280,556, filed on Nov. 17, 2021.
(60) Provisional application No. 63/339,334, filed on May 6, 2022.
(60) Provisional application No. 63/350,392, filed on Jun. 8, 2022.

(51) Int. Cl. C07K 14/705

(52) U.S. Cl. CPC C07K 14/70578; C07K 2319/30

(57) ABSTRACT

Provided are variant B-cell maturation antigen (BCMA) cysteine-rich domain (CRD) molecules capable of binding to ligands B-cell Activating Factor of the TNF family (BAFF) and A Proliferation Inducing Ligand (APRIL) with higher affinity compared to wild-type BCMA. In some aspects, also provided are variant BCMA fusion proteins or constructs comprising an immunoglobulin Fc domain, dimers, conjugates, methods for producing the molecules, and related methods and uses thereof.

### CROSS-REFERENCE TO RELATED APPLICATIONS

This application claims the benefit of U.S. Provisional Patent Application No. 63/280,556, filed Nov. 17, 2021; U.S. Provisional Patent Application No. 63/339,334, filed May 6, 2022; and U.S. Provisional Patent Application No. 63/350,392, filed Jun. 8, 2022, each of which is hereby incorporated by reference herein in its entirety for all purposes.

### SEQUENCE LISTING

This application contains a Sequence Listing which has been submitted electronically in ST.26 formatted XML format. The Sequence Listing XML is incorporated herein by reference. Said XML file, created on Nov. 14, 2022, is named 048475.000014_SL.xml and is 247,536 bytes in size.

### FIELD

The present disclosure relates in some aspects to variant B-cell maturation antigen (BCMA) cysteine-rich domain (CRD) molecules capable of binding to ligands B-cell Activating Factor of the TNF family (BAFF) and A Proliferation Inducing Ligand (APRIL) with higher affinity compared to wild-type BCMA. In some aspects, the disclosure further relates to variant BCMA fusion proteins or constructs comprising an immunoglobulin Fc domain, dimers, conjugates, methods for producing the molecules, and related methods and uses thereof.

### BACKGROUND

Human BCMA binds to TNF-superfamily ligands BAFF and APRIL, which are implicated in immune cell function, such as B-cell differentiation, survival, and proliferation. Improved strategies for the prevention or the treatment of B-cell mediated conditions, are necessary to provide effective molecules with a strong safety profile, such as molecules that provide dual blockade of BAFF and APRIL without inducing a potent cytotoxic response. Provided are compositions, methods, and uses that meet such needs.

### SUMMARY

Provided herein are variant B cell maturation antigen (BCMA) polypeptides. In some of any of the provided embodiments, the variant BCMA polypeptide comprises a variant cysteine rich domain (CRD) comprising at least one amino acid substitution(s) selected from among: (1) a histidine, an arginine, a proline, or an asparagine at position 12 (S12H, S12R, S12P, or S12N); (2) an isoleucine or a valine at position 14 (L14I or L14V); (3) an arginine or an asparagine at position 15 (H15R or H15N); (4) a serine at position 16 (A16S); and (5) a valine, an isoleucine, or an alanine at position 22 (L22V, L22I, or L22A), with reference to the amino acid positions of SEQ ID NO:1.

In some of any embodiments, the variant CRD comprises at least three amino acid substitutions selected from among: (1) a histidine, an arginine, a proline, or an asparagine at position 12 (S12H, S12R, S12P, or S12N); (2) an isoleucine or a valine at position 14 (L14I or L14V); (3) an arginine or an asparagine at position 15 (H15R or H15N); (4) a serine at position 16 (A16S); and (5) a valine, an isoleucine, or an alanine at position 22 (L22V, L22I, or L22A), with reference to the amino acid positions of SEQ ID NO:1. In some of any embodiments, the variant CRD comprises at least three amino acid substitutions selected from among: (1) a histidine or an arginine at position 12 (S12H or S12R); (2) an isoleucine or a valine at position 14 (L14I or L14V); (3) an arginine at position 15 (H15R); and (4) a valine at position 22 (L22V), with reference to the amino acid positions of SEQ ID NO:1. In some of any embodiments, the variant CRD further comprises at least one modification selected from among: (1) a deletion at residue 38 (N38del) or an amino acid residue that is not asparagine at position 38 (N38X, wherein X is any amino acid residue that is not asparagine) and (2) an amino acid residue that is not serine or threonine at position 40 (S40X, wherein X is any amino acid residue that is not serine or threonine), with reference to the amino acid positions of SEQ ID NO:1. In some of any embodiments, the variant CRD further comprises a glycine at residue 40 (S40G), with reference to the amino acid positions of SEQ ID NO:1.

In some of any embodiments, the variant CRD comprises at least at or about 85%, 86%, 87%, 88%, 89%, 90%, 91%, 92%, 93%, 94%, 95%, 96%, 97%, 98%, or 99% sequence identity to SEQ ID NO: 1. In some of any embodiments, the variant CRD comprises at least at or about 90% sequence identity to SEQ ID NO: 1. In some of any embodiments, the variant CRD comprises at least at or about 95% sequence identity to SEQ ID NO: 1. In some of any embodiments, the variant CRD comprises at least at or about 99% sequence identity to SEQ ID NO:1.

In some of any embodiments, the variant CRD comprises at least at or about 90% sequence identity to the sequence set forth in any one of SEQ ID NOs:3-146. In some of any embodiments, the variant CRD comprises at least at or about 90% sequence identity to the sequence set forth in any one of SEQ ID NO:3, SEQ ID NO:4, SEQ ID NO:6, SEQ ID NO:8, SEQ ID NO:9, or SEQ ID NO:19. In some of any embodiments, the variant CRD comprises the sequence set forth in any one of SEQ ID NO: 3, SEQ ID NO:4, SEQ ID NO:6, SEQ ID NO:8, SEQ ID NO:9, or SEQ ID NO:19.

In some of any embodiments, the variant CRD comprises at least three amino acid substitutions selected from the group consisting of: (1) a histidine at position 12 (S12H); (2) an isoleucine at position 14 (L14I); (3) an arginine at position 15 (H15R or H15N); and (4) and a serine to glycine mutation at residue 40 (S40G), with reference to the amino acid positions of SEQ ID NO:1, and wherein the variant CRD comprises at least at or about 90% sequence identity to SEQ ID NO:1. In some of any embodiments, the variant CRD comprises each of the following amino acid substitutions: (1) a histidine at position 12 (S12H); (2) an isoleucine at position 14 (L14I); (3) an arginine at position 15 (H15R); and (4) a serine to glycine mutation at residue 40 (S40G), with reference to the amino acid positions of SEQ ID NO:1, and wherein the variant CRD comprises at least at or about 90% sequence identity to SEQ ID NO:1.

In some of any embodiments, the variant CRD comprises at least at or about 90% sequence identity to SEQ ID NO:3. In some of any embodiments, the variant CRD comprises the sequence set forth in SEQ ID NO:3.

In some of any embodiments, the variant CRD comprises at least at or about 90% sequence identity to SEQ ID NO:4. In some of any embodiments, the variant CRD comprises the sequence set forth in SEQ ID NO:4.

In some of any embodiments, the variant CRD comprises at least at or about 90% sequence identity to SEQ ID NO:8. In some of any embodiments, the variant CRD comprises the sequence set forth in SEQ ID NO:8.

In some of any embodiments, the variant CRD comprises at least at or about 90% sequence identity to SEQ ID NO:9. In some of any embodiments, the variant CRD comprises the sequence set forth in SEQ ID NO: 9.

In some of any embodiments, the variant CRD comprises at least at or about 90% sequence identity to SEQ ID NO:19. In some of any embodiments, the variant CRD comprises the sequence set forth in SEQ ID NO:19.

Also provided herein are fusion polypeptides. In some of any of the provided embodiments, the fusion polypeptide comprises any of the variant BCMA polypeptides provided herein, and an additional polypeptide. In some of any embodiments, the additional polypeptide is an immunoglobulin (Ig) Fc polypeptide.

Also provided herein are the fusion polypeptides that comprise any of the variant BCMA polypeptides provided herein, and an immunoglobulin (Ig) Fc polypeptide.

Also provided herein are the fusion polypeptides that comprise a variant B cell maturation antigen (BCMA) polypeptide, comprising a variant cysteine rich domain (CRD) comprising at least one amino acid substitutions selected from among: (1) a histidine, an arginine, a proline, or an asparagine at position 12 (S12H, S12R, S12P, or S12N); (2) an isoleucine or a valine at position 14 (L14I or L14V); (3) an arginine or an asparagine at position 15 (H15R or H15N); (4) a serine at position 16 (A16S); and (5) a valine, an isoleucine, or an alanine at position 22 (L22V, L22I, or L22A); with reference to the amino acid positions of SEQ ID NO:1; and an immunoglobulin (Ig) Fc polypeptide.

In some of any embodiments, the variant CRD comprises at least three amino acid substitutions selected from among: (1) a histidine, an arginine, a proline, or an asparagine at position 12 (S12H, S12R, S12P, or S12N); (2) an isoleucine or a valine at position 14 (L14I or L14V); (3) an arginine or an asparagine at position 15 (H15R or H15N); (4) a serine at position 16 (A16S); and (5) a valine, an isoleucine, or an alanine at position 22 (L22V, L22I, or L22A), with reference to the amino acid positions of SEQ ID NO:1. In some of any embodiments, the variant CRD comprises least three amino acid substitutions selected from among: (1) a histidine or an arginine at position 12 (S12H or S12R); (2) an isoleucine or a valine at position 14 (L14I or L14V); (3) an arginine at position 15 (H15R); and (4) a valine at position 22 (L22V), with reference to the amino acid positions of SEQ ID NO:1. In some of any embodiments, the variant CRD further comprises at least one modification selected from among: (1) a deletion at residue 38 (N38del) or an amino acid residue that is not asparagine at position 38 (N38X, wherein X is any amino acid residue that is not asparagine) and (2) an amino acid residue that is not serine or threonine at position 40 (S40X, wherein X is any amino acid residue that is not serine or threonine), with reference to the amino acid positions of SEQ ID NO:1. In some of any embodiments, the variant CRD further comprises a glycine at residue 40 (S40G), with reference to the amino acid positions of SEQ ID NO:1.

In some of any embodiments, the variant CRD comprises at least at or about 90% sequence identity to the sequence set forth in any one of SEQ ID NOS:3-146. In some of any embodiments, the variant CRD comprises at least at or about 90% sequence identity to the sequence set forth in SEQ ID NO:3, SEQ ID NO:4, SEQ ID NO:6, SEQ ID NO:8, SEQ ID NO:9, or SEQ ID NO:19. In some of any embodiments, the variant CRD comprises the sequence set forth in SEQ ID NO:3, SEQ ID NO:4, SEQ ID NO:6, SEQ ID NO:8, SEQ ID NO:9, or SEQ ID NO:19.

In some of any embodiments, the variant CRD comprises at least at or about 90% sequence identity to SEQ ID NO:3. In some of any embodiments, the variant CRD comprises the sequence set forth in SEQ ID NO: 3.

In some of any embodiments, the variant CRD comprises at least at or about 90% sequence identity to SEQ ID NO:4. In some of any embodiments, the variant CRD comprises the sequence set forth in SEQ ID NO:4.

In some of any embodiments, the variant CRD comprises at least at or about 90% sequence identity to SEQ ID NO:8. In some of any embodiments, the variant CRD comprises the sequence set forth in SEQ ID NO:8.

In some of any embodiments, the variant CRD comprises at least at or about 90% sequence identity to SEQ ID NO:9. In some of any embodiments, the variant CRD comprises the sequence set forth in SEQ ID NO:9.

In some of any embodiments, the variant CRD comprises at least at or about 90% sequence identity to SEQ ID NO:19. In some of any embodiments, the variant CRD comprises the sequence set forth in SEQ ID NO:19.

In some of any embodiments, the variant BCMA polypeptide is directly or indirectly fused to the N-terminus or the C-terminus of the Ig Fc polypeptide.

In some of any embodiments, the Ig Fc polypeptide is or is derived from an isotype G immunoglobulin (IgG) or a variant thereof.

In some of any embodiments, the Ig Fc polypeptide is or is derived from an IgG1 Fc, an IgG2 Fe, an IgG3 Fe, or an IgG4 Fc. In some of any embodiments, the Ig Fc polypeptide is or is derived from a human IgG Fc. In some of any embodiments, the Ig Fc polypeptide is or is derived from a human IgG1 Fe, a human IgG2 Fe, a human IgG3 Fe, or a human IgG4 Fc.

In some of any embodiments, the Ig Fc polypeptide is or is derived from an IgG1 Fc. In some of any embodiments, the Ig Fc polypeptide is or is derived from a human IgG1 Fc. In some of any embodiments, the Ig Fc polypeptide is a human IgG1 Fc and comprises a sequence set forth in SEQ ID NO:168, SEQ ID NO:169, SEQ ID NO:170, SEQ ID NO:226, SEQ ID NO:227, SEQ ID NO:228, SEQ ID NO:229, or SEQ ID NO:230, or a sequence that has at least 90% sequence identity thereto. In some of any embodiments, the Ig Fc polypeptide is a human IgG1 Fc and comprises a sequence set forth in SEQ ID NO:168, SEQ ID NO:169, SEQ ID NO:170, SEQ ID NO:226, SEQ ID NO:227, SEQ ID NO:228, SEQ ID NO:229, or SEQ ID NO:230.

In some of any embodiments, the Ig Fc polypeptide is or is derived from an IgG2 Fc. In some of any embodiments, the Ig Fc polypeptide is or is derived from a human IgG2 Fc. In some of any embodiments, the Ig Fc polypeptide is a human IgG2 Fc and comprises a sequence set forth in SEQ ID NO:171, SEQ ID NO:235, or SEQ ID NO:236, or a sequence that has at least 90% sequence identity thereto. In some of any embodiments, the Ig Fc polypeptide is a human IgG2 Fc and comprises a sequence set forth in SEQ ID NO:171, SEQ ID NO:235, or SEQ ID NO:236.

In some of any embodiments, the Ig Fc polypeptide is or is derived from an IgG4 Fc. In some of any embodiments, the Ig Fc polypeptide is or is derived from a human IgG4 Fc. In some of any embodiments, the Ig Fc polypeptide is a human IgG4 Fc and comprises a sequence set forth in SEQ ID NO:161, SEQ ID NO:163, SEQ ID NO:172, SEQ ID NO: 231, SEQ ID NO:232, SEQ ID NO:233, or SEQ ID NO:234, or a sequence that has at least 90% sequence identity thereto. In some of any embodiments, the Ig Fc polypeptide is a human IgG4 Fc and comprises a sequence set forth in SEQ ID NO:161, SEQ ID NO:163, SEQ ID NO:172, SEQ ID NO: 231, SEQ ID NO:232, SEQ ID NO:233, or SEQ ID NO:234. In some of any embodiments, the Ig Fc polypeptide is a human IgG4 Fc and comprises the sequence set forth in SEQ ID NO:161, or a sequence that has at least 90% sequence identity thereto. In some of any embodiments, the Ig Fc polypeptide comprises SEQ ID NO:161. In some of any embodiments, the Ig Fc polypeptide is a human IgG4 Fc and comprises the sequence set forth in SEQ ID NO:163, or a sequence that has at least 90% sequence identity thereto. In some of any embodiments, the Ig Fc polypeptide comprises SEQ ID NO:163.

In some of any embodiments, the Ig Fc polypeptide comprises an isoleucine or a valine substituted for one, two, three, four, or more native methionine residues.

In some of any embodiments, the variant BCMA polypeptide comprises SEQ ID NO:3 and the Ig Fc polypeptide comprises SEQ ID NO:161. In some of any embodiments, the variant BCMA polypeptide comprises SEQ ID NO:3 and the Ig Fc polypeptide comprises SEQ ID NO:163. In some of any embodiments, the variant BCMA polypeptide comprises SEQ ID NO:4 and the Ig Fc polypeptide comprises SEQ ID NO:161. In some of any embodiments, the variant BCMA polypeptide comprises SEQ ID NO:4 and the Ig Fc polypeptide comprises SEQ ID NO:163. In some of any embodiments, the variant BCMA polypeptide comprises SEQ ID NO:8 and the Ig Fc polypeptide comprises SEQ ID NO:161. In some of any embodiments, the variant BCMA polypeptide comprises SEQ ID NO:8 and the Ig Fc polypeptide comprises SEQ ID NO:163. In some of any embodiments, the variant BCMA polypeptide comprises SEQ ID NO:9 and the Ig Fc polypeptide comprises SEQ ID NO:161. In some of any embodiments, the variant BCMA polypeptide comprises SEQ ID NO:9 and the Ig Fc polypeptide comprises SEQ ID NO:163. In some of any embodiments, the variant BCMA polypeptide comprises SEQ ID NO:19 and the Ig Fc polypeptide comprises SEQ ID NO:161. In some of any embodiments, the variant BCMA polypeptide comprises SEQ ID NO:19 and the Ig Fc polypeptide comprises SEQ ID NO:163.

In some of any embodiments, the fusion polypeptide further comprises a peptide linker that links the variant BCMA polypeptide to the Ig Fc polypeptide.

In some of any embodiments, the peptide linker is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids in length. In some of any embodiments, the peptide linker is 4, 5, 6, or 7 amino acids in length.

In some of any embodiments, the peptide linker comprises residues selected from the group consisting of glycine, serine, alanine, and threonine. In some of any embodiments, the peptide linker comprises a sequences set forth in any one of SEQ ID NO:156, 158, 175-186, 188-213, GS, GGS and GSA. In some of any embodiments, the peptide linker comprises SEQ ID NO:156. In some of any embodiments, the peptide linker comprises SEQ ID NO:158.

In some of any embodiments, the fusion polypeptide comprises, in N- to C-terminal order: the variant BCMA polypeptide, a peptide linker and an Ig Fc polypeptide.

In some of any embodiments, the fusion polypeptide comprises a variant BCMA polypeptide set forth in SEQ ID NO:3, SEQ ID NO:4, SEQ ID NO:8, SEQ ID NO:9, or SEQ ID NO:19; a peptide linker comprising a sequences set forth in any one of SEQ ID NO:156, 158, 175-186, 188-213, GS, GGS and GSA, and an Ig Fc polypeptide comprising a sequence set forth in SEQ ID NO:161, SEQ ID NO:163, SEQ ID NO:172, SEQ ID NO: 231, SEQ ID NO:232, SEQ ID NO:233, or SEQ ID NO:234.

In some of any embodiments, the fusion polypeptide comprises a variant BCMA polypeptide comprising SEQ ID NO:3, a peptide linker comprising SEQ ID NO:156, and an Ig Fc polypeptide comprising SEQ ID NO:161. In some of any embodiments, the fusion polypeptide comprises a variant BCMA polypeptide comprising SEQ ID NO:3, a peptide linker comprising SEQ ID NO:158, and an Ig Fc polypeptide comprising SEQ ID NO:163. In some of any embodiments, the fusion polypeptide comprises a variant BCMA polypeptide comprising SEQ ID NO:4, a peptide linker comprising SEQ ID NO:156, and an Ig Fc polypeptide comprising SEQ ID NO:161. In some of any embodiments, the fusion polypeptide comprises a variant BCMA polypeptide comprising SEQ ID NO:4, a peptide linker comprising SEQ ID NO:158, and an Ig Fc polypeptide comprising SEQ ID NO:163. In some of any embodiments, the fusion polypeptide comprises a variant BCMA polypeptide comprising SEQ ID NO:8, a peptide linker comprising SEQ ID NO:156, and an Ig Fc polypeptide comprising SEQ ID NO:161. In some of any embodiments, the fusion polypeptide comprises a variant BCMA polypeptide comprising SEQ ID NO:8, a peptide linker comprising SEQ ID NO:158, and an Ig Fc polypeptide comprising SEQ ID NO:163. In some of any embodiments, the fusion polypeptide comprises a variant BCMA polypeptide comprising SEQ ID NO:9, a peptide linker comprising SEQ ID NO:156, and an Ig Fc polypeptide comprising SEQ ID NO:161. In some of any embodiments, the fusion polypeptide comprises a variant BCMA polypeptide comprising SEQ ID NO:9, a peptide linker comprising SEQ ID NO:158, and an Ig Fc polypeptide comprising SEQ ID NO:163. In some of any embodiments, the fusion polypeptide comprises a variant BCMA polypeptide comprising SEQ ID NO:19, a peptide linker comprising SEQ ID NO:156, and an Ig Fc polypeptide comprising SEQ ID NO:161. In some of any embodiments, the fusion polypeptide comprises a variant BCMA polypeptide comprising SEQ ID NO:19, a peptide linker comprising SEQ ID NO:158, and an Ig Fc polypeptide comprising SEQ ID NO:163.

In some of any embodiments, the fusion polypeptide comprises the sequence set forth in SEQ ID NO:167 or a sequence that has at least 90% sequence identity thereto. In some of any embodiments, the fusion polypeptide comprises the sequence set forth in SEQ ID NO:167.

In some of any embodiments, the fusion polypeptide comprises a first monomer comprising a first variant BCMA polypeptide directly or indirectly fused to a first Ig Fc polypeptide, and one or more second monomer comprising a second variant BCMA polypeptide directly or indirectly fused to a second Ig Fc polypeptide, wherein the first monomer and the one or more second monomers are fused in tandem. In some of any embodiments, the first variant BCMA polypeptide and the second variant BCMA polypeptide are the same. In some of any embodiments, the first variant BCMA polypeptide and the second variant BCMA polypeptide are different. In some of any embodiments, the first monomer and the second monomer are the same. In some of any embodiments, the first monomer and the second monomer are different.

Also provided herein are dimers, such as dimers of any of the variant BCMA polypeptides or any of the fusion polypeptides provided herein. In some of any of the provided embodiments, the dimer comprises a first monomer comprising any of the variant BCMA polypeptides or any of the fusion polypeptides provided herein and a second monomer comprising any of the variant BCMA polypeptides or any of the fusion polypeptides provided herein. In some of any embodiments, the first monomer comprises a first variant BCMA polypeptide directly or indirectly fused to a first Ig Fc polypeptide, and the second monomer comprises a second variant BCMA polypeptide directly or indirectly fused to a second Ig Fc polypeptide.

In some of any embodiments, the first variant BCMA polypeptide and the second variant BCMA polypeptide are the same. In some of any embodiments, the first variant BCMA polypeptide and the second variant BCMA polypeptide are different. In some of any embodiments, the first Ig Fc polypeptide and the second Ig Fc polypeptide are the same. In some of any embodiments, the first Ig Fc polypeptide and the second Ig Fc polypeptide are different. In some of any embodiments, the first monomer and the second monomer are the same. In some of any embodiments, the first monomer and the second monomer are different.

In some of any embodiments, the first monomer and the second monomer are linked together by at least one disulfide bond between cysteine residues in the first monomer and the second monomer. In some of any embodiments, the disulfide bond is between cysteine residues of the Ig Fc polypeptide of the first monomer and the Ig Fc polypeptide of the second monomer.

Also provided herein are conjugates. In some of any of the embodiments, the conjugate comprises any of the variant BCMA polypeptides, any of the fusion polypeptides provided herein, or any of the dimers provided herein; and an additional moiety that is covalently bound to the variant BCMA polypeptide, the fusion polypeptide, or the dimer.

In some of any embodiments, the additional moiety is selected from a therapeutic moiety, a polymer moiety, a sugar moiety, and a lipophilic moiety.

In some of any embodiments, the additional moiety is selected from among one or more of a polyalkylene oxide (PAO), a polyalkylene glycol (PAG), a polyethylene glycol (PEG), a monomethoxypolyethylene glycol (mPEG), a polypropylene glycol (PPG), a branched polyethylene glycol having two or more polyethylene glycol chains linked together by a linker group, a polyvinyl alcohol (PVA), a polycarboxylate, a poly(vinylpyrrolidone), a polyethylene-co-maleic acid anhydride, and a dextran.

In some of any of the provided embodiments, the variant BCMA polypeptide, the fusion polypeptide, the dimer, or the conjugate binds B-cell Activating Factor of the TNF family (BAFF) and/or A Proliferation Inducing Ligand (APRIL) or variants thereof.

In some of any of the provided embodiments, the variant BCMA polypeptide, the fusion polypeptide, the dimer, or the conjugate exhibits greater binding affinity for BAFF and/or APRIL, compared to the binding affinity of a reference BCMA polypeptide or a reference binding molecule.

In some of any of the provided embodiments, the variant BCMA polypeptide, the fusion polypeptide, the dimer, or the conjugate exhibits greater inhibition of the activity or function of BAFF and/or APRIL, compared to the inhibition of the activity or function of BAFF and/or APRIL by a reference BCMA polypeptide or a reference binding molecule.

In some of any of the provided embodiments, the variant BCMA polypeptide, the fusion polypeptide, the dimer, or the conjugate reduces proliferation of B cells or reduces BAFF and/or APRIL-mediated proliferation of B cells.

In some of any of the embodiments, the variant BCMA polypeptide, the fusion polypeptide, the dimer, or the conjugate, reduces the production of inflammatory cytokines. In some of the embodiments, the inflammatory cytokine is one or more of IFNγ or IL-17A.

In some of any of the provided embodiments, BAFF is a human BAFF and APRIL is a human APRIL.

In some of any of the provided embodiments, BAFF is a murine BAFF and APRIL is a murine APRIL.

In some of any of the provided embodiments, the reference BCMA polypeptide is a wild-type human BCMA CRD set forth in SEQ ID NO: 1. In some of any of the provided embodiments, the reference BCMA polypeptide is a human BCMA CRD comprising a serine to glycine substitution at position 40 (S40G) comprising the sequence set forth in SEQ ID NO:237.

In some of any of the provided embodiments, the reference binding molecule is selected from among Atacicept, Telitacicept, belimumab, or BION-1301.

In some of any of the provided embodiments, the binding affinity of the variant BCMA polypeptide, the fusion polypeptide, the dimer, or the conjugate to human BAFF is at least at or about 1-, 2-, 3-, 4-, 5-, 10-, 20-, 25-, 50-, 100-, 200-, 250-, or 500-fold greater than that of the reference BCMA polypeptide or the reference binding molecule. In some of any of the provided embodiments, the inhibition of activity or function of human BAFF by the variant BCMA polypeptide, the fusion polypeptide, the dimer, or the conjugate is at least at or about 5-, 10-, 20-, 25-, 50-, 100-, 200-, 250-, or 500-fold greater than that of the reference BCMA polypeptide or the reference binding molecule.

In some of any of the provided embodiments, the binding affinity of the variant BCMA polypeptide, the fusion polypeptide, the dimer, or the conjugate to human APRIL is at least at or about 1-, 2-, 3-, 4-, 5-, 10-, 20-, 25-, 50-, 100-, 200-, 250-, or 500-fold greater than that of the reference BCMA polypeptide or the reference binding molecule. In some of any of the provided embodiments, the inhibition of activity or function of human APRIL by the variant BCMA polypeptide, the fusion polypeptide, the dimer, or the conjugate is at least at or about 5-, 10-, 20-, 25-, 50-, 100-, 200-, 250-, or 500-fold greater than that of the reference BCMA polypeptide or the reference binding molecule.

In some of any of the provided embodiments, the ratio of binding selectivity for human BAFF relative to human APRIL (huBAFF K_{D}/huAPRIL K_{D}) is more than 5, 4, 3, 2 or 1. In some of any of the provided embodiments, the equilibrium dissociation constant (K_{D}) for binding to human BAFF is less than 600 pM, less than 550 pM, less than 500 pM, less than 450 pM, less than 400 pM, less than 350 pM, less than 300 pM, less than 250 pM, less than 200 pM, or less than 150 pM. In some of any of the provided embodiments, the equilibrium dissociation constant (K_{D}) for binding to human BAFF is in the picomolar (pM) range.

In some of any of the provided embodiments, the equilibrium dissociation constant (K_{D}) for binding to human BAFF is in the sub-picomolar (pM) range. In some of any of the provided embodiments, the equilibrium dissociation constant (K_{D}) for binding to human BAFF is less than 1.0 pM, less than 0.9 pM, less than 0.8 pM, less than 0.7 pM, less than 0.6 pM, less than 0.5 pM, less than 0.4 pM, less than 0.3 pM, less than 0.2 pM, or less than 0.1 pM. In some of any of the provided embodiments, the equilibrium dissociation constant (K_{D}) for binding to human APRIL is less than 100 pM, less than 90 pM, less than 80 pM, less than 70 pM, less than 60 pM, less than 50 pM, less than 40 pM, or less than 30 pM.

In some of any of the provided embodiments, the equilibrium dissociation constant (K_{D}) for binding to human APRIL is in the picomolar (pM) range. In some of any of the provided embodiments, the equilibrium dissociation constant (K_{D}) for binding to human APRIL is in the sub-picomolar (pM) range. In some of any of the provided embodiments, the equilibrium dissociation constant (K_{D}) for binding to human APRIL is less than 1.0 pM, less than 0.9 pM, less than 0.8 pM, less than 0.7 pM, less than 0.6 pM, less than 0.5 pM, less than 0.4 pM, less than 0.3 pM, less than 0.2 pM, or less than 0.1 pM. In some of any of the provided embodiments, the equilibrium dissociation constant (K_{D}) for binding to human BAFF and human APRIL both are less than 120 pM. In some of any of the provided embodiments, the equilibrium dissociation constant (K_{D}) for binding to human BAFF and human APRIL both are less than 0.3 pM. In some of any of the provided embodiments, the equilibrium dissociation constant (K_{D}) is measured by a kinetic exclusion assay or surface plasmon resonance (SPR).

In some of any of the provided embodiments, the fusion polypeptide, the dimer, or the conjugate does not substantially bind to heparan sulfate proteoglycans (HSPGs). In some of any of the provided embodiments, the HSPGs are selected from among one or more of syndecan-1 and syndecan-2.

In some of any of the provided embodiments, the variant BCMA polypeptide, the fusion polypeptide, the dimer, or the conjugate inhibits the activity or function of BAFF and/or APRIL or variants thereof. In some of any of the provided embodiments, the activity or function of BAFF and/or APRIL is selected from B-cell survival, B-cell proliferation, and/or immunoglobulin production.

Also provided herein are polynucleotides. In some of any of the provided embodiments, the polynucleotide comprises a nucleotide sequence encoding any of the variant BCMA polypeptide provided herein, any of the fusion polypeptide provided herein, or a first monomer and/or a second monomer of any one of the dimers provided herein.

Also provided are vectors. In some of any of the provided embodiments, the vector comprises any of the polynucleotides provided herein, or a polynucleotide comprising a nucleotide sequence encoding any of the variant BCMA polypeptide provided herein, any of the fusion polypeptide provided herein, or a first monomer and/or a second monomer of any one of the dimers provided herein.

Also provided herein are cells. In some of any of the provided embodiments, the cell comprises any of the polynucleotides or any of the vectors provided herein.

Also provided herein are methods of manufacturing a variant BCMA polypeptide, a fusion polypeptide, or a dimer. In some of any embodiments, the method involves introducing any of the polynucleotides or any of the vectors provided herein into a cell; culturing the host cell under conditions suitable for expression of the polypeptide; and recovering or isolating the polypeptide. In some of any of the embodiments, the methods also involve purifying the polypeptide.

Also provided herein are pharmaceutical compositions. In some of any of the provided embodiments, the pharmaceutical compositions comprise any of the variant BCMA polypeptide provided herein, any of the fusion polypeptide provided herein, any of the dimers provided herein, any of the conjugates provided herein, any of the polynucleotides provided herein, any of the vectors provided herein or any of the cells provided herein.

In some of any embodiments, the pharmaceutical composition also comprises one or more pharmaceutically acceptable excipient(s). In some of any embodiments, the one or more excipient(s) comprises a pharmaceutically acceptable liquid carrier. In some of any embodiments, the one or more excipient(s) comprises a pharmaceutically acceptable processing agents. In some of any embodiments, the pharmaceutical composition is a liquid formulation, a formulation for an intravenous injection, a solid dosage form, or an inhalable preparation.

Also provided herein are any of the provided pharmaceutical compositions for treating a disease or disorder. In some of any embodiments, the pharmaceutical composition is to be administered to a subject having the disease or disorder.

Also provided herein are any of the variant BCMA polypeptide provided herein, any of the fusion polypeptide provided herein, any of the dimers provided herein, any of the conjugates provided herein, any of the polynucleotides provided herein, any of the vectors provided herein or any of the cells provided herein for treating a disease or disorder.

In some of any embodiments, the variant BCMA polypeptide, the fusion polypeptide, the dimer, the conjugate, the polynucleotide, the vector or the cell to be administered to a subject having the disease or disorder.

Also provided herein are methods of treatment. In some of any of the embodiments, the methods involve administering any of the variant BCMA polypeptide provided herein, any of the fusion polypeptide provided herein, any of the dimers provided herein, any of the conjugates provided herein, any of the polynucleotides provided herein, any of the vectors provided herein, any of the cells provided herein or any of the pharmaceutical composition provided herein to a subject having the disease or disorder.

Also provided herein are uses of any of the variant BCMA polypeptide provided herein, any of the fusion polypeptide provided herein, any of the dimers provided herein, any of the conjugates provided herein, any of the polynucleotides provided herein, any of the vectors provided herein, any of the cells provided herein or any of the pharmaceutical composition provided herein in the manufacture of a medicament for the treatment of a disease or disorder.

Also provided herein are uses of any of the variant BCMA polypeptide provided herein, any of the fusion polypeptide provided herein, any of the dimers provided herein, any of the conjugates provided herein, any of the polynucleotides provided herein, any of the vectors provided herein, any of the cells provided herein or any of the pharmaceutical composition provided herein for treating a disease or disorder.

In some of any of the provided embodiments, the variant BCMA polypeptide, the fusion polypeptide, the dimer, the conjugate or the pharmaceutical composition is to be administered to a subject having the disease or disorder.

In some of any of the provided embodiments, the disease or disorder is a B-cell- or antibody-mediated disease or disorder.

In some of any of the provided embodiments, the disease or disorder is an autoimmune disease or disorder. In some of any of the provided embodiments, the autoimmune disease or disorder is an immune-mediated disease or disorder of the subject's tissues, bone, joints, blood vessels, thyroid, kidneys, nervous system, brain, lungs, and/or skin. In some of any of the provided embodiments, the autoimmune disease or disorder is selected from among a renal disease or disorder, lupus, arthritis, a spondyloarthropathic disorder, a vasculitis disorder, a hemolytic anemia disorder, a thrombocytopenia disorder, a thyroiditis disorder, a demyelinating disease of the central and/or peripheral nervous system, inflammatory and/or fibrotic lung disorder, a skin disorder, or an allergic disorder.

In some of any of the provided embodiments, the disease or disorder is selected from among systemic lupus erythematosus (SLE) and/or lupus nephritis (LN), IgA nephropathy (Berger's disease), Goodpasture syndrome, anti-neutrophil cytoplasmic antibody (ANCA) associated vasculitis, Henoch-Schonlein purpura, polyarteritis nodosa (PAN), sarcoidosis of the kidneys, rheumatoid arthritis, juvenile chronic arthritis, arthritis associated with inflammatory bowel disease, ankylosing spondylitis, spondylitis associated with psoriasis, juvenile onset spondyloarthropathy, undifferentiated spondyloarthropathy, Reiter's syndrome, scleroderma, Sjogren's syndrome, systemic necrotizing vasculitis, polyarteritis nodosa, allergic angiitis and granulomatosis, polyangiitis, Wegener's granulomatosis, lymphomatoid granulomatosis, mucocutaneous lymph node syndrome (MLNS or Kawasaki's disease), isolated central nervous system vasculitis, Behet's disease, thromboangiitis obliterans (Buerger's disease), cutaneous necrotizing venulitis, sarcoidosis, autoimmune hemolytic anemia, immune pancytopenia, paroxysmal nocturnal hemoglobinuria, thrombocytopenic purpura, immune-mediated thrombocytopenia, Grave's disease, Hashimoto's thyroiditis, juvenile lymphocytic thyroiditis, atrophic thyroiditis, type I diabetes mellitus, glomerulonephritis and tubulointerstitial nephritis, multiple sclerosis (MS), idiopathic demyelinating polyneuropathy, Guillain-Barre syndrome, chronic inflammatory demyelinating polyneuropathy, eosinophilic pneumonia, idiopathic pulmonary fibrosis, hypersensitivity pneumonitis, bullous skin disease, erythema multiforme, contact dermatitis, asthma, allergic rhinitis, atopic dermatitis, food hypersensitivity, or urticaria.

In some of any of the provided embodiments, the disease or disorder is selected from among systemic lupus erythematosus (SLE) and/or lupus nephritis (LN), IgA nephropathy (Berger's disease), Goodpasture syndrome, anti-neutrophil cytoplasmic antibody (ANCA) associated vasculitis, Henoch-Schonlein purpura, Polyarteritis nodosa (PAN), or sarcoidosis of the kidneys. In some of any of the provided embodiments, the disease or disorder is selected from among systemic lupus erythematosus (SLE) and/or lupus nephritis (LN), or IgA nephropathy (Berger's disease).

In some of any of the provided embodiments, the disease or disorder is a tissue or organ transplant rejection. In some of any of the provided embodiments, the tissue or organ transplant rejection is selected from among acute or chronic B-cell or antibody-mediated rejection of allografts of tissues consisting of bone marrow, stem cell, skin, and solid organs, acute or chronic graft versus host disease (GVHD), antibody-mediated rejection (AMR) of solid organs, hyperacute organ transplant rejection, acute organ transplant rejection, chronic organ transplant rejection.

In some of any of the provided embodiments, the disease or disorder is a B-cell malignancy. In some of any of the provided embodiments, the B-cell malignancy is selected from among non-Hodgkin's lymphoma, multiple myeloma (MM), B-chronic lymphocytic leukemia, plasmacytoma, macroglobulinemia, or Waldenstrom's macroglobulinemia (WM).

In some of any of the provided embodiments, a therapeutically effective amount of the variant BCMA polypeptide, the fusion polypeptide, the dimer, the conjugate, the polynucleotide, the vector, the cell or the pharmaceutical composition is to be administered to the subject.

In some of any of the provided embodiments, the method or the use is a therapeutic use or a prophylactic use. In some of any of the provided embodiments, the therapeutic use is for induction therapy. In some of any of the provided embodiments, the induction therapy spans for up to at or about one week, up to at or about two weeks, up to at or about three weeks, or up to at or about four weeks. In some of any of the provided embodiments, the therapeutic use is for maintenance therapy. In some of any of the provided embodiments, the maintenance therapy spans for up to at or about one week, up to at or about two weeks, up to at or about three weeks, or up to at or about four weeks.

In some of any of the provided embodiments, the administration is selected from among intravenous, oral, parenteral, sublingual, by inhalation, rectal or topical. In some of any of the provided embodiments, the administration is intravenous administration.

### BRIEF DESCRIPTION OF THE DRAWINGS

FIG. 1A shows a polynucleotide sequence (SEQ ID NO:148) encoding full length human BCMA. FIG. 1B depicts the amino acid sequence (SEQ ID NO:149) encoded by the polynucleotide sequence of FIG. 1A, and corresponds to full length human BCMA. The underlined region depicts the BAFF (TALL-1) binding region (amino acid residues 8-46). The bold region indicates an exemplary TNFR (CRD) region (amino acid residues 7-45). The italicized region indicates the transmembrane domain (amino acid residues 55-77).

FIG. 2A depicts a polynucleotide sequence (SEQ ID NO:2) corresponding to the extracellular domain of human BCMA. FIG. 2B depicts an amino acid sequence (SEQ ID NO:152) corresponding to the extracellular domain of human BCMA (corresponding to amino acid residues 5-49 of SEQ ID NO:150).

FIG. 3A-FIG. 3K depict an alignment of amino acid sequences of exemplary variant BCMA polypeptides as described as compared to the amino acid sequence of the extracellular domain of human BCMA (SEQ ID NO:152) and the second cysteine-rich domain of TACI.

FIG. 4 is a schematic diagram of vector CET1019-BCMA-PIg18 which is described in Example 1A.

FIG. 5 provides a schematic diagram of vector CET1019AS-Z-TACI-Ig which is described in Example 1B.

FIG. 6 provides a schematic diagram of pre-BCMA ECD-Pig18 and pre-Z-TACI ECD-Ig.

FIG. 7A and FIG. 7B are sensorgram traces of exemplary variant BCMA polypeptide 233622 (designated “233622-Ig,” variant BCMA ECD component 233622) in fusion with an Ig polypeptide, the [S40G]huBCMA-Ig fusion polypeptide (designated “BCMA-Ig,”), and a TACI-Ig fusion polypeptide (control labeled “Z-TACI-Ig”) binding to cynomolgus APRIL (FIG. 7A) and human BAFF (FIG. 7B).

FIG. 8A and FIG. 8B depict the equilibrium dissociation constants (K_{D}) of representative variant BCMA polypeptides as described and controls in molar concentrations of huBAFF or huAPRIL determined by kinetic exclusion binding assays (KINEXA 3200). Vertical bars represent the 95% confidence interval for the measurements. The X-axis labels represent the variant BCMA-Ig or control Ig fusion polypeptide and ligand (suffix “.A” for huAPRIL or suffix “.B” for huBAFF). FIG. 8A provides a comparison of the equilibrium dissociation constants of variant BCMA-Ig polypeptides with control, (Z-TACI-Ig. FIG. 8B provides a comparison of the various stalk and Ig versions of the BAv9-1 variant as with [S40G]BCMA ECD-pIg18 (designated “BCMA[S40G]-pIg18”) as described in Example 17. A “*” indicates that the value is an average of two or more assays.

FIG. 9 depicts the inhibition curves of representative variant BCMA polypeptides as described and controls as the percent maximum proliferation signal determined by an HEK293-huBCMA NFkappaB luciferase reporter cell assay in response to soluble huBAFF.

FIG. 10 depicts the inhibition curves of representative variant BCMA polypeptides as described and controls as the percent maximum proliferation signal determined in the HEK293-huBCMA NFkappaB luciferase reporter cell assay in response to HEK-293 cells expressing membrane huBAFF.

FIG. 11A depicts the reduction in percentage of splenic marginal zone B lymphocytes (“MZB cells”) from lupus prone NZB/W-F1 mice that stain positive for CD19 B cell marker after 8 weeks of treatment with an exemplary variant BCMA polypeptide as described (BAv9-1-Ig), TACI-Ig, mBR3-Ig, or a PBS control. FIG. 11B shows the percentage of CD19 positive B cells in lupus prone mice treated with fusion proteins BAv9-1-Ig, TACI-Ig, mBR3-Ig, and a PBS control, in a similar experiment.

FIG. 12 depicts the reduction in IgG-producing bone marrow plasma cells (Antibody-secreting cells, “IgG ASCs”) from NZB/W-F1 mice that form ELISPOTS after 8 weeks of treatment with an exemplary variant BCMA polypeptide as described (variant BAv9-1-Ig), TACI-Ig, mBR3-Ig, or PBS.

FIG. 13A depicts the reduction in total serum IgA levels from NZB/W-F1 mice after 8 weeks of treatment with an exemplary variant a BCMA fusion polypeptide as described (BAv9-1-Ig), TACI-Ig, mBR3-Ig, or PBS. FIG. 13B depicts total IgA serum levels in lupus-prone mice following treatment with a BCMA fusion polypeptide as described (BAv9-1-Ig), TACI-Ig, mBR3-Ig, or PBS.

FIG. 14A depicts the reduction in anti-dsDNA IgM levels from NZB/W-F1 mice prior to treatment (Pre) and after 4 weeks of treatment (W4) with an exemplary variant BCMA polypeptide as described (BAv9-1-Ig), Z-TACI-Ig, mBR3-Ig, or PBS. FIG. 14B depicts the nucleosome ASCs measured in NZB/W-F1 mice after 8 weeks of treatment with an exemplary variant BCMA polypeptide as described (BAv9-1-Ig), TACI-Ig, mBR3-Ig, or PBS.

FIG. 15 depicts the reduction in kidney pathology in NZB/W-F1 mice as measured by renal histology after 8 weeks of treatment with an exemplary variant BCMA polypeptide as described (BAv9-1-Ig), TACI-Ig, mBR3-Ig, or PBS.

FIG. 16 depicts mean proteinuria (mg/dL) in mice over 16 weeks in response to treatment with IgG1 fusion proteins BAv9-1-Ig (CRD SEQ ID NO:3), TACI-Ig, mBR3-Ig, or a PBS control.

FIG. 17 depicts the reduction in percentage of peripheral blood B lymphocytes from cynomolgus monkeys that stain positive for CD19 B cell marker over 28 days after single intravenous treatments with an exemplary variant BCMA polypeptide as described (BAv9-1-Ig at 0.3 and 1.0 mg/kg) and TACI-Ig (at 1.0 mg/kg).

FIG. 18 depicts the reduction in total serum IgG from cynomolgus monkeys over the course of 28 days after intravenous treatments with an exemplary variant BCMA polypeptide as described (BAv9-1-Ig at 0.1, 0.3 and 1.0 mg/kg) and TACI-Ig (at 0.1 and 1.0 mg/kg).

FIG. 19 depicts the reduction in total serum IgA from cynomolgus monkeys over the course of 28 days after intravenous treatments with an exemplary variant BCMA polypeptide as described (BAv9-1-Ig at 0.1, 0.3 and 1.0 mg/kg) and TACI-Ig (at 0.1 and 1.0 mg/kg).

FIG. 20 depicts the reduction in total serum IgM from cynomolgus monkeys over the course of 14 days after intravenous treatments with an exemplary variant BCMA polypeptide as described (BAv9-1-Ig at 0.1, 0.3 and 1.0 mg/kg) and TACI-Ig (at 0.1 and 1.0 mg/kg).

FIG. 21A depicts the reduction in serum anti-tetanus toxoid (TTx) IgG antibodies from cynomolgus monkeys at day 7 after intravenous treatment with an exemplary variant BCMA polypeptide as described (BAv9-1-Ig at 0.1, 0.3 and 1.0 mg/kg) and TACI-Ig (at 0.1 and 1.0 mg/kg). FIG. 21B depicts the reduction in serum anti-tetanus toxoid (TTx) IgG antibodies from cynomolgus monkeys at day 14 after intravenous treatment with an exemplary variant BCMA polypeptide as described (BAv9-1-Ig at 0.1, 0.3 and 1.0 mg/kg) and Z-TACI-Ig (0.1 and 1.0 mg/kg).

FIG. 22 depicts the reduction in serum anti-tetanus toxoid (TTx) IgM antibodies from cynomolgus monkeys at day 7 after intravenous treatment with an exemplary variant BCMA polypeptide as described (BAv9-1-Ig at 0.1, 0.3 and 1.0 mg/kg) and TACI-Ig (at 0.1 and 1.0 mg/kg).

FIG. 23 depicts the reduction in percentage of B220 B cells of the marginal zone, transitional, and follicular B cell phenotype from spleens from Balb/c mice analyzed by FACS staining after 1 and 3 weeks of “induction” treatment with high doses (10 mg/kg) of an exemplary BAFF-selective variant BCMA polypeptide as described (variant 238833-Ig, “238833-Ig”) or PBS (“vehicle”). Right graph depicts the B220 B cell results from induction animals treated for an additional 3 weeks with “maintenance” treatment (Variant 238833-Ig/mBR3-Ig group) of mBR3-Ig (50 mg/kg) or continued induction treatment (variant 238833-Ig/variant 238833-Ig group).

FIG. 24 depicts the reduction in IgG-secreting bone marrow plasma cells after 1 and 3 weeks of “induction” treatment with high doses (10 mg/kg) of an exemplary BAFF-selective variant BCMA polypeptide as described (variant 238833-Ig) or PBS (left and middle graphs). Right graph depicts the bone marrow plasma cell results from induction animals treated for an additional 3 weeks with “maintenance” treatment (variant 238833-Ig/mBR3-Ig group) of mBR3-Ig (50 mg/kg) or continued induction treatment (variant 238833-Ig/variant 238833-Ig group).

FIG. 25 depicts the reduction in serum IgA after 1 and 3 weeks of “induction” treatment with high doses (10 mg/kg) of an exemplary BAFF-selective variant BCMA polypeptide as described (variant 238833-Ig) or PBS (left and middle graphs). The right graph (labeled “Week 6”) depicts the serum IgA results from induction animals treated for an additional 3 weeks with “maintenance” treatment (variant 238833-Ig/mBR3-Ig group) of mBR3-Ig (50 mg/kg) or continued induction treatment (variant 238833-Ig/variant 238833-Ig group).

FIG. 26 depicts the reduction in percentage of B220 B cells of the marginal zone, transitional, and follicular B cell phenotype from spleens from Balb/c mice analyzed by FACS staining after 3 weeks of treatment with titrated doses (150 or 500 μg/mouse) of an exemplary BAFF-selective variant BCMA polypeptides as described (variant BAv66-Ig) or PBS.

FIG. 27 depicts the reduction in IgG-secreting bone marrow plasma cells from Balb/c mice analyzed by FACS staining after 3 weeks of treatment with titrated doses (150 or 500 g/mouse) of an exemplary BAFF-selective variant BCMA polypeptide as described (variant BAv66-Ig) or PBS.

FIG. 28 depicts the reduction in serum IgA from Balb/c mice after 3 weeks of treatment with titrated doses (150 or 500 μg/mouse) of an exemplary BAFF-selective variant BCMA polypeptides as described (variant BAv66-Ig) or PBS.

FIG. 29 depicts the free drug concentration in mice after single injection with variant BCMA BAv9-1 fused to three different IgG2 Fc variants.

FIG. 30 depicts the SDS-PAGE banding pattern of two variant BCMAs with N-linked glycosylation sites retained (Gly+) or removed by mutation of S40G (Gly−).

FIG. 31 depicts the preservation of kidney function observed in NZB/W F1 lupus prone mice treated with an exemplary variant BCMA polypeptide as described (variant BAv9-1-5L-pIg22 [K278]del, labeled “BAv9-1-Ig”) but not with human BCMA surrogate control protein (labeled “BCMA-Ig”) or control Fc protein.

FIG. 32 depicts the reduction in splenic B cells observed in NZB/W F1 lupus prone mice treated with an exemplary variant BCMA polypeptide as described (variant BAv9-1-5L-pIg22 [K278]del, labeled “BAv9-1-Ig” but not with human BCMA surrogate control protein (labeled “BCMA-Ig”) or control Fc protein.

FIG. 33A and FIG. 33B show comparative IC₅₀ of RC18 (Telitacicept), Atacicept, and an exemplary variant BCMA CRD-IgG4 Fc fusion protein to human BAFF (FIG. 33A) and human APRIL (FIG. 33B).

FIG. 34A and FIG. 34B show comparative IC₅₀ of RC18 (Telitacicept), Atacicept, and an exemplary variant BCMA CRD-IgG4 Fc fusion protein to murine BAFF (FIG. 34A) and murine APRIL (FIG. 34B).

FIG. 35A and FIG. 35B show comparative EC₅₀ of RC18 (Telitacicept), Atacicept, and an exemplary variant BCMA CRD-IgG4 Fc fusion protein to heparan sulfate proteoglycans Syndecan 1 (FIG. 35A) and Syndecan 2 (FIG. 35A).

FIG. 36 depicts flow cytometry results for assessing B cell enrichment efficiency, by staining for CD19, gated on lymphocytes. PBMCs before enrichment were used as controls. The percentage of CD19+ cells are indicated.

FIG. 37 depicts flow cytometry results assessing BAFF-R expression of the enriched B cell populations for all three Donors. Unstained cells were used as controls. The percentage of BAFF-R+ cells are indicated.

FIG. 38 depicts flow cytometry results assessing the TACI expression of the enriched B cell populations for all three Donors. Unstained cells were used as controls. The percentage of TACI+ cells are indicated.

FIG. 39 depicts flow cytometry results assessing BCMA expression of the enriched B cell populations for all three Donors. Unstained cells were used as controls. The percentage of BCMA+ cells are indicated.

FIG. 40 depicts flow cytometry histogram overlays, showing the expression of BAFF-R, TACI, and BCMA on the enriched B cell populations for Donors 1, 2, and 3. Mean fluorescence intensity (MFI) values are denoted near each peak.

FIG. 41 depicts the stimulation and proliferation of the enriched B cell population in the presence of stimulatory ligands (BAFF, APRIL, or BAFF-60mer), with or without plate bound anti-IgM antibody for all three donors: no stimulation (“no stim”), anti-IgM antibody alone (“aIgM alone”), anti-IgM antibody with ligand (“aIgM+BAFF,” “aIgM+APRIL,” or “aIgM+BAFF-60mer”) and ligand alone (“BAFF,” “APRIL,” or “BAFF-60mer”), for all three donors, as represented by Relative Luminescence Units (RLU) from the CellTiter-Glo® Luminescent Cell Viability Assay.

FIGS. 42A-42C depict the inhibition curves of the three inhibitors BAv9-1 CRD-IgG4 Fc, Atacicept, and RC18 (Telitacicept), for proliferation of the enriched B cell population stimulated with human BAFF, human APRIL, or human BAFF-60mer and anti-IgM antibodies, for Donor 1 (FIG. 42A), Donor 2 (FIG. 42B), and Donor 3 (FIG. 42C), as represented by plotting the RLU from the CellTiter-Glo® Luminescent Cell Viability Assay over a serial dilution of concentration of the inhibitor. The determined IC₅₀ values for each are denoted in the plots.

### DETAILED DESCRIPTION

Provided herein are variant B-cell Maturation Antigen (BCMA) polypeptides, such as a fragment of a variant BCMA, that bind to ligands B-cell Activating Factor of the TNF family (BAFF) and A Proliferation Inducing Ligand (APRIL), and related fusion proteins, compositions, nucleic acids methods and uses. The provided variant BCMA polypeptides comprise a mutated cysteine rich domain (CRD), and can bind, such as specifically bind, to ligands BAFF and APRIL, providing dual blockade of the B-cell stimulatory cytokines. In some cases, the variant BCMA polypeptides are linked to an immunoglobulin Fc region. Such variant BCMA-Fc fusion proteins can further assemble to form dimers. Expression constructs encoding the variant BCMAs and fusion proteins can be used to manufacture the polypeptides. The various provided polypeptides and expression constructs can be used in prophylactic or therapeutic methods, such as in the prevention or the treatment of an immune cell mediated disease or condition by dual blockade of BAFF and APRIL.

Autoimmune disorders can result from a cycle of immune cell stimulation, mediated in part by the interaction between stimulatory cytokines and immune cell receptors. Plasma B cells and plasmablasts secrete self-reactive antibodies, which attack tissues. Tissue damage results in the release of autoantigens, which bind to the autoreactive antibodies, thereby triggering the production of more plasma B cells secreting the autoantibodies via T cell-dependent and T-cell independent mechanisms.

Tumor necrosis factor (TNF) family receptors B cell maturation antigen (BCMA), transmembrane activator and CAML interactor (TACI), and BAFF receptor (BR3) play complementary roles in regulating immune cell activity (See Pelletier et al., J. Biol. Chem. 2003; 278, 33127-33133; Treml et al., Cell Biochem Biophys. 2009; 53(1):1-16; and Bischof et al, Blood. 2006; 107(8):3235-42). All three receptors are expressed on the surface of normal B cells, in later stages of development, and on malignant B cells. Together, BCMA, TACI, and BR3 regulate B cell survival, function and differentiation (Cancro, D'Cruz and Khamashta J. Clinical Invest. 2009; 119:1066-1073. Dillon, S. et al. (2006) Nature Reviews 2006; 5:235-246). TACI is also expressed on activated T cells (Khare et al. Proc. Natl. Acad. Sci. USA 2000; 97(7):3370-3375).

Both BCMA and TACI bind to ligands BAFF and APRIL, while BR3 binds solely to BAFF. BAFF and APRIL are members of the TNF family of ligands that enhance the differentiation of B cells to pathogenic plasma cells and prolong the survival of such plasma cells. BAFF is believed to be a key costimulatory molecule for mature B cell proliferation and survival, including survival of autoreactive B cells (Day et al., Biochemistry 2005; 44:1919-1931 and Liu, Trends Immunol. 2011; 32(8): 388-394). In addition to its relevance to the autoimmune response cycle, BAFF has also been implicated in the modulation of the proliferative capacity and survival of multiple myeloma cells (Novak, et al., Blood 2004:103(2):689-694).

BAFF and APRIL are implicated in the establishment and/or maintenance of certain autoimmune diseases such as systemic lupus erythematosus (SLE), lupus nephritis, rheumatoid arthritis (RA), multiple sclerosis, and Sjögren's syndrome (see, e.g., Dillon, S. et al. (2006) Nature Reviews 2006; 5:235-246; Gross et al. Nature 2000; 404(6782):995-999; MacKay et al. Annu. Rev. Immunol. 1999; 21:231-2264 (2004); and Khare et al. Proc. Natl. Acad. Sci. USA 2000; 97(7):3370-3375). Pre-clinical and clinical studies have demonstrated that antagonists of BAFF and/or APRIL can reduce autoantibody levels and control autoimmune disease activity. Animal models support the conclusion that antagonism of both BAFF and APRIL is desirable for optimal blockade of the B-cell autoimmune response cycle, by reducing or eliminating production of pathogenic antibody producing cells, reducing tissue damage and subsequent formation of autoimmune complexes, and thus blocking the further production of new pathogenic plasma cells and plasmablasts. Compared to inhibition of either BAFF or APRIL alone, blockade of BAFF and APRIL is a more effective means of rapidly depleting autoreactive plasmablasts and plasma cells.

Strategies for the prevention or the treatment of B-cell mediated conditions, such as autoimmune diseases, include administration of molecules to interfere with the signaling of either ligand alone or both BAFF and APRIL. Molecules that reportedly modulate B cell function by interfering with BAFF and/or APRIL signaling include antibodies, such as the anti-BAFF antibody BENLYSTA®, previously known as Lymphostat-B (belimumab; Baker K. P. et al. Arthritis Rheum. 2003; 48:3253-3265), and receptor extracellular domain-Fc domain fusion proteins, such as Atacicept, a TACI-Ig fusion protein with specificity for BAFF and APRIL (Baker J. A. et al. Nature 2000; 404:995-999 and Gross, J. A. et al. (2000) Immunity 15:289-302), BR3-Fc (Pelletier et al., J. Biol. Chem. 2003; 278, 33127-33133), and BCMA-Ig (Melchers Ann. Rheum. Dis. 2003; 62(Suppl.2):ii25-ii27; Patel, D. R. et al. J. Biol. Chem. 2004; 279:16727-16735; WO 03/072713; and US 2009/0297504). However, the efficacy and safety of existing molecules demonstrate the need for improved treatment options. For example, rheumatoid arthritis clinical trials showed that dual BAFF/APRIL inhibitor Atacicept provided no significant benefit over placebo (van Vollenhoven et al., Rheumatoid Arthritis 2011; 63(7):1782-1792; Genovese et al., Rheumatoid Arthritis 2011; 63(7):1793-1803; Nanda, Nature Reviews Rheumatology 2011; 7:313).

Provided herein are variant BCMA polypeptides, fusion polypeptides, dimers, conjugates, polynucleotides, vectors, cells, pharmaceutical compositions, and related methods and uses that meet such needs. In some aspects, provided are exemplary fusion proteins that comprise a variant BCMA CRD and another polypeptide, such as an immunoglobulin Fc region. In some aspects, the immunoglobulin Fc region is an IgG4 Fc region. In some aspects, existing therapeutic antibodies and Fc fusion proteins often include an IgG1 Fc region (Santos et al., Braz. J. Pharm. Sci. 2018; 54(Special):e01007 and Duivelshof et al., J. Sep. Sci 2021; 44(1):35-62). IgG1 Fc polypeptides produce strong cytotoxic responses from high affinity interactions with IgG Fc receptors (FcγRs) (Kang, Exp. Mol. Medicine 2019; 51:1-9). Resultant Fc-mediated effector functions include antibody-dependent cellular cytotoxicity (ADCC), antibody-dependent cellular phagocytosis (ADCP), and complement-dependent cytotoxic (CDC) effects. While inducing a potent cytotoxic response may be advantageous for some indications, such as in the treatment of cancer or infectious diseases, treatment of autoimmune disease requires a strategy of targeted immunosuppression (Kellner et al., Transfus Med Hemother 2017; 44:327-336).

Provided herein are variant BCMA polypeptides comprising mutated cysteine rich domain (CRD) that bind to both BAFF and APRIL to provide dual blockade of the B cell stimulatory cytokine, and provide improved properties and benefits over existing molecules that target BAFF and/or APRIL. For example, as described in the application, the provided variant BCMA polypeptides and related molecules (e.g., fusion proteins) and compositions offer improved binding, such as with substantially higher affinity, improved specificity, and reduced, limited or no heparin sulfate proteoglycan (HSPG) binding. In some aspects, the provided variant BCMA polypeptides comprise an engineered BCMA binding pocket, which contain specific modifications resulting in substantial enhancement of binding and substantially higher binding affinity. In some aspects, the structural modifications to the binding pocket result in a stronger conformational fit, which, as shown in the application, leads to extremely high binding affinity and potency. In some aspects, the improved affinity, specificity and reduction of HSPG binding can offer advantages of improved efficacy and/or for a reduced dose level or frequency, and preserve T cell independent responses to infections, which in turn could result in higher patient compliance and efficacy, and an improved safety profile. Further, the provided variant BCMA polypeptides, related molecules and compositions also exhibit reduced or minimal effector function and permit improved safety profile.

Compared to existing molecules, the provided variant BCMA molecules bind to BAFF and APRIL with greater affinity. In some aspects, provided are fusion proteins comprising a variant BCMA CRD and an IgG4 Fc region. In some aspects, the provided embodiments, offer an advantage of a muted cytotoxic response, such as by selecting IgG4 Fc, which is a poor inducer of Fc-mediated effector function (Kang, Exp. Mol. Medicine 2019; 51:1-9 and WO 2021068752), such as ADCC or CDC. In some cases, ADCC and CDC can cause undesirable side effects, such as toxicity, or shorten the efficacy of a fusion protein (Kang, Exp. Mol. Medicine 2019; 51:1-9). In some aspects, the provided embodiments are based on an observation of an effective and high affinity binding and inhibition of BAFF and APRIL, while exhibiting reduced or minimized effector function. In some aspects, the Fc region provides enhanced stability and uniform expression of a fusion protein, which is desirable for production and quality control.

Also provided are polynucleotides encoding the variant BCMA polypeptides or fusion polypeptides, one or more monomers of dimers, vectors comprising such polynucleotides, pharmaceutical compositions, such as compositions for therapy, that comprise any of such provided molecules, cells, and methods of treatment involving administering such variant BCMA polypeptides, fusion polypeptides, dimers, conjugates, polynucleotides, vectors, cells, pharmaceutical compositions.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. BCMA VARIANT MOLECULES

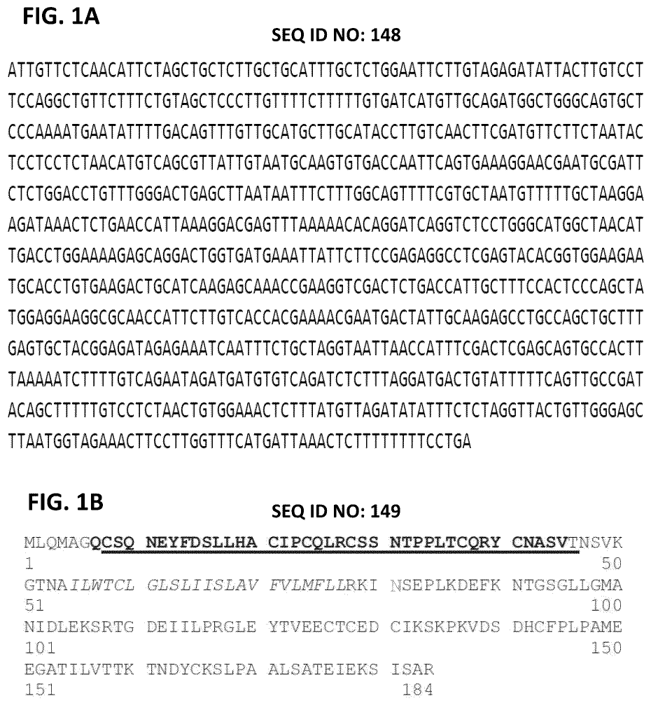

Human BCMA (huBCMA), also known as TNFRS17, is a 184 amino acid type III membrane protein of the tumor necrosis factor receptor superfamily (SEQ ID NO:149 and SEQ ID NO:150). HuBCMA comprises an extracellular domain (ECD) as set forth in SEQ ID NO:152 (corresponding to amino acid residues 5-54 of SEQ ID NO:149), a transmembrane domain (amino acid residues 55-77 of SEQ ID NO:149, the italicized region in FIG. 1B), and a cytoplasmic domain (amino acid residues 78-184 of SEQ ID NO:149). The ECD comprises a cysteine-rich domain (CRD) spanning residues 7-41 of SEQ ID NO:149, a motif of TNF receptors. An exemplary huBCMA CRD is set forth in SEQ ID NO:1. The BCMA stalk region is set forth in SEQ ID NO:152 (amino acid residues 51-55).

In some aspects, the provided variant BCMA molecules comprise a variant BCMA polypeptide comprising a mutated CRD relative to SEQ ID NO:1. In some examples, variant BCMA molecules comprise variant BCMA fusion polypeptides comprising a mutated CRD relative to SEQ ID NO:1. Non-limiting examples of variant BCMA fusion polypeptides include, for example, bispecific fusion polypeptides, and variant BCMA-Ig fusion polypeptides. Further non-limiting examples include dimers, such as dimers of variant BCMA-Ig fusion polypeptides covalently linked via disulfide bonds through the Ig component of the fusion polypeptide, as well as conjugates thereof. In some embodiments, provided herein are variant BCMA polypeptides comprising a mutated CRD relative to the corresponding residues of wild-type huBCMA (SEQ ID NO:1) and variant BCMA molecules comprising the provided variant BCMA polypeptides.

### A. BCMA Variant Polypeptides

Provided herein are variant BCMA polypeptides comprising a mutated CRD relative to wild-type huBCMA CRD (SEQ ID NO:1).

In some embodiments, the mutated CRD of the variant BCMA polypeptide at least at or about 80%, 81%, 82%, 83%, 84%, 85%, 86%, 87%, 88%, 89%, 90%, 91%, 92%, 93%, 94%, 95%, 96%, 97%, 98%, or 99% sequence identity to huBCMA CRD (SEQ ID NO:1). In some embodiments, the mutated CRD of the variant BCMA polypeptide has at least 80%, 81%, 82%, 83%, 84%, 85%, 86%, 87%, 88%, 89%, 90%, 91%, 92%, 93%, 94%, 95%, 96%, 97%, 98%, or 99% sequence identity to huBCMA CRD (SEQ ID NO:1). In some of any of the embodiments, the variant CRD comprises at least at or about 85% sequence identity to the human BCMA CRD sequence set forth in SEQ ID NO:1.

In some embodiments, the variant BCMA CRD comprises one or more amino acid substitutions selected from among: (1) a histidine, an arginine, a proline, or an asparagine at position 12 (S12H, S12R, S12P, or S12N); (2) an isoleucine or a valine at position 14 (L14I or L14V); (3) an arginine or an asparagine at position 15 (H15R or H15N); (4) a serine at position 16 (A16S); (5) a valine, an isoleucine, or an alanine at position 22 (L22V, L22I, or L22A), with reference to the amino acid positions of a human BCMA CRD sequence set forth in SEQ ID NO:1. In some aspects, the variant BCMA CRD comprises such amino acid substitutions and comprises at least at or about 85% sequence identity, such as at least 95% sequence identity, to the human BCMA CRD sequence set forth in SEQ ID NO:1.

In some embodiments, the variant BCMA CRD comprises at least two sequence characteristics selected from the group consisting of: (1) a histidine, an arginine, a proline, or an asparagine at position 12 (S12H, S12R, S12P, or S12N); (2) an isoleucine or a valine at position 14 (L14I or L14V); (3) an arginine or an asparagine at position 15 (H15R or H15N); (4) a serine at position 16 (A16S); and (5) a valine, an isoleucine, or an alanine at position 22 (L22V, L22I, or L22A), with reference to the amino acid positions of the human BCMA CRD sequence set forth in SEQ ID NO:1. In some aspects, the variant BCMA CRD comprises such amino acid substitutions and comprises at least at or about 85% sequence identity, such as at least 95% sequence identity, to the human BCMA CRD sequence set forth in SEQ ID NO:1.

In some embodiments, the variant BCMA CRD comprises at least three sequence characteristics selected from the group consisting of: (1) a histidine or an arginine at position 12 (S12H or S12R); (2) an isoleucine or a valine at position 14 (L14I or L14V); (3) an arginine at position 15 (H15R); (4) a serine at position 16 (A16S); and (5) a valine at position 22 (L22V), with reference to the amino acid positions of the human BCMA CRD sequence set forth in SEQ ID NO:1. In some aspects, the variant BCMA CRD comprises such amino acid substitutions and comprises at least at or about 85% sequence identity, such as at least 95% sequence identity, to the human BCMA CRD sequence set forth in SEQ ID NO:1.

In some embodiments, the variant BCMA comprises one or more amino acid substitutions selected from among: (1) a histidine, an arginine, a proline, or an asparagine at position 12 (S12H, S12R, S12P, or S12N); (2) an isoleucine or a valine at position 14 (L14I or L14V); (3) an arginine or an asparagine at position 15 (H15R or H15N); and (4) a valine, an isoleucine, or an alanine at position 22 (L22V, L22I, or L22A), with reference to the amino acid positions of a human BCMA CRD sequence set forth in SEQ ID NO:1. In some aspects, the variant BCMA CRD comprises such amino acid substitutions and comprises at least at or about 85% sequence identity, such as at least 95% sequence identity, to the human BCMA CRD sequence set forth in SEQ ID NO:1.

In some embodiments, the variant BCMA comprises at least two three sequence characteristics selected from the group consisting of: (1) a histidine, an arginine, a proline, or an asparagine at position 12 (S12H, S12R, S12P, or S12N); (2) an isoleucine or a valine at position 14 (L14I or L14V); (3) an arginine or an asparagine at position 15 (H15R or H15N); and (4) a valine, an isoleucine, or an alanine at position 22 (L22V, L22I, or L22A), with reference to the amino acid positions of the human BCMA CRD sequence set forth in SEQ ID NO:1. In some aspects, the variant BCMA CRD comprises such amino acid substitutions and comprises at least at or about 85% sequence identity, such as at least 95% sequence identity, to the human BCMA CRD sequence set forth in SEQ ID NO:1.

In some embodiments, the variant BCMA comprises least three sequence characteristics selected from the group consisting of: (1) a histidine or an arginine at position 12 (S12H or S12R); (2) an isoleucine or a valine at position 14 (L14I or L14V); (3) an arginine at position 15 (H15R); and (4) a valine at position 22 (L22V), with reference to the amino acid positions of the human BCMA CRD sequence set forth in SEQ ID NO:1. In some aspects, the variant BCMA CRD comprises such amino acid substitutions and comprises at least at or about 85% sequence identity, such as at least 95% sequence identity, to the human BCMA CRD sequence set forth in SEQ ID NO:1.

In some embodiments, the variant BCMA CRD comprises the following sequence characteristics: a histidine at position 12 (S12H), an isoleucine at position 14 (L14I), and an arginine at position 15 (H15R). In some embodiments, the variant BCMA CRD comprises the following sequence characteristics: an arginine at position 12 (S12R), an isoleucine at position 14 (L14I), and an arginine at position 15 (H15R). In some embodiments, the variant BCMA CRD comprises the following sequence characteristics: a histidine at position 12 (S12H), an arginine at position 15 (H15R), and a valine at position 22 (L22V). In some embodiments, the variant BCMA CRD comprises the following sequence characteristics: a histidine at position 12 (S12H) and an arginine at position 15 (H15R). In some embodiments, the variant BCMA CRD comprises the following sequence characteristics: an arginine at position 12 (S12R), a valine at position 14 (L14V), an asparagine at position 15 (H15N). In some embodiments, the variant BCMA CRD comprises the following sequence characteristics: an arginine at position 12 (S12R), a valine at position 14 (L14V), an asparagine at position 15 (H15N), and a serine at position 16 (A16S). In some aspects, the variant BCMA CRD comprises such amino acid substitutions and comprises at least at or about 85% sequence identity, such as at least 95% sequence identity, to the human BCMA CRD sequence set forth in SEQ ID NO:1.

In some embodiments, the variant BCMA polypeptide also comprises at least one sequence characteristic selected from the group consisting of: (1) a deletion at residue 38 (N38del) or an amino acid residue that is not asparagine at position 38 (N38X, where X is any amino acid residue that is not asparagine) and (2) an amino acid residue that is not serine or threonine at position 40 (S40X, where X is any amino acid that is not serine or threonine) with reference to the amino acid positions of the human BCMA CRD sequence set forth in SEQ ID NO:1. In some embodiments, the variant BCMA polypeptide further comprises a serine to glycine mutation at residue 40 (S40G). In some aspects, the variant BCMA CRD comprises such amino acid substitutions and comprises at least at or about 85% sequence identity, such as at least 95% sequence identity, to the human BCMA CRD sequence set forth in SEQ ID NO:1.

In some embodiments, the variant BCMA comprises an amino acid sequence that has at least at or about 85%, 86%, 87%, 88%, 89%, 90%, 91%, 92%, 93%, 94%, 95%, 96%, 97%, 98%, 99% or more sequence identity to a sequence selected from SEQ ID NOs: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 80, 81, 82, 83, 84, 85, 86, 87, 88, 89, 90, 91, 92, 93, 94, 95, 96, 97, 98, 100, 101, 102, 103, 104, 105, 106, 107, 108, 109, 110, 112, 113,114, 115, 116, 117, 118, 119, 120, 121, 122, 123, 124, 125, 126, 127, 128, 129, 130, 131, 132, 133, 134, 135, 136, 137, 138, 139, 140, 141, 142, and 143.

In some embodiments, the variant BCMA comprises one or more sequences selected from SEQ ID NOs: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 80, 81, 82, 83, 84, 85, 86, 87, 88, 89, 90, 91, 92, 93, 94, 95, 96, 97, 98, 100, 101, 102, 103, 104, 105, 106, 107, 108, 109, 110, 112, 113,114, 115, 116, 117, 118, 119, 120, 121, 122, 123, 124, 125, 126, 127, 128, 129, 130, 131, 132, 133, 134, 135, 136, 137, 138, 139, 140, 141, 142, and 143, or a sequence that comprises at least 95% sequence identity thereto. In some embodiments, the variant BCMA comprises one or more sequences selected from SEQ ID NOs: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 80, 81, 82, 83, 84, 85, 86, 87, 88, 89, 90, 91, 92, 93, 94, 95, 96, 97, 98, 100, 101, 102, 103, 104, 105, 106, 107, 108, 109, 110, 112, 113,114, 115, 116, 117, 118, 119, 120, 121, 122, 123, 124, 125, 126, 127, 128, 129, 130, 131, 132, 133, 134, 135, 136, 137, 138, 139, 140, 141, 142, and 143.

In some aspects, the variant BCMA comprises SEQ ID NO:3. In some aspects, the variant BCMA comprises SEQ ID NO:4. In some aspects, the variant BCMA comprises SEQ ID NO:5. In some aspects, the variant BCMA comprises SEQ ID NO:6. In some aspects, the variant BCMA comprises SEQ ID NO:7. In some aspects, the variant BCMA comprises SEQ ID NO:8. In some aspects, the variant BCMA comprises SEQ ID NO:9. In some aspects, the variant BCMA comprises SEQ ID NO:10. In some aspects, the variant BCMA comprises SEQ ID NO:11. In some aspects, the variant BCMA comprises SEQ ID NO:12. In some aspects, the variant BCMA comprises SEQ ID NO:13. In some aspects, the variant BCMA comprises SEQ ID NO:14. In some aspects, the variant BCMA comprises SEQ ID NO:15. In some aspects, the variant BCMA comprises SEQ ID NO:16. In some aspects, the variant BCMA comprises SEQ ID NO:17. In some aspects, the variant BCMA comprises SEQ ID NO:18. In some aspects, the variant BCMA comprises SEQ ID NO:19. In some aspects, the variant BCMA comprises SEQ ID NO:20. In some aspects, the variant BCMA comprises SEQ ID NO:21. In some aspects, the variant BCMA comprises SEQ ID NO:22. In some aspects, the variant BCMA comprises SEQ ID NO:23. In some aspects, the variant BCMA comprises SEQ ID NO:24. In some aspects, the variant BCMA comprises SEQ ID NO:25. In some aspects, the variant BCMA comprises SEQ ID NO:26. In some aspects, the variant BCMA comprises SEQ ID NO:27. In some aspects, the variant BCMA comprises SEQ ID NO:28. In some aspects, the variant BCMA comprises SEQ ID NO:29. In some aspects, the variant BCMA comprises SEQ ID NO:30. In some aspects, the variant BCMA comprises SEQ ID NO:31. In some aspects, the variant BCMA comprises SEQ ID NO:32. In some aspects, the variant BCMA comprises SEQ ID NO:33. In some aspects, the variant BCMA comprises SEQ ID NO:34. In some aspects, the variant BCMA comprises SEQ ID NO:35. In some aspects, the variant BCMA comprises SEQ ID NO:36. In some aspects, the variant BCMA comprises SEQ ID NO:37. In some aspects, the variant BCMA comprises SEQ ID NO:38. In some aspects, the variant BCMA comprises SEQ ID NO:39. In some aspects, the variant BCMA comprises SEQ ID NO:40, 41. In some aspects, the variant BCMA comprises SEQ ID NO:42. In some aspects, the variant BCMA comprises SEQ ID NO:43. In some aspects, the variant BCMA comprises SEQ ID NO:44. In some aspects, the variant BCMA comprises SEQ ID NO:45. In some aspects, the variant BCMA comprises SEQ ID NO:46. In some aspects, the variant BCMA comprises SEQ ID NO:47. In some aspects, the variant BCMA comprises SEQ ID NO:48. In some aspects, the variant BCMA comprises SEQ ID NO:49. In some aspects, the variant BCMA comprises SEQ ID NO:50. In some aspects, the variant BCMA comprises SEQ ID NO:51. In some aspects, the variant BCMA comprises SEQ ID NO:52. In some aspects, the variant BCMA comprises SEQ ID NO:53. In some aspects, the variant BCMA comprises SEQ ID NO:54. In some aspects, the variant BCMA comprises SEQ ID NO:55. In some aspects, the variant BCMA comprises SEQ ID NO:56. In some aspects, the variant BCMA comprises SEQ ID NO:57. In some aspects, the variant BCMA comprises SEQ ID NO:58. In some aspects, the variant BCMA comprises SEQ ID NO:59. In some aspects, the variant BCMA comprises SEQ ID NO:60. In some aspects, the variant BCMA comprises SEQ ID NO:61. In some aspects, the variant BCMA comprises SEQ ID NO:62. In some aspects, the variant BCMA comprises SEQ ID NO:63. In some aspects, the variant BCMA comprises SEQ ID NO:64. In some aspects, the variant BCMA comprises SEQ ID NO:65. In some aspects, the variant BCMA comprises SEQ ID NO:66. In some aspects, the variant BCMA comprises SEQ ID NO:67. In some aspects, the variant BCMA comprises SEQ ID NO:68. In some aspects, the variant BCMA comprises SEQ ID NO:69. In some aspects, the variant BCMA comprises SEQ ID NO:70. In some aspects, the variant BCMA comprises SEQ ID NO:71. In some aspects, the variant BCMA comprises SEQ ID NO:72. In some aspects, the variant BCMA comprises SEQ ID NO:73. In some aspects, the variant BCMA comprises SEQ ID NO:74. In some aspects, the variant BCMA comprises SEQ ID NO:75. In some aspects, the variant BCMA comprises SEQ ID NO:76. In some aspects, the variant BCMA comprises SEQ ID NO:77. In some aspects, the variant BCMA comprises SEQ ID NO:78. In some aspects, the variant BCMA comprises SEQ ID NO:80. In some aspects, the variant BCMA comprises SEQ ID NO:81. In some aspects, the variant BCMA comprises SEQ ID NO:82. In some aspects, the variant BCMA comprises SEQ ID NO:83. In some aspects, the variant BCMA comprises SEQ ID NO:84. In some aspects, the variant BCMA comprises SEQ ID NO:85. In some aspects, the variant BCMA comprises SEQ ID NO:86. In some aspects, the variant BCMA comprises SEQ ID NO:87. In some aspects, the variant BCMA comprises SEQ ID NO:88. In some aspects, the variant BCMA comprises SEQ ID NO:89. In some aspects, the variant BCMA comprises SEQ ID NO:90. In some aspects, the variant BCMA comprises SEQ ID NO:91. In some aspects, the variant BCMA comprises SEQ ID NO:92. In some aspects, the variant BCMA comprises SEQ ID NO:93. In some aspects, the variant BCMA comprises SEQ ID NO:94. In some aspects, the variant BCMA comprises SEQ ID NO:95. In some aspects, the variant BCMA comprises SEQ ID NO:96. In some aspects, the variant BCMA comprises SEQ ID NO:97. In some aspects, the variant BCMA comprises SEQ ID NO:98. In some aspects, the variant BCMA comprises SEQ ID NO:100. In some aspects, the variant BCMA comprises SEQ ID NO:101. In some aspects, the variant BCMA comprises SEQ ID NO:102. In some aspects, the variant BCMA comprises SEQ ID NO:103. In some aspects, the variant BCMA comprises SEQ ID NO:104. In some aspects, the variant BCMA comprises SEQ ID NO:105. In some aspects, the variant BCMA comprises SEQ ID NO:106. In some aspects, the variant BCMA comprises SEQ ID NO:107. In some aspects, the variant BCMA comprises SEQ ID NO:108. In some aspects, the variant BCMA comprises SEQ ID NO:109. In some aspects, the variant BCMA comprises SEQ ID NO:110. In some aspects, the variant BCMA comprises SEQ ID NO:112. In some aspects, the variant BCMA comprises SEQ ID NO:113,114. In some aspects, the variant BCMA comprises SEQ ID NO:115. In some aspects, the variant BCMA comprises SEQ ID NO:116. In some aspects, the variant BCMA comprises SEQ ID NO:117. In some aspects, the variant BCMA comprises SEQ ID NO:118. In some aspects, the variant BCMA comprises SEQ ID NO:119. In some aspects, the variant BCMA comprises SEQ ID NO:120. In some aspects, the variant BCMA comprises SEQ ID NO:121. In some aspects, the variant BCMA comprises SEQ ID NO:122. In some aspects, the variant BCMA comprises SEQ ID NO:123. In some aspects, the variant BCMA comprises SEQ ID NO:124. In some aspects, the variant BCMA comprises SEQ ID NO:125. In some aspects, the variant BCMA comprises SEQ ID NO:126. In some aspects, the variant BCMA comprises SEQ ID NO:127. In some aspects, the variant BCMA comprises SEQ ID NO:128. In some aspects, the variant BCMA comprises SEQ ID NO:129. In some aspects, the variant BCMA comprises SEQ ID NO:130. In some aspects, the variant BCMA comprises SEQ ID NO:131. In some aspects, the variant BCMA comprises SEQ ID NO:132. In some aspects, the variant BCMA comprises SEQ ID NO:133. In some aspects, the variant BCMA comprises SEQ ID NO:134. In some aspects, the variant BCMA comprises SEQ ID NO:135. In some aspects, the variant BCMA comprises SEQ ID NO:136. In some aspects, the variant BCMA comprises SEQ ID NO:137. In some aspects, the variant BCMA comprises SEQ ID NO:138. In some aspects, the variant BCMA comprises SEQ ID NO:139. In some aspects, the variant BCMA comprises SEQ ID NO:140. In some aspects, the variant BCMA comprises SEQ ID NO:141. In some aspects, the variant BCMA comprises SEQ ID NO:142. In some aspects, the variant BCMA comprises SEQ ID NO:143.

Libraries of these BCMA variant polypeptides may be generated and screened using, for example, the BIACORE assay of Example 9 or the KINEXA assay of Example 11 to determine binding to huBAFF and huAPRIL. Methods for generating variant libraries are known. For example, mutagenesis and directed evolution methods can be readily applied to polynucleotides (such as, for example, a huBCMA ECD encoding polynucleotides, e.g., SEQ ID NO:3), polynucleotides of the present disclosure (described herein below), or parts thereof, to generate variant libraries that can be expressed, screened, and assayed using the methods described herein. Mutagenesis and directed evolution methods are known. See, e.g., Ling, et al., Anal. Biochem. 1997; 254(2):157-78; Dale et al., Methods Mol. Biol., 1996; 57:369-74; Smith, Ann. Rev. Genet., 1985; 19:423-462; Botstein et al., Science, 1985; 229:1193-1201; Carter, Biochem. J., 1986; 237:1-7; Kramer et al., Cell 1984:38:879-887; Wells, et al., Gene, 1999; 34:284-290; Christians, et al., Nature Biotechnology, 1999; 17:259-264 (1999), Crameri, et al., Nature, 391:288-291; Crameri et al., Nature Biotechnology, 1997; 15:436-438; Zhang, et al., Proceedings of the National Academy of Sciences, U.S.A., 94:4504-4509; Crameri, et al., Nature Biotechnology, 1996; 14:315-319 (1996); Stemmer, Nature, 1994; 370-389-391; Stemmer, Proceedings of the National Academy of Sciences, U.S.A., 1994; 91:10747-10751; WO 95/22625, WO 97/0078, WO 97/35966, WO 98/27230, WO 00/42651, WO 01/75767, US 2009/0312196, and WO 2009/152336, all of which are incorporated herein by reference.

### B. BCMA Variant Fusion Polypeptides

Provided herein are variant BCMA fusion polypeptides comprising a variant CRD, e.g., comprising a mutation relative to huBCMA CRD (SEQ ID NO:1), and an additional polypeptide or a domain or a region, such as an immunoglobulin Fc region. In some aspects, the variant BCMA fusion protein comprises any one or more of the variant BCMA CRD provided herein. In some aspects, the Fc region can be selected according to the desired immune response, such as Fc-mediated effector functions. In some embodiments, the Fc region is or is derived from IgG1, IgG2, IgG3, or IgG4. In some embodiments, the Fc region is of human origin, murine origin, chimeric, and/or humanized.

In some aspects, the variant BCMA fusion polypeptides comprise a mutated CRD relative to huBCMA CRD (SEQ ID NO:1) and an immunoglobulin Fc region. In some embodiments, the Fc region is of the subclass IgG1, IgG2, or IgG4.

Certain therapeutic antibodies and Fc fusion proteins include an IgG1 Fc region (Santos et al., Braz. J. Pharm. Sci. 2018; 54(Special):e01007 and Duivelshof et al., J. Sep. Sci 2021; 44(1):35-62). IgG1 Fc polypeptides produce strong cytotoxic responses from high affinity interactions with IgG Fc receptors (FcγRs) (Kang, Exp. Mol. Medicine 2019; 51:1-9). Resultant Fc-mediated effector functions include antibody-dependent cellular cytotoxicity (ADCC), antibody-dependent cellular phagocytosis (ADCP), and complement-dependent cytotoxic (CDC) effects. While inducing a potent cytotoxic response may be advantageous for some indications, such as in the treatment of cancer or infectious diseases, treatment of autoimmune disease requires a strategy of targeted immunosuppression (Kellner et al., Transfus Med Hemother 2017; 44:327-336).

Compared to existing molecules, the provided variant BCMA molecules bind to BAFF and APRIL with greater affinity. Further, the Fc region of variant BCMA-Fc fusion proteins can be exchanged for an Fc of a different subclass without altering binding affinity to BAFF and APRIL. Exchanging different subclasses of the Fc region allows for selection of a desired immune response. For example, exchanging the Fc region can facilitate a potent cytotoxic response, such as by selecting IgG1 Fc, or a muted cytotoxic response, such as by selecting IgG4 Fc, which is a poor inducer of Fc-mediated effector function (Kang, Exp. Mol. Medicine 2019; 51:1-9 and WO 2021068752).

The potency of effector functions, such as ADCC and CDC, vary among the four subtypes of human IgG Fc regions. IgG1 and IgG3 subtypes display strong effector functions, while the ADCC and CDC effects of IgG2 and IgG4 subtypes are relatively weak. In some cases ADCC and CDC can cause undesirable side effects, such as toxicity, or shorten the efficacy of a fusion protein (Kang, Exp. Mol. Medicine 2019; 51:1-9). In some embodiments, the selected Fc region provides enhanced stability of the fusion protein. In some embodiments, the selected Fc region provides a uniform fusion protein, which is desirable for production and quality control.

In some embodiments, the provided BCMA fusion proteins comprise an Ig Fc polypeptide that is or is derived from an isotype G immunoglobulin (IgG) or a variant thereof. In some embodiments, the provided BCMA fusion proteins comprise an Ig Fc polypeptide that is an isotype G immunoglobulin (IgG).

In some embodiments, the provided BCMA fusion proteins comprise an IgG1, an IgG2, and IgG3, or an IgG4 Fc region. In some embodiments, the provided BCMA fusion proteins comprise a human IgG1, a human IgG2, a human IgG3, or a human IgG4 Fc region.

In some embodiments, the provided fusion proteins comprise a human IgG1 Fc region, such as an IgG1 Fc region having at least 80%, at least 85%, at least 90%, or at least 95% sequence identity to the amino acid sequence set forth in any one of SEQ ID NO: 168, 169, 170, 226, 227, 228, 229, and 230. In some embodiments, the provided fusion proteins comprise an IgG1 Fc region set forth in any one of SEQ ID NO: 168, 169, 170, 226, 227, 228, 229, and 230.

In some embodiments, the provided BCMA fusion proteins comprise a human IgG2 Fc region, such as an IgG2 Fc region having at least 80%, at least 85%, at least 90%, or at least 95% sequence identity to the amino acid sequence set forth in SEQ ID NO: 171, 235, and 236. In some embodiments, the provided fusion proteins comprise an IgG2 Fc region set forth in any one of SEQ ID NO: 171, 235, and 236.

In some embodiments, the provided BCMA fusion proteins comprise a human IgG4 Fc region, such as an IgG4 Fc region having at least 80%, at least 85%, at least 90%, or at least 95% sequence identity to the amino acid sequence set forth in set forth in any one of SEQ ID NOS:161, 163, 72, 231, 232, 233, 234. In some embodiments, the provided fusion proteins comprise an IgG4 Fc region set forth in any one of SEQ ID NOS: 161, 163, 72, 231, 232, 233, 234.

In some aspects, the provided BCMA fusion proteins comprise a variant of a human IgG4 Fc region. In some aspects, the variant human IgG4 Fc region comprises one or more amino acid substitutions, deletions and/or additions compared to a wild-type or unmodified human IgG4 Fc region, such as a wild-type or unmodified IgG4 Fc region sequence set forth in SEQ ID NO:172. In some aspects, exemplary substitutions, deletions and/or additions of the human IgG4 Fc region includes those described in, for example, US 2015/0104410, US 2022/0033476, Dumet et al., (2019) MAbs DOI: 10.1080/19420862.2019.1664365, and Xu et al., (2019) MAbs 10.1080/19420862.2019.1631116. In some aspects, exemplary substitutions, deletions and/or additions of the human IgG4 Fc region comprises one or more substitutions selected from among S228P, F234A, L235A, and/or L445P, with reference to the positions by EU numbering. In some aspects, exemplary substitutions, deletions and/or additions of the human IgG4 Fc region comprises S228P, F234A, L235A, and L445P, with reference to the positions by EU numbering. In some embodiments, the provided BCMA fusion proteins comprise a human IgG4 Fc region, such as an IgG4 Fc region having at least 80%, at least 85%, at least 90%, or at least 95% sequence identity to the amino acid sequence set forth in set forth in SEQ ID NO:161. In some embodiments, the provided fusion proteins comprise an IgG4 Fc region set forth in SEQ ID NO: 161. In some embodiments, the provided BCMA fusion proteins comprise a human IgG4 Fc region, such as an IgG4 Fc region having at least 80%, at least 85%, at least 90%, or at least 95% sequence identity to the amino acid sequence set forth in set forth in SEQ ID NO:163. In some embodiments, the provided fusion proteins comprise an IgG4 Fc region set forth in SEQ ID NO: 163.

In some embodiments, the human Fc polypeptide comprises an isoleucine or a valine substituted for one, two, three, four, or more native methionine residues.

In some embodiments, the variant BCMA polypeptide and additional polypeptide, such as an Fc region, in the variant BCMA fusion polypeptides described herein are indirectly linked. In some embodiments, the variant BCMA fusion polypeptides and the Fc region, for example, an IgG1, IgG2, or IgG4 Fc region, are linked via a peptide linker. In some embodiments, the peptide linker comprises a sequences set forth in any one of SEQ ID NO:156, 158, 175-186, 188-213, GS, GGS and GSA. In some embodiments, the peptide linker comprises SEQ ID NO:156. In some embodiments, the peptide linker comprises SEQ ID NO:158.

In some embodiments, the fusion polypeptide comprises, in N- to C-terminal order: the variant BCMA polypeptide, a peptide linker and an Ig Fc polypeptide. In some embodiments, the fusion polypeptide comprises a variant BCMA polypeptide set forth in SEQ ID NO:3, SEQ ID NO:4, SEQ ID NO:8, SEQ ID NO:9, or SEQ ID NO:19; a peptide linker comprising a sequences set forth in any one of SEQ ID NO:156, 158, 175-186, 188-213, GS, GGS and GSA, and an Ig Fc polypeptide comprising a sequence set forth in SEQ ID NO:161, SEQ ID NO:163, SEQ ID NO:172, SEQ ID NO: 231, SEQ ID NO:232, SEQ ID NO:233, or SEQ ID NO:234.

In some embodiments, the fusion polypeptide comprises a variant BCMA polypeptide comprising SEQ ID NO:3, a peptide linker comprising SEQ ID NO:156, and an Ig Fc polypeptide comprising SEQ ID NO:161. In some embodiments, the fusion polypeptide comprises a variant BCMA polypeptide comprising SEQ ID NO:3, a peptide linker comprising SEQ ID NO:158, and an Ig Fc polypeptide comprising SEQ ID NO:163. In some embodiments, the fusion polypeptide comprises a variant BCMA polypeptide comprising SEQ ID NO:4, a peptide linker comprising SEQ ID NO:156, and an Ig Fc polypeptide comprising SEQ ID NO:161. In some embodiments, the fusion polypeptide comprises a variant BCMA polypeptide comprising SEQ ID NO:4, a peptide linker comprising SEQ ID NO:158, and an Ig Fc polypeptide comprising SEQ ID NO:163. In some embodiments, the fusion polypeptide comprises a variant BCMA polypeptide comprising SEQ ID NO:8, a peptide linker comprising SEQ ID NO:156, and an Ig Fc polypeptide comprising SEQ ID NO:161. In some embodiments, the fusion polypeptide comprises a variant BCMA polypeptide comprising SEQ ID NO:8, a peptide linker comprising SEQ ID NO:158, and an Ig Fc polypeptide comprising SEQ ID NO: 163. In some embodiments, the fusion polypeptide comprises a variant BCMA polypeptide comprising SEQ ID NO:9, a peptide linker comprising SEQ ID NO:156, and an Ig Fc polypeptide comprising SEQ ID NO:161. In some embodiments, the fusion polypeptide comprises a variant BCMA polypeptide comprising SEQ ID NO:9, a peptide linker comprising SEQ ID NO:158, and an Ig Fc polypeptide comprising SEQ ID NO:163. In some embodiments, the fusion polypeptide comprises a variant BCMA polypeptide comprising SEQ ID NO:19, a peptide linker comprising SEQ ID NO:156, and an Ig Fc polypeptide comprising SEQ ID NO:161. In some embodiments, the fusion polypeptide comprises a variant BCMA polypeptide comprising SEQ ID NO:19, a peptide linker comprising SEQ ID NO:158, and an Ig Fc polypeptide comprising SEQ ID NO:163.

In some embodiments, the fusion polypeptide comprises the sequence set forth in SEQ ID NO:167 or a sequence that has at least 90% sequence identity thereto. In some embodiments, the fusion polypeptide comprises the sequence set forth in SEQ ID NO:167.

### C. BCMA Variant Polypeptide Conjugate

Provided herein are variant BCMA polypeptides comprising a mutated CRD relative to huBCMA CRD (SEQ ID NO:1) or variant BCMA-Ig-Fc-fusion polypeptides, or dimers thereof, covalently bound to at least one non-polypeptide conjugation moiety. In some embodiments, the polypeptide variant conjugate comprises a variant BCMA polypeptide comprising a mutated CRD relative to huBCMA CRD (SEQ ID NO:1) covalently bound to one or more non-polypeptide conjugation moieties.

In some embodiments, a non-polypeptide conjugation moiety is or comprises a non-polypeptide polymer, a sugar moiety, and/or a non-polymeric lipophilic moiety. In some embodiments, a non-polypeptide polymer is or comprises a water soluble polymer that may be a natural or synthetic polymer (for example, homopolymer, copolymer, terpolymer), that is not a peptide, polypeptide, or protein. In some embodiments, a sugar moiety is or comprises a carbohydrate molecule attached by an in vivo or in vitro glycosylation process, such as an N- or O-glycosylation process.

In some embodiments, non-polypeptide conjugation moieties are typically selected to alter specific attributes of the provided variant BCMA molecules. Non-limiting examples of such attributes include in vivo serum half-life or functional in vivo half-life, stability, immunogenicity. In some embodiments, in vivo serum half-life refers to the time at which 50% of the compound of interest circulates in the bloodstream of a human or a non-human mammal such as a rat, mouse, rabbit, or monkey. Serum refer to its normal meaning, i.e., as blood plasma without fibrinogen and other clotting factors. The term “functional in vivo half-life” refers herein to the time at which 50% of the biological activity of the compound of interest is still present in the body or target organ, or the time at which the activity of the compound of interest is 50% of the initial value.

In some embodiments, the conjugation moiety may be covalently bound to the polypeptide either directly or indirectly via a linker. In some embodiments, suitable linker moieties include, for example, a peptide, a non-peptidic, non-polymeric aliphatic moiety, an oligonucleotide. In some embodiments, a linker peptide is covalently attached to the N-terminus of the variant BCMA polypeptide, or fusion polypeptide thereof, and a non-polypeptide conjugation moiety is covalently attached to the N-terminus of the attached linker peptide. In some embodiments, a linker peptide is attached to the N-terminus or the C-terminus of the variant BCMA polypeptide or fusion polypeptide thereof, and a non-polypeptide conjugation moiety is covalently attached to an attachment group (such as, a Cys or a Lys residue) in the linker peptide. In some embodiments, glycosylation sites, may also be incorporated into the linker peptide sequence.

In some embodiments, exemplary polymers for use in accordance with the provided embodiments may be branched (i.e., having two or more linear polymer chains linked together by a linker group) or linear and typically have an average molecular weight in the range of from at or about 300 to at or about 100,000 daltons (Da) and typically from at or about 1,000 Da to at or about 80,000 Da, or from at or about 2,000 to at or about 60,000 or 50,000 or 40,000 or 30,000 or 20,000, or 10,000 Da, and in some embodiments from at or about 1,000 Da to at or about 5,000 Da. More particularly, the polymer molecule will typically have an average molecular weight of about 2,000 Da, 5,000 Da, 10,000 Da, 12,000 Da, 15,000 Da, 20,000 Da, 30,000 Da, 40,000 Da, 50,000 Da, 60,000 Da or 80,000 Da.

In some embodiments, exemplary polymers for use in accordance with the provided embodiments include polyalkylene oxides (PAO), such as a polyalkylene glycol (PAG) which may, for example, be a polyethylene glycol (PEG), a monomethoxypolyethylene glycol (mPEG), a polypropylene glycol (PPG), a branched polyethylene glycol having two or more polyethylene glycol chains linked together by a linker group (a linker, such as, for example, lysine, glycerol), polyvinyl alcohol (PVA), polycarboxylate, poly(vinylpyrrolidone), polyethylene-co-maleic acid anhydride, a dextran (such as, for example, carboxymethyl dextran), and other like polymers. Exemplary polymers and methods for their covalent attachment to protein molecules are known.

Provided herein is a method of producing a variant BCMA molecule conjugate, the method comprising: (a) providing a variant BCMA polypeptide, fusion polypeptide or dimer thereof as provided herein; and (b) covalently attaching at least one polyethylene glycol (PEG) moiety to any of the provided variant BCMA polypeptide, fusion polypeptide or dimer thereof comprising a mutated CRD relative huBCMA CRD (SEQ ID NO:1).

In some embodiments, a hydroxylated polymer is covalently attached to a variant BCMA molecule provided herein. In some embodiments, a hydroxylated polymer, such as polyethylene glycol, is covalently attached to a variant BCMA molecule provided herein. In some embodiments, at least one terminal hydroxyl group of the polymer molecule is provided in activated form, i.e., derivatized with functional groups that are reactive with the target attachment group in the polypeptide. Exemplary reactive functional groups include primary amino groups, hydrazide (HZ), thiol, succinate (SUC), succinimidyl succinate (SS), succinimidyl succinamide (SSA), succinimidyl proprionate (SPA), succinimidyl carboxymethylate (SCM), benzotriazole carbonate (BTC), N-hydroxysuccinimide (NHS), aldehyde, nitrophenylcarbonate (NPC), maleimide (MAL) and tresylate (TRES).

In some embodiments, variant BCMA conjugates may comprise two or more variant BCMA polypeptides comprising a mutated CRD relative to huBCMA CRD (SEQ ID NO:1) covalently bonded to one or more bi-functional PEG. In some embodiments, variant BCMA conjugates may comprise two variant BCMA polypeptides comprising a mutated CRD relative to huBCMA CRD (SEQ ID NO:1) covalently bonded to one bi-functional PEG. As used herein, the term “bi-functional peg” refers to a polyethylene glycol moiety that is derivatized with two functional groups that are reactive with the target attachment group in the polypeptides as described.

The following publications exemplify polymers and/or PEGylation chemistries that are can be used in accordance with the provided embodiments: U.S. Pat. Nos. 5,824,778, 5,476,653, 6,875,841, 5,872,191, 5,767,284, EP 0 839 850, WO 97/32607, EP 229,108, EP 402,378, U.S. Pat. Nos. 4,902,502, 5,281,698, 5,122,614, 5,219,564, WO 92/16555, WO 94/04193, WO 94/14758, WO 94/17039, WO 94/18247, WO 94/28024, WO 95/00162, WO 95/11924, WO95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. Nos. 5,473,034, 5,516,673, EP 605 963, U.S. Pat. No. 5,382,657, EP 510 356, EP 400 472, EP 183 503 and EP 154 316, all of which are incorporated herein by reference.

In some embodiments, the conjugation of an invention polypeptide and an activated polymer molecule(s) is conducted in accordance with any conventional method, e.g., as described in the following references (which also describe suitable methods for activation of polymer molecules): Harris and Zalipsky, eds., Poly(ethylene glycol) Chemistry and Biological Applications, AZC, Washington; R. F. Taylor, (1991), “Protein Immobilisation: Fundamentals and Applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y., all of which are incorporated herein by reference.

In some embodiments, variant BCMA polypeptides comprising a mutated CRD relative to huBCMA (SEQ ID NO:1), fusion polypeptides (and dimers thereof), may be glycosylated in vivo by introducing a polynucleotide encoding a variant BCMA molecule as provided herein having one or more N- or O-glycosylation sites into a glycosylating eukaryotic expression host cell. In some embodiments, the glycosylating eukaryotic expression host cell may be selected from cells such as a fungal cell (e.g., a filamentous fungal cell or a yeast cell), an insect cell, a mammalian cell, a plant cell, or any other glycosylating eukaryotic expression host cell.

In some embodiments, non-polypeptide lipophilic moieties that are suitable for conjugation to variant BCMA molecules comprising a mutated CRD relative to huBCMA CRD (SEQ ID NO:1) include a natural compound such as a saturated or an unsaturated fatty acid, a fatty acid diketone, a terpene, a prostaglandin, a vitamin, a carotenoid or a steroid, a phospholipid, or alternatively, a synthetic compound, such as a linear or branched aliphatic, aryl, alkaryl acid (e.g., carboxylic, sulphonic), alcohol, amine. Conjugation to non-polypeptide lipophilic moieties may take place at any one of the following exemplary attachment sites: the N-terminus or the C-terminus of a provided variant BCMA molecule, the hydroxyl groups of the amino acid residues Ser, Thr or Tyr, the £-amino group of Lys, the SH group of Cys or the carboxyl group of Asp and Glu. In some embodiments, the provided variant BCMA molecules and the non-polypeptide lipophilic moiety may be conjugated to each other either directly or indirectly via a linker in accordance with known methods, such as those described in Bodanszky, “Peptide Synthesis”, John Wiley, New York (1976) and WO 96/12505, both of which are incorporated herein by reference.

### D. Features of BCMA Variant Polypeptides

Provided herein are variant BCMA polypeptides comprising a mutated CRD relative to wild-type human BCMA CRD (SEQ ID NO:1). In some embodiments, the variant BCMA polypeptides provided herein and molecules comprising such variant BCMA polypeptides, such as fusion polypeptides and dimers, exhibit particular features, including, but not limited to particular affinity to ligands BAFF and/or APRIL, inhibition of activity or function of BAFF and/or APRIL, and/or reduced binding to heparan sulfate proteoglycans (HSPGs). In some embodiments, the variant BCMA polypeptides provided herein exhibit particular features, including, but not limited to particular affinity to ligands BAFF and APRIL. In some embodiments, the variant BCMA polypeptides provide herein and molecules comprising such variant BCMA polypeptides, such as fusion polypeptides and dimers, exhibit reduced glycosylation and/or enhanced molecular weight homogeneity. In some embodiments, the variant BCMA polypeptides provided herein display reduced glycosylation, such as by glycosylating host cells, resulting in enhanced molecular weight homogeneity. In some embodiments, the variant BCMA polypeptides provided herein exhibit reduced binding to or do not bind, such as specifically do not bind, to heparan sulfate proteoglycans (HSPGs).

### 1. BAFF and APRIL Binding Affinity

Provided herein are BCMA variant BCMA polypeptides comprising a mutated CRD relative to wild-type human BCMA CRD (SEQ ID NO:1) that bind, such as specifically bind, to BAFF and APRIL. In some embodiments, the provided variant BCMA polypeptides bind, such as specifically bind, to mammalian BAFF and APRIL. In some aspects, the variant BCMA polypeptide, the fusion polypeptide, or the dimer, exhibits greater binding affinity for BAFF and/or APRIL, compared to the binding affinity of a reference BCMA polypeptide or a reference binding molecule. In some embodiments, the provided variant BCMA polypeptides bind, such as specifically bind, to human BAFF and APRIL (huBAFF and huAPRIL, respectively). In some embodiments, the provided variant BCMA polypeptides bind, such as specifically bind, to murine BAFF and APRIL.

In some aspects, binding affinity or binding avidity is dependent on the format of the assayed molecule and the assay used to assess binding activity. The Examples section herein describes exemplary assays. For example, huBAFF and huAPRIL binding activities or murine BAFF and murine APRIL binding activities can be assessed using the phage display, surface plasmon resonance (SPR), BIACORE™, Kinetic Exclusion Assay (KINEXA™), and cell-based binding assay methods, as described in Examples. Other binding assays are known. In some aspects, the K_{D} is measured using surface plasmon resonance (SPR). In some aspects, the K_{D} is measured using Kinetic Exclusion Assay (KINEXA™)

BAFF (B cell-activating factor of the TNF family) is also known as B-lymphocyte stimulator (BLyS), TALL-1, THANK, and zTNF4). In some embodiments, huBAFF comprises the amino acid sequence of a mature human BAFF protein, such as set forth in SEQ ID NO:214, or isoforms thereof. APRIL (a proliferation-inducing ligand) is also known as TALL-2. In some embodiments, huAPRIL comprises the amino acid sequence of mature huAPRIL or isoforms thereof. In some embodiments, the mature huAPRIL protein lacks a signal peptide or a propeptide. In some embodiments, huAPRIL comprises amino acid residues 105-250 of SEQ ID NO:215.

In some embodiments, the variant BCMA polypeptides comprising a mutated CRD relative to wild-type huBCMA (SEQ ID NO:1) bind, such as specifically bind, to BAFF with higher affinity than a protein comprising a wild-type huBCMA CRD (SEQ ID NO:1). In some embodiments, the provided variant BCMA polypeptides bind to BAFF, such as huBAFF, with at least at or about 2-fold, 3-fold, 4-fold, 5-fold, 10-fold, 20-fold, 30-fold, 40-fold, 50-fold, 60-fold, 70-fold, 80-fold, 90-fold, or 100-fold higher affinity relative to a reference BCMA polypeptide or a reference binding molecule, such as a protein comprising a wild-type huBCMA CRD (SEQ ID NO:1), such as huBCMA ECD (SEQ ID NO:152).

In some embodiments, variant BCMA polypeptides comprising a mutated CRD relative to wild-type huBCMA (SEQ ID NO:1) bind, such as specifically bind, to APRIL with higher affinity than a protein comprising a wild-type huBCMA CRD (SEQ ID NO:1). In some embodiments, the provided variant BCMA polypeptides bind to APRIL, such as huAPRIL, with at least at or about 2-fold, 3-fold, 4-fold, 5-fold, 10 fold, 20-fold, 30-fold, 40-fold, or 50-fold higher affinity relative to a reference BCMA polypeptide or a reference binding molecule, such as a protein comprising a wild-type huBCMA CRD (SEQ ID NO:1), such as huBCMA ECD (SEQ ID NO:152).

In some embodiments, the provided variant BCMA molecules exhibit huAPRIL binding activity that is less than that of a reference BCMA polypeptide or a reference binding molecule, such as the corresponding control protein. In these embodiments, it is typically about 0.1% or less, about 0.2% or less, about 0.5% or less, about 1% or less, about 5% or less or 10% or less, or about 80% or less, or about 90% or less, or about 95% or less, or about 99% or less, and typically in the range of from at or about 0.05% to at or about 99% or from at or about 0.5% to at or about 95% of the huAPRIL binding activity of the corresponding control protein, and more typically in the range of from at or about 0.05% or from at or about 0.1% or from at or about 1%, up to at or about 70% or up to at or about 60% or up to at or about 50% or up to at or about 40% of the huAPRIL binding activity of the corresponding control protein, as measured, for example, in the BIACORE assay of Example 9 or the KINEXA assay of Example 11.

### 2. Glycosylation and Molecular Weight Homogeneity

Glycosylation is a post-translational modification that can affect the structure and function of proteins. Wild-type huBCMA is a glycoprotein comprising an N-glycosylation site (Huang et al., PNAS 2013; 110(27):10928-10933). Glycosylating cells, such as a glycosylating host cell expressing huBCMA or variants thereof, can produce various glycoforms of a protein, such as huBCMA or variants thereof. Glycoform variability can result in heterogeneous protein characteristics, such as molecular weight, structure, and function. In some embodiments, glycosylation of variant BCMA polypeptides comprising a serine 40 to glycine 40 (S40G) mutation is inhibited compared to variant BCMA polypeptides and wild-type huBCMA comprising a serine at residue 40 (determined relative to SEQ ID NO:1). In some embodiments, the provided variant BCMA polypeptides, such as the variant BCMA polypeptides comprising an S40G mutation, are more homogeneous, in terms of molecular weight, than a protein comprising the wild-type serine 40 residue, such as a reference BCMA polypeptide, such as huBCMA CRD (SEQ ID NO:1).

In some embodiments, the sequence characteristics that promote protein greater molecular weight homogeneity can be combined with one or more of any of the provided sequence characteristics that appear to correlate with the phenotypes of improved huBAFF binding activity. In some embodiments, the sequence characteristics that promote protein greater molecular weight homogeneity can be combined with one or more of any of the provided sequence characteristics that appear to correlate with the phenotypes of improved APRIL binding activity. In some embodiments, the sequence characteristics that promote protein greater molecular weight homogeneity can be combined with one or more of any of the provided sequence characteristics that appear to correlate with the phenotypes of improved huBAFF and/or huAPRIL binding activity. In some embodiments, the sequence characteristics that promote protein greater molecular weight homogeneity can be combined with one or more of any of the provided sequence characteristics that appear to correlate with the phenotypes of greater binding affinity for huBAFF relative to huAPRIL. In some embodiments, the sequence characteristics that promote protein greater molecular weight homogeneity can be combined with one or more of any of the provided sequence characteristics that appear to correlate with the greater binding affinity for huAPRIL relative to huBAFF.

### 3. Heparan Sulfate Proteoglycan (HSPG) Binding Affinity

Heparan sulfate proteoglycans (HSPGs), composed of a core protein covalently linked to glycosaminoglycan (GAG) chains formed by unbranched sulfated anionic polysaccharides, known as heparan sulfates, are widely expressed and mediate various biological activities (Cagno et al., Viruses. 2019 July; 11(7):596) and Sarrazin et al., Cold Spring Harb Perspect Biol. 2011; 3(7): a004952). It may be advantageous to reduce or minimize HSPG binding, thereby preserving HSPG-mediated activities, which have effects at the cellular, tissue, and organismal level.

In some embodiments, provided herein are variant BCMA polypeptides that exhibit reduced binding to, or do not bind to HSPGs. In some embodiments, the variant BCMA polypeptides provided herein exhibit reduced binding to or lack affinity for HSPGs, for example syndecan-1 and syndecan-2. In some embodiments, provided herein are variant BCMA polypeptides that exhibit reduced binding to HSPGs compared to the binding of a reference BCMA polypeptide or a reference binding molecule to HSPGs.

4. Inhibition of BAFF and/or APRIL

In some aspects, the provided variant BCMA polypeptide, fusion polypeptide, or dimer inhibits the activity or function of BAFF and/or APRIL. In some aspects, the provided variant BCMA polypeptide, fusion polypeptide, or dimer, exhibits greater inhibition of the activity or function of BAFF and/or APRIL, compared to the inhibition of the activity or function of BAFF and/or APRIL by a reference BCMA polypeptide or a reference binding molecule. In some aspects, the reference BCMA polypeptide includes a corresponding control protein, or a corresponding wild-type BCMA polypeptide. In some aspects, the reference binding molecule is selected from among atacicept, telitacicept, belimumab, or BION-1301. In some aspects, the activity or function of BAFF and/or APRIL is selected from B-cell survival, B-cell proliferation, and/or immunoglobulin production.

In some aspects, the inhibition of the activity or function of BAFF and/or APRIL is assessed using any known assays for measuring the activity or function of BAFF and/or APRIL, for example, binding assays, cell-based expression assays, cell proliferation assays, cell-based effector assays, immunoglobulin production measurements, and/or reporter assays, for example, including those described in the Examples herein. In some aspects, the activity or function of BAFF and/or APRIL assessed includes binding to cells, such as B cells, that express their corresponding receptors, such as BAFF-R, TACI and BCMA. In some aspects, the inhibition of the activity or function of BAFF and/or APRIL is assessed by measuring the blocking of binding of BAFF and/or APRIL to cells that express such receptors.

In some aspects, the activity or function of BAFF and/or APRIL assessed includes stimulating the growth and/or proliferation of cells, such as B cells, that express the corresponding receptors, such as BAFF-R, TACI and BCMA. In some embodiments, the proliferation of B cells in the presence of BAFF and/or APRIL, and the inhibition of proliferation of B cells by the provided variant BCMA polypeptide, fusion polypeptide, or dimer, is assessed.

In some aspects, the inhibition of activity or function of human BAFF by the variant BCMA polypeptide, the fusion polypeptide, or the dimer is at least at or about 5-, 10-, 20-, 25-, 50-, 100-, 200-, 250-, or 500-fold greater than that of the reference BCMA polypeptide or the reference binding molecule. In some embodiments, the inhibition of activity or function of human APRIL by the variant BCMA polypeptide, the fusion polypeptide, or the dimer is at least at or about 5-, 10-, 20-, 25-, 50-, 100-, 200-, 250-, or 500-fold greater than that of the reference BCMA polypeptide or the reference binding molecule.

### II. METHODS OF MAKING BCMA VARIANT MOLECULES

Also provided are methods, polynucleotides, compositions, and kits, for expressing the variant BCMA polypeptides and fusion proteins, and for producing the cells expressing such molecules. In some embodiments, one or more variant BCMA polypeptides and fusion proteins can be genetically engineered into cells such as host cells. The genetic engineering generally involves introduction of a nucleic acid encoding the recombinant or engineered component into the cell, such as by retroviral transduction, transfection, or transformation.

### A. Polynucleotides Encoding BCMA Variant Molecules

Provided herein are polynucleotides encoding any of the variant BCMA polypeptides, fusion proteins, conjugates, dimers, multimers or components thereof, described herein. In some embodiments, the provided polynucleotides encoding variant BCMA molecules can be engineered to target polypeptide expression to a desired cellular compartment, membrane, or organelle of a cell, or to direct polypeptide secretion to the periplasmic space or into the cell culture media In some embodiments, polynucleotides encoding variant BCMA molecules can be fused in-frame to nucleic acids encoding a signal sequence, such as a secretion or localization sequence. Exemplary signal sequences are known. Non-limiting examples of signal sequences include secretion leader peptides, organelle targeting sequences (e.g., nuclear localization sequences, endoplasmic reticulum (ER) retention signals, mitochondrial transit sequences, chloroplast transit sequences), membrane localization or anchor sequences (e.g., stop transfer sequences, GPI anchor sequences).

In some aspects, provided herein are polynucleotides encoding any of the variant BCMA polypeptides or fusion proteins provided herein. In some embodiments, the polynucleotides described herein encode variant BCMA polypeptides comprising a mutated CRD relative to wild-type human BCMA CRD (SEQ ID NO:1), which is encoded by SEQ ID NO:2. Full length huBCMA (SEQ ID NO:149) is encoded by a polynucleotide sequence as set forth in SEQ ID NO:148. HuBCMA ECD (SEQ ID NO:152) is encoded by a polynucleotide sequence as set forth in SEQ ID NO:151. Due to the degeneracy of the genetic code, it can be understood that a multitude of polynucleotide sequences encoding the provided variant BCMA polypeptides exist. For example, the codons AGA, AGG, CGA, CGC, CGG, and CGU all encode the amino acid arginine. Thus, at every position in the polynucleotides of the invention where an arginine is specified by a codon, the codon can be altered to any of the corresponding codons described above without altering the encoded polypeptide. It is understood that U in an RNA sequence corresponds to T in a DNA sequence.

Such “silent variations” are one species of “conservative” variation. It can be recognized that each codon in a polynucleotide sequence (except AUG, which is ordinarily the only codon for methionine, and UGG, which is ordinarily the only codon for tryptophan) can be modified by standard techniques to encode a functionally identical polypeptide. Accordingly, each silent variation of a polynucleotide which encodes a polypeptide is implicit in any described sequence. Contemplated and provided herein is every possible variation of polynucleotide sequence encoding a polypeptide of the invention that could be made by selecting combinations based on possible codon choices. These combinations are made in accordance with the standard triplet genetic code, as applied to the provided polynucleotide sequences encoding variant BCMA polypeptides.

A group of two or more different codons that, when translated in the same context, all encode the same amino acid, are referred to herein as “synonymous codons.” variant BCMA polypeptides as described may be codon optimized for expression in a particular host organism by modifying the polynucleotides to conform with the optimum codon usage of the desired host organism. It can be recognized that tables and other references providing preference information for a wide range of organisms are readily available. See e.g., Henaut and Danchin in “Escherichia coli and Salmonella,” Neidhardt, et al. Eds., ASM Press, Washington D.C. (1996), pp. 2047-2066, which is incorporated herein by reference.

In some embodiments, the nucleotide sequence encoding the variant BCMA CRD has at least at or about 85%, 86%, 87%, 88%, 89%, 90%, 91%, 92%, 93%, 94%, 95%, 96%, 97%, 98%, or 99% sequence identity to the sequence set forth in SEQ ID NO:159. In some embodiments, the nucleotide sequence encoding the variant BCMA CRD comprises SEQ ID NO:159.

In some embodiments, the nucleotide sequence encoding the peptide linker has at least at or about 80%, 81%, 82%, 83%, 84%, 85%, 86%, 87%, 88%, 89%, 90%, 91%, 92%, 93%, 94% or 95% sequence identity to the sequence set forth in SEQ ID NO:155. In some embodiments, the nucleotide sequence encoding the peptide linker has at least at or about 80%, 81%, 82%, 83%, 84%, 85%, 86%, 87%, 88%, 89%, 90%, 91%, 92%, 93%, 94% or 95% sequence identity to the sequence set forth in SEQ ID NO:157. In some embodiments, the nucleotide sequence encoding the linker comprises SEQ ID NO: 155. In some embodiments, the nucleotide sequence encoding the linker comprises SEQ ID NO:157.

In some embodiments, the nucleotide sequence encoding the IgG4 Fc region has least at or about 85%, 86%, 87%, 88%, 89%, 90%, 91%, 92%, 93%, 94%, 95%, 96%, 97%, 98%, or 99% sequence identity to the sequence set forth in SEQ ID NO:238. In some embodiments, the nucleotide sequence encoding the IgG4 Fc region comprises SEQ ID NO:238. In some embodiments, the nucleotide sequence encoding the IgG4 Fc region has least at or about 85%, 86%, 87%, 88%, 89%, 90%, 91%, 92%, 93%, 94%, 95%, 96%, 97%, 98%, or 99% sequence identity to the sequence set forth in SEQ ID NO:160. In some embodiments, the nucleotide sequence encoding the IgG4 Fc region comprises SEQ ID NO:160. In some embodiments, the nucleotide sequence encoding the IgG4 Fc region has least at or about 85%, 86%, 87%, 88%, 89%, 90%, 91%, 92%, 93%, 94%, 95%, 96%, 97%, 98%, or 99% sequence identity to the sequence set forth in SEQ ID NO: SEQ ID NO:162. In some embodiments, the nucleotide sequence encoding the IgG4 Fc region comprises SEQ ID NO:162.

In some embodiments, provided herein are polynucleotides encoding variant BCMA polypeptides, such as variant BCMA fusion polypeptides, can be designed to include signal sequences, which direct secretion of mature polypeptides through a prokaryotic or eukaryotic cell membrane.

In some embodiments, the nucleotide sequence encoding the signal peptide has at least at or about 85%, 86%, 87%, 88%, 89%, 90%, 91%, 92%, 93%, 94% or 95%, 96%, 97%, or 98% sequence identity to SEQ ID NO:153. In some embodiments, the nucleotide sequence encoding the signal peptide comprises SEQ ID NO:153. In some embodiments, the nucleotide sequence encoding the signal peptide has at least at or about 85%, 86%, 87%, 88%, 89%, 90%, 91%, 92%, 93%, 94% or 95%, 96%, 97%, or 98% sequence identity to SEQ ID NO:249. In some embodiments, the nucleotide sequence encoding the signal peptide comprises SEQ ID NO:249. It is understood that also included are mature sequences thereof that lack a signal peptide following cleavage of the signal peptide when expressed and produced from a cell. In some embodiments, the signal peptide has at least at or about 85%, 86%, 87%, 88%, 89%, 90%, 91%, 92%, 93%, 94% or 95%, 96%, 97%, or 98% sequence identity to SEQ ID NO:154. In some embodiments, the signal peptide comprises SEQ ID NO:154. In some embodiments, the signal peptide has at least at or about 85%, 86%, 87%, 88%, 89%, 90%, 91%, 92%, 93%, 94% or 95%, 96%, 97%, or 98% sequence identity to SEQ ID NO:250. In some embodiments, the signal peptide comprises SEQ ID NO:250. It is understood that also included are mature sequences thereof that lack a signal peptide following cleavage of the signal peptide when expressed and produced from a cell.

In some embodiments, the nucleotide sequence encoding the BCMA variant fusion polypeptide including the signal sequence has at least at or about 85%, 86%, 87%, 88%, 89%, 90%, 91%, 92%, 93%, 94% or 95%, 96%, 97%, or 98% sequence identity to SEQ ID NO:164. In some embodiments the polynucleotide sequence encoding the BCMA variant fusion polypeptide including the signal sequence comprises SEQ ID NO:164. In some embodiments, the nucleotide sequence encoding the BCMA variant fusion polypeptide including the signal sequence has at least at or about 85%, 86%, 87%, 88%, 89%, 90%, 91%, 92%, 93%, 94% or 95%, 96%, 97%, or 98% sequence identity to SEQ ID NO:239. In some embodiments the polynucleotide sequence encoding the BCMA variant fusion polypeptide including the signal sequence comprises SEQ ID NO:239. It is understood that also included are mature sequences thereof that lack a signal peptide following cleavage of the signal peptide when expressed and produced from a cell.

In some embodiments, the nucleotide sequence encoding the BCMA variant fusion polypeptide lacking the signal sequence has at least at or about 85%, 86%, 87%, 88%, 89%, 90%, 91%, 92%, 93%, 94% or 95%, 96%, 97%, or 98% sequence identity to SEQ ID NO:166. In some embodiments the polynucleotide sequence encoding the BCMA variant fusion polypeptide lacking the signal sequence comprises SEQ ID NO:166.

### B. Expression Vectors

Provided herein are vectors, such as expression vectors comprising polynucleotides encoding any of the variant BCMA polypeptides, fusion proteins, conjugates, dimers, multimers or components thereof, described herein, or any of the polynucleotides described herein.

Polynucleotides encoding the provided variant BCMA molecules comprising a mutated CRD relative to huBCMA CRD (SEQ ID NO:1) can be incorporated into any one of a variety of known expression vectors. Vectors can be employed to transform a host, such as a host cell, and facilitate expression of a provided polynucleotide encoding the provided variant BCMA molecules comprising a mutated CRD relative to huBCMA CRD (SEQ ID NO:1). In some embodiments, expression vectors compatible with prokaryotic host cells may be used, such as prokaryotic expression vectors. Non-limiting examples of expression vectors compatible with prokaryotic host cells include pUC vectors (e.g., New England BioLabs), BLUESCRIPT vector (e.g., Stratagene), T7 expression vector (e.g., Invitrogen), pET vector (e.g., Novagen), multifunctional E. coli cloning and expression vectors.

In some embodiments, expression vectors compatible with eukaryotic host cells may be used, such as known eukaryotic expression vectors. Non-limiting examples of expression vectors compatible with eukaryotic host cells include pCMV vectors (e.g., Invitrogen), pTRES vector (e.g., Clontech), pSG5 vector (e.g., Stratagene), pCDNA3.1 (e.g., Invitrogen Life Technologies), pCDNA3 (e.g., Invitrogen Life Technologies), and Ubiquitous Chromatin Opening Element (UCOE) expression vector (e.g., Millipore).

In some embodiments, suitable expression vectors include chromosomal, non-chromosomal, and synthetic DNA sequences. In some embodiments, suitable expression vectors include a bacterial artificial chromosome (BAC), a yeast artificial chromosome (YAC), a plasmid, such as, for example, a bacterial plasmid or a yeast plasmid, a cosmid, or a phage. In some embodiments, a suitable expression vector is derived from viral DNA, such as, for example vaccinia, adenovirus, fowl pox virus, pseudorabies, adenovirus, adeno-associated virus, retroviruses as well as vectors derived from combinations of plasmids and phage DNA. Any vector that transduces genetic material into a cell, and if replication is desired, which is replicable and viable in the relevant host can be used. In some embodiments, an expression vector as provided herein comprises a pUC vector sequence. In some embodiments, the expression vector comprises a sequence having at least at or about 80%, 85%, 90%, 95%, or 99% sequence identity to SEQ ID NO:219. In some embodiments, the expression vector is a pUC vector. In some embodiments, the expression vector comprises the sequence as set forth in SEQ ID NO:219.

In some embodiments, an expression vector comprises one or more selectable marker gene(s) to provide a phenotypic trait for selection of transformed host cells. In some embodiments, selectable marker genes include those coding for resistance to the antibiotic spectinomycin or streptomycin (e.g., the aadA gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance. In some embodiments, selectable marker genes include dihydrofolate reductase or neomycin resistance for eukaryotic cell culture, and tetracycline or ampicillin resistance in E. coli.

In some embodiments, an expression vector as provided herein comprises a puromycin resistance gene. In some embodiments, the puromycin resistance gene comprises a nucleotide sequence having at least at or about 80%, 85%, 90%, 95%, or 99% sequence identity to SEQ ID NO:220. In some embodiments, the puromycin resistance gene comprises a nucleotide sequence as set forth in SEQ ID NO:220. In some embodiments, the puromycin resistance gene comprises an amino acid sequence having at least 85%, at least 90%, at least 95%, or at least 99% sequence identity to the sequence set forth in SEQ ID NO:221. In some embodiments, the puromycin resistance gene comprises an amino acid sequence as set forth in SEQ ID NO:221. In some embodiments, an expression vector as provided herein comprises an ampicillin resistance gene. In some embodiments, the ampicillin resistance gene comprises a nucleotide sequence having at least at or about 80%, 85%, 90%, 95%, or 99% sequence identity to SEQ ID NO:222. In some embodiments, the ampicillin resistance gene comprises a nucleotide sequence as set forth in SEQ ID NO:222. In some embodiments, the ampicillin resistance gene comprises an amino acid sequence having at least at or about 80%, 85%, 90%, 95%, or 99% sequence identity to SEQ ID NO:223. In some embodiments, the ampicillin resistance gene comprises an amino acid sequence as set forth in SEQ ID NO:223. In some embodiments, an expression vector as provided herein comprises a puromycin resistance gene and an ampicillin resistance gene.

### C. Regulatory Elements

In some embodiments, an expression vector or construct as provided herein comprises one or more regulatory elements or sequences that direct expression of one or more the polynucleotides described herein. Such regulatory elements or sequences include, but are not limited to, a leader, a polyadenylation sequence, a propeptide sequence, a promoter, a signal peptide sequence, and a transcription terminator. In some embodiments, the regulatory elements or sequences comprise a promoter and a transcriptional and a translational stop signal. In some embodiments, the regulatory elements or sequences comprise additional sequences that introduce specific restriction sites. In some embodiments, the introduced specific restriction sites may facilitate ligation of the elements or sequences with the coding sequence(s) of the nucleotide sequence encoding the BCMA variant polypeptides described herein.

In some embodiments, one or more of a promoter, an enhancer, and a regulatory element regulate expression of the encoded variant BCMA molecule. In some embodiments, the promoter and/or enhancer or regulatory elements can be condition-dependent promoters, enhancers, and/or regulatory elements. In some embodiments, these elements drive expression of a transgene. In some embodiments, the expression vector comprises sequences for amplifying expression, such as an enhancer. In some embodiments, when incorporated into an expression vector, a polynucleotide encoding a variant BCMA molecule comprising a mutated CRD relative to huBCMA CRD (SEQ ID NO:1) is operatively linked to an appropriate transcription control sequence (promoter) to direct mRNA synthesis. Methods of effecting this operative linking, either before or after the DNA molecule is inserted into a vector, are well known. Expression control sequences include promoters, activators, enhancers, operators, ribosomal binding sites, start signals, stop signals, cap signals, polyadenylation signals, and other signals involved with the control of transcription or translation.

### 1. Promoters

The polynucleotides described herein can be driven by a promoter or enhancer to control or regulate expression. In some embodiments, the promoter is operably linked to the coding region of a nucleic acid of which expression is desired. In some embodiments, other promoters known to control expression of genes in prokaryotic or eukaryotic cells or their viruses can be used. Non-limiting examples of promoters that can be operatively linked to the provided polynucleotides, such as those encoding variant BCMA molecules, in an expression construct include, for example, an EF1alpha promoter with an HTLV1 enhancer, Caviid herpesvirus 2 promoter A, SV40 promoter, E. coli lac or trp promoter, phage lambda PL promoter, tac promoter, or T7 promoter. In some embodiments, a polynucleotide encoding a variant BCMA molecule comprising a mutated CRD relative to huBCMA CRD (SEQ ID NO:1) is operatively linked to a T5 promoter.

In some embodiments, promoters can be tissue specific. A tissue specific promoter allows for the production of a protein in a certain population of cells that have the appropriate transcriptional factors to activate the promoter. Numerous promoters are commercially available and widely known in the art. In some embodiments, the promoter is selected from the group of a cytomegalovirus promoter (CMV), a bla promoter, promoter A from Caviid herpesvirus 2, a phosphoglycerate kinase (PGK) promoter, a simian virus 40 early promoter (SV40), or a Rous sarcoma virus LTR promoter (RSV). The promoter can be a constitutive promoter, such as a CMV promoter, a tissue-specific promoter, an inducible or regulatable promoter. In some embodiments, the polynucleotide contains an inducible promoter operably linked to the coding region, such that expression of the nucleic acid is controllable by controlling the presence or absence of the appropriate inducer of transcription. An exemplary promoter, such as an SV40 promoter, comprises the sequence set forth in SEQ ID NO:247, or a sequence having at least at or about 80%, 85%, 90%, 95%, or 99% sequence identity to SEQ ID NO:247.

In some embodiments, the promoter is a constitutive promoter. Exemplary promoters include, but are not limited to, a CMV promoter, a truncated CMV promoter, a PGK promoter, such as a mouse PGK promoter, a bla promoter, a human serum albumin promoter or an C-1-antitrypsin promoter. In some embodiments, the promoter is a CMV promoter. In some embodiments, the promoter is a truncated CMV promoter in which binding sites for known transcriptional repressors have been deleted. CMV-derived promoters can be human or of simian origin. In some embodiments, the promoter is an inducible promoter. For example, the promoter is the inducible ecdysone promoter. Other examples of promoters include steroid promoters, such as estrogen and androgen promoters, and metallothionein promoters. In some embodiments, the enhancer can be a tissue specific- or nonspecific enhancer. For example, the enhancer is a liver-specific enhancer element. Exemplary enhancer elements include, but are not limited to, human serum albumin (HSA) enhancers, human prothrombin (HPrT) enhancers, C-1-microglobulin enhancers, intronic aldolase enhancers and apolipoprotein E hepatic control region.

In some embodiments, the vector or construct can contain a single promoter that drives the expression of one or more nucleic acid molecules. In some embodiments, such nucleic acid molecules, e.g., transcripts, can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g. encoding a first and second chimeric receptor) by a message from a single promoter. An exemplary IRES sequence comprises the sequence set forth in SEQ ID NO:244, or a sequence having at least at or about 80%, 85%, 90%, 95%, or 99% sequence identity to SEQ ID NO:244.

Alternatively, in some cases, a single promoter may direct expression of an RNA that contains, in a single open reading frame (ORF), two or three genes (e.g. encoding a first and second binding molecules, e.g., antibody recombinant receptor) separated from one another by sequences encoding a self-cleavage peptide (e.g., 2A cleavage sequences) or a protease recognition site (e.g., furin). The ORF thus encodes a single polypeptide, which, either during (in the case of T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and polynucleotides disclosed herein, without limitation, 2A sequences from the foot-and-mouth disease virus (F2A), equine rhinitis A virus (E2A), Thosea asigna virus (T2A), and porcine teschovirus-1 (P2A) as described in U.S. Patent Publication No. 20070116690. In some embodiments, the one or more different or separate promoters drive the expression of one or more nucleic acid molecules encoding the one or more BCMA variant molecules.

In some embodiments, an expression vector as provided herein comprises a human eukaryotic translation elongation factor 1 promoter. In some embodiments, the hEF1 promoter is a human eukaryotic translation elongation factor 1 alpha (hEF1alpha or hEF1α) promoter. In some embodiments, an hEF1α promoter comprises a sequence having at least at or about 80%, 85%, 90%, 95%, or 99% sequence identity to SEQ ID NO: 225. In some embodiments, the hEF1α promoter comprises a sequence as set forth in SEQ ID NO:225.

### 2. Bicistronic or IRES Element

In some embodiments, expression cassettes containing encoding polynucleotide can be multicistronic (bicistronic or tricistronic, see e.g., U.S. Pat. No. 6,060,273). In some embodiments, transcription units can be engineered as a bicistronic unit containing a bicistronic element, which allows co-expression of gene products by a message from a single promoter. In some embodiments, the bicistronic element is an IRES (internal ribosome entry site). In some embodiments, the bicistronic element may be a self-cleavage sequence, such as 2A sequence (e.g. P2A, FTA or T2A). An exemplary IRES sequence comprises the sequence set forth in SEQ ID NO:244, or a sequence having at least at or about 80%, 85%, 90%, 95%, or 99% sequence identity to SEQ ID NO:244.

Internal ribosome entry sites (IRES) are sequences which initiate translation from an internal initiation codon (usually AUG) within a bi- or multi-cistronic RNA transcript continuing multiple protein coding regions. IRES have been characterized in encephalomyocarditis virus and related picornaviruses (e.g., Jackson et al., RNA 1995; 1:985-1000 and Herman, Trends in Biochemical Sciences 1989; 14(6):219-222). IRES sequences are also detected in mRNAs from other viruses such as cardiovirus, rhinovirus, aphthovirus, hepatitis C virus (HCV), Friend murine leukemia virus (FrMLV) and Moloney murine leukemia virus (MoMLV). The presence of RES in cellular RNAs has also been described. Expression vectors containing IRES elements have been described. See, for example, PCT/US98/03699 and PCT/EP98/07380. In some embodiments, an expression vector comprising the provided polynucleotides encoding variant BCMA molecules further comprises a ribosome binding site for translation initiation.

### 3. 3′ Untranslated Region (UTR)

A 3′-untranslated region (3′-UTR) is typically the part of an mRNA which is located between the protein coding region (i.e., the open reading frame) and the poly(A) sequence of the mRNA. A 3′-UTR of the mRNA is not translated into an amino acid sequence. The 3′-UTR sequence is generally encoded by the gene which is transcribed into the respective mRNA during the gene expression process. The genomic sequence is first transcribed into premature mRNA, which comprises optional introns. The pre-mature mRNA is then further processed into mature mRNA in a maturation process. This maturation process comprises the steps of 5′-capping, splicing the pre-mature mRNA to excise optional introns and modifications of the 3′-end, such as polyadenylation of the 3′-end of the pre-mature mRNA and optional endo- or exonuclease cleavages etc.

In some embodiments, a 3′-UTR corresponds to the sequence of a mature mRNA, which is located 3′ to the stop codon of the protein coding region, for example, immediately 3′ to the stop codon of the protein coding region, and which extends to the 5′-side of the poly(A) sequence, for example, to the nucleotide immediately 5′ to the poly(A) sequence. The term “corresponds to” indicates that the 3′-UTR sequence may be an RNA sequence, such as in the mRNA sequence used for defining the 3′-UTR sequence, or a DNA sequence which corresponds to such RNA sequence. In some embodiments, the term “a 3′-UTR” is the sequence that corresponds to the 3′-UTR of the mature mRNA derived from this gene, i.e., the mRNA obtained by transcription of the gene and maturation of the pre-mature mRNA. In some embodiments, the term “3′-UTR of a gene” encompasses the DNA sequence and the RNA sequence of the 3′-UTR.

In some embodiments, the 3′-UTR sequence comprises a transcription termination sequence. In some embodiments, the transcription termination sequence comprises a poly-A tail, also called a 3′-poly(A) tail or poly(A) sequence. A poly-A tail is a long sequence of adenosine nucleotides added to the 3′-end of an RNA molecule. Polyadenylation is the addition of a poly(A) sequence to a nucleic acid molecule, such as an RNA molecule, e.g., to a premature mRNA. Polyadenylation may be induced by a polyadenylation signal. This signal is preferably located within a stretch of nucleotides at the 3′-end of a nucleic acid molecule, such as an RNA molecule, to be polyadenylated. A polyadenylation signal typically comprises a hexamer consisting of adenine and uracil/thymine nucleotides, preferably the hexamer sequence AAUAAA. Other sequences, preferably hexamer sequences, are also conceivable. Polyadenylation typically occurs during processing of a pre-mRNA (also called premature-mRNA). Typically, RNA maturation (from premRNA to mature mRNA) comprises the step of polyadenylation.

In some embodiments, the provided expression vector comprises a transcription termination sequence comprising an SV40 polyadenylation signal sequence. In some embodiments, an exemplary SV40 polyadenylation signal sequence comprises a sequence having at least at or about 80%, 85%, 90%, 95%, or 99% sequence identity to SEQ ID NO:248. In some embodiments, an exemplary SV40 polyadenylation signal sequence comprises the sequence set forth in the in SEQ ID NO:248. In some embodiments, the provided expression vector comprises a transcription termination sequence comprising a potato proteinase inhibitor II (Pin II) gene (Xing et al., Plant Biotechnol J. 2010; 8(7):772-82). In some embodiments, the provided expression vector comprises a transcription termination sequence comprising a bovine growth hormone (bGH) polyadenylation signal sequence. In some embodiments, the bGH polyadenylation signal sequence comprises a sequence having at least at or about 80%, 85%, 90%, 95%, or 99% sequence identity to SEQ ID NO:218. In some embodiments, the bGH polyadenylation signal sequence comprises the sequence set forth in the in SEQ ID NO:218. In some embodiments, the bGH polyadenylation signal sequence comprises a sequence having at least at or about 80%, 85%, 90%, 95%, or 99% sequence identity to SEQ ID NO:240. In some embodiments, the bGH polyadenylation signal sequence comprises the sequence set forth in the in SEQ ID NO:240.

### D. BCMA Variant Polypeptide Production and Recovery

Provided herein is a method of making any of the provided variant BCMA polypeptides, fusion proteins and/or dimers. In some embodiments, the method comprises: (1) selecting an expression host, such as a host cell, (2) introducing a recombinant polynucleotide encoding the provided variant BCMA molecules into the host cell, (3) culturing the host cell comprising the recombinant polynucleotide in a culture medium under conditions suitable for expression of the encoded variant BCMA molecule, and (4) recovering the variant BCMA molecule from the culture medium or from the cultured host cell.

### 1. Expression Hosts and Methods of Introducing Recombinant Polynucleotides

An expression host, such as a host cell, is any cell type that is susceptible to introduction of a recombinant polynucleotide described herein, such as a vector or a construct comprising polynucleotides encoding any of the variant BCMA polypeptides, fusion proteins and/or dimers provided herein. In some embodiments, the host cell is an isolated host cell. In some embodiments, the isolated host cell is a eukaryotic cell. In some embodiments, the isolated host cell is a mammalian cell, such as a Chinese hamster ovary (CHO) cell, a yeast cell, or a plant cell. In some embodiments, the isolated host cell is a prokaryotic cell. In some embodiments, the isolated host cell is a bacterial cell, such as an E. coli cell, a Bacillus spp. cell, a Streptomyces spp. cell).

In some embodiments, a host cell strain is chosen for its ability to modulate the expression of the inserted sequences or to process, such as post-translationally modify, the expressed protein. Such modifications of the protein include, but are not limited to: acetylation, carboxylation, glycosylation, phosphorylation, lipidation and acylation. Post-translational modifications (PTMs) can influence correct insertion, folding and/or protein function. In one example of PTM, cleavage of a preprotein forms a mature protein, which consequently lacks amino acid residues encoded by a signal sequence. Different host cells such as E. coli, Bacillus spp., yeast or mammalian cells, for example, CHO, HeLa, BHK, MDCK, HEK 293, WI38 have specific cellular machinery and characteristic mechanisms for such post-translational activities. In some embodiments, a host cell may be chosen to ensure the correct modification and processing of the encoded recombinant protein comprising a variant BCMA molecule as provided herein. In some aspects, the host cell is a CHO cell.

Provided herein is an expression host, such as a host cell, comprising any of the provided polynucleotides encoding a variant BCMA molecule comprising a mutated CRD relative to huBCMA CRD (SEQ ID NO:1), such as any provided herein. In some embodiments, a host cells is transduced with a vector or a construct comprising polynucleotides encoding a variant BCMA molecule comprising a mutated CRD relative to huBCMA CRD (SEQ ID NO:1), such as any provided herein. In some embodiments, a host cells is transformed with a vector or a construct comprising polynucleotides encoding a variant BCMA molecule comprising a mutated CRD relative to huBCMA CRD (SEQ ID NO:1), such as any provided herein. In some embodiments, a host cells is transfected with a vector or a construct comprising polynucleotides encoding a variant BCMA molecule comprising a mutated CRD relative to huBCMA CRD (SEQ ID NO:1), such as any provided herein.

Introduction of the nucleic acid construct into the host cell can be effected by calcium phosphate transfection, DEAE-Dextran mediated transfection, electroporation, gene or vaccine gun, injection, or other common techniques (see, e.g., Davis, L., Dibner, M., and Battey, I. (1986) Basic Methods in Molecular Biology), which is incorporated herein by reference, for in vivo, ex vivo or in vitro methods.

### 2. Culture of an Expression Host and Polypeptide Production

Provided herein are methods of culturing an expression host, such as a host cell, comprising a recombinant polynucleotide encoding a variant BCMA molecule comprising a mutated CRD relative to huBCMA CRD (SEQ ID NO:1), such as any provided herein, to facilitate production of the provided variant BCMA molecules.

The culture and/or the production of expression hosts, such as a host cells, is known. Culture and/or production of cells of bacterial, plant, animal (especially mammalian), and archebacterial origin are described in, for example, Sambrook, Joseph. Molecular Cloning: a Laboratory Manual. Cold Spring Harbor, N.Y.: Cold Spring Harbor Laboratory Press, 2001; Freshney (1994) Culture of Animal Cells, a Manual of Basic Technique, third edition, Wiley-Liss, New York (and the references cited therein); Doyle and Griffiths (1997) Mammalian Cell Culture: Essential Techniques John Wiley and Sons, NY; Humason (1979) Animal Tissue Techniques, fourth edition, W.H. Freeman and Company; and Ricciardelli, et al. In vitro Cell Dev. Biol. 1989; 25:1016-1024, all of which are incorporated herein by reference. References that describe plant cell culture and regeneration include, Payne, et al. (1992) Plant Cell and Tissue Culture in Liquid Systems, John Wiley & Sons, Inc. New York, NY; Gamborg and Phillips (Eds) (1995) Plant Cell, Tissue and Organ Culture; Fundamental Methods Springer Lab Manual, Springer-Verlag (Berlin Heidelberg, New York); Jones, Ed. (1984) Plant Gene Transfer and Expression Protocols, Humana Press, Totowa, New Jersey and Plant Molecular Biology (1993) R. R. D. Croy, Ed. Bios Scientific Publishers, Oxford, U.K. ISBN 0 12 198370 6, all of which are incorporated herein by reference.

Cell culture media in general is described in Atlas and Parks (Eds.) The Handbook of Microbiological Media (1993) CRC Press, Boca Raton, FL, which is incorporated herein by reference. Additional information for cell culture is found in available commercial literature such as the Life Science Research Cell Culture Catalogue (1998) from Sigma-Aldrich, Inc. (St. Louis, MO) and The Plant Culture Catalogue and supplement (1997) from Sigma-Aldrich, Inc. (St. Louis, MO), both of which are incorporated herein by reference.

In some embodiments, the variant BCMA molecule, such as any variant BCMA polypeptide, fusion proteins and/or dimers described herein, produced by the host cell may be secreted, membrane-bound, or contained intracellularly, depending on the sequence and/or the vector used. As will be understood by those of skill in the art, expression vectors comprising recombinant polynucleotides encoding the provided variant BCMA molecules can be designed to include signal sequences which direct secretion of mature polypeptides through a prokaryotic or eukaryotic cell membrane.

In some embodiments, stable expression can be used for long-term, high-yield production of recombinant proteins. In some embodiments, host cell lines that stably express a polypeptides and fusion proteins as described herein, are transduced using expression vectors or constructs that contain viral origins of replication, endogenous expression elements, and/or a selectable marker gene. In some embodiments, following the introduction of the vector or construct, host cells may be allowed to grow for 1-2 days in an enriched media before they are switched to selective media. The purpose of the selectable marker is to confer resistance to selection, and its presence allows growth and recovery of cells which successfully express the introduced recombinant polynucleotides. In some embodiments, resistant clumps of stably transformed cells can be proliferated using tissue culture techniques appropriate to the cell type.

### 3. Polypeptide Recovery

Provided herein are methods of recovering a variant BCMA molecule comprising a mutated CRD relative to huBCMA CRD (SEQ ID NO:1), such as any provided herein. In some embodiments, host cells comprising a recombinant polynucleotide encoding a variant BCMA molecule as provided herein are cultured under conditions to facilitate recovery of the encoded variant BCMA protein. In some embodiments, the variant BCMA molecule is recovered from host cell culture medium. In some embodiments, the variant BCMA molecule is recovered from the host cell. In some embodiments, the variant BCMA molecule is recovered from the host cell culture medium and the host cell.

In another embodiment, provided herein are methods for making a variant BCMA fusion polypeptide dimer. In some aspects, the methods comprise: culturing a host cell transformed or transfected with a vector comprising a polynucleotide encoding a variant BCMA fusion polypeptide as described herein in a culture medium under conditions suitable for expression and dimerization of the encoded fusion polypeptide to produce a fusion polypeptide dimer; and recovering the fusion polypeptide dimer from the culture medium or from the cultured host cells. Typically, the polynucleotide also encodes a secretory or signal peptide operably linked to the encoded fusion polypeptide. In this embodiment, the fusion polypeptide is typically secreted from the host cell as a disulfide-bonded fusion polypeptide dimer.

In some embodiments, the host cell is a mammalian cell, such as a CHO cell. In some embodiments, host cells employed in the production and recovery of polypeptides as provided are isolated host cells, as compared to higher order organisms, such as plants or animals. In some methods, the BCMA variant polypeptides, fusion polypeptides, and fusion polypeptide dimers are recovered from the culture medium, host cell, or host cell periplasm.

In some embodiments, following transduction of a suitable host strain and growth of the host strain to an appropriate cell density, the selected promoter is induced by appropriate means, such as, for example, by temperature shift or chemical induction, and cells are cultured for an additional period. In some embodiments, cells are harvested by centrifugation, disrupted by physical or chemical means, and the resulting crude extract retained for further purification. In some embodiments, microbial cells employed in expression of proteins can be disrupted by any convenient method, including freeze-thaw cycling, sonication, mechanical disruption, or use of cell lysing agents, or other known or exemplary methods.

In some embodiments, the BCMA variant polypeptides as described (variant polypeptides, fusion polypeptides (and fusion polypeptide dimers) may be recovered/isolated and optionally purified from recombinant cell cultures by any of a number of methods, such as, for example, ammonium sulfate or solvent precipitation (such as, for example, by using a solvent like ethanol, acetone), acid extraction, ion (anion or cation) exchange chromatography, high performance liquid chromatography (HPLC), phosphocellulose chromatography, hydrophobic interaction chromatography, affinity chromatography, hydroxylapatite chromatography, lectin chromatography and size-exclusion chromatography. Suitable protein purification methods are described in Sandana (1997) Bioseparation of Proteins, Academic Press, Inc.; Bollag et al. (1996) Protein Methods, 2nd Edition, Wiley-Liss, NY; Walker (1996) The Protein Protocols Handbook, Humana Press, NJ; Harris and Angal (1990) Protein Purification Applications: A Practical Approach, TRL Press at Oxford, Oxford, England; Harris and Angal, Protein Purification Methods: A Practical Approach, IRL Press at Oxford, Oxford, England; Scopes (1993) Protein Purification: Principles and Practice, 3rd Edition, Springer Verlag, NY; Janson and Ryden (1998) Protein Purification: Principles, High Resolution Methods and Applications, Second Edition, Wiley-VCH, NY; and Walker (1998) Protein Protocols on CD-ROM, Humana Press, NJ, all of which are incorporated herein by reference. In some embodiments, the purification step comprises purification by a chromatography method, employing an eluant mixture that may comprise arginine, glycine, citrate, or mixtures thereof.

In some embodiments, bacterially-produced polypeptides may form inclusion bodies (IBs), which require further processing steps to generate active polypeptides. In some embodiments, this further processing may entail the isolation and solubilization of the inclusion bodies, unfolding the polypeptide, then refolding the polypeptide into the correct biologically active tertiary structure. In some embodiments, provided are methods of producing a polypeptide as described herein. In some aspects, the methods involve: culturing a host cell transformed with a polynucleotide encoding a polypeptide as described under conditions suitable for expression of the polypeptide as inclusion bodies; recovering inclusion bodies comprising the encoded polypeptide from the transformed and cultured host cells; solubilizing (denaturing) the recovered inclusion bodies comprising the polypeptide with a solubilizing agent; purifying the solubilized polypeptide; allowing the polypeptide to refold; and purifying the refolded polypeptide. In some embodiments, inclusion bodies may be solubilized in solvents such as urea. In some embodiments, refolding may be accomplished, for example, by incubating solubilized polypeptide in a solution of dilute urea and glutathione.

### III. COMPOSITIONS OF BCMA VARIANT MOLECULES

Provided herein are compositions of variant BCMA molecules, such as pharmaceutical compositions, comprising any of the variant BCMA polypeptides, fusion proteins, dimers, conjugates, polynucleotides, vectors, and cells described herein, including variant BCMA molecules comprising a mutated CRD relative to huBCMA CRD (SEQ ID NO:1). In some embodiments, the provided composition, such as a pharmaceutical composition, comprises a variant BCMA polypeptide, a BCMA-Ig-Fc fusion polypeptide, polynucleotides, vectors, or cells. In some embodiments, the provided composition, such as a pharmaceutical composition, comprises a BCMA-Ig-Fc fusion polypeptide dimer, a BCMA-Ig-Fc fusion polypeptide conjugate polynucleotides, vectors, and/or cells. In some embodiments, provided herein are compositions, such as pharmaceutical compositions, comprising any of the provided variant BCMA molecules in any combination.

### A. Dosage Forms and Administration

In some embodiments, compositions comprising variant BCMA molecules, fusion polypeptides, dimers, conjugates, polynucleotides, vectors, or cells, such as pharmaceutical compositions comprising a comprising a variant BCMA CRD polypeptide, mutated relative to huBCMA CRD (SEQ ID NO:1), may be in any form suitable for the intended method of administration. In some embodiments, the compositions, such as pharmaceutical compositions comprising the provided variant BCMA molecules, are formulated for administration by inhalation, parenteral administration, sublingual administration, rectal administration, or topical administration. In some embodiments, topical administration may also involve the use of transdermal administration, such as transdermal patches or iontophoretic devices. In some embodiments, the compositions, such as pharmaceutical compositions such as pharmaceutical compositions comprising the provided variant BCMA molecules, fusion polypeptides, dimers, conjugates, polynucleotides, vectors, or cells, are formulated to provide unit dosage forms, such as single or multiple unit dosage forms.

### 1. Liquid Dosage Forms

In some embodiments, a provided composition, such as a pharmaceutical composition comprising a variant BCMA molecule, fusion polypeptides, dimers, conjugates, polynucleotides, vectors, or cells, can be in the form of a solution, a suspension, or an emulsion. In some embodiments, non-limiting examples of liquid carriers include water, saline, pharmaceutically acceptable organic solvent(s), pharmaceutically acceptable oils and fats as well as mixtures of any two or more thereof. In some embodiments, the liquid carrier comprises other suitable pharmaceutically acceptable excipients such as solubilizers, emulsifiers, nutrients, buffers, preservatives, suspending agents, thickening agents, viscosity regulators, stabilizers. Non-limiting examples of suitable organic solvents include monohydric alcohols, such as ethanol, and polyhydric alcohols, such as glycols. Non-limiting examples of suitable oils include, soybean oil, coconut oil, olive oil, safflower oil, cottonseed oil. In some embodiments, particularly when parenteral administration is contemplated, the carrier can be an oily ester such as ethyl oleate, isopropyl myristate. Parenteral administration includes subcutaneous injections, intravenous administration, intramuscular administration, intrasternal injections, transdermal or transmucosal administration, or infusion techniques. In some aspects, any of the provided pharmaceutical compositions, such as pharmaceutical compositions comprising a variant BCMA molecule, fusion polypeptides, dimers, conjugates, polynucleotides, vectors, or cells, such as any provided herein, are formulated for intravenous administration.

Parenteral administration includes subcutaneous injections, intravenous administration, intramuscular administration, intrasternal injections, transdermal or transmucosal administration, or infusion techniques.

In some embodiments, the provided compositions, such as pharmaceutical compositions comprising the provided variant BCMA molecules, are in the form of microparticles, microcapsules, liposomal encapsulates as well as combinations of any two or more thereof. In some embodiments, the provided compositions, such as pharmaceutical compositions comprising the provided variant BCMA molecules, comprise liposomes. In some aspects, liposomes are generally derived from phospholipids or other lipid substances. In some embodiments, liposomes are formed by mono- or multilamellar hydrated liquid crystals that are dispersed in an aqueous medium. In some embodiments, any non-toxic, physiologically acceptable and metabolizable lipid capable of forming liposomes can be used in formulation liposomal variant BCMA molecules comprising a mutated CRD relative to huBCMA CRD (SEQ ID NO:1). In some embodiments, the provided compositions, such as pharmaceutical compositions comprising lipids or liposomes, further comprise stabilizers, preservatives, excipients. Typical lipids are the phospholipids and phosphatidyl cholines (lecithins), both natural and synthetic. Methods of forming liposomes are known in the art and are described in Prescott, Ed., “Methods in Cell Biology”, Volume XIV, Academic Press, New York, N.Y., p. 33 et seq. (1976), which is incorporated herein by reference.

Injectable preparations (such as, for example, sterile injectable aqueous or oleaginous suspensions) may be formulated using exemplary methods and materials, such as, for example, suitable dispersing, wetting, and suspension agents. The sterile injectable preparation may also be a solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

### 2. Solid Dosage Forms

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, such as, for example, lubricating agents (e.g., magnesium stearate). In the case of capsules, tablets, and pills, the dosage forms may also comprise buffering agents. Tablets and pills can additionally be prepared with enteric coatings.

### B. Carriers, Vehicles, and Excipients

In some embodiments, the provided compositions, such as pharmaceutical compositions, further comprise a carrier or an excipient, such as a pharmaceutically acceptable carrier (or vehicle) or excipient. In some embodiments, the pharmaceutically acceptable carrier (or vehicle) or excipient comprises one or more conventional nontoxic carrier(s) (or vehicle(s)) or excipient(s). Exemplary pharmaceutically acceptable carriers and excipients are known. Non-limiting examples include processing agents and drug delivery modifiers, such as calcium phosphate, magnesium stearate, talc, monosaccharides, disaccharides, starch, gelatin, cellulose, methyl cellulose, sodium carboxymethyl cellulose, dextrose, cyclodextrins, such as hydroxypropyl-β-cyclodextrin, polyvinylpyrrolidone, low melting waxes, ion exchange resins as well as combinations of any two or more thereof. Pharmaceutically acceptable excipients are described in “Remington's Pharmaceutical Sciences”, 18th edition, A. R. Gennaro, Ed., Mack Pub. Co. New Jersey (1991), “Pharmaceutical Formulation Development of Peptides and Proteins, S. Frokjaer and L. Hovgaard, Eds., Taylor & Francis (2000), and “Handbook of Pharmaceutical Excipients, 3rd edition, A. Kibbe, Ed., Pharmaceutical Press (2000), all of which are incorporated herein by reference.

### IV. USE OF BCMA VARIANT MOLECULES

Provided herein are methods of using any of the provided variant BCMA molecules, such as those comprising variant BCMA molecules comprising a mutated CRD relative to huBCMA CRD (SEQ ID NO:1). In some embodiments, provided herein are methods of using pharmaceutical compositions comprising variant BCMA molecules comprising a mutated CRD relative to huBCMA CRD (SEQ ID NO:1). In some embodiments, provided herein are methods of using a variant BCMA polypeptide and/or a BCMA-Ig-Fc fusion polypeptide. In some embodiments, provided herein are methods of using a BCMA-Ig-Fc fusion polypeptide dimer, and/or a BCMA-Ig-Fc fusion polypeptide conjugate. In some embodiments, provided herein are methods of using any of the provided variant BCMA molecules and/or any combination thereof.

In some embodiments, the provided variant BCMA molecules, and compositions comprising the same, are used to inhibit interactions of one or more of receptor(s) BCMA, TACI, and BR3 with ligands BAFF and APRIL. In some embodiments, the provided variant BCMA molecules, and compositions comprising the same, are used to inhibit interactions of one or more of endogenous receptor(s) BCMA, TACI, and BR3 with endogenous ligands BAFF and APRIL. In some embodiments, the provided variant BCMA molecules, and compositions comprising the same, are used to inhibit interactions of one or more of endogenous human receptor(s) BCMA, TACI, and BR3 with endogenous human ligands BAFF and APRIL. In some embodiments, inhibiting interactions between the one or more of the described receptor(s) with ligands BAFF and APRIL suppresses the activity of BAFF and APRIL.

In some embodiments, the provided variant BCMA molecules, and compositions of the same, are used to bind, such as specifically bind to BAFF and APRIL. In some embodiments, the provided variant BCMA molecules, and compositions of the same, are used to bind, such as specifically bind to endogenous BAFF and APRIL. In some embodiments, the provided variant BCMA molecules, and compositions of the same, are used to bind, such as specifically bind to endogenous human BAFF and APRIL. In some embodiments, binding to BAFF and APRIL, such as specifically binding, suppresses the activity of BAFF and APRIL.

### A. Method of Treatment and Therapeutic Uses

In some aspects, provided herein are methods of administering and uses, such as therapeutic and prophylactic uses, of any of the variant BCMA-binding molecules provided herein, including a fusion protein, a dimer, and a conjugate thereof and/or compositions comprising the same, or polynucleotides, vectors, or cells as provided herein. In some embodiments, the provided therapeutic methods and uses, for example, involving administration of the molecules or compositions containing the same, to a subject having a disease, condition, or disorder associated with BCMA, such as a disease, condition, or disorder associated with BCMA expression, and/or in which cells or tissues express, e.g., specifically express, BCMA. In some embodiments, the variant BCMA molecules, compositions, polynucleotides, vectors, or cells as provided herein are administered in an effective amount to effect treatment of the disease or disorder.

Also provided herein are uses of BCMA variant molecules, including a fusion protein, a dimer, and a conjugate thereof, or polynucleotides, vectors, or cells as provided herein, in such methods and treatments, and in the preparation of a medicament in order to carry out such therapeutic methods. In some embodiments, the methods are carried out by administering the variant BCMA molecules, or compositions comprising the same or polynucleotides, vectors, or cells as provided herein, to the subject having, having had, or suspected of having the disease or condition. In some embodiments, the methods thereby treat the disease or condition or disorder in the subject. Also provided herein are use of any of the compositions, such as pharmaceutical compositions provided herein, for the treatment of a disease or disorder associated with BCMA, such as for use in a treatment regimen.

In some embodiments, the provided variant BCMA molecules are used for therapeutic treatment of a subject. In some embodiments, the subject to receive therapeutic treatment displays symptoms or signs of pathology, disease, or disorder, in which treatment is administered to the subject for the purpose of diminishing or eliminating those signs or symptoms. In some embodiments, the provided variant BCMA molecules are used for prophylactic treatment of a subject. In some embodiments, the subject to receive prophylactic treatment does not display signs or symptoms of, or displays only early signs or symptoms of, a disease, pathology, or disorder. In some embodiments, prophylactic treatment is administered for the purpose of preventing or decreasing the risk of developing the disease, pathology, or disorder. In some embodiments, the provided variant BCMA molecules are administered in therapeutically effective amount. In some embodiments, a therapeutically effective amount is a dosage or amount of a substance sufficient to produce a desired result. In some embodiments, the desired result may comprise an objective or subjective improvement in the recipient of the dosage or amount. In some embodiments, the desired result may comprise a measurable, detectable or testable induction, promotion, enhancement, or modulation of an immune response in a subject.

Suppression of BAFF and APRIL affects B cell development, survival, and function. Provided herein are methods of preventing or treating a B-cell or antibody-mediated disorder, comprising administering to a subject any of the provided variant BCMA molecules, such as polypeptides, fusion polypeptides, dimers, conjugates, polynucleotides, vectors, cells, pharmaceutical compositions. In some embodiments, the variant BCMA molecule is or comprises a variant BCMA polypeptide, a BCMA-Ig-Fc fusion protein or dimer, or a conjugate thereof. In some embodiments, provided are methods of administering a composition, such as a pharmaceutical composition, comprising any of the described variant BCMA molecules and/or any combination thereof. In some embodiments, the variant BCMA molecules is administered at a therapeutically effective amount. In some embodiments, a therapeutically effective amount is a dosage or amount of a substance sufficient to produce a desired result. In some embodiments, the desired result may comprise an objective or subjective improvement in the recipient of the dosage or amount. In some embodiments, the desired result may comprise a measurable, detectable or testable induction, promotion, enhancement, or modulation of an immune response in a subject. In some embodiments, the subject is a non-human mammal or is human. In some embodiments, the subject has a B-cell or antibody-mediated disorder. In some embodiments, the B-cell or antibody-mediated disorder is an autoimmune disease, a transplant rejection, or a B-cell malignancy.

In some embodiments, provided herein is a method of treatment of a subject having a B-cell- or antibody-mediated disease or disorder comprising administering a therapeutically effective amount of a provided variant BCMA molecules, polypeptides, fusion polypeptides, dimers, conjugates, polynucleotides, vectors, cells, or pharmaceutical compositions. In some embodiments, provided herein is use of a provided variant BCMA molecule in the manufacture of a medicament in a treatment of a subject having a B-cell- or antibody-mediated disease or disorder. In some embodiments, provided herein is use of a provided variant BCMA molecule to treat a subject having a B-cell- or antibody-mediated disease or disorder. In some embodiments, the variant BCMA molecule is or comprises a variant BCMA polypeptide, a BCMA-Ig-Fc fusion protein or dimer, or a conjugate thereof. In some embodiments, provided are methods of administering a composition, such as a pharmaceutical composition, comprising any of the described variant BCMA molecules and/or any combination thereof.

In some embodiments, the B-cell- or antibody-mediated disease or disorder is an autoimmune disease or disorder. In some embodiments, the autoimmune disease or disorder is an immune-mediated disease or disorder of the subject's tissues, bone, joints, blood vessels, thyroid, kidneys, nervous system, brain, lungs, and/or skin. In some embodiments, the autoimmune disease or disorder is a renal disease or disorder, lupus, arthritis, a spondyloarthropathic disorder, a vasculitis disorder, a hemolytic anemia disorder, a thrombocytopenia disorder, a thyroiditis disorder, a demyelinating disease of the central and/or peripheral nervous system, inflammatory and/or fibrotic lung disorder, a skin disorder, or an allergic disorder.

In some embodiments, the autoimmune disease or disorder is systemic lupus erythematosus (SLE) and/or lupus nephritis (LN), IgA nephropathy (Berger's disease), Goodpasture syndrome, anti-neutrophil cytoplasmic antibody (ANCA) associated vasculitis, Henoch-Schonlein purpura, polyarteritis nodosa (PAN), sarcoidosis of the kidneys, rheumatoid arthritis, juvenile chronic arthritis, arthritis associated with inflammatory bowel disease, ankylosing spondylitis, spondylitis associated with psoriasis, juvenile onset spondyloarthropathy, undifferentiated spondyloarthropathy, Reiter's syndrome, scleroderma, Sjogren's syndrome, systemic necrotizing vasculitis, polyarteritis nodosa, allergic angiitis and granulomatosis, polyangiitis, Wegener's granulomatosis, lymphomatoid granulomatosis, mucocutaneous lymph node syndrome (MLNS or Kawasaki's disease), isolated central nervous system vasculitis, Behet's disease, thromboangiitis obliterans (Buerger's disease), cutaneous necrotizing venulitis, sarcoidosis, autoimmune hemolytic anemia, immune pancytopenia, paroxysmal nocturnal hemoglobinuria, thrombocytopenic purpura, immune-mediated thrombocytopenia, Grave's disease, Hashimoto's thyroiditis, juvenile lymphocytic thyroiditis, atrophic thyroiditis, type I diabetes mellitus, glomerulonephritis and tubulointerstitial nephritis, multiple sclerosis (MS), idiopathic demyelinating polyneuropathy, Guillain-Barre syndrome, chronic inflammatory demyelinating polyneuropathy, eosinophilic pneumonia, idiopathic pulmonary fibrosis, hypersensitivity pneumonitis, bullous skin disease, erythema multiforme, contact dermatitis, asthma, allergic rhinitis, atopic dermatitis, food hypersensitivity, or urticaria.

In some embodiments, the renal disease or disorder is selected from systemic lupus erythematosus (SLE) and/or lupus nephritis (LN), IgA nephropathy (Berger's disease), Goodpasture syndrome, anti-neutrophil cytoplasmic antibody (ANCA) associated vasculitis, Henoch-Schonlein purpura, Polyarteritis nodosa (PAN), or sarcoidosis of the kidneys. In some embodiments, the renal disease or disorder is selected from systemic lupus erythematosus (SLE) and/or lupus nephritis (LN), or IgA nephropathy (Berger's disease).

In some embodiments, the B-cell- or antibody-mediated disease or disorder is a tissue or organ transplant rejection. In some embodiments, the tissue or organ transplant rejection is acute or chronic B-cell or antibody-mediated rejection of allografts of tissues consisting of bone marrow, stem cell, skin, and solid organs, acute or chronic graft versus host disease (GVHD), antibody-mediated rejection (AMR) of solid organs, hyperacute organ transplant rejection, acute organ transplant rejection, and/or chronic organ transplant rejection.

In some embodiments, wherein the B-cell- or antibody-mediated disease or disorder is a B-cell malignancy. In some embodiments, the B-cell malignancy is non-Hodgkin's lymphoma, multiple myeloma (MM), B-chronic lymphocytic leukemia, plasmacytoma, macroglobulinemia, or Waldenstrom's macroglobulinemia (WM).

### B. Dosing

In some embodiments, the provided variant BCMA molecules and/or compositions thereof can be administered as a sole active agent. In some aspects, the specific dose level for any particular subject or patient may depend upon a variety of factors including the activity of the specific compound employed, the age, body weight, general health, route of administration, severity of the disorder, rate of excretion. The therapeutically effective amount for a given situation can be readily determined by routine experimentation and is within the skill and judgment of the ordinary clinician. The terms “therapeutically effective amount” and “therapeutically effective dose” are used interchangeably herein to refer to an amount of a compound that results in prevention or amelioration of systems in a patient or a desired biological outcome. The terms “subject” and “patient” are used interchangeably herein to refer to a mammal, such as a human, non-human primate (e.g., a baboon, an orangutan, a monkey, a gorilla), or a non-primate mammal (e.g., a mouse, a rat, a dog, a pig).

In some embodiments, a therapeutically effective dose of a provided variant BCMA molecule ranges from at or about 0.1 μg/kg/day to at or about 20 mg/kg/day, about 10 μg/kg/day to at or about 1 mg/kg/day, or from at or about 100 μg/kg/day to at or about 1 mg/kg/day. In some embodiments, the variant BCMA molecule is a variant BCMA polypeptide, a fusion protein or dimer thereof, or conjugate or composition, such as a pharmaceutical composition, thereof. In some embodiments, any combination of the aforementioned molecules is administered at the provided doses. In some embodiments, one dose of the provided variant BCMA molecules is delivered to a subject. In some embodiments, more than one dose of the provided variant BCMA molecules is delivered to a subject.

In some embodiments, a provided variant BCMA molecule can be administered at a recommended maximum clinical dosage and/or at lower dosages. In some embodiments, dosage levels of the any of the provided variant BCMA molecules can be varied to obtain a desired therapeutic response depending, for example, on the route of administration, severity of the disease, the response of the patient. In some embodiments, the BCMA molecule is a variant BCMA polypeptide, a fusion protein or dimer thereof, or conjugate or composition, such as a pharmaceutical composition, thereof. In some embodiments, any combination of the aforementioned molecules is administered at the provided dosages.

In some embodiments, the provided variant BCMA molecules and/or compositions thereof can be administered in combination with one or more other agents. See, e.g., US 2008/0260737, which is incorporated herein by reference. In some embodiments, suitable agents include non-steroidal anti-inflammatory drugs (NSAIDs), glucocorticoid, prednisone, a disease-modifying anti-rheumatic drug (DMARD) (for example, hydroxychloroquine, sulfasalazine, methotrexate, leflunomide, etanercept, infliximab, rituximab, azathioprine, D-penicillamine, Gold (oral or intramuscular), minocycline, cyclosporine, retinoids, Staphylococcal protein A immunoadsorption, topical treatments (such as, for example, steroids, anthralin, calcipotriene, clobetasol, tazarotene) and the like. In some embodiments, the combination comprising a variant BCMA molecule comprising a mutated CRD relative to huBCMA CRD (SEQ ID NO:1) can be administered as separate compositions. In some embodiments, the separate compositions may be administered at the same time or at different times. In some embodiments, the combination comprising a variant BCMA molecule comprising a mutated CRD relative to huBCMA CRD (SEQ ID NO:1) can be administered as a single dosage form containing the two or more agents.

### V. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”. The term “about” can also encompass variations, which can be up to ±5%, but can also be ±4%, 3%, 2%, 1%, etc. Whether or not modified by the term “about,” the claims include equivalents to the quantities.

As used herein, “domain” (typically a sequence of three or more, generally 5 or 7 or more amino acids, such as 10 to 200 amino acid residues) refers to a portion of a molecule, such as a protein or encoding nucleic acid, that is structurally and/or functionally distinct from other portions of the molecule and is identifiable. For example, domains include those portions of a polypeptide chain that can form an independently folded structure within a protein made up of one or more structural motifs and/or that is recognized by virtue of a functional activity, such as binding activity. A protein can have one, or more than one, distinct domains. For example, a domain can be identified, defined or distinguished by homology of the primary sequence or structure to related family members, such as homology to motifs, such as the cysteine rich motif of TNF receptors. In another example, a domain can be distinguished by its function, such as an ability to interact with a biomolecule. A domain independently can exhibit a biological function or activity such that the domain independently or fused to another molecule can perform an activity, such as, for example binding. A domain can be a linear sequence of amino acids or a non-linear sequence of amino acids. Many polypeptides contain a plurality of domains. For exemplification herein, definitions are provided, but particular domains can be recognized in some aspects by name. If needed appropriate software can be employed to identify domains.

The term “extracellular domain” or “ectodomain,” which can be used interchangeably, as used herein refers to the region of a membrane protein, such as a transmembrane protein, that lies outside the vesicular membrane. Extracellular domains often comprise binding domains that specifically bind to ligands or cell surface receptors, such as via a binding domain that specifically binds to the ligand or cell surface receptor.

The term “endodomain” or “intracellular domain,” which can be used interchangeably, as used herein refers to the region found in some membrane proteins, such as transmembrane proteins, that extends into the interior space defined by the cell surface membrane. In mammalian cells, the endodomain is the cytoplasmic region of the membrane protein. In cells, the endodomain interacts with intracellular constituents and can be play a role in signal transduction and thus, in some cases, can be an intracellular signaling domain. The endodomain of a cellular transmembrane protein is alternately referred to as a cytoplasmic domain, which, in some cases, can be a cytoplasmic signaling domain.

The term “transmembrane domain” as used herein means a domain found in a membrane protein that substantially or completely spans a lipid bilayer such as those lipid bilayers found in a biological membrane such as a mammalian cell, or in an artificial construct such as a liposome. Transmembrane domains are generally predictable from their amino acid sequence via any number of commercially available bioinformatics software applications on the basis of their elevated hydrophobicity relative to regions of the protein that interact with aqueous environments (e.g., cytosol, extracellular fluid). A transmembrane domain is often a hydrophobic alpha helix that spans the membrane. A transmembrane protein can pass through the both layers of the lipid bilayer once or multiple times.

The term “specifically binds” as used herein means the ability of a protein, under specific binding conditions, to bind to a target protein such that its affinity or avidity is at least 5 times as great, but optionally at least 10, 20, 30, 40, 50, 100, 250 or 500 times as great, or even at least 1000 times as great as the average affinity or avidity of the same protein to a collection of random peptides or polypeptides of sufficient statistical size. A specifically binding protein need not bind exclusively to a single target molecule but may specifically bind to a non-target molecule due to similarity in structural conformation between the target and non-target (e.g., paralogs or orthologs). Those of skill will recognize that specific binding to a molecule having the same function in a different species of animal (i.e., ortholog) or to a non-target molecule having a substantially similar epitope as the target molecule (e.g., paralog) is possible and does not detract from the specificity of binding which is determined relative to a statistically valid collection of unique non-targets (e.g., random polypeptides). Thus, a polypeptide may specifically bind to more than one distinct species of target molecule due to cross-reactivity. Solid-phase ELISA immunoassays or Biacore measurements can be used to determine specific binding between two proteins.

As used herein, “variant BCMA polypeptide” or “protein” refers to a polypeptide comprising a BCMA cysteine rich domain (CRD) amino acid sequence that differs in sequence with respect to one or more amino acid position(s) from the corresponding CRD amino acid sequence of wild-type human BCMA extracellular domain SEQ ID NO:1), e.g., by substitution, deletion, or insertion. The terms “polypeptide,” “peptide,” and “protein,” are used interchangeably herein to refer to a polymer of amino acids that may be naturally occurring amino acids or artificial amino acid analogues.

As used herein, the term “variant BCMA molecule” refers to molecules comprising a provided BCMA polypeptide variant and includes variant BCMA polypeptides, variant BCMA fusion polypeptides (including, for example, bispecific fusion polypeptides, variant BCMA-Ig fusion polypeptides) and dimers thereof (e.g., a dimer of variant BCMA-Ig fusion polypeptides covalently linked via disulfide bonds through the Ig component of the fusion polypeptide) as well as conjugates thereof. In various embodiments, the term “variant BCMA molecule” refers to a polypeptide comprising a provided variant BCMA CRD or variant BCMA ECD

As used herein, the terms “mutant” and “variant” with respect to a polypeptide, both refer to a polypeptide sequence that differs in one or more amino acid residues from the polypeptide sequence of a parent or reference polypeptide (such as, e.g., a wild-type (WT) polypeptide sequence). In one aspect, a mutant polypeptide comprises a polypeptide sequence which differs from the polypeptide sequence of a parent or reference polypeptide in from at or about 1%, 2%, 3%, 4%, 5%, 6%, 7%, 8%, 9%, 10%, 11%, 12%, 13%, 14%, 15%, 20%, 30%, 40%, 50% or more of the total number of residues of the parent or reference polypeptide sequence. In another aspect, a mutant polypeptide comprises a polypeptide sequence that has at least at or about 50%, 60%, 70%, 80%, 85%, 86%, 87%, 88%, 89%, 90%, 91%, 92%, 93%, 94%, 95%, 96%, 97%, 98%, or 99% sequence identity to the polypeptide sequence of a parent or reference polypeptide. In another aspect, a mutant polypeptide comprises a polypeptide sequence that differs from the polypeptide sequence of a parent or reference polypeptide in from 1 to 50 or more amino acid residues (e.g., 1, 2, 3, 4, 5, 6, 7, 8, 9, 10, 11, 12, 13, 14, 15, 16, 17, 18, 19, 20, 25, 30, 35, 40, 45 or 50 amino acid residues). A mutant polypeptide may comprise a polypeptide sequence that differs from the polypeptide sequence of a parent or reference polypeptide by, e.g., the deletion, addition, or substitution of one or more amino acid residues (e.g., 1, 2, 3, 4, 5, 6, 7, 8, 9, 10, 11, 12, 13, 14, 15, 16, 17, 18, 19, 20 or more amino acid residues) of the parent or reference polypeptide, or any combination of such deletion(s), addition(s), and/or substitution(s). The reference or parent polypeptide may itself be a mutant polypeptide.

With respect to the numbering of positions in a given amino acid polymer or nucleic acid polymer, the term “corresponds to,” is in “reference to,” or is “relative to” the numbering of a selected amino acid polymer or nucleic acid polymer refers to the position of any given polymer component (e.g., amino acid, nucleotide, also referred to generically as a “residue”) as designated by reference to the same or to an equivalent position in the selected amino acid or nucleic acid polymer, rather than by the actual numerical position of the component in the given polymer. Thus, for example, the numbering of a given amino acid position in a given polypeptide sequence corresponds to the same or equivalent amino acid position in a selected polypeptide sequence used as a reference sequence.

An “equivalent position” (for example, an “equivalent amino acid position” or “equivalent nucleic acid position” or “equivalent residue position”) is defined herein as a position (such as, an amino acid position or nucleic acid position or residue position) of a test polypeptide (or test polynucleotide) sequence which aligns with a corresponding position of a reference polypeptide (or reference polynucleotide) sequence, when optimally aligned using an alignment algorithm as described herein. The equivalent amino acid position of the test polypeptide need not have the same numerical position number as the corresponding position of the reference polypeptide; likewise, the equivalent nucleic acid position of the test polynucleotide need not have the same numerical position number as the corresponding position of the reference polynucleotide.

Two polypeptide sequences are “optimally aligned” or in “optimal alignment” when they are aligned using defined parameters, i.e., a defined amino acid substitution matrix, gap existence penalty (also termed gap open penalty), and gap extension penalty, so as to arrive at the highest similarity score possible for that pair of sequences. The BLOSUM62 matrix (Henikoff and Henikoff Proc. Natl. Acad. Sci. USA 1992; 89(22):10915-10919) is often used as a default scoring substitution matrix in polypeptide sequence alignment algorithms (such as BLASTP). The gap existence penalty is imposed for the introduction of a single amino acid gap in one of the aligned sequences, and the gap extension penalty is imposed for each residue position in the gap. Unless otherwise stated, alignment parameters employed herein are: BLOSUM62 scoring matrix, gap existence penalty=11, and gap extension penalty=1. The alignment score is defined by the amino acid positions of each sequence at which the alignment begins and ends (e.g. the alignment window), and optionally by the insertion of a gap or multiple gaps into one or both sequences, so as to arrive at the highest possible similarity score.

With respect to the determination of an amino acid position by optimal alignment with a reference sequence, the amino acid position in a test amino acid sequence corresponds to the position in the reference sequence with which the residue is paired in the alignment. The “position” is denoted by a number that sequentially identifies each amino acid in the reference sequence based on its position relative to the N-terminus. Owing to deletions, insertions, truncations, fusions, and similar modifications that must be taken into account when determining an optimal alignment, in general the amino acid residue number in a test sequence is determined by simply counting from the N-terminal will not necessarily be the same as the number of its corresponding position in the reference sequence. For example, in a case where there is a deletion in an aligned test sequence, there will be no amino acid that corresponds to a position in the reference sequence at the site of deletion. Where there is an insertion in an aligned reference sequence, that insertion will not correspond to any amino acid position in the reference sequence. In the case of truncations or fusions, there can be stretches of amino acids in either the reference or aligned sequence that do not correspond to any amino acid in the corresponding sequence.

The term “corresponding control protein” or “reference polypeptide” refers herein to a protein that has an amino acid sequence that is the same as the variant BCMA polypeptide amino acid sequence with the exception that the BCMA ECD component comprises either the [S40G]huBCMA CRD (SEQ ID NO:237) or the wild-type huBCMA CRD (SEQ ID NO:1); or the [S40G]huBCMA ECD (SEQ ID NO:224) or the wild-type huBCMA ECD (SEQ ID NO:152). The terms “[S40G]huBCMA”, “[S40G]huBCMA ECD”, “BCMA[S40G]”, and “[S40G]BCMA” are used interchangeably herein to refer to a polypeptide comprising SEQ ID NO:224. The term “[S40G]huBCMA CRD” is used interchangeably herein to refer to a polypeptide comprising SEQ ID NO:237. As described above, a corresponding control protein may therefore be a corresponding BCMA polypeptide, a corresponding BCMA fusion polypeptide, a corresponding BCMA fusion polypeptide dimer, or a corresponding BCMA conjugate, in which the BCMA ECD component is either human wild-type BCMA ECD (SEQ ID NO:152) or the [S40G]huBCMA ECD amino acid sequence (SEQ ID NO:224); or in which the BCMA CRD component is either the [S40G]huBCMA CRD (SEQ ID NO:237) or the wild-type huBCMA CRD (SEQ ID NO:1). Control protein comprising the [S40G]huBCMA ECD or CRD is also referred to herein by the terms “human BCMA surrogate control protein” and “human BCMA surrogate control” which refer to a polypeptide comprising an amino acid sequence that encodes the [S40G] substituted form of human BCMA extracellular domain. [S40G]huBCMA ECD or CRD contains a single mutation ([S40G]) within the BCMA ECD or CRD amino acid sequence relative to the wild-type huBCMA ECD or CRD. A further illustrative control protein comprising the [S40G]huBCMA ECD or CRD includes mature [S40G]BCMA ECD or CRD-IgG2 Fc fusion protein (PIg18). As described in more detail below, the [S40G] substitution removes an N-glycosylation site that might be recognized by a glycosylating host cell and appears to yield a more homogeneous protein due to the absence of different glycoforms. The [S40G] substitution appears to not impact huBAFF and huAPRIL binding properties as described herein.

The term “non-polypeptide conjugation moiety” refers herein to a non-polypeptide polymer, a sugar moiety, or a non-polymeric lipophilic moiety.

The term “non-polypeptide polymer” refers herein to a water soluble polymer that may be a natural or synthetic polymer (e.g., homopolymer, copolymer, terpolymer), that is not a peptide, polypeptide, or protein.

The term “sugar moiety” refers herein to a carbohydrate molecule attached by an in vivo or in vitro glycosylation process, such as an N- or O-glycosylation process.

The term “therapeutic treatment” refers herein to a treatment administered to a subject who displays symptoms or signs of pathology, disease, or disorder, in which treatment is administered to the subject for the purpose of diminishing or eliminating those signs or symptoms.

The term “prophylactic treatment” refers herein to a treatment administered to a subject who does not display signs or symptoms of, or displays only early signs or symptoms of, a disease, pathology, or disorder. Such treatment is administered for the purpose of preventing or decreasing the risk of developing the disease, pathology, or disorder.

The term “therapeutically effective amount” refers herein to a dosage or amount of a substance sufficient to produce a desired result. The desired result may comprise an objective or subjective improvement in the recipient of the dosage or amount. For example, the desired result may comprise a measurable, detectable or testable induction, promotion, enhancement, or modulation of an immune response in a subject.

### VI. EXEMPLARY EMBODIMENTS

Among the provided embodiments are:
1. A variant B cell maturation antigen (BCMA) polypeptide, comprising a variant cysteine rich domain (CRD) comprising at least one amino acid substitution(s) selected from among: (1) a histidine, an arginine, a proline, or an asparagine at position 12 (S12H, S12R, S12P, or S12N); (2) an isoleucine or a valine at position 14 (L14I or L14V); (3) an arginine or an asparagine at position 15 (H15R or H15N); (4) a serine at position 16 (A16S); and (5) a valine, an isoleucine, or an alanine at position 22 (L22V, L22I, or L22A), with reference to the amino acid positions of SEQ ID NO:1.
2. The variant BCMA polypeptide of embodiment 1, wherein the variant CRD comprises at least three amino acid substitutions selected from among: (1) a histidine, an arginine, a proline, or an asparagine at position 12 (S12H, S12R, S12P, or S12N); (2) an isoleucine or a valine at position 14 (L14I or L14V); (3) an arginine or an asparagine at position 15 (H15R or H15N); (4) a serine at position 16 (A16S); and (5) a valine, an isoleucine, or an alanine at position 22 (L22V, L22I, or L22A), with reference to the amino acid positions of SEQ ID NO:1.
3. The variant BCMA polypeptide of embodiment 1 or 2, wherein the variant CRD comprises at least three amino acid substitutions selected from among: (1) a histidine or an arginine at position 12 (S12H or S12R); (2) an isoleucine or a valine at position 14 (L14I or L14V); (3) an arginine at position 15 (H15R); and (4) a valine at position 22 (L22V), with reference to the amino acid positions of SEQ ID NO:1.
4. The variant BCMA polypeptide of any one of embodiments 1-3, wherein the variant CRD further comprises at least one modification selected from among: (1) a deletion at residue 38 (N38del) or an amino acid residue that is not asparagine at position 38 (N38X, wherein X is any amino acid residue that is not asparagine) and (2) an amino acid residue that is not serine or threonine at position 40 (S40X, wherein X is any amino acid residue that is not serine or threonine), with reference to the amino acid positions of SEQ ID NO:1.
5. The variant BCMA polypeptide of any one of embodiments 1-4, wherein the variant CRD further comprises a glycine at residue 40 (S40G), with reference to the amino acid positions of SEQ ID NO:1.
6. The variant BCMA polypeptide of any one of embodiments 1-5, wherein the variant CRD comprises at least at or about 85%, 86%, 87%, 88%, 89%, 90%, 91%, 92%, 93%, 94%, 95%, 96%, 97%, 98%, or 99% sequence identity to SEQ ID NO:1.
7. The variant BCMA polypeptide of any one of embodiments 1-6, wherein the variant CRD comprises at least at or about 90% sequence identity to SEQ ID NO:1.
8. The variant BCMA polypeptide of any one of embodiments 1-7, wherein the variant CRD comprises at least at or about 95% sequence identity to SEQ ID NO:1.
9. The variant BCMA polypeptide of any one of embodiments 1-8, wherein the variant CRD comprises at least at or about 99% sequence identity to SEQ ID NO:1.
10. The variant BCMA polypeptide of any one of embodiments 1-5, wherein the variant CRD comprises at least at or about 90% sequence identity to the sequence set forth in any one of SEQ ID NOs:3-146.
11. The variant BCMA polypeptide of any one of embodiments 1-5, wherein the variant CRD comprises at least at or about 90% sequence identity to the sequence set forth in any one of SEQ ID NO:3, SEQ ID NO:4, SEQ ID NO:6, SEQ ID NO:8, SEQ ID NO:9, or SEQ ID NO:19.
12. The variant BCMA polypeptide of any one of embodiments 1-5, wherein the variant CRD comprises the sequence set forth in any one of SEQ ID NO:3, SEQ ID NO:4, SEQ ID NO:6, SEQ ID NO:8, SEQ ID NO:9, or SEQ ID NO:19.
13. The variant BCMA polypeptide of any one of embodiments 1-5, wherein the variant CRD comprises at least three amino acid substitutions selected from the group consisting of: (1) a histidine at position 12 (S12H); (2) an isoleucine at position 14 (L14I); (3) an arginine at position 15 (H15R or H15N); and (4) and a serine to glycine mutation at residue 40 (S40G), with reference to the amino acid positions of SEQ ID NO:1, and wherein the variant CRD comprises at least at or about 90% sequence identity to SEQ ID NO:1.
14. The variant BCMA polypeptide of any one of embodiments 1-5, wherein the variant CRD comprises each of the following amino acid substitutions: (1) a histidine at position 12 (S12H); (2) an isoleucine at position 14 (L14I); (3) an arginine at position 15 (H15R); and (4) a serine to glycine mutation at residue 40 (S40G), with reference to the amino acid positions of SEQ ID NO:1, and wherein the variant CRD comprises at least at or about 90% sequence identity to SEQ ID NO: 1.
15. The variant BCMA polypeptide of any one of embodiments 1-14, wherein the variant CRD comprises at least at or about 90% sequence identity to SEQ ID NO:3.
16. The variant BCMA polypeptide of any one of embodiments 1-15, wherein the variant CRD comprises the sequence set forth in SEQ ID NO:3.
17. The variant BCMA polypeptide of any one of embodiments 1-12, wherein the variant CRD comprises at least at or about 90% sequence identity to SEQ ID NO:4.
18. The variant BCMA polypeptide of any one of embodiments 1-12 and 17, wherein the variant CRD comprises the sequence set forth in SEQ ID NO:4.
19. The variant BCMA polypeptide of any one of embodiments 1-12, wherein the variant CRD comprises at least at or about 90% sequence identity to SEQ ID NO:8.
20. The variant BCMA polypeptide of any one of embodiments 1-12 and 19, wherein the variant CRD comprises the sequence set forth in SEQ ID NO:8.
21. The variant BCMA polypeptide of any one of embodiments 1-12, wherein the variant CRD comprises at least at or about 90% sequence identity to SEQ ID NO:9.
22. The variant BCMA polypeptide of any one of embodiments 1-12 and 21, wherein the variant CRD comprises the sequence set forth in SEQ ID NO:9.
23. The variant BCMA polypeptide of any one of embodiments 1-12, wherein the variant CRD comprises at least at or about 90% sequence identity to SEQ ID NO:19.
24. The variant BCMA polypeptide of any one of embodiments 1-12 and 23, wherein the variant CRD comprises the sequence set forth in SEQ ID NO:19.
25. A fusion polypeptide, comprising the variant BCMA polypeptide of any one of embodiments 1-24, and an additional polypeptide.
26. The fusion polypeptide of embodiment 25, wherein the additional polypeptide is an immunoglobulin (Ig) Fc polypeptide.
27. A fusion polypeptide, comprising the variant BCMA polypeptide of any one of embodiments 1-24, and an immunoglobulin (Ig) Fc polypeptide.
28. A fusion polypeptide, comprising: a variant B cell maturation antigen (BCMA) polypeptide, comprising a variant cysteine rich domain (CRD) comprising at least one amino acid substitutions selected from among: (1) a histidine, an arginine, a proline, or an asparagine at position 12 (S12H, S12R, S12P, or S12N); (2) an isoleucine or a valine at position 14 (L14I or L14V); (3) an arginine or an asparagine at position 15 (H15R or H15N); (4) a serine at position 16 (A16S); and (5) a valine, an isoleucine, or an alanine at position 22 (L22V, L22I, or L22A); with reference to the amino acid positions of SEQ ID NO:1; and an immunoglobulin (Ig) Fc polypeptide.
29. The fusion polypeptide of embodiment 28, wherein the variant CRD comprises at least three amino acid substitutions selected from among: (1) a histidine, an arginine, a proline, or an asparagine at position 12 (S12H, S12R, S12P, or S12N); (2) an isoleucine or a valine at position 14 (L14I or L14V); (3) an arginine or an asparagine at position 15 (H15R or H15N); (4) a serine at position 16 (A16S); and (5) a valine, an isoleucine, or an alanine at position 22 (L22V, L22I, or L22A), with reference to the amino acid positions of SEQ ID NO:1.
30. The fusion polypeptide of embodiment 28 or 29, wherein the variant CRD comprises least three amino acid substitutions selected from among: (1) a histidine or an arginine at position 12 (S12H or S12R); (2) an isoleucine or a valine at position 14 (L14I or L14V); (3) an arginine at position 15 (H15R); and (4) a valine at position 22 (L22V), with reference to the amino acid positions of SEQ ID NO:1.
31. The fusion polypeptide of any one of embodiments 28-30, wherein the variant CRD further comprises at least one modification selected from among: (1) a deletion at residue 38 (N38del) or an amino acid residue that is not asparagine at position 38 (N38X, wherein X is any amino acid residue that is not asparagine) and (2) an amino acid residue that is not serine or threonine at position 40 (S40X, wherein X is any amino acid residue that is not serine or threonine), with reference to the amino acid positions of SEQ ID NO:1.
32. The fusion polypeptide of any one of embodiments 28-31, wherein the variant CRD further comprises a glycine at residue 40 (S40G), with reference to the amino acid positions of SEQ ID NO:1.
33. The fusion polypeptide of any one of embodiments 28-32, wherein the variant CRD comprises at least at or about 90% sequence identity to the sequence set forth in any one of SEQ ID NOS:3-146.
34. The fusion polypeptide of any one of embodiments 28-33, wherein the variant CRD comprises at least at or about 90% sequence identity to the sequence set forth in SEQ ID NO:3, SEQ ID NO:4, SEQ ID NO:6, SEQ ID NO:8, SEQ ID NO:9, or SEQ ID NO:19.
35. The fusion polypeptide of any one of embodiments 28-34, wherein the variant CRD comprises the sequence set forth in SEQ ID NO:3, SEQ ID NO:4, SEQ ID NO:6, SEQ ID NO:8, SEQ ID NO:9, or SEQ ID NO:19.
36. The fusion polypeptide of any one of embodiments 28-35, wherein the variant CRD comprises at least at or about 90% sequence identity to SEQ ID NO:3.
37. The fusion polypeptide of any one of embodiments 28-36, wherein the variant CRD comprises the sequence set forth in SEQ ID NO:3.
38. The fusion polypeptide of any one of embodiments 28-35, wherein the variant CRD comprises at least at or about 90% sequence identity to SEQ ID NO:4.
39. The fusion polypeptide of any one of embodiments 28-35 and 38, wherein the variant CRD comprises the sequence set forth in SEQ ID NO:4.
40. The fusion polypeptide of any one of embodiments 28-35, wherein the variant CRD comprises at least at or about 90% sequence identity to SEQ ID NO:8.
41. The fusion polypeptide of any one of embodiments 28-35 and 40, wherein the variant CRD comprises the sequence set forth in SEQ ID NO:8.
42. The fusion polypeptide of any one of embodiments 28-35, wherein the variant CRD comprises at least at or about 90% sequence identity to SEQ ID NO:9.
43. The fusion polypeptide of any one of embodiments 28-35 and 42, wherein the variant CRD comprises the sequence set forth in SEQ ID NO:9.
44. The fusion polypeptide of any one of embodiments 28-35, wherein the variant CRD comprises at least at or about 90% sequence identity to SEQ ID NO:19.
45. The fusion polypeptide of any one of embodiments 28-35 and 44, wherein the variant CRD comprises the sequence set forth in SEQ ID NO:19.
46. The fusion polypeptide of any one of embodiments 26-45, wherein the variant BCMA polypeptide is directly or indirectly fused to the N-terminus or the C-terminus of the Ig Fc polypeptide.
47. The fusion polypeptide of any one of embodiments 26-46, wherein the Ig Fc polypeptide is or is derived from an isotype G immunoglobulin (IgG) or a variant thereof.
48. The fusion polypeptide of any one of embodiments 26-47, wherein the Ig Fc polypeptide is or is derived from an IgG1 Fc, an IgG2 Fc, an IgG3 Fc, or an IgG4 Fc.
49. The fusion polypeptide of any one of embodiments 26-48, wherein the Ig Fc polypeptide is or is derived from a human IgG Fc.
50. The fusion polypeptide of any one of embodiments 26-49, wherein the Ig Fc polypeptide is or is derived from a human IgG1 Fe, a human IgG2 Fe, a human IgG3 Fe, or a human IgG4 Fc.
51. The fusion polypeptide of any one of embodiments 26-50, wherein the Ig Fc polypeptide is or is derived from an IgG1 Fc.
52. The fusion polypeptide of any one of embodiments 26-51, wherein the Ig Fc polypeptide is or is derived from a human IgG1 Fc.
53. The fusion polypeptide of any one of embodiments 26-52, wherein the Ig Fc polypeptide is a human IgG1 Fc and comprises a sequence set forth in SEQ ID NO:168, SEQ ID NO:169, SEQ ID NO:170, SEQ ID NO:226, SEQ ID NO:227, SEQ ID NO:228, SEQ ID NO:229, or SEQ ID NO:230, or a sequence that has at least 90% sequence identity thereto.
54. The fusion polypeptide of any one of embodiments 26-53, wherein the Ig Fc polypeptide is a human IgG1 Fc and comprises a sequence set forth in SEQ ID NO:168, SEQ ID NO:169, SEQ ID NO:170, SEQ ID NO:226, SEQ ID NO:227, SEQ ID NO:228, SEQ ID NO:229, or SEQ ID NO:230.
55. The fusion polypeptide of any one of embodiments 26-50, wherein the Ig Fc polypeptide is or is derived from an IgG2 Fc.
56. The fusion polypeptide of any one of embodiments 26-50 and 55, wherein the Ig Fc polypeptide is or is derived from a human IgG2 Fc.
57. The fusion polypeptide of any one of embodiments 26-50, 55, and 56, wherein the Ig Fc polypeptide is a human IgG2 Fc and comprises a sequence set forth in SEQ ID NO:171, SEQ ID NO:235, or SEQ ID NO:236, or a sequence that has at least 90% sequence identity thereto.
58. The fusion polypeptide of any one of embodiments 26-50 and 55-57, wherein the Ig Fc polypeptide is a human IgG2 Fc and comprises a sequence set forth in SEQ ID NO:171, SEQ ID NO:235, or SEQ ID NO:236.
59. The fusion polypeptide of any one of embodiments 26-50, wherein the Ig Fc polypeptide is or is derived from an IgG4 Fc.
60. The fusion polypeptide of any one of embodiments 26-50 and 59, wherein the Ig Fc polypeptide is or is derived from a human IgG4 Fc.
61. The fusion polypeptide of any one of embodiments 26-50, 59, and 60, wherein the Ig Fc polypeptide is a human IgG4 Fc and comprises a sequence set forth in SEQ ID NO:161, SEQ ID NO:163, SEQ ID NO:172, SEQ ID NO: 231, SEQ ID NO:232, SEQ ID NO:233, or SEQ ID NO:234, or a sequence that has at least 90% sequence identity thereto.
62. The fusion polypeptide of any one of embodiments 26-50 and 59-61, wherein the Ig Fc polypeptide is a human IgG4 Fc and comprises a sequence set forth in SEQ ID NO:161, SEQ ID NO:163, SEQ ID NO:172, SEQ ID NO: 231, SEQ ID NO:232, SEQ ID NO:233, or SEQ ID NO:234.
63. The fusion polypeptide of any one of embodiments 26-50 and 59-62, wherein the Ig Fc polypeptide is a human IgG4 Fc and comprises the sequence set forth in SEQ ID NO:161, or a sequence that has at least 90% sequence identity thereto.
64. The fusion polypeptide of any one of embodiments 26-50 and 59-63, wherein the Ig Fc polypeptide comprises SEQ ID NO:161.
65. The fusion polypeptide of any one of embodiments 26-50 and 59-62, wherein the Ig Fc polypeptide is a human IgG4 Fc and comprises the sequence set forth in SEQ ID NO:163, or a sequence that has at least 90% sequence identity thereto.
66. The fusion polypeptide of any one of embodiments 26-50, 59-62 and 65, wherein the Ig Fc polypeptide comprises SEQ ID NO:163.
67. The fusion polypeptide of any one of embodiments 26-66, wherein the Ig Fc polypeptide comprises an isoleucine or a valine substituted for one, two, three, four, or more native methionine residues.
68. The fusion polypeptide of any one of embodiments 26-37, 46-50, and 59-64, wherein the variant BCMA polypeptide comprises SEQ ID NO:3 and the Ig Fc polypeptide comprises SEQ ID NO:161.
69. The fusion polypeptide of any one of embodiments 26-37, 46-50, 59-62, 65, and 66, wherein the variant BCMA polypeptide comprises SEQ ID NO:3 and the Ig Fc polypeptide comprises SEQ ID NO:163.
70. The fusion polypeptide of any one of embodiments 26-35, 38, 39, 46-50, and 59-64, wherein the variant BCMA polypeptide comprises SEQ ID NO:4 and the Ig Fc polypeptide comprises SEQ ID NO:161.
71. The fusion polypeptide of any one of embodiments 26-35, 38, 39, 46-50, 59-62, 65, and 66, wherein the variant BCMA polypeptide comprises SEQ ID NO:4 and the Ig Fc polypeptide comprises SEQ ID NO:163.
72. The fusion polypeptide of any one of embodiments 26-35, 40, 41, 46-50, and 59-64, wherein the variant BCMA polypeptide comprises SEQ ID NO:8 and the Ig Fc polypeptide comprises SEQ ID NO:161.
73. The fusion polypeptide of any one of embodiments 26-35, 40, 41, 46-50, 59-62, 65, and 66, wherein the variant BCMA polypeptide comprises SEQ ID NO:8 and the Ig Fc polypeptide comprises SEQ ID NO:163.
74. The fusion polypeptide of any one of embodiments 26-35, 42, 43, 46-50, and 59-64, wherein the variant BCMA polypeptide comprises SEQ ID NO:9 and the Ig Fc polypeptide comprises SEQ ID NO:161.
75. The fusion polypeptide of any one of embodiments 26-35, 42, 43, 46-50, 59-62, 65, and 66, wherein the variant BCMA polypeptide comprises SEQ ID NO:9 and the Ig Fc polypeptide comprises SEQ ID NO:163.
76. The fusion polypeptide of any one of embodiments 26-35, 44-50, and 59-64, wherein the variant BCMA polypeptide comprises SEQ ID NO:19 and the Ig Fc polypeptide comprises SEQ ID NO:161.
77. The fusion polypeptide of any one of embodiments 26-35, 44-50, 59-62, 65, and 66, wherein the variant BCMA polypeptide comprises SEQ ID NO:19 and the Ig Fc polypeptide comprises SEQ ID NO:163.
78. The fusion polypeptide of any one of embodiments 27-77, further comprising a peptide linker that links the variant BCMA polypeptide to the Ig Fc polypeptide.
79. The fusion polypeptide of embodiment 78, wherein the peptide linker is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids in length.
80. The fusion polypeptide of embodiment 78 or 79, wherein the peptide linker is 4, 5, 6, or 7 amino acids in length.
81. The fusion polypeptide of any one of embodiments 78-80, wherein the peptide linker comprises residues selected from the group consisting of glycine, serine, alanine, and threonine.
82. The fusion polypeptide of any one of embodiments 78-81, wherein the peptide linker comprises a sequences set forth in any one of SEQ ID NO:156, 158, 175-186, 188-213, GS, GGS and GSA.
83. The fusion polypeptide of any one of embodiments 78-82, wherein the peptide linker comprises SEQ ID NO:156.
84. The fusion polypeptide of any one of embodiments 78-82, wherein the peptide linker comprises SEQ ID NO:158.
85. The fusion polypeptide of any one of embodiments 25-84, wherein the fusion polypeptide comprises, in N- to C-terminal order: the variant BCMA polypeptide, a peptide linker and an Ig Fc polypeptide.
86. The fusion polypeptide of any one of embodiments 25-85, wherein the fusion polypeptide comprises a variant BCMA polypeptide set forth in SEQ ID NO:3, SEQ ID NO:4, SEQ ID NO:8, SEQ ID NO:9, or SEQ ID NO:19; a peptide linker comprising a sequences set forth in any one of SEQ ID NO:156, 158, 175-186, 188-213, GS, GGS and GSA, and an Ig Fc polypeptide comprising a sequence set forth in SEQ ID NO:161, SEQ ID NO:163, SEQ ID NO:172, SEQ ID NO: 231, SEQ ID NO:232, SEQ ID NO:233, or SEQ ID NO:234.
87. The fusion polypeptide of any one of embodiments 25-86, wherein the fusion polypeptide comprises a variant BCMA polypeptide comprising SEQ ID NO:3, a peptide linker comprising SEQ ID NO:156, and an Ig Fc polypeptide comprising SEQ ID NO:161.
88. The fusion polypeptide of any one of embodiments 25-86, wherein the fusion polypeptide comprises a variant BCMA polypeptide comprising SEQ ID NO:3, a peptide linker comprising SEQ ID NO:158, and an Ig Fc polypeptide comprising SEQ ID NO:163.
89. The fusion polypeptide of any one of embodiments 25-86, wherein the fusion polypeptide comprises a variant BCMA polypeptide comprising SEQ ID NO:4, a peptide linker comprising SEQ ID NO:156, and an Ig Fc polypeptide comprising SEQ ID NO:161.
90. The fusion polypeptide of any one of embodiments 25-86, wherein the fusion polypeptide comprises a variant BCMA polypeptide comprising SEQ ID NO:4, a peptide linker comprising SEQ ID NO: 158, and an Ig Fc polypeptide comprising SEQ ID NO: 163.
91. The fusion polypeptide of any one of embodiments 25-86, wherein the fusion polypeptide comprises a variant BCMA polypeptide comprising SEQ ID NO:8, a peptide linker comprising SEQ ID NO:156, and an Ig Fc polypeptide comprising SEQ ID NO:161.
92. The fusion polypeptide of any one of embodiments 25-86, wherein the fusion polypeptide comprises a variant BCMA polypeptide comprising SEQ ID NO:8, a peptide linker comprising SEQ ID NO:158, and an Ig Fc polypeptide comprising SEQ ID NO:163.
93. The fusion polypeptide of any one of embodiments 25-86, wherein the fusion polypeptide comprises a variant BCMA polypeptide comprising SEQ ID NO:9, a peptide linker comprising SEQ ID NO:156, and an Ig Fc polypeptide comprising SEQ ID NO:161.
94. The fusion polypeptide of any one of embodiments 25-86, wherein the fusion polypeptide comprises a variant BCMA polypeptide comprising SEQ ID NO:9, a peptide linker comprising SEQ ID NO: 158, and an Ig Fc polypeptide comprising SEQ ID NO: 163.
95. The fusion polypeptide of any one of embodiments 25-86, wherein the fusion polypeptide comprises a variant BCMA polypeptide comprising SEQ ID NO:19, a peptide linker comprising SEQ ID NO:156, and an Ig Fc polypeptide comprising SEQ ID NO:161.
96. The fusion polypeptide of any one of embodiments 25-86, wherein the fusion polypeptide comprises a variant BCMA polypeptide comprising SEQ ID NO:19, a peptide linker comprising SEQ ID NO:158, and an Ig Fc polypeptide comprising SEQ ID NO:163.
97. The fusion polypeptide of any one of embodiments 25-37, 46-50, 59-69, and 78-88 comprising the sequence set forth in SEQ ID NO: 167 or a sequence that has at least 90% sequence identity thereto.
98. The fusion polypeptide of any one of embodiments 25-37, 46-50, 59-69, and 78-88, comprising the sequence set forth in SEQ ID NO: 167.
99. The fusion polypeptide of any one of embodiments 26-98, comprising a first monomer comprising a first variant BCMA polypeptide directly or indirectly fused to a first Ig Fc polypeptide, and one or more second monomer comprising a second variant BCMA polypeptide directly or indirectly fused to a second Ig Fc polypeptide, wherein the first monomer and the one or more second monomers are fused in tandem.
100. The fusion polypeptide of embodiment 99, wherein the first variant BCMA polypeptide and the second variant BCMA polypeptide are the same.
101. The fusion polypeptide of embodiment 99, wherein the first variant BCMA polypeptide and the second variant BCMA polypeptide are different.
102. The fusion polypeptide of embodiment 99, wherein the first monomer and the second monomer are the same.
103. The fusion polypeptide of embodiment 99, wherein the first monomer and the second monomer are different.
104. A dimer, comprising a first monomer comprising the variant BCMA polypeptide of any of one embodiments 1-24 or the fusion polypeptide of any one of embodiments 25-98 and a second monomer comprising the variant BCMA polypeptide of any one of embodiments 1-24 or the fusion polypeptide of any one of embodiments 25-98.
105. The dimer of embodiment 104, wherein the first monomer comprises a first variant BCMA polypeptide directly or indirectly fused to a first Ig Fc polypeptide, and the second monomer comprises a second variant BCMA polypeptide directly or indirectly fused to a second Ig Fc polypeptide.
106. The dimer of embodiment 104 or 105, wherein the first variant BCMA polypeptide and the second variant BCMA polypeptide are the same.
107. The dimer of embodiment 104 or 105, wherein the first variant BCMA polypeptide and the second variant BCMA polypeptide are different.
108. The dimer of any one of embodiments 105-107, wherein the first Ig Fc polypeptide and the second Ig Fc polypeptide are the same.
109. The dimer of any one of embodiments 105-107, wherein the first Ig Fc polypeptide and the second Ig Fc polypeptide are different.
110. The dimer of any one of embodiments 104-109, wherein the first monomer and the second monomer are the same.
111. The dimer of any one of embodiments 104-109, wherein the first monomer and the second monomer are different.
112. The dimer of any one of embodiments 104-111, wherein the first monomer and the second monomer are linked together by at least one disulfide bond between cysteine residues in the first monomer and the second monomer.
113. The dimer of embodiment 112, wherein the disulfide bond is between cysteine residues of the Ig Fc polypeptide of the first monomer and the Ig Fc polypeptide of the second monomer.
114. A conjugate, comprising the variant BCMA polypeptide of any one of embodiments 1-24, the fusion polypeptide of any one of embodiments 25-103, or the dimer of any one of embodiments 104-113; and an additional moiety that is covalently bound to the variant BCMA polypeptide, the fusion polypeptide, or the dimer.
115. The conjugate of embodiment 114, wherein the additional moiety is selected from a therapeutic moiety, a polymer moiety, a sugar moiety, and a lipophilic moiety.
116. The conjugate of embodiment 114 or 115, wherein the additional moiety is selected from among one or more of a polyalkylene oxide (PAO), a polyalkylene glycol (PAG), a polyethylene glycol (PEG), a monomethoxypolyethylene glycol (mPEG), a polypropylene glycol (PPG), a branched polyethylene glycol having two or more polyethylene glycol chains linked together by a linker group, a polyvinyl alcohol (PVA), a polycarboxylate, a poly(vinylpyrrolidone), a polyethylene-co-maleic acid anhydride, and a dextran.
117. The variant BCMA polypeptide of any one of embodiments 1-24, the fusion polypeptide of any one of embodiments 25-103, the dimer of any one of embodiments 104-113, or the conjugate of any one of embodiments 113-116, wherein the variant BCMA polypeptide, the fusion polypeptide, the dimer, or the conjugate binds B-cell Activating Factor of the TNF family (BAFF) and/or A Proliferation Inducing Ligand (APRIL) or variants thereof.
118. The variant BCMA polypeptide of any one of embodiments 1-24, the fusion polypeptide of any one of embodiments 25-103, the dimer of any one of embodiments 104-113, or the conjugate of any one of embodiments 113-116, or variant BCMA polypeptide, the fusion polypeptide, the dimer, or the conjugate of embodiment 117, wherein:
the variant BCMA polypeptide, the fusion polypeptide, the dimer, or the conjugate, exhibits greater binding affinity for BAFF and/or APRIL, compared to the binding affinity of a reference BCMA polypeptide or a reference binding molecule; and/or
the variant BCMA polypeptide, the fusion polypeptide, the dimer, or the conjugate, exhibits greater inhibition of the activity or function of BAFF and/or APRIL, compared to the inhibition of the activity or function of BAFF and/or APRIL by a reference BCMA polypeptide or a reference binding molecule.
119. The variant BCMA polypeptide of any one of embodiments 1-24, the fusion polypeptide of any one of embodiments 25-103, the dimer of any one of embodiments 104-113, or the conjugate of any one of embodiments 113-116, or variant BCMA polypeptide, the fusion polypeptide, the dimer, or the conjugate of embodiment 117 or 118, wherein:
the variant BCMA polypeptide, the fusion polypeptide, the dimer, or the conjugate, reduces proliferation of B cells or reduces BAFF and/or APRIL-mediated proliferation of B cells.
120. The variant BCMA polypeptide of any one of embodiments 1-24, the fusion polypeptide of any one of embodiments 25-103, the dimer of any one of embodiments 104-113, or the conjugate of any one of embodiments 113-116, or variant BCMA polypeptide, the fusion polypeptide, the dimer, or the conjugate of any one of embodiments 117-119, wherein:
the variant BCMA polypeptide, the fusion polypeptide, the dimer, or the conjugate, reduces the production of inflammatory cytokines.
121. The variant BCMA polypeptide, the fusion polypeptide, the dimer, or the conjugate of embodiment 120, wherein the inflammatory cytokine is one or more of IFNγ or IL-17A.
122. The variant BCMA polypeptide, the fusion polypeptide, the dimer, or the conjugate of any one of embodiments 117-121, wherein BAFF is a human BAFF and APRIL is a human APRIL.
123. The variant BCMA polypeptide, the fusion polypeptide, the dimer, or the conjugate of any one of embodiments 117-121, wherein BAFF is a murine BAFF and APRIL is a murine APRIL.
124. The variant BCMA polypeptide, the fusion polypeptide, the dimer, or the conjugate of any one of embodiments 118-122, wherein the reference BCMA polypeptide is a wild-type human BCMA CRD set forth in SEQ ID NO: 1.
125. The variant BCMA polypeptide, the fusion polypeptide, the dimer, or the conjugate of any one of embodiments 118-122 and 124, wherein the reference BCMA polypeptide is a human BCMA CRD comprising a serine to glycine substitution at position 40 (S40G) comprising the sequence set forth in SEQ ID NO:237.
126. The variant BCMA polypeptide, the fusion polypeptide, the dimer, or the conjugate of any one of embodiments 118-125, wherein the reference binding molecule is selected from among Atacicept, Telitacicept, belimumab, or BION-1301.
127. The variant BCMA polypeptide, the fusion polypeptide, the dimer, or the conjugate of any one of embodiments 117-126, wherein:
the binding affinity of the variant BCMA polypeptide, the fusion polypeptide, the dimer, or the conjugate to human BAFF is at least at or about 1-, 2-, 3-, 4-, 5-, 10-, 20-, 25-, 50-, 100-, 200-, 250-, or 500-fold greater than that of the reference BCMA polypeptide or the reference binding molecule; and/or
the inhibition of activity or function of human BAFF by the variant BCMA polypeptide, the fusion polypeptide, the dimer, or the conjugate is at least at or about 5-, 10-, 20-, 25-, 50-, 100-, 200-, 250-, or 500-fold greater than that of the reference BCMA polypeptide or the reference binding molecule.
128. The variant BCMA polypeptide, the fusion polypeptide, the dimer, or the conjugate of any one of embodiments 117-127, wherein:
the binding affinity of the variant BCMA polypeptide, the fusion polypeptide, the dimer, or the conjugate to human APRIL is at least at or about 1-, 2-, 3-, 4-, 5-, 10-, 20-, 25-, 50-, 100-, 200-, 250-, or 500-fold greater than that of the reference BCMA polypeptide or the reference binding molecule; and/or
the inhibition of activity or function of human APRIL by the variant BCMA polypeptide, the fusion polypeptide, the dimer, or the conjugate is at least at or about 5-, 10-, 20-, 25-, 50-, 100-, 200-, 250-, or 500-fold greater than that of the reference BCMA polypeptide or the reference binding molecule.
129. The variant BCMA polypeptide, the fusion polypeptide, the dimer, or the conjugate of any one of embodiments 1-130, wherein the ratio of binding selectivity for human BAFF relative to human APRIL (huBAFF K_{D}/huAPRIL K_{D}) is more than 5, 4, 3, 2 or 1.
130. The variant BCMA polypeptide, the fusion polypeptide, the dimer, or the conjugate of any one of embodiments 1-129, wherein the equilibrium dissociation constant (K_{D}) for binding to human BAFF is less than 600 pM, less than 550 pM, less than 500 pM, less than 450 pM, less than 400 pM, less than 350 pM, less than 300 pM, less than 250 pM, less than 200 pM, or less than 150 pM.
131. The variant BCMA polypeptide, the fusion polypeptide, the dimer, or the conjugate of any one of embodiments 1-130, wherein the equilibrium dissociation constant (K_{D}) for binding to human BAFF is in the picomolar (pM) range.
132. The variant BCMA polypeptide, the fusion polypeptide, the dimer, or the conjugate of any one of embodiments 1-130, wherein the equilibrium dissociation constant (K_{D}) for binding to human BAFF is in the sub-picomolar (pM) range.
133. The variant BCMA polypeptide, the fusion polypeptide, the dimer, or the conjugate of any one of embodiments 1-129 and 132, wherein the equilibrium dissociation constant (K_{D}) for binding to human BAFF is less than 1.0 pM, less than 0.9 pM, less than 0.8 pM, less than 0.7 pM, less than 0.6 pM, less than 0.5 pM, less than 0.4 pM, less than 0.3 pM, less than 0.2 pM, or less than 0.1 pM.
134. The variant BCMA polypeptide, the fusion polypeptide, the dimer, or the conjugate of any one of embodiments 1-133, wherein the equilibrium dissociation constant (K_{D}) for binding to human APRIL is less than 100 pM, less than 90 pM, less than 80 pM, less than 70 pM, less than 60 pM, less than 50 pM, less than 40 pM, or less than 30 pM.
135. The variant BCMA polypeptide, the fusion polypeptide, the dimer, or the conjugate of any one of embodiments 1-134, wherein the equilibrium dissociation constant (K_{D}) for binding to human APRIL is in the picomolar (pM) range.
136. The variant BCMA polypeptide, the fusion polypeptide, the dimer, or the conjugate of any one of embodiments 1-134, wherein the equilibrium dissociation constant (K_{D}) for binding to human APRIL is in the sub-picomolar (pM) range.
137. The variant BCMA polypeptide, the fusion polypeptide, the dimer, or the conjugate of any one of embodiments 1-133 and 136, wherein the equilibrium dissociation constant (K_{D}) for binding to human APRIL is less than 1.0 pM, less than 0.9 pM, less than 0.8 pM, less than 0.7 pM, less than 0.6 pM, less than 0.5 pM, less than 0.4 pM, less than 0.3 pM, less than 0.2 pM, or less than 0.1 pM.
138. The variant BCMA polypeptide, the fusion polypeptide, the dimer, or the conjugate of any one of embodiments 1-137, wherein the equilibrium dissociation constant (K_{D}) for binding to human BAFF and human APRIL both are less than 120 pM.
139. The variant BCMA polypeptide, the fusion polypeptide, the dimer, or the conjugate of any one of embodiments 1-138, wherein the equilibrium dissociation constant (K_{D}) for binding to human BAFF and human APRIL both are less than 0.3 pM.
140. The variant BCMA polypeptide, the fusion polypeptide, the dimer, or the conjugate of any one of embodiments 1-139, wherein the equilibrium dissociation constant (K_{D}) is measured by a kinetic exclusion assay or surface plasmon resonance (SPR).
141. The variant BCMA polypeptide, the fusion polypeptide, the dimer, or the conjugate of any one of embodiments 1-140, wherein the variant BCMA polypeptide, the fusion polypeptide, the dimer, or the conjugate does not substantially bind to heparan sulfate proteoglycans (HSPGs).
142. The variant BCMA polypeptide, the fusion polypeptide, the dimer, or the conjugate of any one of embodiments 1-141, wherein the HSPGs are selected from among one or more of syndecan-1 and syndecan-2.
143. The variant BCMA polypeptide, the fusion polypeptide, the dimer, or the conjugate of any one of embodiments 1-142, wherein the variant BCMA polypeptide, the fusion polypeptide, the dimer, or the conjugate inhibits the activity or function of BAFF and/or APRIL or variants thereof.
144. The variant BCMA polypeptide, the fusion polypeptide, the dimer, or the conjugate of embodiment 143, wherein the activity or function of BAFF and/or APRIL is selected from B-cell survival, B-cell proliferation, and/or immunoglobulin production.
145. A polynucleotide comprising a nucleotide sequence encoding the variant BCMA polypeptide of any one of embodiments 1-24 and 117-144, the fusion polypeptide of any one of embodiment 25-103 and 117-144, or a first monomer and/or a second monomer of the dimer of any one of embodiments 104-113 and 117-144.
146. A vector comprising the polynucleotide of embodiment 136, or a polynucleotide comprising a nucleotide sequence encoding the variant BCMA polypeptide of any one of embodiments 1-24 and 117-144, the fusion polypeptide of any one of embodiment 25-103 and 117-144, or a first monomer and/or a second monomer of the dimer of any one of embodiments 104-113 and 117-144.
147. A cell comprising the polynucleotide of embodiment 145 or the vector of embodiment 146.
148. A method of manufacturing a variant BCMA polypeptide, a fusion polypeptide, or a dimer, wherein the method comprises:
1) introducing the polynucleotide of embodiment 145 or the vector of embodiment 146 into a cell;
2) culturing the host cell under conditions suitable for expression of the polypeptide;
3) recovering or isolating the polypeptide; and optionally
4) purifying the polypeptide.
149. A pharmaceutical composition comprising the variant BCMA polypeptide of any one of embodiments 1-24 and 117-144, the fusion polypeptide of any one of embodiments 25-103 and 117-144, the dimer of any one of embodiments 104-113 and 117-144, the conjugate of any one of embodiments 114-144, the polynucleotide of embodiment 145, the vector of embodiment 146, or the cell of embodiment 147.
150. The pharmaceutical composition of embodiment 149, further comprising one or more pharmaceutically acceptable excipient(s).
151. The pharmaceutical composition of embodiment 149 or 150, wherein the one or more excipient(s) comprises a pharmaceutically acceptable liquid carrier.
152. The pharmaceutical composition of any one of embodiments 149-151, wherein the one or more excipient(s) comprises a pharmaceutically acceptable processing agents.
153. The pharmaceutical composition of any one of embodiments 149-152, wherein the pharmaceutical composition is a liquid formulation, a formulation for an intravenous injection, a solid dosage form, or an inhalable preparation.
154. The pharmaceutical composition of any one of embodiments 149-153, for treating a disease or disorder.
155. The pharmaceutical composition for use of embodiment 154, wherein the pharmaceutical composition is to be administered to a subject having the disease or disorder.
156. The variant BCMA polypeptide of any one of embodiments 1-24 and 117-144, the fusion polypeptide of any one of embodiments 25-103 and 117-144, the dimer of any one of embodiments 104-113 and 117-144, the conjugate of any one of embodiments 114-144, the polynucleotide of embodiment 145, the vector of embodiment 146, or the cell of embodiment 147, for treating a disease or disorder.
157. The variant BCMA polypeptide, the fusion polypeptide, the dimer, the conjugate, the polynucleotide, the vector or the cell for use of embodiment 156, wherein the variant BCMA polypeptide, the fusion polypeptide, the dimer, the conjugate, the polynucleotide, the vector or the cell is to be administered to a subject having the disease or disorder.
158. A method of treatment of a disease or disorder, the method comprising administering the variant BCMA polypeptide of any one of embodiments 1-24 and 117-144, the fusion polypeptide of any one of embodiments 25-103 and 117-144, the dimer of any one of embodiments 104-113 and 117-144, the conjugate of any one of embodiments 114-144, the polynucleotide of embodiment 145, the vector of embodiment 146, the cell of embodiment 147, or the pharmaceutical composition of any one of embodiments 149-155 to a subject having the disease or disorder.
159. Use of variant BCMA polypeptide of any one of embodiments 1-24 and 117-144, the fusion polypeptide of any one of embodiments 25-103 and 117-144, the dimer of any one of embodiments 104-113 and 117-144, the conjugate of any one of embodiments 114-144, the polynucleotide of embodiment 145, the vector of embodiment 146, the cell of embodiment 147, or the pharmaceutical composition of any one of embodiments 149-155 in the manufacture of a medicament for the treatment of a disease or disorder.
160. Use of the variant BCMA polypeptide of any one of embodiments 1-24 and 117-144, the fusion polypeptide of any one of embodiments 25-103 and 117-144, the dimer of any one of embodiments 104-113 and 117-144, the conjugate of any one of embodiments 114-144, the polynucleotide of embodiment 145, the vector of embodiment 146, the cell of embodiment 147, or the pharmaceutical composition of any one of embodiments 149-155 for treating a disease or disorder.
161. The use of embodiment 159 or 160, wherein the variant BCMA polypeptide, the fusion polypeptide, the dimer, the conjugate or the pharmaceutical composition is to be administered to a subject having the disease or disorder.
162. The pharmaceutical composition, the variant BCMA polypeptide, the fusion polypeptide, the dimer, the conjugate, the polynucleotide, the vector or the cell for use, the method or the use of any one of embodiments 158-161, wherein the disease or disorder is a B-cell- or antibody-mediated disease or disorder.
163. The pharmaceutical composition, the variant BCMA polypeptide, the fusion polypeptide, the dimer, the conjugate, the polynucleotide, the vector or the cell for use, the method or the use of any one of embodiments 158-162, wherein the disease or disorder is an autoimmune disease or disorder.
164. The pharmaceutical composition, the variant BCMA polypeptide, the fusion polypeptide, the dimer, the conjugate, the polynucleotide, the vector or the cell for use, the method or the use of any one of embodiments 158-163, wherein the autoimmune disease or disorder is an immune-mediated disease or disorder of the subject's tissues, bone, joints, blood vessels, thyroid, kidneys, nervous system, brain, lungs, and/or skin.
165. The pharmaceutical composition, the variant BCMA polypeptide, the fusion polypeptide, the dimer, the conjugate, the polynucleotide, the vector or the cell for use, the method or the use of any one of embodiment 163 or 164, wherein the autoimmune disease or disorder is selected from among a renal disease or disorder, lupus, arthritis, a spondyloarthropathic disorder, a vasculitis disorder, a hemolytic anemia disorder, a thrombocytopenia disorder, a thyroiditis disorder, a demyelinating disease of the central and/or peripheral nervous system, inflammatory and/or fibrotic lung disorder, a skin disorder, or an allergic disorder.
166. The pharmaceutical composition, the variant BCMA polypeptide, the fusion polypeptide, the dimer, the conjugate, the polynucleotide, the vector or the cell for use, the method or the use of any one of embodiments 158-165, wherein the disease or disorder is selected from among systemic lupus erythematosus (SLE) and/or lupus nephritis (LN), IgA nephropathy (Berger's disease), Goodpasture syndrome, anti-neutrophil cytoplasmic antibody (ANCA) associated vasculitis, Henoch-Schonlein purpura, polyarteritis nodosa (PAN), sarcoidosis of the kidneys, rheumatoid arthritis, juvenile chronic arthritis, arthritis associated with inflammatory bowel disease, ankylosing spondylitis, spondylitis associated with psoriasis, juvenile onset spondyloarthropathy, undifferentiated spondyloarthropathy, Reiter's syndrome, scleroderma, Sjogren's syndrome, systemic necrotizing vasculitis, polyarteritis nodosa, allergic angiitis and granulomatosis, polyangiitis, Wegener's granulomatosis, lymphomatoid granulomatosis, mucocutaneous lymph node syndrome (MLNS or Kawasaki's disease), isolated central nervous system vasculitis, Behet's disease, thromboangiitis obliterans (Buerger's disease), cutaneous necrotizing venulitis, sarcoidosis, autoimmune hemolytic anemia, immune pancytopenia, paroxysmal nocturnal hemoglobinuria, thrombocytopenic purpura, immune-mediated thrombocytopenia, Grave's disease, Hashimoto's thyroiditis, juvenile lymphocytic thyroiditis, atrophic thyroiditis, type I diabetes mellitus, glomerulonephritis and tubulointerstitial nephritis, multiple sclerosis (MS), idiopathic demyelinating polyneuropathy, Guillain-Barre syndrome, chronic inflammatory demyelinating polyneuropathy, eosinophilic pneumonia, idiopathic pulmonary fibrosis, hypersensitivity pneumonitis, bullous skin disease, erythema multiforme, contact dermatitis, asthma, allergic rhinitis, atopic dermatitis, food hypersensitivity, or urticaria.
167. The pharmaceutical composition, the variant BCMA polypeptide, the fusion polypeptide, the dimer, the conjugate, the polynucleotide, the vector or the cell for use, the method or the use of any one of embodiments 158-166, wherein the disease or disorder is selected from among systemic lupus erythematosus (SLE) and/or lupus nephritis (LN), IgA nephropathy (Berger's disease), Goodpasture syndrome, anti-neutrophil cytoplasmic antibody (ANCA) associated vasculitis, Henoch-Schonlein purpura, Polyarteritis nodosa (PAN), or sarcoidosis of the kidneys.
168. The pharmaceutical composition, the variant BCMA polypeptide, the fusion polypeptide, the dimer, the conjugate, the polynucleotide, the vector or the cell for use, the method or the use of any one of embodiments 158-167, wherein the disease or disorder is selected from among systemic lupus erythematosus (SLE) and/or lupus nephritis (LN), or IgA nephropathy (Berger's disease).
169. The pharmaceutical composition, the variant BCMA polypeptide, the fusion polypeptide, the dimer, the conjugate, the polynucleotide, the vector or the cell for use, the method or the use of any one of embodiments 158-161, wherein the disease or disorder is a tissue or organ transplant rejection.
170. The pharmaceutical composition, the variant BCMA polypeptide, the fusion polypeptide, the dimer, the conjugate, the polynucleotide, the vector or the cell for use, the method or the use of embodiment 169, wherein the tissue or organ transplant rejection is selected from among acute or chronic B-cell or antibody-mediated rejection of allografts of tissues consisting of bone marrow, stem cell, skin, and solid organs, acute or chronic graft versus host disease (GVHD), antibody-mediated rejection (AMR) of solid organs, hyperacute organ transplant rejection, acute organ transplant rejection, chronic organ transplant rejection.
171. The pharmaceutical composition, the variant BCMA polypeptide, the fusion polypeptide, the dimer, the conjugate, the polynucleotide, the vector or the cell for use, the method or the use of any one of embodiments 158-161, wherein the disease or disorder is a B-cell malignancy.
172. The pharmaceutical composition, the variant BCMA polypeptide, the fusion polypeptide, the dimer, the conjugate, the polynucleotide, the vector or the cell for use, the method or the use of embodiment 171, wherein the B-cell malignancy is selected from among non-Hodgkin's lymphoma, multiple myeloma (MM), B-chronic lymphocytic leukemia, plasmacytoma, macroglobulinemia, or Waldenstrom's macroglobulinemia (WM).
173. The pharmaceutical composition, the variant BCMA polypeptide, the fusion polypeptide, the dimer, the conjugate, the polynucleotide, the vector or the cell for use, the method or the use of any one of embodiments 158-172, wherein a therapeutically effective amount of the variant BCMA polypeptide, the fusion polypeptide, the dimer, the conjugate, the polynucleotide, the vector, the cell or the pharmaceutical composition is to be administered to the subject.
174. The pharmaceutical composition, the variant BCMA polypeptide, the fusion polypeptide, the dimer, the conjugate, the polynucleotide, the vector or the cell for use, the method or the use of any one of embodiments 158-173, wherein the method or the use is a therapeutic use or a prophylactic use.
175. The pharmaceutical composition, the variant BCMA polypeptide, the fusion polypeptide, the dimer, the conjugate, the polynucleotide, the vector or the cell for use, the method or the use of embodiment 174, wherein the therapeutic use is for induction therapy.
176. The pharmaceutical composition, the variant BCMA polypeptide, the fusion polypeptide, the dimer, the conjugate, the polynucleotide, the vector or the cell for use, the method or the use of embodiment 175, wherein the induction therapy spans for up to at or about one week, up to at or about two weeks, up to at or about three weeks, or up to at or about four weeks.
177. The pharmaceutical composition, the variant BCMA polypeptide, the fusion polypeptide, the dimer, the conjugate, the polynucleotide, the vector or the cell for use, the method or the use of embodiment 174, wherein the therapeutic use is for maintenance therapy.
178. The pharmaceutical composition, the variant BCMA polypeptide, the fusion polypeptide, the dimer, the conjugate, the polynucleotide, the vector or the cell for use, the method or the use of embodiment 177, wherein the maintenance therapy spans for up to at or about one week, up to at or about two weeks, up to at or about three weeks, or up to at or about four weeks.
179. The pharmaceutical composition, the variant BCMA polypeptide, the fusion polypeptide, the dimer, the conjugate, the polynucleotide, the vector or the cell for use, the method or the use of any one of embodiments 158 and 161-178, wherein the administration is selected from among intravenous, oral, parenteral, sublingual, by inhalation, rectal or topical.
180. The pharmaceutical composition, the variant BCMA polypeptide, the fusion polypeptide, the dimer, the conjugate, the polynucleotide, the vector or the cell for use, the method or the use of embodiment 179, wherein the administration is intravenous administration.

### VII. EXAMPLES

The following examples are included for illustrative purposes only and are not intended to limit the scope of the invention.

### Example 1: Construction of Plasmid Vectors Encoding Z-TACI-Ig and BCMA ECD-Ig Fusion Proteins

This example provides a description of the generation of an [S40G]huBCMA ECD-Ig fusion protein (i.e., the huBCMA surrogate control protein) and a TACI ECD-Ig fusion protein (“Z-TACI-Ig”). These fusion proteins were used as controls and for comparative purposes in BIAcore™ and KinExA™ binding assays, cell-based activity assays, and in vivo experiments. Both huBCMA ECD and TACI ECD have been reported as exhibiting binding activity for both BAFF and APRIL.

As described in Example 18, removal of the N-glycosylation site at positions 38-40 (i.e., by the S40G substitution, with reference to amino acid positions of wild-type huBCMA ECD CRD, SEQ ID NO:1) appeared to lead to a protein preparation having a more homogeneous molecular weight distribution as compared to protein not having this substitution. This substitution did not appear to have an impact on huBAFF and huAPRIL binding activities. This molecule was used as a control and as a benchmark to quantify the binding activities of the variant BCMA polypeptides described herein.

### A. Construction of DNA Plasmid Vectors Encoding [S40G]huBCMA ECD-Ig Fusion Protein

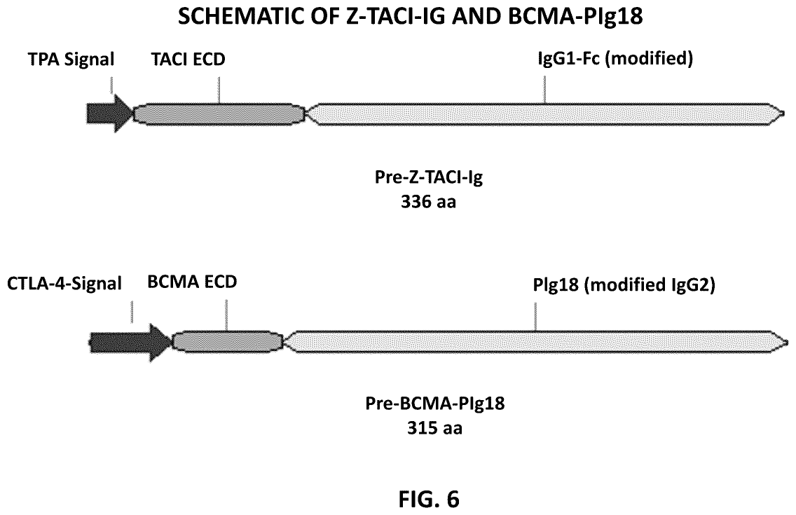

This example describes the making of a DNA plasmid vector that encodes the [S40G]huBCMA ECD-Ig fusion protein, as shown in FIG. 6. In this fusion protein, the [S40G]huBCMA extracellular domain (ECD) is covalently linked at its C-terminus to the N-terminus of a mutated human IgG2 Fc domain. The mutated human IgG2 Fc domain has three of the four hinge cysteines mutated to serine (referred to herein as “PIg18”).

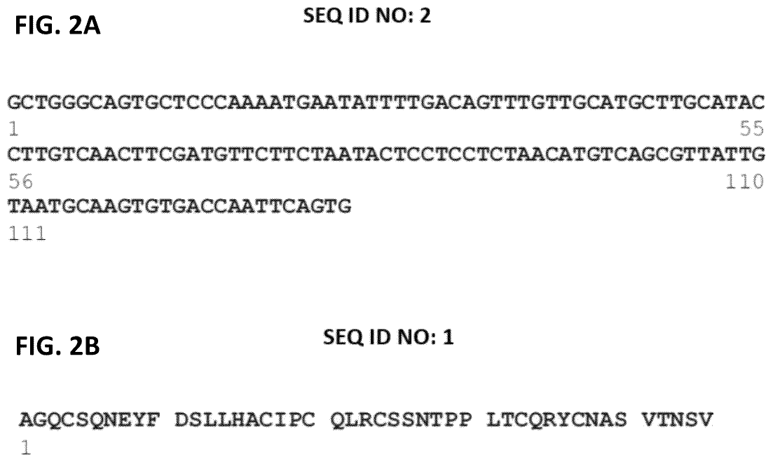

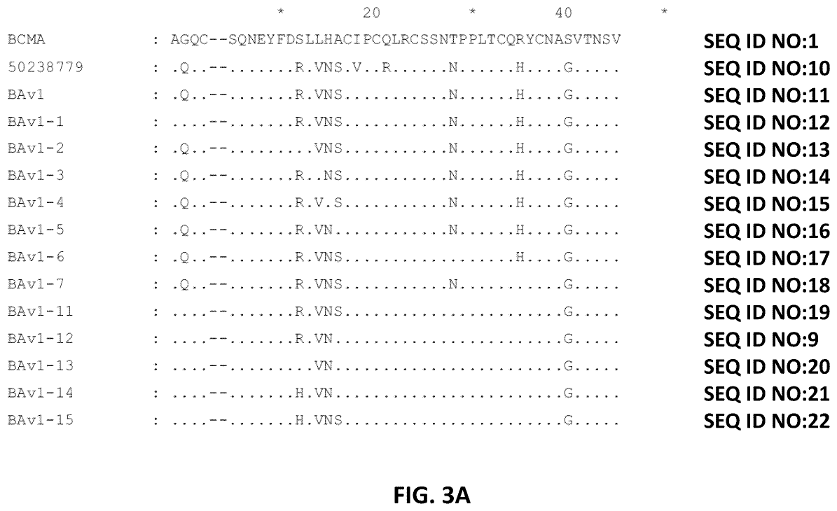

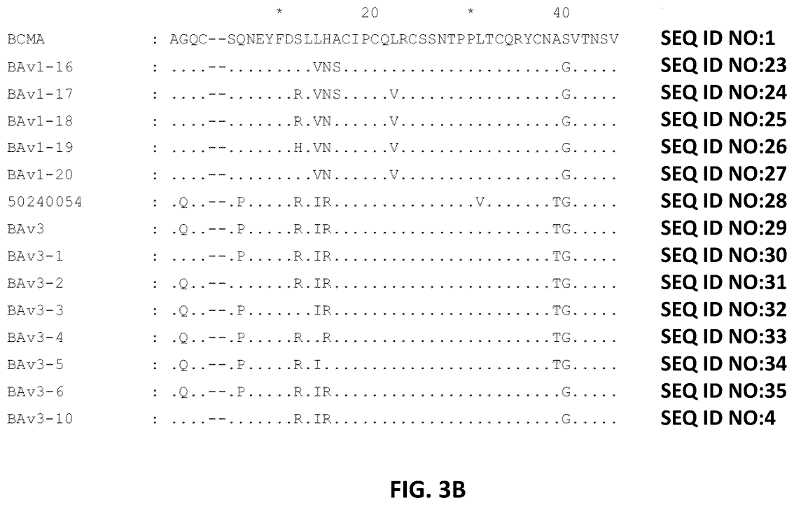

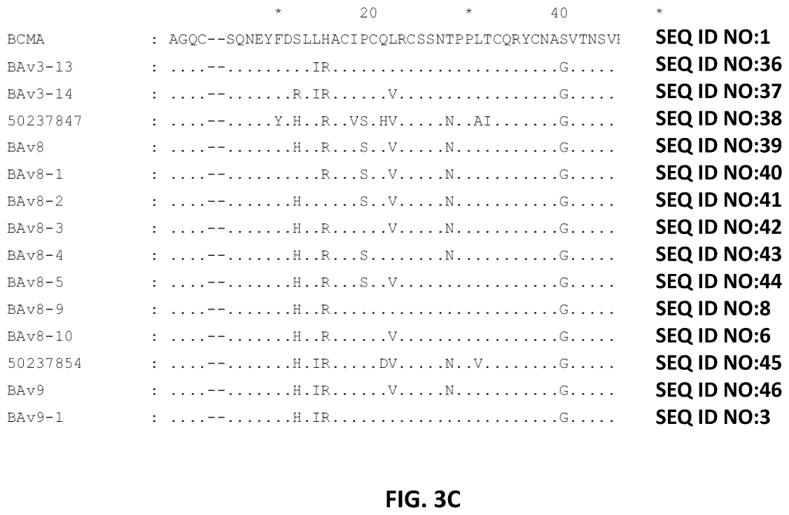

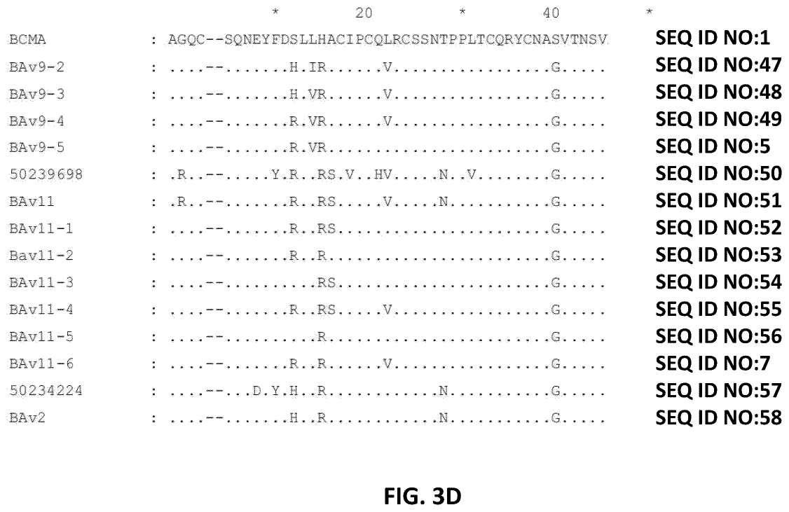

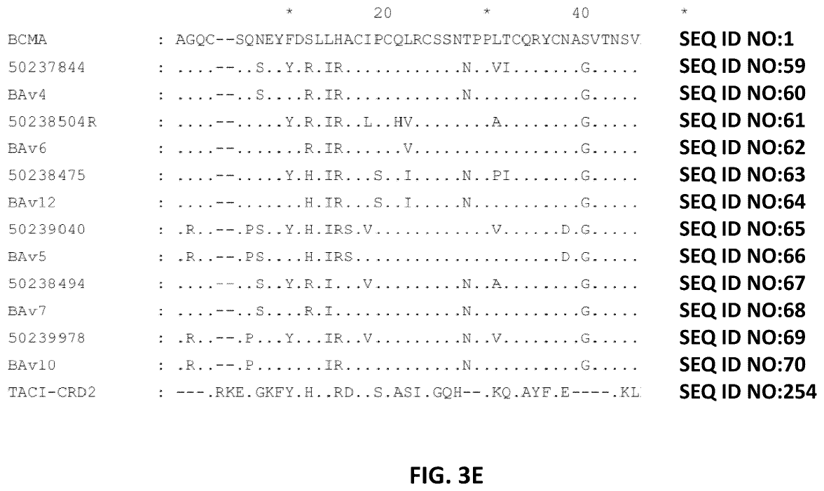

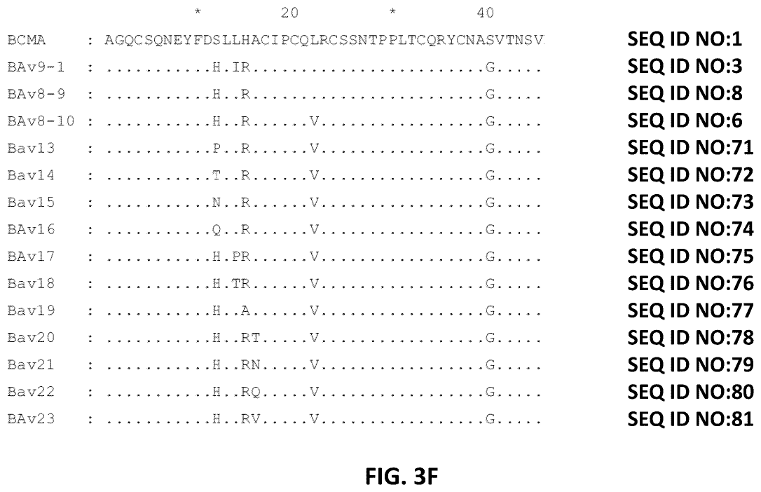

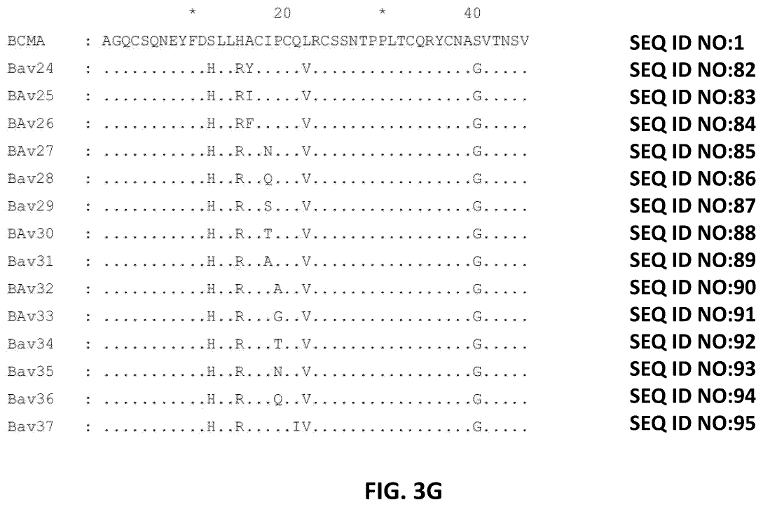

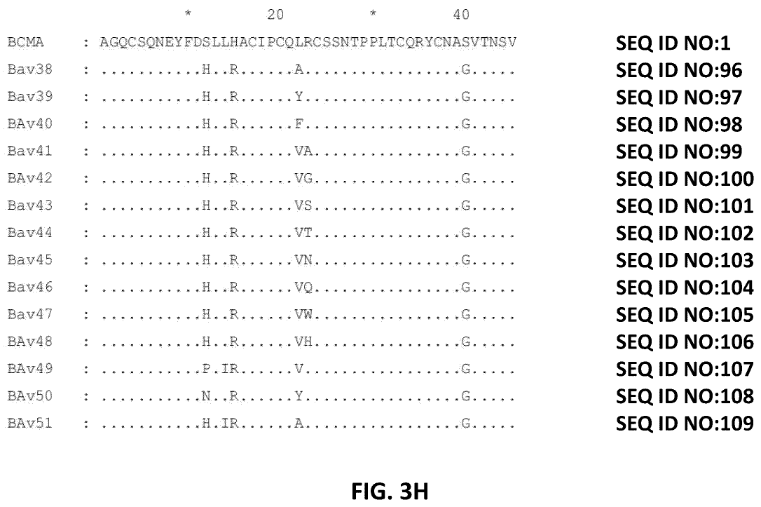

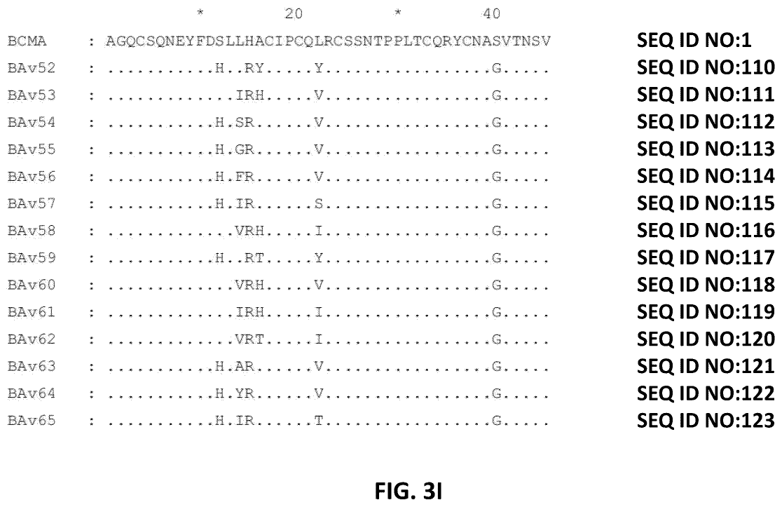

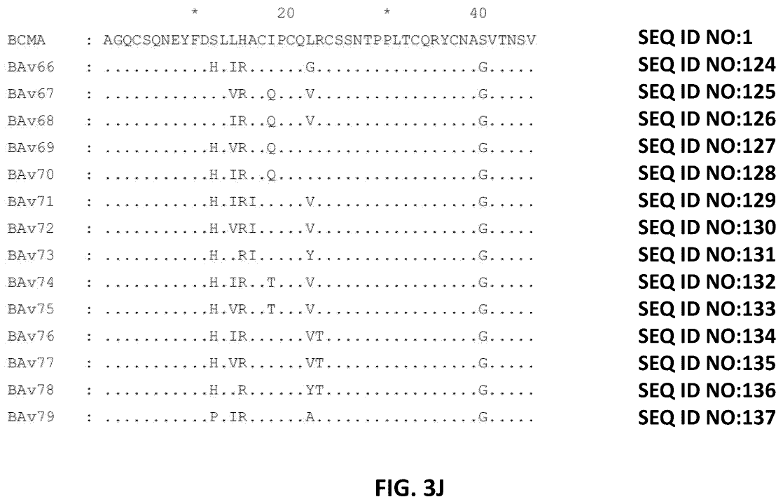

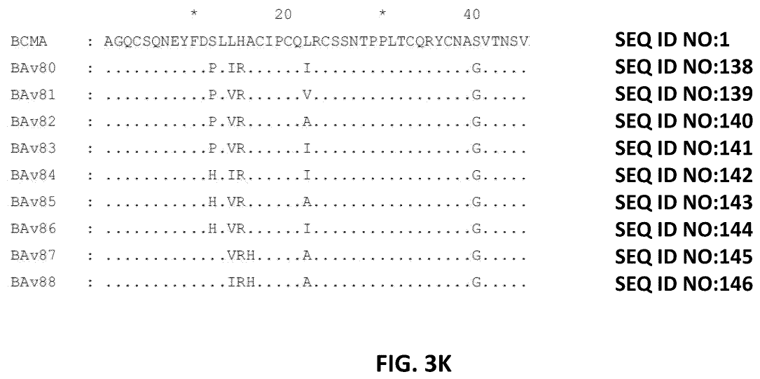

The methods described for making and purifying the [S40G]huBCMA ECD-Ig fusion protein were similarly used to prepare and purify other variants of huBCMA ECD/huBCMA ECD-Ig described herein, unless otherwise noted. DNA encoding a polypeptide comprising [S40G]huBCMA ECD (SEQ ID NO:224), e.g., the native human BCMA ECD amino acid sequence depicted in FIG. 2B with the substitution S40G, which was included to remove a glycosylation site) was created by PCR assembly using overlapping oligonucleotides designed based on the sequence of the BCMA gene (NCBI AB052772.1 (CDS sequence); BAB60895.1 (polypeptide sequence)). The oligonucleotides were designed, made, and assembled using standard procedures and included stop and start codons and restriction sites as necessary. See, e.g., Sambrook, Joseph. Molecular Cloning: a Laboratory Manual. Cold Spring Harbor, N.Y.: Cold Spring Harbor Laboratory Press, 2001; Verma and Eckstein, Annu. Rev. Biochem. 1998; 67:99-134; Tang et al., “Gene Assembly,” Synthetic Biology, Academic Press, 2013:3-21; Ulyanov and James “Chemical Synthesis of Oligonucleotides,” Comprehensive Natural Products II, American Chemical Society, 2010; 10:247-278. Specifically, the oligonucleotides were assembled in a 100 μl PCR reaction with 1 pM oligonucleotides, 1× Taq buffer (Qiagen; #201225) and 200 μM dNTPs for 30 amplification cycles (94° C., 30 s; 60° C., 30 s; 72° C., 60 s). Nucleic acid sequence encoding the CTLA-4 signal peptide was appended to the 5′ end of the BCMA-ECD-coding fragment using the splicing by overlap extension (SOE) method described, for example, in Horton et al., Gene 1989; 77:61-68, which is incorporated herein by reference. Similarly, nucleic acid sequence encoding a variant form of human IgG2-Fc peptide “PIg18” was fused to the 3′ end of the CTLA-4 signal-BCMA-ECD-encoding fragment and the resulting DNA fragment was subcloned into vector CET1019AS (EMD Millipore, a division of Merck KgaA) to generate the vector CET1019-BCMA-PIg18. PIg18 differs from the corresponding native human IgG2-Fc in that it has a serine at position 4, a serine at position 5, and a serine at position 11. In contrast, the corresponding native IgG2 Fe has a cysteine at each of these positions, The resulting plasmid was sequenced to confirm the DNA sequence encoding the entire fusion polypeptide.

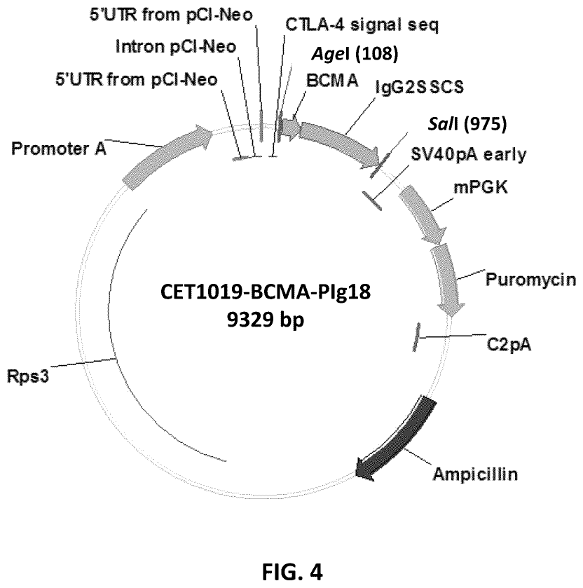

A schematic diagram of CET1019-BCMA-PIg18 is provided in FIG. 4. This vector includes Promoter A from Caviid herpesvirus 2 directing expression of a transcription cassette including the 5′ UTR/intron from pCI-Neo (Promega) and a nucleic acid sequence encoding the CTLA-4 signal peptide fused in-frame to BCMA-PIg18, an SV40 early poly A termination signal sequence, a mouse PGK promoter driving expression of the puromycin resistance gene, a Bla promoter driving expression of the ampicillin resistant gene, a ColE1 origin, and a UCOE (ubiquitous chromatin-opening element) sequence (Rps3) that results in high-expression of polypeptides encoded on the plasmid.

The polynucleotide and amino acid sequences encoding the predicted pre-form of the [S40G]BCMA ECD-Ig fusion protein, including the CTLA-4 signal peptide coding sequence. The predicted amino acid sequence includes the CTLA-4 signal peptide, the [S40G] human BCMA ECD (SEQ ID NO:224), and the mutant human IgG2 heavy chain Fc domain polypeptide (PIg18).

The signal peptide is typically cleaved during processing and thus the secreted protein (i.e., the mature form) of [S40G]BCMA ECD-Ig fusion protein does not ordinarily contain the signal peptide sequence. In some instances, the expressed [S40G]BCMA ECD-Ig fusion protein may not include the C-terminal lysine (K) residue, as the C-terminal lysine may be cleaved during processing or secretion. In some instances, the penultimate glycine (G) residue is missing.

In some embodiments, BCMA ECD-Ig, and variants thereof, typically exist in solution as dimeric fusion proteins comprising two identical monomeric polypeptides. In some embodiments, the two monomeric BCMA ECD-Ig polypeptides are covalently linked together by disulfide bonds formed between cysteine residues in each monomer, thereby forming the BCMA ECD-Ig fusion protein dimers. The BCMA ECD-Ig dimers, and variants thereof, are the form of the fusion protein molecules used in the assays described herein, unless explicitly stated otherwise.

### B. Creation of DNA Plasmid Vectors Encoding Z-TACI-Ig Fusion Protein

This example describes the making of a DNA plasmid vector that encodes the Z-TACI-Ig fusion protein, which is depicted in FIG. 6. Z-TACI-Ig comprises a portion of the human TACI extracellular domain (ECD) that contains both CRD1 and CRD2 (SEQ ID NO:147), covalently linked at its C-terminus to the N-terminus of a mutant human IgG1 Fc polypeptide. ZIg differs from the corresponding native human IgG1-Fc in that it has a serine at position 5, an alanine at position 19, a glutamic acid at position 20, an alanine at position 22, a serine at position 115, and a serine at position 116. These correspond to the following substitutions relative to the corresponding native IgG1 Fc: C5S+L19A+L20E+G22A+A115S+P116S. The predicted amino acid sequence of the pre-form of Z-TACI-Ig includes the TPA signal peptide, a portion of the human TACI ECD domain (the sequence has a polymorphism from the reference at position 3 in CRD1 which is a K instead of E) and ZIg.

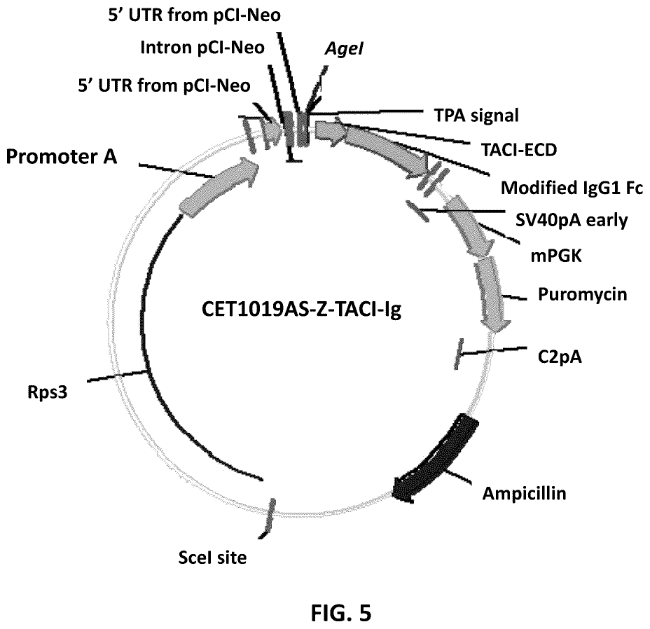

To produce this fusion protein, plasmid vector CET1019AS-Z-TACI-Ig was constructed as follows. DNA sequence encoding a fusion of the human tissue plasminogen activator (TPA) signal peptide, the Z-TACI-ECD, and the mutated IgG1 Fc was synthesized (based on the polypeptide sequence in publication: International Nonproprietary Names for Pharmaceutical Substances (INN) Recommended INN: List 57, WHO Drug Information, Vol. 21, No. 1, 2007). The synthetic gene contained appropriate start and stop codons, and introduced flanking AgeI and SalI sites restriction sites that were used to subclone into the expression vector. The DNA was digested with AgeI and SalI. The resulting fragments were separated by agarose-gel electrophoresis, purified using Qiaquick™ Gel Extraction Kit (Qiagen, Catalog No. 28704) as per manufacturer's recommendation, and ligated into similarly digested plasmid CET1019AS (EMD Millipore, a division of Merck KGAa). Ligations were transformed into TOP10™ E. coli cells (Invitrogen Catalog No. C404010) as per manufacturer's recommendations. The resulting cells were incubated overnight at 37° C. in LB medium containing 50 μg/ml carbenicillin with shaking at 250 rpm and then subjected to Maxiprep™ (Qiagen; Catalog No. 12362) purification to prepare stocks of plasmid DNA. A schematic diagram of this vector is provided in FIG. 5.

A schematic diagram of CET1019-BCMA-PIg18 is provided in FIG. 4. This vector includes Promoter A from Caviid herpesvirus 2 directing expression of a transcription cassette including the 5′UTR/intron from pCI-Neo (Promega) and a nucleic acid sequence encoding the TPA signal peptide fused in-frame to Z-TACI-Ig, an SV40 early poly A termination signal sequence, a mouse PGK promoter driving expression of the puromycin resistance gene, a Bla promoter driving expression of the ampicillin resistant gene, a ColE1 origin, and a UCOE (ubiquitous chromatin-opening element) sequence (Rps3) that results in high-expression of polypeptides encoded on the plasmid.

The signal peptide is typically cleaved during processing and thus the secreted fusion protein (i.e., mature form) of Z-TACI-Ig does not ordinarily contain the signal peptide sequence. In some instances, the expressed Z-TACI-Ig polypeptide sequence may not include the C-terminal lysine (K) residue, as the C-terminal lysine may be cleaved during processing or secretion. In some instances, the penultimate glycine (G) residue is missing.

In some embodiments, the Z-TACI-Ig exists in solution as dimeric fusion proteins comprising two identical monomeric polypeptides. In some embodiments, the two monomeric Z-TACI-Ig polypeptides are covalently linked together by disulfide bonds formed between cysteine residues in each monomer, thereby forming the Z-TACI-Ig fusion protein dimers. The Z-TACI-Ig dimers are the form of the fusion protein molecules used in the assays described in these Examples, unless explicitly stated otherwise.

### Example 2: Creation of Stable Cell Lines Expressing Z-TACI-Ig and BCMA-PIg18 Fusion Proteins and Protein Expression and Purification

This example describes the creation of stable cell lines for generating multi-milligram quantities of the Z-TACI-Ig and BCMA-PIg18 fusion proteins discussed above.

### A. Transfection of CHO-K1 Cells

To generate multi-milligram quantities of the [S40G]huBCMA ECD-Ig and Z-TACI-Ig fusion proteins, stable CHO-K1 cell lines adapted for suspension growth (see “Animal Cell Technology: Products from Cells, Cells as Products”, Proceedings of the 16th ESACT Meeting Apr. 25-29, 1999, Lugano, Switzerland, edited by A. Bernard, et al. (2002)) were created by transfecting CHO-K1 cells (ATCC CCL-61) with the plasmid vectors described above. Naïve Opti-CHO-K1 cells were maintained in 500 ml shake flasks (SF500 flasks; VWR #29445-058) containing 100 ml Growth Medium (OptiCHO medium Invitrogen Catalog No. 12681) supplemented with 4 mM L-glutamine (Invitrogen Cat. No. 25031). For each electroporation, 2×10⁶ cells were suspended in 400 μL growth medium in a 0.4 cm cuvette (Bio-Rad Cat. No. 165-2088) and 20 μg maxiprep plasmid DNA (e.g., plasmid vectors encoding Z-TACI-Ig and [S40G]huBCMA ECD-Ig as described above) was added to the cells. Electroporations were performed in duplicate using a Gene Xcell Pulser (Bio-Rad Cat. No. 165-2661) at voltage 320V and square wave pulse length of 15 ms, and the cells were pooled, transferred to a T25 flask containing 5 mL of growth medium, and incubated at 37° C. with 5% CO2 for 48 hours without selection. The cells were harvested by centrifugation, resuspended in selective medium (growth medium containing 9% Puromycin), and incubated for 8-10 days at which time pools of stably transfected cells (“stable pools”) were obtained. Protein expression was determined by Protein A HPLC performed on culture supernatants from stable pools. For Z-TACI-Ig production, the stable pools were further subcloned to identify appropriate clonal cell-lines, whereas good quality [S40G]huBCMA ECD-Ig protein was obtained from stable pool and no further subcloning was needed.

B. Separation of Unique Clones expressing Z-TACI-Ig

The stable pool of Z-TACI-Ig-expressing cells was subcloned by limited dilution. The stably transfected cells were resuspended at a final density of 3.8 cells/ml in conditioned medium (medium collected from parental CHO-K1 cell culture) and seeded onto 96 well plates with flat bottom at 200 μL per well (75 cells per plate). The plates were examined after 10-12 days incubation and 52 wells that showed colonies were expanded to 12 well plates. After 5 days incubation, forty clones were chosen for further expansion to 125 ml shake flasks and the culture supernatants were assayed for protein expression using Protein A HPLC and cell-density to measure their productivity in PCD (pg/cell/day). The protein expressed from eighteen clones that showed good productivity was further analyzed by Western blot analysis. Equal amounts (6 μl) of medium from each cell culture was separated by SDS-PAGE on 4-12% Bis-Tris NuPAGE gels (Invitrogen Cat. No. NP0322BOX) in MOPS running buffer (Invitrogen) as per the manufacturer's recommended conditions. The proteins were transferred from gels to nitrocellulose membranes (Invitrogen Cat. No. LC2001) by electro-transfer as per the manufacturer's recommended conditions. Z-TACI-Ig fusion protein was visualized by probing the nitrocellulose membrane with horseradish peroxidase (HRP)-conjugated goat anti-human Ig antibody (Bethyl Cat. No. A80-104P) diluted to 1:5000 and detecting the signal using ECL Western Blot Detection Reagent (Amersham Cat. No. RPN2132) as per the manufacturer's recommended conditions. Clones that showed good productivity and high percentage of full-length Z-TACI-Ig protein were used for protein production.

### C. Protein a HPLC to Measure Protein Expression

Protein A HPLC was used for quantitating the amount of Fc-fusion protein in culture supernatants. The culture supernatants were loaded onto a Poros Protein A A/20 column using a 42%/58% ratio of 50 mM phosphoric acid, 150 mM potassium chloride, pH 7.5 and 50 mM phosphoric acid, 150 mM potassium chloride, pH 2.5, respectively. Fc-fusion protein was separated from process impurities by lowering the pH of the mobile phase with a gradient elution to 12%/88% of 50 mM phosphoric acid, 150 mM potassium chloride, pH 7.5 and 50 mM phosphoric acid, 150 mM potassium chloride, pH 2.5, respectively. The elution profile was monitored for 6 min with detection by absorbance (A) at 220 nanometers (nm). For a given peak area, the amount of Fc-fusion protein was determined from a standard curve of peak area versus amount, generated by injecting set amounts of a known concentration of a reference control.

### D. Purification of Proteins

Supernatants from the cultures of pools or clones (e.g., comprising cells expressing Z-TACI-Ig or [S40G]huBCMA ECD-Ig) were collected by centrifugation at 1000×g for 10 min at room temperature and filtration through 0.2 μm membranes (Nalgene, VWR Catalog No. 73520-982). Proteins were purified by Protein-A affinity chromatography using Hitrap MAb Select Sure (GE Healthcare Cat No. 11-0034-93 or 11-0034-95) column with an AKTA Explorer HPLC system (GE Healthcare). In brief, the column was equilibrated with 1×PBS/pH 7.4., clarified harvests were applied to the column, which was subsequently washed with 1×PBS/pH 7.4 (Invitrogen). The bound protein was eluted with elution buffer (80 mM citric acid/160 mM NaCl/pH 3.5) and the eluted protein was immediately neutralized by addition of 1/10 volume of 2M Tris base/pH 8.6. The eluted protein was dialyzed into PBS pH7.4 buffer using 20 kDa MWCO membranes (Pierce Cat. No. 66003 for 0.5-3 mL or No. 66012 for 3-12 mL or 66030 for 12-30 mL). The protein was stored in PBS pH7.4 at −80° C.

### Example 3: Protein Characterization: Z-TACI-Ig, [S40G]BCMA ECD-Ig, and BCMA Variant Molecules

This example describes the evaluation of protein quality.

### A. SDS/PAGE Analysis

The apparent molecular weights (MW) of purified Z-TACI-Ig and [S40G]huBCMA ECD-Ig fusion proteins (described below) were measured by SDS/PAGE analysis under non-reducing conditions. Under non-reducing conditions, these fusion proteins typically exist in the form of dimeric fusion proteins comprising two (mature) monomeric fusion polypeptides. The two monomeric fusion polypeptides are linked together by disulfide bonds formed between cysteine residues in each monomer. The data shown in the Examples that follow pertain to the homodimer form of the molecules, unless stated otherwise.

SDS/PAGE analyses were performed as follows. For each fusion protein, two pg of purified fusion protein was added to 20 μl LDS Sample Buffer (Invitrogen Cat. No. NP0007) and run through NuPAGE 4-12% Bis-Tris gels (Invitrogen Cat. No. NP0321BOX) in 1× Tris-Glycine sodium dodecyl sulfate (SDS)/PAGE running buffer (Invitrogen Cat. No. NP0002) following the manufacturer's recommended conditions. Gels were stained by incubation in 50 ml SimplyBlue SafeStain (Invitrogen Cat. No. LC6060) for 1 hr with gentle agitation at RT. Gels were de-stained by two incubations with 200 ml water for 1 hr with gentle agitation at RT and processed in drying buffer (Bio-Rad Cat. No. 161-0752) according to the manufacturer's recommended conditions.

An analysis of the gel indicated that the Z-TACI-Ig and [S40G]huBCMA ECD-Ig fusion protein dimers were each made up of two identical (with respect to size) Z-TACI-Ig or [S40G]huBCMA ECD-PIg18 protein monomers that are linked by disulfide bonds formed between cysteine residues in each monomer. SDS/PAGE analyses were performed on all protein preparations to verify protein quality in terms of apparent molecular weight, protein concentration, and purity. The results of the SDS/PAGE analyses were similar for all protein preparations. Based on the gel results, purified Z-TACI-Ig or [S40G]huBCMA ECD-Ig fusion protein dimers have an apparent MW of approximately 70 or 62 kDa, respectively, which is consistent with the predicted MW of the exemplary homodimeric protein.

### B. Endotoxin Analysis

Endotoxin levels of the fusion protein preparations were measured at Nelson Laboratories, Inc. (Taylorsville, UT) using a QCL-1000 Limulus Amoebocyte Lysate (LAL) assay kit. The maximum endotoxin level for proteins used in cell-based assays was set at 10 endotoxin units (EU)/mg protein.

### C. Size Exclusion Chromatography (SEC) Analysis

Protein aggregation levels (including aggregation levels of Z-TACI-Ig or [S40G]huBCMA ECD-Ig fusion proteins) were measured by size exclusion chromatography using a HPLC system. Protein (20 μg) was run through a TSK 3000 SWxL column (Tosoh Bioscience) fitted with a TSK Guard SWxL column using a mobile phase of 200 mM potassium phosphate, 150 mM potassium chloride, pH 6.8. The elution profile was monitored for 30 min with detection by absorbance (A) at 220 nanometers (nm). The maximum level of aggregation for protein used in further assays was set at 10%.

SEC analysis was performed on all protein preparations to verify protein quality in terms of protein aggregation levels. The results indicated that purified Z-TACI-Ig dimer is largely homogeneous in size and does not contain high levels of aggregated species (data not shown).

### Example 4: Construction, Production, and Detection of Phage Displaying Variant BCMA Polypeptides

This example describes exemplary methods used to create and screen libraries of BCMA ECD variants for altered human BAFF and/or human APRIL binding activities by phage display.

A portion of the ECD of BCMA was displayed on the surface of filamentous phage using the monovalent phage-display vector pSB0124. pSB0124 is a helper-dependent phagemid vector containing the bla gene for ampicillin resistance selection in E. coli, the filamentous phage M13 origin of replication, the STII secretory signal sequence, a 6-His Tag (SEQ ID NO: 253), a suppressible amber codon (TAG), and the C-terminal portion of the M13 gene III. Unique SfiI and NotI sites engineered between the STII secretory signal sequence and the suppressible amber codon permit cloning of a DNA sequence encoding the polypeptide to be displayed to generate an in-frame fusion protein for display along with a His tag on copies of the pIII coat protein expressed from the phagemid vector in an amber-suppressor E. coli strain (such as TG1).

The double stranded phagemid DNA was transformed into TG1 E. coli cells (Stratagene Cat. No. 200123), which carry an F plasmid and thus display an F pilus and were permissive for phage infection. The resulting transformants were infected with M13 helper phage, and phage were produced, which carry a mature recombinant coat protein consisting of the polypeptide for display fused to the M13 protein pIII, in addition to full-length pIII derived from the helper phage.

DNA encoding the human BCMA ECD (SEQ ID NO:152, corresponding to amino acids residues 5-54 of huBCMA set forth in SEQ ID NO:149) was prepared by PCR assembly using overlapping oligonucleotides. These oligonucleotides included flanking SfiI and NotI sites restriction sites which were used to subclone the PCR product into pSB0124. The assembled BCMA PCR product was digested with restriction enzymes SfiI and NotI, fragments were separated by agarose-gel electrophoresis, the BCMA fragment was purified using a Qiaquick™ Gel Extraction Kit (Qiagen, #28704) and ligated into similarly digested vector pSB0124 to generate pSB0124-BCMA. The DNA was transformed into E. coli TOP10 cells, DNA was prepared and transformed into TG1 cells, and the transformants were used for phage production as described in Example 5 below.

The phage-displayed variant BCMAs described below were tested for function using a phage ELISA. NUNC MaxiSorp™ plates were coated overnight at 4° C. with 50 μl/well of BAFF (R&D Systems, Catalog Number 2149-BF/CF) or APRIL (R&D Systems Catalog Number 884-AP/CF) at a concentration of 0.5 and 4 μg/ml (respectively) in PBS. After washing with PBS-T (PBS plus 0.02% TWEEN-20), the plates were blocked with PBS containing 3% milk (nonfat dried milk powder; Sigma-Aldrich). The block was washed and phage dilutions (2-fold titrations in PBS-T plus 1% milk) were added to the coated plates and incubated at room temperature. After 2 h of incubation, the plates were washed with PBS-T and bound phage was detected by incubating with horseradish peroxidase (HRP)-anti M13 antibody conjugate (GE Healthcare) for 30 min., washing with PBS-T, and detecting the immobilized HRP using TMB-H₂O₂ reagent (Pierce). The reaction was stopped after color development using 2M H₂SO₄ and the plate was read for absorbance at 450 nM using a spectrophotometer and SoftMaxPro™ software (Molecular Devices, Sunnyvale CA). The absorbance was plotted against phage dilution to obtain a binding curve to compare the binding of different variants (data not shown).

### Example 5: Generation and Screening of BCMA Variant Polypeptides by Phage Display

This example describes the generation of libraries of variant BCMA polypeptides and screen these libraries for altered human BAFF and/or human APRIL binding activities by phage display.

### A. DNA Sequences Encoding BCMA Variant Polypeptides

Directed evolution methods were used to generate libraries of recombinant non-naturally-occurring polynucleotides encoding BCMA ECD variant polypeptides. Directed evolution procedures included, e.g., in vitro recombination and mutagenesis procedures as substantially described in Stemmer, Proc. Natl. Acad. Sci. USA 1994; 91:10747-10751; Chang et al., Nature Biotech. 1999; 17:793-797; International Patent Application Publication No. WO 98/27230; and U.S. Pat. Nos. 6,117,679 and 6,537,776, all of which are incorporated herein by reference.

DNA sequences encoding recombinant variant BCMA polypeptides were then amplified from assembly reactions by PCR using forward and reverse primers designed based on sequence homology. Exemplary forward and reverse primers included the forward primer and the reverse primer. 5 μl of assembly reaction was used as template in a 100 μl PCR reaction with 1 pM forward and reverse primers, Taq buffer (Qiagen; Cat. No. 201225) and 200 pM dNTPs for 15 amplification cycles (94° C. 30 s; 50° C. 30 s; 72° C. 40 s). Amplified DNA encoding the variant BCMA polypeptides was digested with restriction enzymes (SfiI and NotI) and the fragments were separated by agarose-gel electrophoresis, purified using the Qiaquick™ Gel Extraction Kit (Qiagen, Catalog No. 28704) as per manufacturer's recommendation, and ligated into similarly digested phage-display vector pSB0124 (Chang et al., Nature Biotech 1999; 17:793-797). The resulting library ligation was transformed by electroporation into TOP10 E. coli cells (Invitrogen, Inc. Cat. No. C4040-50) following the manufacturer's recommended conditions. Transformed cells were incubated in LB (Luria broth media) containing 50 μg/ml carbenicillin at 250 rpm overnight at 37° C. and then used to make a maxiprep (Qiagen Cat. No. 12362) stock of library DNA as per the manufacturer's recommended conditions.

### B. Generation of Phage Displaying Libraries of BCMA Variant Polypeptides

Library DNA (e.g., a library of DNA sequences encoding BCMA ECD variant polypeptides in a variant BCMA-pIII fusion) was transformed by electroporation into TG-1 E. coli cells (Stratagene Cat. No. 200123) as per the manufacturer's recommended conditions. The culture was grown under phagemid-selection conditions (LB medium containing carbenicillin at 50 μg/ml) for 1-2 generations, infected with helper phage M13KO7 (at a multiplicity of infection level of 5-10), and incubated with shaking at 250 rpm overnight at 37° C. under dual selection for phagemid (carbenicillin at 50 μg/ml) and helper phage (kanamycin at 70 μg/ml). The cultures were clarified by centrifugation (6000 rpm, 15 min, 4° C. in a Sorvall 600TC rotor) and phage particles were precipitated by incubating 32 ml culture supernatant with 8 ml PEG/NaCl solution (20% PEG-8000; 2.5 M NaCl) on ice for 30 min followed by centrifugation (9500 rpm, 40 min, 4° C. in a Sorvall 600TC rotor). The phage pellet was suspended in 1 ml PBS containing 1% BSA (bovine serum albumin, Sigma Cat. No. A7906), transferred to a microfuge tube, and clarified by centrifugation (max speed, 5 min, RT in an Eppendorf table-top centrifuge). The resulting phage library consisted of phage that displayed variants of the BCMA ECD fused to the N-terminus of the pIII minor phage-coat protein. These variant BCMA polypeptides were therefore not constructed as an Ig-fusion protein and did not exist in dimeric form.

### C. Panning of BCMA Variant Polypeptide Phage Libraries

Phage libraries were panned in up to five alternating or sequential rounds against BAFF or APRIL proteins using standard conditions. See, e.g., Lowman, et al., Biochemistry 1991; 30(45):10832-10838; Smith, G. P. et al., Chem. Rev. 1997; 97:391-410, each of which is incorporated herein by reference. Each round of panning included: binding of phage displaying the BCMA ECD variant polypeptides to BAFF (Recombinant Human BAFF/BLyS/TNFSF13B, CF; R&D Systems Catalog No. 2149-BF/CF; Accession No. Q9Y275, SEQ ID NO:214) at 0.5 or 0.05 μg/ml, or APRIL (Recombinant Human APRIL/TNFSF13, CF; R&D Systems Catalog No. 884-AP; Accession No. Q8NFH7, SEQ ID NO:217, amino acid residues 110-250) at a concentration of 4 μg/ml; (b) removal of unbound phage; (c) elution of bound phage; and (d) amplification of eluted phage for the next round of panning. An aliquot of phage from each round was transduced into E. coli cells to obtain individual transductant colonies.

In some instances, the above procedure selected phage that had duplicated the variant BCMA ECD-coding sequence resulting in tandem variant BCMA polypeptides being fused to the phage pIII protein. To prevent such phage from dominating the selection, the following series of steps was introduced after step (c) above: (i) the variant BCMA ECD-coding sequences were PCR amplified from the eluted phage with the same primers that were used for the original library cloning; (ii) the amplified fragments were digested with SfiI and NotI, separated by gel electrophoresis, and only those fragments that had the expected size for a single copy of a variant BCMA ECD sequence were re-cloned into the phagemid vector; (iii) the resulting phagemid library was used to make a phage library as described above and used for the next round of selection.

D. Identification of BCMA Variant Polypeptides having Improved Binding to Human BAFF and/or Human APRIL by Phage ELISA

Individual colonies obtained from each round of panning were inoculated into 96-well culture plates (NUNC, Cat. No. 243656) containing 150 μl/well of 2×YT (yeast-tryptone) media containing 50 μg/ml carbenicillin and incubated at 250 rpm overnight at 37° C. The overnight cultures were used to inoculate deepwell blocks (Scienceware, Cat. No. 378600001) containing 600 μl/well of the same media. Cultures were incubated at 250 rpm for 2 hrs at 37° C., infected with M13K07 helper phage (multiplicity of infection (moi) 5-10) and then incubated at 250 rpm at overnight at 37° C. under dual selection for phagemid and helper phage markers (carbenicillin at 50 μg/ml and kanamycin at 70 μg/ml, respectively). Cultures were clarified by centrifugation at 4000 rpm for 20 min at 4° C. in a Beckman GH 3.8 rotor. ELISA plates (NUNC, Cat. No. 449824) were coated by addition of 50 μl/well TBS containing BAFF (R&D Systems, Cat. No. 2149-BF/CF) or APRIL (R&D Systems, Cat. No. 884-AP-010/CF) at a concentration of 0.5 or 4 μg/ml, respectively, and incubated overnight at 4° C. Plates were washed three times with 200 μl/well TBST and blocked by addition of 200 μl/well TBS containing 3% non-fat dry milk and incubation at RT for 1 hour (hr). 25 μl/well of phage supernatants from the deepwell block was transferred to ELISA plates containing 25 μl/well 6% non-fat dry milk and plates were incubated for 1 hr at room temperature (RT). Plates were washed three times with 200 μl/well TBST and incubated with 50 μl/well HRP-conjugated anti-M13 monoclonal antibody (GE Healthcare, Cat. No. 27-9421-01) diluted 1:5000 in TBST containing 3% non-fat dry milk for 1 hr at RT. Plates were washed three times with 200 μl/well TBST and signal was detected using a TMB substrate kit (Pierce, Cat. No. 34021) according to manufacturer's recommended conditions.

Variant BCMA-phage-displayed polypeptides that exhibited increased binding to human BAFF and/or human APRIL, compared to the binding of phage-displayed human BCMA (Example 4) to human BAFF and/or human APRIL, were selected for further analysis.

### Example 6: Cloning of Nucleotide Sequences Encoding BCMA Variant Polypeptides into pcDNA-PIg18 Fusion Vector

This Example describes generation of variant BCMA fusion polypeptides. Plasmids and post-translational modifications of the encoded polypeptides are also described.

To produce variant BCMA polypeptides as soluble Fc fusion proteins, DNA sequences encoding variant BCMA-ECDs that exhibited improved binding to human BAFF and/or human APRIL (relative to native human BCMA-ECD) from the phage library screening were each subcloned from the phagemid vector into a modified-PIg18-Fc fusion vector. A schematic of this fusion is provided in FIG. 6. The DNA sequence encoding the entire CTLA-4-signal peptide-BCMA ECD variant-PIg18 fusion described above was subcloned into the pcDNA3.1 vector (Invitrogen). Plasmid pcDNA3.1 is a mammalian expression vector that contains an expression cassette controlled by a CMV promoter and bGH (bovine growth hormone) terminator, and neomycin-resistance and ampicillin-resistance selectable markers for mammalian and bacterial selection, respectively. The DNA sequence encoding the fusion polypeptide contains two unique restriction sites, AgeI and KpnI, located in the CTLA-4-signal encoding region and the 3′ end of the BCMA ECD variant-encoding sequence, respectively. Plasmid pcDNA-BCMA-PIg18 was digested with AgeI and KpnI to release the BCMA ECD variant-encoding fragment and the pcDNA3.1-PIg18-Fc fusion vector fragment. The latter was used below for cloning the BCMA ECD variant-encoding fragments.

The BCMA ECD variant-encoding sequence was amplified from the phage clones that were selected as described above, by PCR with forward and reverse primers that introduced the AgeI and KpnI sites at the 5′ and 3′ ends, respectively. The DNA fragment was digested with AgeI and KpnI, gel-purified, and ligated to the pCDNA3.1-PIg18-fusion vector generated above. The plasmids were sequenced to confirm correct DNA sequence of the entire coding sequence for the fusion polypeptide consisting of the CTLA-4 signal peptide, BCMA ECD variant, and PIg18-Fc (mutant IgG2-Fc).

The resulting plasmid expression vector comprised a nucleic acid fragment encoding a fusion polypeptide consisting of the CTLA-4 signal sequence, a BCMA ECD variant, and PIg18-Fc region, whose expression is driven by the CMV promoter. A bovine growth hormone (bGH) poly A termination signal sequence is located 3′ of the DNA encoding the fusion polypeptide. The vector also includes a Bla promoter; ampicillin resistant gene; pUC origin; SV40 polyadenylation (poly A) signal sequence; fl origin; SV40 promoter; and neomycin resistance gene. The signal peptide is typically cleaved during processing and thus the secreted fusion protein (i.e., mature form) of BCMA ECD variant-PIg18 does not ordinarily contain the signal peptide sequence. The BCMA ECD variant-Ig fusion protein typically exists in solution as a dimeric fusion protein. In this instance, mature BCMA ECD variant-Ig fusion protein comprises a BCMA ECD variant fused at its C-terminus to the N-terminus of a human PIg18-Fc forming the BCMA ECD variant-Ig fusion protein dimer. The C-terminal lysine of the fusion protein may also be missing as a result of post-translational processing in the host cell. The BCMA ECD variant-Ig fusion protein dimer is the form of the fusion protein molecule used in the assays described in these Examples, unless stated otherwise.

### Example 7: Transient Expression of BCMA ECD Variant-PIg18 Polypeptides in CHO-S Cells

In order to harvest BCMA ECD variant-Ig polypeptides, plasmid expression vectors comprising the polynucleotide sequence encoding each BCMA ECD variant-Ig fusion polypeptides were transfected into host cells. FREESTYLE™ CHO-S cells (Life Technologies, Cat. No. R80007) were derived from CHO-K1 cells and adapted to serum-free suspension culture in FREESTYLE™ culture medium (FREESTYLE™ medium, Life Technologies Cat. No. 12651 supplemented with 4 mM L-Glutamine, Life Technologies, Cat. No. 25031). The CHO-S cells were transfected with the BCMA ECD variant-Ig plasmids using FREESTYLE MAX Reagent (Life Technologies, Cat. No. 16447-100) according to the manufacturer's recommended conditions. The transfected cells were grown in 80 mls of culture medium in 250 ml shake flasks for 5 days at 37° C., 8% CO₂. Protein expression was assessed on day 5 by Protein A HPLC (as described in Example 2C) and the cultures that showed good expression were harvested on day 6 and purified by protein A affinity chromatography as described below.

### Example 8: Purification and Characterization of BCMA ECD Variant Polypeptides

Supernatants harvested from the transient transfections described above were clarified by centrifugation at 1000×g for 10 min at RT and filtration through 0.2 μm membranes (Nalgene, VWR, Cat. No. 73520-982). The BCMA ECD variant-Ig fusion polypeptides were purified by Protein-A affinity chromatography using an AKTA (HiTrap) Explorer HPLC system (GE Healthcare). Supernatants were loaded onto Hitrap Protein A FF columns (GE Healthcare, Cat. No. 17-5079-01) in PBS buffer, washed with the same buffer, and the fusion proteins were eluted with 100 mM citric acid buffer (pH 4.0), and then neutralized by addition of 1/10 volume of 2M Tris base. The proteins were dialyzed into PBS buffer using 10 kDa MWCO membranes (Pierce, Cat. No. PI66810). The BCMA ECD variant-pIg18 fusion polypeptides were analyzed as described in Examples 3A and 3C.

### Example 9: Measuring Binding Activities of Variant BCMA ECD-Ig Fusion Polypeptides to BAFF and APRIL Using Surface Plasmon Resonance (SPR) (BIACORE Analysis)

This example describes a procedure for screening BCMA ECD variants as Ig fusion polypeptides (BCMA ECD variant-pIg18 fusion polypeptides) for improved binding activity to human BAFF, cynomolgus monkey APRIL, mouse BAFF, or mouse APRIL ligands using a Surface Plasmon Resonance (BIACORE™) interaction analysis. The cynomolgus monkey APRIL ligand was used as a surrogate for the human form in this screening assay. In the nomenclature used to describe this type of analysis, the immobilized binding partner is referred to as the “ligand”, and the binding partner in the mobile phase is referred to as the “analyte.” Fusion polypeptides containing Fc-region Ig domains typically exist as dimeric structures in solution by virtue of strong association between the Ig domains. Unless indicated otherwise, such dimeric conformations are expected to exist for the fusion polypeptides described in this Example (e.g., BCMA ECD variant-Ig polypeptides and Z-TACI-Ig). Trimeric conformations are expected to exist for human BAFF, cynomolgus monkey APRIL, mouse BAFF, and mouse APRIL as the native cytokines exist in trimeric states, the binding sites for the receptors TACI and BCMA occur at the interface between subunits, and the binding by receptors is expected only for trimers or higher order form of BAFF and APRIL.

The term “avidity” typically relates to the strength of binding between dimeric analytes (e.g., variant BCMA-Ig fusion proteins) and trimeric ligands (e.g., human BAFF, cynomolgus monkey APRIL, mouse BAFF, and mouse APRIL). The greater binding avidity exhibited by the variant BCMA-Ig fusion proteins (relative to surrogate control [S40G]huBCMA ECD-Ig) can be attributed to greater binding activity between each variant BCMA ECD domain and its corresponding ligand. The strength of binding avidity is typically described in terms of the equilibrium dissociation constant (K_{D}), which describes the molar concentration of analyte at which 50% of available ligand is bound at equilibrium.

In this screening method, BIACORE sensor chips were derivatized with antibody which captured BAFF or APRIL ligands, and variant BCMA-Ig fusion proteins in buffer were allowed to flow over the ligand-coated sensor chips. The ability of a variant BCMA-Ig fusion polypeptide to bind to a specific binding partner (i.e., human BAFF, cynomolgus monkey APRIL, mouse BAFF, or mouse APRIL) was evaluated. Control fusion polypeptide (i.e., human Z-TACI-Ig or [S40G]huBCMA ECD-Ig fusion protein) was also allowed to flow over the ligand-coated sensor chips and the ability of this molecule to bind to human BAFF, cynomolgus monkey APRIL, mouse BAFF, or mouse APRIL was similarly evaluated for comparison. Using the BIACORE system, the association (kₒₙ) and dissociation (k_{off}) rate constant of a protein of interest binding human BAFF, cynomolgus monkey APRIL, mouse BAFF, and mouse APRIL ligands can be evaluated and used to calculate the equilibrium dissociation constant, K_{D}. The human Z-TACI-Ig fusion protein, which comprises the wild-type human TACI ECD polypeptide fused to the human IgG1 Fc polypeptide, serves as a wild-type human TACI “control”. In this manner, variant BCMA-Ig fusion polypeptides having increased binding avidities for human BAFF, cynomolgus monkey APRIL, mouse BAFF, and/or mouse APRIL, compared to the human Z-TACI-Ig and [S40G]huBCMA ECD-Ig fusion polypeptides, were identified.

All BIACORE analyses were performed on a BIACORE™ 2000 or BIACORE™ 3000 system (GE Healthcare) at room temperature (RT, 25° C.). HBS-EP buffer (10 mM HEPES (pH 7.4), 150 mM NaCl, 3 mM EDTA, 0.005% surfactant P20) was used as the flow buffer in all experiments.

The standard kinetic assay measures binding kinetics of trimeric ligand (e.g., human BAFF, cynomolgus monkey APRIL, mouse BAFF, or mouse APRIL) coated on sensor chips and dimeric analytes (e.g., variant BCMA-Ig fusion proteins) in the mobile phase. For mouse BAFF measurements, mouse monoclonal antibody (IgG1) directed against the pentaHis tag (Novagen, Cat. No. 70796) was immobilized on CM3 sensor chips (GE Healthcare, Cat. No. BR-1005-41) according to the manufacturer's protocol. For human BAFF, cynomolgus monkey APRIL, and mouse APRIL measurements, mouse monoclonal antibody (IgG1) anti-FLAG M2 (Sigma Aldrich, Cat. No. F1804) was immobilized on CM3 sensor chips (GE Healthcare, Cat. No. BR-1005-41) according to the manufacturer's protocol. Antibody was diluted to 30 μg/ml in immobilization buffer (10 mM pH 5±0.20; GE Healthcare; GE Healthcare, Catalog No. BR-1003-51). At a flow rate of 5 μl/minute, sensor chip CM3 was activated with a 35 μl injection of a mixture of 1-Ethyl-3-[3-dimethylaminopropyl]carbodiimide hydrochloride (EDC) and 30 N-hydroxysuccinimide (NHS) (made by mixing equal volumes of 11.5 mg/ml EDC and 75 mg/ml NHS; GE Healthcare, Cat. No. BR-1000-50), followed by a 35 μl injection of undiluted antibody. Unreacted sites were quenched with 35 μl of 1M ethanolamine-HCl, pH 8.5 (GE Healthcare, GE Healthcare, Cat. No. BR-1000-50). This procedure typically yielded 3,000-4,000 response units (RU) of coupled antibody.

Ligand 6HIS-tagged murine BAFF (R&D Systems, Cat. No. 2106-BF; Accession No. Q9WU72; amino acid residues A127-L309) were bound to anti-pentaHis antibody-coated sensor chips by injection of 5 μl, ligand solution (2 μg/ml protein in HBS-EP buffer [10 mM HEPES pH 7.4, 150 mM NaCl, 3 mM EDTA, 0.005% (v/v) surfactant P-20; GE Healthcare, Cat. No. BR-1001-88]) at a flow rate of 10 μl/min. Ligand capture levels were typically 30-60 RU. Ligands FLAG-tagged murine megaAPRIL (Alexis Biochemicals, Cat. No. ALX-522-092-3010; Alexis cat no. ALX-522-092-3010; Accession No. AAG22534; amino acid residues 98-232), FLAG-tagged human BAFF expressed as a fusion with the TPA-signal peptide, or FLAG-tagged cynomolgous APRIL expressed as a fusion with the TPA-signal peptide, were bound to anti-FLAG M2 antibody-coated sensor chips by injection of 5 μl, ligand solution (1 μg/ml protein in HBS-EP buffer [10 mM HEPES pH 7.4, 150 mM NaCl, 3 mM EDTA, 0.005% (v/v) surfactant P-20; GE Healthcare, Catalog No. BR-1001-88]) at a flow rate of 10 μl/min. Ligand capture levels were typically 30-60 RU.

The fusion polypeptides were diluted in HBS-EP buffer and flowed over ligand-coated sensor chips. The variant BCMA-Ig and surrogate control fusion proteins were injected at three concentrations (1.5 nM, 15 nM, and 150 nM at 30 μl/min for 2 minutes per association phase) in a single binding cycle, followed by a 20 minute incubation with HBS-EP buffer containing no protein at the same flow rate. A similar protocol was followed for Z-TACI-Ig, except that the injections were at 4.5 nM, 45 nM, and 450 nM.

Rₘₐₓ signal levels for variant BCMA ECD-Ig fusion proteins ranged from approximately 15-120 RU. Regeneration between cycles was performed by 2.5 min incubation with 1 M arginine, 0.1 M acetic acid buffer (pH 3.6) at 50 μl/min. New chips were subjected to 4-5 cycles of capture/binding/regeneration prior to use in actual experiments. Data from a reference cell containing mouse anti-pentaHis or mouse anti-FLAG M2 capture antibody alone was subtracted from data obtained from flow cells containing captured human BAFF, cynomolgous APRIL, mouse BAFF or mouse APRIL.

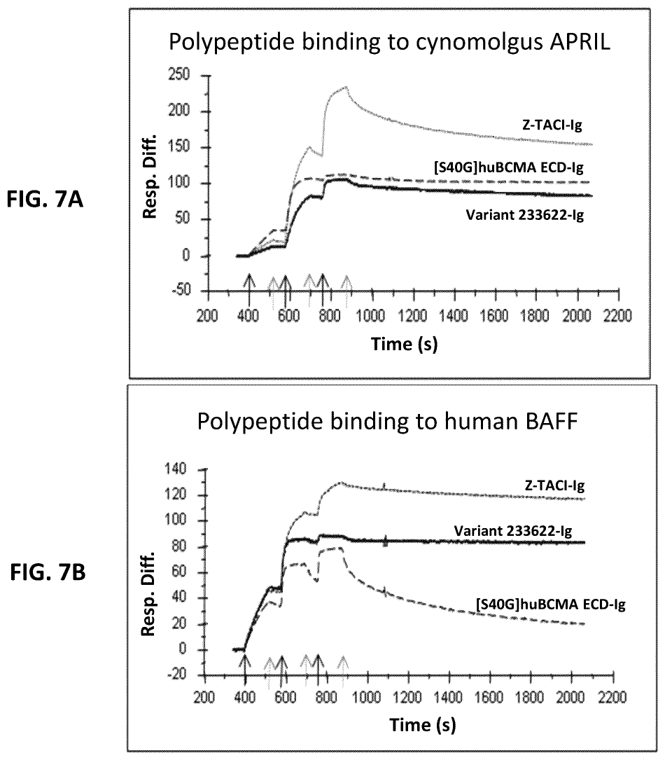

Sensorgram traces of an exemplary variant BCMA ECD-Ig fusion polypeptide Variant 233622-Ig (solid curve), [S40G]huBCMA ECD-Ig fusion protein (dashed curve), and Z-TACI-Ig fusion protein (dotted curve) binding to cynomolgous APRIL and human BAFF are shown in FIG. 7A and FIG. 7B, respectively. The solid arrows mark the beginning of each association phase, the dotted (gray) arrows mark the beginning of each disassociation phase.

The shape of the association phase and the concentration of analyte influence the kₒₙ, whereas the shape of the dissociation phase determines the k_{off}. The data were analyzed using BIAevaluation™ software (v4.1, available from GE Healthcare) which calculated the kₒₙ, k_{off}, and K_{D} binding parameters by curve-fitting.

Recombinant cyAPRIL (GenBank Accession No. XM_001109101.2; Rhesus TNF domain is from amino acid residues 190-330) was used in the BIACORE studies instead of recombinant huAPRIL because cyAPRIL showed more stable BIACORE™ FLAG capture levels as compared to huAPRIL. Natural, secreted cyAPRIL is identical to rhesus APRIL but differs from secreted huAPRIL by only two amino acids, neither of which are near the binding pocket for BCMA or TAC. As expected, similar binding activities of variant BCMA proteins were observed with both recombinant cyAPRIL and recombinant huAPRIL. Furthermore, activity of variant BCMA proteins on huAPRIL was confirmed by the Kinetic Exclusion Assay described in Example 11.

The analyses described above were performed on protein preparations of representative variant BCMA ECD-Ig fusion polypeptides, as well as the Z-TACI-Ig and [S40G]huBCMA ECD-Ig control fusion polypeptides, and the data is presented in Table 1.

TABLE 1
Binding activities determined by Surface Plasmon Resonance (BIACORE) analysis of Z-TACI-
Ig, [S40G]huBCMA ECD-Ig and representative variant BCMA ECD-Ig fusion polypeptides*

| | SEQ ID | | Fold | | Fold | | |
|---|---|---|---|---|---|---|---|
| | NO. | | Improvement | | improvement | | |
| | (CRD of | | K_{D}, | | K_{D}, | | |
| | ECD | | huBAFF, | | cyAPRIL, | | |
| | component of | K_{D}, | relative | K_{D}, | relative | K_{D}, | K_{D}, |
| | BCMA | huBAFF | to | cyAPRIL | to | mBAFF | mAPRIL |
| Clone Name | ECD-Ig) | (pM) | control | (pM) | Control | (pM) | (pM) |
| [S40G]huBCMA | 224 | 121 | 1 | 15.1 | 1 | 66.9 | 21.6 |
| ECD-Ig Control | | | | | | | |
| Z-TACI-Ig | | 74.5 | 1.6 | 1210 | 0.01 | 229 | 421 |
| Variant 238833-Ig | | — | | — | | 50.9 | 242 |
| Variant 238475-Ig | | 55.9 | 2.2 | 78.9 | 0.20 | 26.4 | 80.2 |
| Variant BAv66-Ig | 124 | 92.2 | 1.3 | 62600 | 2.4e−4 | 62.3 | 4900 |
| Variant BAv1-12- | 9 | 12.3 | 9.8 | 10.1 | 1.5 | 33.9 | 28.1 |
| Ig | | | | | | | |
| Variant BAv3-10- | 4 | 4.92 | 24.6 | 13.9 | 1.1 | 14.9 | 29.3 |
| Ig | | | | | | | |
| Variant BAv9-1- | 3 | 8.17 | 14.8 | 21.4 | 0.7 | 15 | 38.4 |
| Ig | | | | | | | |
| Variant BAv8-10- | 6 | 22.9 | 5.3 | 53.9 | 0.3 | 17.1 | 47.8 |
| Ig | | | | | | | |
| Variant BAv8-9- | 8 | 9.63 | 12.6 | 22.6 | 0.7 | 14.9 | 44.2 |
| Ig | | | | | | | |
| Variant BAv9-5- | 5 | 11.1 | 10.9 | 28.4 | 0.5 | 21.6 | 45.2 |
| Ig | | | | | | | |
| Variant BAv11-6- | 7 | 15.9 | 7.6 | 24.4 | 0.6 | 18.1 | 47.4 |
| Ig | | | | | | | |

*Binding activities for human BAFF (“huBAFF”), cynomolgous APRIL (“cyAPRIL”), mouse BAFF (“mBAFF”), and mouse MegaAPRIL (“mAPRIL”) are shown as equilibrium dissociation constants (K_{D}s).

In Table 1, each K_{D} value is calculated by the formula K_{D}=kd/ka. Average ka and kd values from independent determinations were used. For human BAFF and cynomolgous APRIL ka determinations n=1 or 2 except for Z-TACI-Ig and [S40G]huBCMA ECD-Ig, n=3. For human BAFF and cynomolgous APRIL kd values represent 1-2 independent experiments for each compound except for Z-TACI-Ig and [S40G]huBCMA ECD-Ig, n=4. For mouse BAFF and MegaA-PRTL, kinetics, 2 ka determinations and 2 kd determinations were done for each compound.

### Example 10: Sequence Optimization

Using available structures, sequence analysis, and information from published literature, the variant BCMA ECD polypeptide sequences of the variant BCMA ECD-Ig fusion polypeptides exhibiting the best BAFF and APRIL avidities were sequence optimized to minimize the mutation load whilst maintaining the improved binding to both ligands. Sequence optimization involved generating revertants from the variant BCMA ECD-Ig molecules using standard site-directed mutagenesis methods such as by SOEing as described herein above. Single as well as multiple revertants from twelve different variant-BCMA-pIg18 were generated, cloned in the pcDNA3.1 vector (Life Technologies, as described in Example 6), expressed and purified as described in Examples 6-8. The resulting proteins were assayed for their binding characteristics using Surface Plasmon Resonance as described in Example 9. Binding activities of the derivatives relative to the parents are shown in Table 2. Comparison of various derivatives allows one to deduce the relative contribution of individual mutations to the BAFF and APRIL binding of the variant BCMA ECD-Ig polypeptide fusion. For example, reverting 114 and N15 to the corresponding native residues (i.e., 114L and N15H) resulted in decreased BAFF binding activity, indicating that these mutations are beneficial for improving BAFF binding activity. The +/− nomenclature used the following method. The SPR curves were compared with the curves from the parent molecule in the dissociation region (Example 9, FIG. 7A and FIG. 7B) and the curves were categorized as being similar to parent if they remained parallel, − if the curve had a steeper slope than parent (dissociated faster), or + if it had a shallower slope than parent (dissociated slower).

TABLE 2
Sequence variation of representative variant BCMA polypeptides as described and huBAFF
and cyAPRIL binding activity

| | SEQ ID | | | |
|---|---|---|---|---|
| | NO. (CRD | | Binding Activity | |
| | of ECD | | huBAFF binding, | cyAPRIL binding, |
| | component | | fold improvement | fold improvement |
| | of BCMA | Substitutions Indicated Relative to | over control | over control |
| Clone Name | ECD-Ig) | huBCMA ECD (SEQ ID NO: 152) | (K_{D}, pM) | (K_{D}, pM) |
| [S40G]- | 224 | S40G | 1 (121) | 1 (15.1) |
| BCMA ECD- | | | | |
| Ig Control | | | | |
| Variant | 10 | G2Q + S12R + L14V + H15N + | 6.4 (19) | 0.5 (28) |
| 50238779-Ig | Parent 1 | A16S + I18V + Q21R + T28N + | | |
| | | R35H + S40G | | |
| Variant 1-Ig | 11 | G2Q + S12R + L14V + H15N + | Similar to parent 1 | Similar to parent 1 |
| | | A16S + T28N + R35H + S40G | | |
| Variant 1-1-Ig | 12 | S12R + L14V + H15N + A16S + | Similar to parent 1 | Similar to parent 1 |
| | | T28N + R35H + S40G | | |
| Variant 1-2-Ig | 13 | G2Q + L14V + H15N + A16S + | − | Similar to parent 1 |
| | | T28N + R35H + S40G | | |
| Variant 1-3-Ig | 14 | G2Q + S12R + H15N + A16S + | − | Similar to parent 1 |
| | | T28N + R35H + S40G | | |
| Variant 1-4-Ig | 15 | G2Q + S12R + L14V + A16S + | − | Similar to parent 1 |
| | | T28N + R35H + S40G | | |
| Variant 1-5-Ig | 16 | G2Q + S12R + L14V + H15N + | Similar to parent 1 | Similar to parent 1 |
| | | T28N + R35H + S40G | | |
| Variant 1-6-Ig | 17 | G2Q + S12R + L14V + H15N + | + | Similar to parent 1 |
| | | A16S + R35H + S40G | | |
| Variant 1-7-Ig | 18 | G2Q + S12R + L14V + H15N + | Similar to parent 1 | Similar to parent 1 |
| | | A16S + T28N + S40G | | |
| Variant 1-11-Ig | 19 | S12R + L14V + H15N + A16S + | Similar to parent 1 | Similar to parent 1 |
| | | S40G | | |
| Variant 1-12-Ig | 9 | S12R + L14V + H15N + S40G | Similar to parent 1 | Similar to parent 1 |
| Variant 1-13-Ig | 20 | L14V + H15N + S40G | − | Similar to parent 1 |
| Variant 1-14-Ig | 21 | S12H + L14V + H15N + S40G | − | Similar to parent 1 |
| Variant 1-15-Ig | 22 | S12H + L14V + H15N + A16S + | − | Similar to parent 1 |
| | | S40G | | |
| Variant 1-16-Ig | 23 | L14V + H15N + A16S + S40G | − | − |
| Variant 1-17-Ig | 24 | S12R + L14V + H15N + A16S + | + | − |
| | | L22V + S40G | | |
| Variant 1-18-Ig | 25 | S12R + L14V + H15N + L22V + | + | − |
| | | S40G | | |
| Variant 1-19-Ig | 26 | S12H + L14V + H15N + L22V + | + | − |
| | | S40G | | |
| Variant 1-20-Ig | 27 | L14V + H15N + L22V + S40G | + | − |
| Variant | 28 | G2Q + Q6P + S12R + L14I + | 6.4 (19) | 0.7 (23) |
| 50240054-Ig | Parent 2 | H15R + L31V + A39T + S40G | | |
| Variant 3-Ig | 29 | G2Q + Q6P + S12R + L14I + | Similar to parent 2 | Similar to parent 2 |
| | | H15R + A39T + S40G | | |
| Variant 3-1-Ig | 30 | Q6P + S12R + L14I + H15R + | Similar to parent 2 | Similar to parent 2 |
| | | A39T + S40G | | |
| Variant 3-2-Ig | 31 | G2Q + S12R + L14I + H15R + | Similar to parent 2 | Similar to parent2 |
| | | A39T + S40G | | |
| Variant 3-3-Ig | 32 | G2Q + Q6P + L14I + H15R + | − | Similar to parent 2 |
| | | A39T + S40G | | |
| Variant 3-4-Ig | 33 | G2Q + Q6P + S12R + H15R + | − | Similar to parent 2 |
| | | A39T + S40G | | |
| Variant 3-5-Ig | 34 | G2Q + Q6P + S12R + L14I + | − | Similar to parent 2 |
| | | A39T + S40G | | |
| Variant 3-6-Ig | 35 | G2Q + Q6P + S12R + L14I + | Similar to parent 2 | Similar to parent 2 |
| | | H15R + S40G | | |
| Variant 3-10-Ig | 4 | S12R + L14I + H15R + S40G | + | Similar to parent 2 |
| Variant 3-13-Ig | 36 | L14I + H15R + S40G | − | Similar to parent 2 |
| Variant 3-14-Ig | 37 | S12R + L14I + H15R + L22V + | + | Similar to parent 2 |
| | | S40G | | |
| Variant | 38 | F10Y + S12H + H15R + I18V + | 7.1 (17) | 0.4 (37) |
| 50237847-Ig | Parent 3 | P19S + Q21H + L22V + T28N + | | |
| | | L31A + T32I + S40G | | |
| Variant 8-Ig | 39 | S12H + H15R + P19S + L22V + | Similar to parent 3 | Similar to parent 3 |
| | | T28N + S40G | | |
| Variant 8-1-Ig | 40 | H15R + P19S + L22V + T28N + | − | Similar to parent 3 |
| | | S40G | | |
| Variant 8-2-Ig | 41 | S12H + P19S + L22V + T28N + | − | − |
| | | S40G | | |
| Variant 8-3-Ig | 42 | S12H + H15R + L22V + T28N + | + | Similar to parent 3 |
| | | S40G | | |
| Variant 8-4-Ig | 43 | S12H + H15R + P19S + T28N + | − | Similar to parent3 |
| | | S40G | | |
| Variant 8-5-Ig | 44 | S12H + H15R + P19S + L22V + | + | Similar to parent3 |
| | | S40G | | |
| Variant 8-9-Ig | 8 | S12H + H15R + S40G | Similar to parent3 | Similar to parent 3 |
| Variant 8-10-Ig | 6 | S12H + H15R + L22V + S40G | + | Similar to parent 3 |
| Variant | 45 | S12H + L14I + H15R + Q21D + | 5.5 (22) | 0.3 (59) |
| 50237854-Ig | Parent 4 | L22V + T28N + L31V + S40G | | |
| Variant 9-Ig | 46 | S12H + L14I + H15R + L22V + | Similar to parent 4 | Similar to parent4 |
| | | T28N + S40G | | |
| Variant 9-1-Ig | 3 | S12H + L14I + H15R + S40G | − (slight) | Similar to parent 4 |
| Variant 9-2-Ig | 47 | S12H + L14I + H15R + L22V + | Similar to parent 4 | Similar to parent 4 |
| | | S40G | | |
| Variant 9-3-Ig | 48 | S12H + L14V + H15R + L22V + | Similar to parent 4 | − |
| | | S40G | | |
| Variant 9-4-Ig | 49 | S12R + L14V + H15R + L22V + | Similar to parent 4 | − |
| | | S40G | | |
| Variant 9-5-Ig | 5 | S12R + L14V + H15R + S40G | Similar to parent 4 | Similar to parent 4 |
| Variant | 50 | G2R + F10Y + S12R + H15R + | 5.5 (22) | 0.9 (17) |
| 50239698-Ig | Parent 5 | A16S + I18V + Q21H + L22V + | | |
| | | T28N + L31V + S40G | | |
| Variant 11-Ig | 51 | G2R + S12R + H15R + A16S + | Similar to parent 5 | Similar to parent 5 |
| | | L22V + T28N + S40G | | |
| Variant 11-1-Ig | 52 | S12R + H15R + A16S + S40G | − | Similar to parent 5 |
| Variant 11-2-Ig | 53 | S12R + H15R + S40G | − | Similar to parent 5 |
| Variant 11-3-Ig | 54 | H15R + A16S + S40G | − | Similar to parent 5 |
| Variant 11-4-Ig | 55 | S12R + H15R + A16S + L22V + | + | Similar to parent 5 |
| | | S40G | | |
| Variant 11-5-Ig | 56 | H15R + S40G | − | Similar to parent 5 |
| Variant 11-6-Ig | 7 | S12R + H15R + L22V + S40G | Similar to parent 5 | Similar to parent 5 |
| Variant 11-7-Ig | | H15R + L22V + S40G | − | Similar to parent 5 |
| Variant | 57 | E8D + F10Y + S12H + H15R + | 4.2 (29) | 0.4 (39) |
| 50234224-Ig | Parent 6 | T28N + S40G | | |
| Variant 2-Ig | 58 | S12H + H15R + T28N + S40G | Similar to parent 6 | Similar to parent 6 |
| Variant | 59 | N7S + F10Y + S12R + L14I + | 5.5 (22) | 0.5 (28) |
| 50237844-Ig | Parent 7 | H15R + T28N + L31V + T32I + | | |
| | | S40G | | |
| Variant 4-Ig | 60 | N7S + S12R + L14I + H15R + | Similar to parent 7 | Similar to parent 7 |
| | | T28N + S40G | | |
| Variant | 61 | F10Y + S12R + L14I + H15R + | 4.5 (27) | 0.2 (72) |
| 50238504R-Ig | Parent 8 | I18L + Q21H + L22V + L31A + | | |
| | | S40G | | |
| Variant 6-Ig | 62 | S12R + L14I + H15R + L22V + | Similar to parent 8 | Similar to parent 8 |
| | | S40G | | |
| Variant | 63 | F10Y + S12H + L14I + H15R + | 4.7 (26) | 0.4 (42) |
| 50238475-Ig | Parent 9 | P19S + L221 + T28N + L31P + | | |
| | | T32I + S40G | | |
| Variant 12-Ig | 64 | S12H + L14I + H15R + P19S + | Similar to parent 9 | Similar to parent 9 |
| | | L22I + T28N + S40G | | |
| Variant | 65 | G2R + Q6P + N7S + F10Y + S12H + | 9.3 (13) | 0.3 (54) |
| 50239040-Ig | Parent 10 | L14I + H15R + A16S + I18V + | | |
| | | L31V + N38D + S40G | | |
| Variant 5-Ig | 66 | G2R + Q6P + N7S + S12H + L14I + | − | Similar to parent 10 |
| | | H15R + A16S + N38D + S40G | | |
| Variant | 67 | N7S + F10Y + S12R + L14I + | 7.6 (16) | 1.3 (12) |
| 50238494-Ig | Parent 11 | I18V + T28N + L31A + S40G | | |
| Variant 7-Ig | 68 | N7S + S12R + L14I + T28N + | − | Similar to parent 11 |
| | | S40G | | |
| Variant | 69 | G2R + Q6P + F10Y + L14I + | 10.1 (12) | 0.6 (24) |
| 50239978-Ig | Parent 12 | H15R + I18V + T28N + L31V + | | |
| | | S40G | | |
| Variant 10-Ig | 70 | G2R + Q6P + L14I + H15R + | − | Similar to parent 12 |
| | | T28N + S40G | | |

The sequence optimization procedure described above was also used to generate derivatives of variant BCMA polypeptides that displayed a BAFF-selective phenotype characterized by a BAFF binding activity in the low pM range and an APRIL affinity in the 100-500 pM range. The molecule BAv8-10 (SEQ TD NO:66) was used as the starting point for this sequence optimization process because it showed a preference for BAFF over APRIL (see, Table 4). Using available information from the structures and published literature, several residues in BAv8-10 were targeted for substitution with conserved as well as non-conserved residues and their effect on BAFF and APRIL binding was assessed by BIACORE. Mutations that reduced APRIL, binding (+/− or − in the table below) but did not appreciably affect BAFF binding (+), such as S12P, were subsequently combined and the process was iteratively applied. On the other hand, mutations such as L14S that reduced BAFF binding were left out of further iterations. The BIACORE™ binding activities of the derivatives relative to the parent BAv8-10 are shown in Table 3.

TABLE 3
Sequence variation of representative variant BCMA polypeptides as
described and huBAFF and cyAPRIL binding activities*

| | | | Binding Activity | |
|---|---|---|---|---|
| | | | K_{D}, huBAFF | K_{D}, cyAPRIL |
| | | | (pM)(fold | (pM)(fold |
| | SEQ ID | | improvement over | improvement over |
| | NO. (CRD | | control) [huBAFF | control [cyAPRIL |
| | of ECD | Substitutions | binding activity | binding Activity |
| | component | Indicated Relative | relative to BAv8- | relative to BAv8- |
| | of BCMA | to huBCMA ECD | 10 (SEQ ID | 10 (SEQ ID |
| Clone Name | ECD-Ig) | (SEQ ID NO: 152) | NO: 6)] | NO: 6)] |
| [S40G] | 224 | S40G | 173 (1.00) | 57 (1.00) |
| huBCMA-Ig | | | | |
| BAv8-10-Ig | 6 | S12H + H15R + L22V + | 13 (13.11) | 76 (0.75) |
| | | S40G | | |
| BAv13-Ig | 71 | S12P + H15R + L22V + | [=] | [−] |
| | | S40G | | |
| BAv14-Ig | 72 | S12T + H15R + L22V + | [=] | [+/−] |
| | | S40G | | |
| BAv15-Ig | 73 | S12N + H15R + L22V + | [=] | [=] |
| | | S40G | | |
| BAv16-Ig | 74 | S12Q + H15R + L22V + | [=] | [=] |
| | | S40G | | |
| BAv17-Ig | 75 | S12H + L14P + H15R + | [−] | [−] |
| | | L22V + S40G | | |
| BAv18-Ig | 76 | S12H + L14T + H15R + | [=] | [−] |
| | | L22V + S40G | | |
| BAv19-Ig | 77 | S12H + H15A + L22V + | [=] | [+] |
| | | S40G | | |
| BAv20-Ig | 78 | S12H + H15R + A16T + | [=] | [=] |
| | | L22V + S40G | | |
| BAv21-Ig | 79 | S12H + H15R + A16N + | [=] | [=] |
| | | L22V + S40G | | |
| BAv22-Ig | 80 | S12H + H15R + A16Q + | [=] | [=] |
| | | L22V + S40G | | |
| BAv23-Ig | 81 | S12H + H15R + A16V + | [=] | [=] |
| | | L22V + S40G | | |
| BAv24-Ig | 82 | S12H + H15R + A16Y + | [=] | [+/−] |
| | | L22V + S40G | | |
| BAv25-Ig | 83 | S12H + H15R + A16I + | [=] | [=] |
| | | L22V + S40G | | |
| BAv26-Ig | 84 | S12H + H15R + A16F + | [ND] | [ND] |
| | | L22V + S40G | | |
| BAv27-Ig | 85 | S12H + H15R + I18N + | [−] | [−] |
| | | L22V + S40G | | |
| BAv28-Ig | 86 | S12H + H15R + I18Q + | [−] | [−] |
| | | L22V + S40G | | |
| BAv29-Ig | 87 | S12H + H15R + I18S + | [−] | [−] |
| | | L22V + S40G | | |
| BAv30-Ig | 88 | S12H + H15R + I18T + | [=] | [−] |
| | | L22V + S40G | | |
| BAv31-Ig | 89 | S12H + H15R + I18A + | [=] | [−] |
| | | L22V + S40G | | |
| BAv32-Ig | 90 | S12H + H15R + P19A + | [=] | [=] |
| | | L22V + S40G | | |
| BAv33-Ig | 91 | S12H + H15R + P19G + | [=] | [=] |
| | | L22V + S40G | | |
| BAv34-Ig | 92 | S12H + H15R + P19T + | [=] | [+/−] |
| | | L22V + S40G | | |
| BAv35-Ig | 93 | S12H + H15R + P19N + | [=] | [=] |
| | | L22V + S40G | | |
| BAv36-Ig | 94 | S12H + H15R + P19Q + | [=] | [+/−] |
| | | L22V + S40G | | |
| BAv37-Ig | 95 | S12H + H15R + Q21I + | [=] | [=] |
| | | L22V + S40G | | |
| BAv38-Ig | 96 | S12H + H15R + L22A + | [=] | [+/−] |
| | | S40G | | |
| BAv39-Ig | 97 | S12H + H15R + L22Y + | [=] | [=] |
| | | S40G | | |
| BAv40-Ig | 98 | S12H + H15R + L22F + | [=] | [=] |
| | | S40G | | |
| BAv41-Ig | 99 | S12H + H15R + L22V + | [=] | [−] |
| | | R23A + S40G | | |
| BAv42-Ig | 100 | S12H + H15R + L22V + | [−] | [−] |
| | | R23G + S40G | | |
| BAv43-Ig | 101 | S12H + H15R + L22V + | [=] | [−] |
| | | R23S + S40G | | |
| BAv44-Ig | 102 | S12H + H15R + L22V + | [=] | [−] |
| | | R23T + S40G | | |
| BAv45-Ig | 103 | S12H + H15R + L22V + | [−] | [NB] |
| | | R23N + S40G | | |
| BAv46-Ig | 104 | S12H + H15R + L22V + | [+/−] | [−] |
| | | R23Q + S40G | | |
| BAv47-Ig | 105 | S12H + H15R + L22V + | [−] | [−] |
| | | R23W + S40G | | |
| BAv48-Ig | 106 | S12H + H15R + L22V + | [+/−] | [−] |
| | | R23H + S40G | | |
| BAv49-Ig | 107 | S12P + L14I + H15R + | [=] | [−] |
| | | L22V + S40G | | |
| BAv50-Ig | 108 | S12N + H15R + | [=] | [+/−] |
| | | L22Y + S40G | | |
| BAv51-Ig | 109 | S12H + L14I + H15R + | [=] | [−] |
| | | L22A + S40G | | |
| BAv52-Ig | 110 | S12H + H15R + A16Y + | [=] | [−] |
| | | L22Y + S40G | | |
| BAv53-Ig | 111 | L14I + H15R + A16H + | [=] | [−] |
| | | L22V + S40G | | |
| BAv54-Ig | 112 | S12H + L14S + H15R + | [NB] | [NB] |
| | | L22V + S40G | | |
| BAv55-Ig | 113 | S12H + L14G + H15R + | [−] | [NB] |
| | | L22V + S40G | | |
| BAv56-Ig | 114 | S12H + L14F + H15R + | [=] | [−] |
| | | L22V + S40G | | |
| BAv57-Ig | 115 | S12H + L14I + H15R + | [=] | [−] |
| | | L22S + S40G | | |
| BAv58-Ig | 116 | L14V + H15R + A16H + | [=] | [−] |
| | | L22I + S40G | | |
| BAv59-Ig | 117 | S12H + H15R + A16T + | [=] | [+/−] |
| | | L22Y + S40G | | |
| BAv60-Ig | 118 | L14V + H15R + A16H + | [=] | [−] |
| | | L22V + S40G | | |
| BAv61-Ig | 119 | L14I + H15R + A16H + | [=] | [−] |
| | | L22I + S40G | | |
| BAv62-Ig | 120 | L14V + H15R + A16T + | [=] | [−] |
| | | L22I + S40G | | |
| BAv63-Ig | 121 | S12H + L14A + H15R + | [−] | [NB] |
| | | L22V + S40G | | |
| BAv64-Ig | 122 | S12H + L14Y + H15R + | [=] | [NB] |
| | | L22V + S40G | | |
| BAv65-Ig | 123 | S12H + L14I + H15R + | [=] | [−] |
| | | L22T + S40G | | |
| BAv66-Ig | 124 | S12H + L14I + H15R + | [−] | [−] |
| | | L22G + S40G | | |
| BAv67-Ig | 125 | L14V + H15R + I18Q + | [NB] | [NB] |
| | | L22V + S40G | | |
| BAv68-Ig | 126 | L14I + H15R + I18Q + | [NB] | [NB] |
| | | L22V + S40G | | |
| BAv69-Ig | 127 | S12H + L14V + H15R + | [NB] | [NB] |
| | | I18Q + S40G | | |
| BAv70-Ig | 128 | S12H + L14I + H15R + | [NB] | [NB] |
| | | I18Q + S40G | | |
| BAv71-Ig | 129 | S12H + L14I + H15R + | [=] | [−] |
| | | A16I + L22V + S40G | | |
| BAv72-Ig | 130 | S12H + L14V + H15R + | [ND] | [ND] |
| | | A16I + L22V + S40G | | |
| BAv73-Ig | 131 | S12H + H15R + A16I + | [=] | [+/−] |
| | | L22Y + S40G | | |
| BAv74-Ig | 132 | S12H + L14I + H15R + | [=] | [−] |
| | | I18T + L22V + S40G | | |
| BAv75-Ig | 133 | S12H + L14V + H15R + | [=] | [−] |
| | | I18T + L22V + S40G | | |
| BAv76-Ig | 134 | S12H + L14I + H15R + | [NB] | [NB] |
| | | L22V + R23T + S40G | | |
| BAv77-Ig | 135 | S12H + L14V + H15R + | [NB] | [NB] |
| | | L22V + R23T + S40G | | |
| BAv78-Ig | 136 | S12H + H15R + L22Y + | [=] | [NB] |
| | | R23T + S40G | | |
| BAv79-Ig | 137 | S12P + L14I + H15R + | [=] | [NB] |
| | | L22A + S40G | | |
| BAv80-Ig | 138 | S12P + L14I + H15R + | [=] | [−] |
| | | L22I + S40G | | |
| BAv81-Ig | 139 | S12P + L14V + H15R + | [=] | [−] |
| | | L22V + S40G | | |
| BAv82-Ig | 140 | S12P + L14V + H15R + | [=] | NB |
| | | L22A + S40G | | |
| BAv83-Ig | 141 | S12P + L14V + H15R + | [=] | [−] |
| | | L22I + S40G | | |
| BAv84-Ig | 142 | S12H + L14I + H15R + | [=] | [=] |
| | | L22I + S40G | | |
| BAv85-Ig | 143 | S12H + L14V + H15R + | [=] | [−] |
| | | L22A + S40G | | |
| BAv86-Ig | 144 | S12H + L14V + H15R + | [=] | [=] |
| | | L22I + S40G | | |
| BAv87-Ig | 145 | L14V + H15R + A16H + | [=] | [−] |
| | | L22A + S40G | | |
| BAv88-Ig | 146 | L14I + H15R + A16H + | [=] | [−] |
| | | L22A + S40G | | |

*[code]: [−] poorer binding than BAv8-10; [=] equivalent binding to BAv8-10; [+] better binding than BAv8-10; [+/−] within two-fold weaker binding than BAv8-10; NB = No binding; ND = not determined because of yield/quality issues with protein

An analysis of this sequence-binding activity data in Tables 2 and 3 showed that certain substitutions relative to the wild-type huBCMA ECD protein appeared to be particularly beneficial with respect to increasing the huBAFF selectivity (relative to huAPRIL) of the variants relative to the surrogate control ([S40G]huBCMA ECD-Ig). These substitutions appeared at positions 12, 14, 15, and 22 relative to wild-type BCMA ECD (SEQ ID NO:152). Specific beneficial substitutions, with respect to huBAFF selectivity, were S12P/N, L14I/V, H15R, and L22V/I/A. Reverting H12 or R12 back to the native S12 appeared to reduce huBAFF binding activity more than it affected huAPRIL binding activity. This result indicated that H12 and R12 contribute more to improving huBAFF binding activity than they do to improving huAPRIL binding activity. When the native S12 was changed to P12 or N12, both huBAFF and huAPRIL binding activities were reduced, but the reduction in huAPRIL binding activity was greater than the reduction in huBAFF binding activity. Both 114 and V14 appeared to improve huBAFF binding activity with little to a slightly adverse effect on huAPRIL binding activity. Reverting N15 and R15 back to histidine reduced huBAFF binding activity more than it did huAPRIL binding activity. This indicated that N15 and R15 contribute more to improve huBAFF binding activity than they do to improving huAPRIL binding activity. V22 by itself improved huBAFF binding activity but appeared to reduce huAPRIL binding activity. 122 and A22 appeared to behave similarly to V22. All of the variants had the S40G substitution in the ECD component of the fusion protein, which appeared effective at improving molecular weight homogeneity of the protein preparation. All the variant proteins showed sharp bands when assayed by gel electrophoresis (data not shown).

### Example 11: Measuring Binding Activities of Variant BCMA-Ig Fusion Polypeptides to BAFF and APRIL Using a Kinetic Exclusion Assay

This example describes further analysis of binding activity of variant BCMA-Ig fusion proteins to human BAFF and human APRIL using a kinetic exclusion assay. In the nomenclature used to describe this type of analysis, the free ligand (BAFF or APRIL) is referred to as the “ligand,” and the receptor Ig fusion binding partners as the “receptor.” As described above, fusion proteins containing Fc-region Ig domains typically exist as dimeric structures in solution by virtue of strong association between the Ig domains and a covalent bond between the hinge cysteine(s). Unless indicated otherwise, such dimeric conformations are expected to exist for the fusion proteins described in this example. Trimeric conformations are expected to exist for human BAFF and human APRIL as the native cytokines exist in trimeric states, the binding sites for the receptors TACI and BCMA occur at the interface between subunits, and the binding by receptors is expected only for trimers or higher order form of BAFF and APRIL. The increases in binding activity described for variant BCMA-Ig fusion proteins result from increases in binding activity between each variant BCMA ECD domain and its corresponding ligand. The strength of binding activity is typically described in terms of the equilibrium dissociation constant (K_{D}), which describes the molar concentration of receptor at which 50% of available ligand is bound at equilibrium.

The kinetic exclusion assay determines K_{D} values by determining free ligand concentration without perturbing an equilibrium of unmodified molecules, unfettered, in solution. For precise K_{D} determination the concentration of ligand in the samples should be near or below the K_{D}. Therefore, with tight binding molecules, the measurements were made at very low sample concentrations. The advantage of a kinetic exclusion assay is that it allows quantitative measurements at very low concentrations, which in turn, results in precise K_{D} measurements, even for very tightly binding molecules.

In this analysis method, polystyrene solid phase beads were coated with variant BCMA ECD-Ig fusion protein, Variant 50238475-PIg18 (ECD SEQ ID NO:63). The coated beads were separately incubated with ligands (huBAFF and huAPRIL) and allowed to reach equilibrium so that any free ligand could be captured by the beads and subsequently detected by fluorescently-labeled detection antibodies. The ability of a variant BCMA ECD-Ig variant to block free ligands (i.e., huBAFF or huAPRIL) was evaluated. Control fusion proteins (i.e., human Z-TACI-Ig or [S40G]huBCMA ECD-Ig fusion protein) were also evaluated for their ability to block huBAFF and huAPRIL for comparison. The kinetic exclusion assays were performed on a KINEXA 3200 Instrument (Sapidyne Instruments Inc.) at room temperature (RT, 25° C.), and equilibrium dissociation constants (K_{D}) were calculated.

### A. Equilibrium Assay

Solid phase beads (200 mg of Polymethyl methacrylate (PMMA), were coated with 1.0 mL of a 30 μg/ml solution of Variant 50238475-Ig in coating buffer (1×PBS, pH 7.4). Beads were tumbled at room temperature for 2 hours and then allowed to settle. Coating solution was removed and beads were blocked in 1 ml of blocking solution (1×PBS, pH 7.4, 10 mg/ml BSA) at room temperature for 2 hours. The 200 mg of beads in 1 ml of blocking buffer were then transferred to a vial containing 29 ml of 1×PBS for use on the KINEXA 3200 Instrument (Sapidyne Instruments Inc.).

Equilibrium measurements were performed using FLAG-tagged, recombinant huBAFF and huAPRIL ligands. Fixed ligand concentrations of human BAFF or human APRIL were incubated with serial dilutions of the variant BCMA ECD-Ig fusion polypeptides, and free ligand was detected in a KINEXA 3200 Instrument. variant BCMA ECD-Ig fusion polypeptides were first diluted in equilibration buffer (1×PBS, pH 7.4, 1 mg/ml BSA) to a range of concentrations from 10 nM to 100 fM. The linear portion of the dilution series was typically 2-fold. Ligands were diluted in equilibration buffer to fixed concentrations ranging from 5 to 20 pM and then incubated with the variant BCMA-Ig dilutions in total reaction volumes ranging from 4.5 to 15 ml. Samples were incubated at room temperature for 48-72 hours to reach equilibrium. For each data point, a fresh column of 850 μl of bead slurry coated with Variant BAv-238475-Ig was introduced into the KINEXA flow cell. Equilibrated samples ranging from 1 to 5 ml reaction volume were injected at a flow rate of 0.25 ml/min. Following a 30 second wash with equilibration buffer at a flow rate of 0.25 ml/min, captured free ligand was detected first by injection of 500 μl mouse monoclonal antibody (IgG1) anti-FLAG M2 (Sigma Aldrich, Catalog No. F1804) diluted to 1 μg/ml in equilibration buffer followed by injection of 500 μl DyLight 649 AffiniPure Goat Anti-Mouse IgG (Jackson ImmunoResearch, Cat. No. 115-495-062) diluted to 0.42 μg/ml in equilibration buffer. The DyLight 649 fluorescent signal was detected using the red filter set (620/30 nanometer excitation filter, 670/40 nanometer emission filter, 645 nanometer dichroic filter) on the KinExA instrument and converted to a voltage signal that was directly proportional to the concentration of free ligand in the equilibrated sample.

### B. KINEXA™ Data Analyses

Binding curves are dependent on K_{D} and receptor concentration. At equilibrium the total association events equal the total dissociation events, so equation 1 may be written: kₒₙ[R][L]=k_{off}[RL]. The rate equations for the reaction are kₒₙ=association rate constant and k_{off}=dissociation rate constant. The ligand and receptor concentrations are [R]=free receptor site concentration, [L]=free ligand site concentration, and [RL]=concentration of complex. Since K_{D} is k_{off}/kₒₙ, equation 1 may be rewritten: K_{D}=([R]·[L])/[RL]. Receptor and ligand bind 1:1, such that total receptor equals receptor in complex plus free receptor.

A binding curve was generated by making a series of samples with constant ligand concentration and a titration of receptor. After equilibrium is reached, a KINEXA™ measurement is made of the free ligand concentration in each sample of the titration series. The binding curve generated was then analyzed to find the K_{D}. Multiple curves with different ligand concentrations may be analyzed together to get both K_{D} and active ligand concentrations. huAPRIL and huBAFF binding curves were plotted with a non-linear regression curve fit. The 95% confidence intervals were computed from the best-fit values and the standard error of the best-fit values.

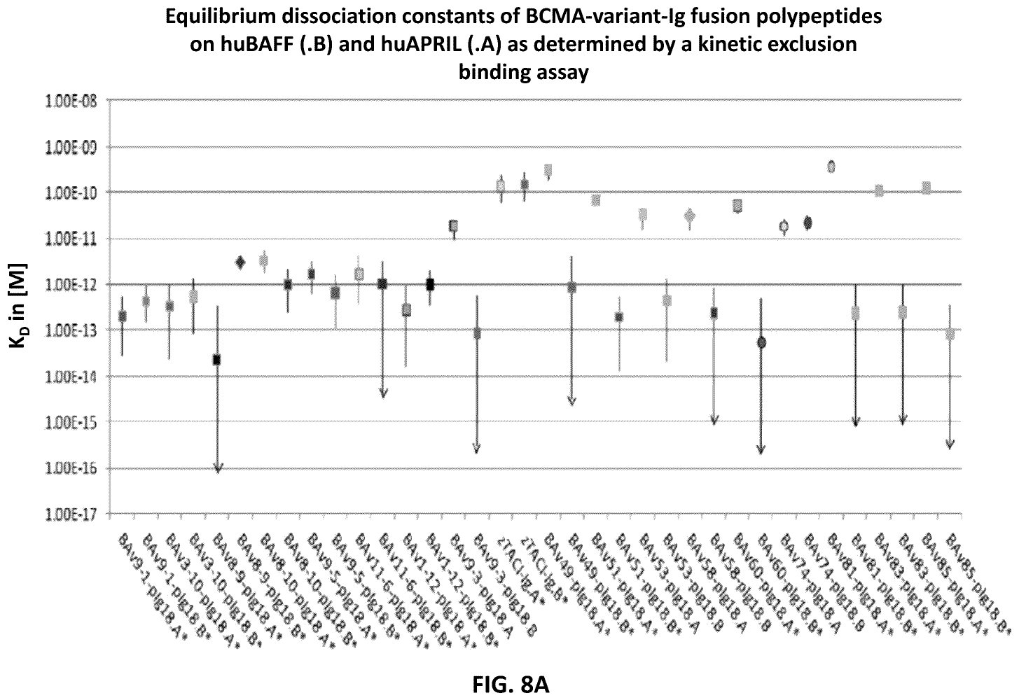

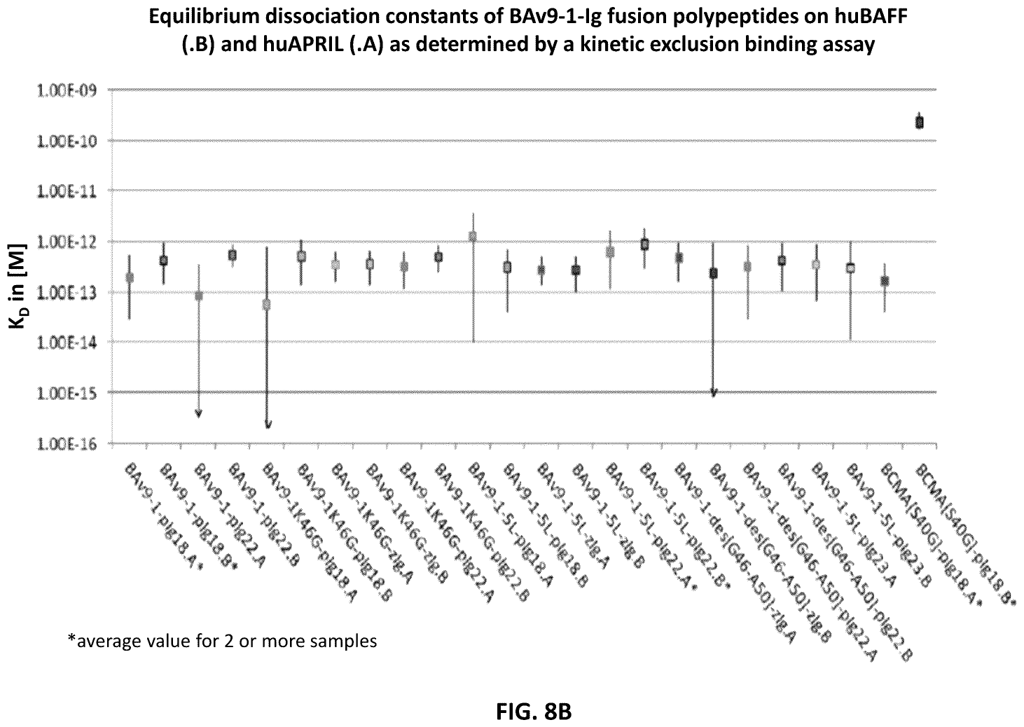

The standard kinetic exclusion assays and data analyses described above were performed on protein preparations of various variant BCMA ECD-Ig fusion polypeptides, as well as Z-TACI-Ig and [S40G]huBCMA ECD-Ig fusion polypeptides. FIG. 8A, FIG. 8B, and Table 4 summarize the binding data for representative variant BCMA ECD-Ig polypeptides and controls in molar concentrations of huBAFF or huAPRIL determined by KINEXA binding assays. The representative variant BCMA ECD-Ig polypeptides are listed in Tables 6 and 7.

The mean values from 2-3 experiments are graphed along with the high and low 950 confidence intervals (error bars). FIG. 8A provides a comparison of the equilibrium dissociation constants of variant BCMA-g polypeptides with controls (BCMA-Cg-Z-TACI-Ig and anti-BAFF monoclonal antibody). FIG. 8B provides a comparison of the various stalk and Ig version of the variant BAv9-1-Ig, which are described in Example 17. The results of the KINEXA assays confirm the binding activity results of BIACORE. Furthermore, the KINEXA assays demonstrate resolution for measuring binding activity below 30 pM. Accordingly, the KINEXA assays are useful for determining the huBAFF selectivity of various variant BCMA ECD polypeptides and variant BCMA ECD-Mg fusion polypeptides.

TABLE 4
Equilibrium dissociation constants for exemplary variant BCMA ECD-Ig
(PIg18) fusion polypeptides as measured by a kinetic exclusion assay*

| | SEQ | | | | | | | | |
|---|---|---|---|---|---|---|---|---|---|
| | ID NO. | | | | | | | | |
| | (CRD | | | | | | | | |
| | of ECD | | | | | | | | |
| | compo | huBAFF | | | | huAPRIL | | | |
| | nent of | | | | Fold | | | | Fold |
| | BCMA | | 95% | 95% | Improvement | | 95% | 95% | Improvement |
| Clone | ECD- | K_{D} | CL | CL | over | K_{D} | CL | CL | over |
| Name | Ig) | (pM) | High | Low | Control | (pM) | High | Low | Control |
| [S40] | 224 | 226 | 369 | 158 | 1.0 | 0.159 | 0.366 | 0.0391 | 1.0 |
| huBCMA- | | | | | | | | | |
| Ig Control | | | | | | | | | |
| 9-1-Ig | 3 | 0.424 | 0.949 | 0.137 | 533.0 | 0.198 | 0.537 | 0.0279 | 0.8 |
| 3-10-Ig | 4 | 0.565 | 1.41 | 0.0821 | 400.0 | 0.337 | 1.00 | 0.0227 | 0.47 |
| 8-9-Ig | 8 | 3.00 | 4.43 | 1.99 | 75.3 | 0.0224 | 0.321 | 8.00E−5 | 7.1 |
| 8-10-Ig | 6 | 0.971 | 2.14 | 0.236 | 232.7 | 3.23 | 5.39 | 1.7 | 0.05 |
| 9-5-Ig | 5 | 0.632 | 1.56 | 0.111 | 357.6 | 1.64 | 3.23 | 0.615 | 0.1 |
| 11-6 -Ig | 7 | 1.01 | 3.14 | 0.00365 | 223.84 | 1.63 | 4.49 | 0.371 | 0.1 |
| 1-12-Ig | 9 | 0.978 | 1.99 | 0.343 | 231.1 | 0.275 | 0.942 | 0.0152 | 0.68 |
| 9-3-Ig | 48 | 0.085 | 0.579 | <0.0003 | 2658 | 18.9 | 25.35 | 9.2 | 0.008 |
| Z-TACI- | | 151 | 287 | 62.2 | 1.5 | 131 | 238 | 59.4 | 0.001 |
| Ig | | | | | | | | | |
| BAv49 | 107 | 0.84234 | 4.2 | 0.00304 | 267.8 | 313.1 | 399.74 | 188.26 | 0.00051 |
| (3)-Ig | | | | | | | | | |
| BAv51 | 109 | 0.19196 | 0.55309 | 0.01247 | 1175.3 | 70.82 | 85.02 | 48.99 | 0.00225 |
| (2)-Ig | | | | | | | | | |
| BAv53-Ig | 111 | 0.462 | 1.38 | 0.02 | 488.4 | 34 | 47.7 | 15.87 | 0.00469 |
| BAv58-Ig | 116 | 0.24 | 0.832 | 0.00087 | 940.1 | 31 | 44 | 14 | 0.00514 |
| BAv60 | 118 | 0.05367 | 0.50132 | 0.00019 | 4203.8 | 51.37 | 65.95 | 33.09 | 0.0031 |
| (2)-Ig | | | | | | | | | |
| BAv74-Ig | 132 | 21.78 | 30.08 | 14.85 | 10.4 | 18.3 | 26.06 | 11.25 | 0.00871 |
| BAv81 | 139 | 0.24603 | 1.02 | 0.00089 | 917 | 363.57 | 419.2 | 269.94 | 0.00044 |
| (2)-Ig | | | | | | | | | |
| BAv83 | 141 | 0.25352 | 1.03 | 0.00092 | 889.9 | 113.73 | 136.58 | 84.4 | 0.00140 |
| (2)-Ig | | | | | | | | | |
| BAv85 | 143 | 0.08473 | 0.37382 | 0.0003 | 2662.8 | 132.28 | 165.85 | 94.77 | 0.00120 |
| (2)-Ig | | | | | | | | | |

*95% confidence intervals (CI) low values marked in italics may be lower than indicated. (n) = average of n assays.

Table 5 provides the selectivities of exemplary variant BCMA polypeptides with respect to BAFF and APRIL.

TABLE 5
BAFF and APRIL selectivity of exemplary
variant BCMA Ig (PIg18) fusion polypeptides*

| | SEQ ID NO. | Selectivity | Selectivity |
|---|---|---|---|
| | (CRD of ECD | for huBAFF | for huAPRIL |
| | component of | (K_{D} APRIL/ | (K_{D} BAFF/ |
| Clone Name | BCMA ECD-Ig) | K_{D} BAFF) | K_{D} APRIL) |
| [S40G]huBCMA | 224 | 0.0007 | 1421.4 |
| ECD-Ig | | | |
| 9-1-Ig | 3 | 0.47 | 2.1 |
| 3-10-Ig | 4 | 0.60 | 1.7 |
| 8-9-Ig | 8 | 0.0075 | 133.9 |
| 8-10-Ig | 6 | 3.33 | 0.30 |
| 9-5-Ig | 5 | 2.59 | 0.39 |
| 11-6-Ig | 7 | 1.6 | 0.62 |
| 1-12-Ig | 9 | 0.3 | 3.56 |
| 9-3-Ig | 48 | 221.2 | 0.005 |
| Z-TACI-Ig | | 0.9 | 1.15 |
| BAv49 (3)-Ig | 107 | 371.703 | 0.003 |
| BAv51 (2)-Ig | 109 | 368.931 | 0.003 |
| BAv53-Ig | 111 | 73.593 | 0.014 |
| BAv58-Ig | 116 | 129.167 | 0.008 |
| BAv60-Ig | 118 | 957.146 | 0.001 |
| BAv74-Ig | 132 | 0.840 | 1.190 |
| BAv81 (2)-Ig | 139 | 1477.747 | 0.001 |
| BAv83 (2)-Ig | 141 | 448.604 | 0.002 |
| BAv85 (2)-Ig | 143 | 1561.194 | 0.001 |

*Table 95% confidence intervals (CI) low values marked in italics may be lower than indicated.

### Example 12: Measuring the Blocking by BCMA Variant Polypeptides of the Binding of Membrane and Soluble huBAFF Using Cells Expressing Surface BCMA Receptors

To assess blockade of surface-expressed huBAFF, 50 μl per well of human embryonic kidney 293 (HEK293) cells expressing the native, full-length huBAFF (herein referred to as membrane-huBAFF (16.82×10⁵ cells/ml in DMEM+10% FBS) were plated on collagen-coated 96 well clear plates and incubated at 37° C. for 2 hrs. To assay for blockade of soluble huBAFF, 50 μl per well of huBAFF (diluted to 200 ng/ml in DMEM+10% FBS) was added to collagen-coated 96 well clear plates. Control proteins (Z-TACI-Ig and [S40]huBCMA ECD-Ig) and variant BCMA ECD-Ig were diluted in a 4-fold dilution series in DMEM+10% FBS. 10 μl per cell well of dilutions were added to the membrane-huBAFF expressing HEK293 cells or the plates containing soluble huBAFF. Reactions were incubated at 37° C. for 30 min. HEK293 cells expressing the native, full-length huBCMA (herein referred to as membrane huBCMA) and the NFkappaB luciferase reporter cassette (an NFkappaB response element promoter operably linked to a coding sequence for luciferase, Clonetech) were resuspended in DMEM+10% FBS at a density of 2.5×10⁵ cells/ml and 40 μl was added per well. Cells were incubated at 37° C. for 16 hrs. A volume of ONE-Glo™ Luciferase Assay Substrate equal to that of the culture medium was added in each well and mixed for optimal consistency. For 96-well plates, typically 100 μl of reagent is added to the cells grown in 100 μl of medium. After 3 minutes to allow complete cell lysis the luciferase activity was measured in a Luminometer.

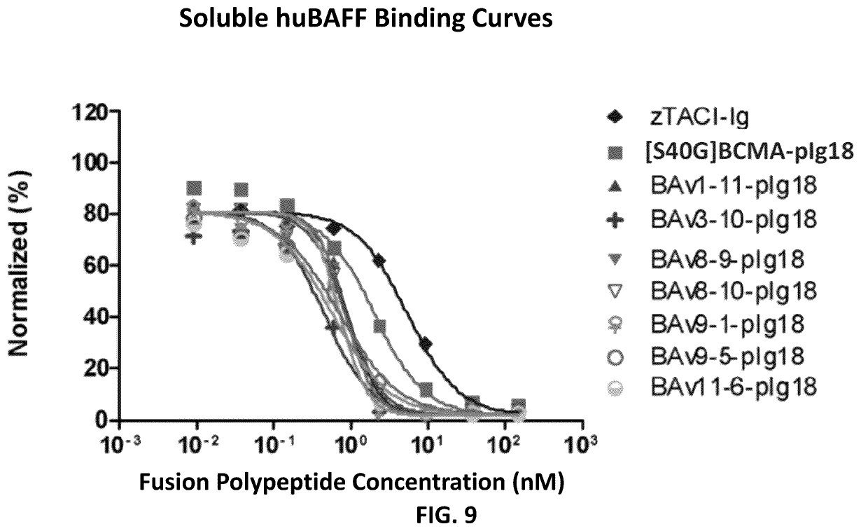

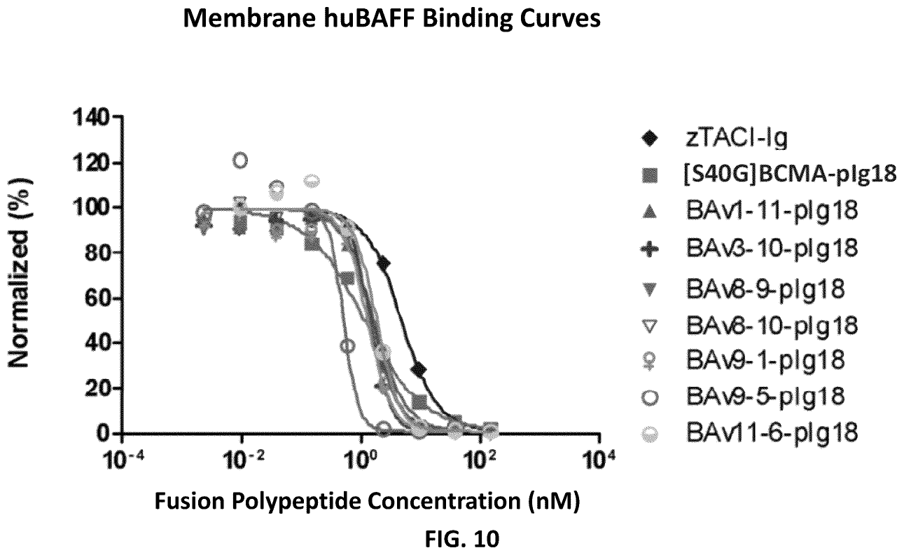

Assays were performed using protein preparations of various variant BCMA ECD-Ig fusion proteins, as well as Z-TACI-Ig and [S40G]huBCMA ECD-Ig fusion proteins. The variant BCMA ECD-Igs included: BAv1-11-PIg18 (CRD SEQ ID NO:19); BAv3-10-PIg18 (CRD SEQ ID NO: 4); BAv8-9-PIg18 (CRD SEQ ID NO: 8); BAv8-10-PIg18 (CRD SEQ ID NO:6); BAv9-1-PIg18 (CRD SEQ ID NO:3); BAv9-5-PIg18 (CRD SEQ ID NO:5); and BAv11-6-PIg18 (CRD SEQ ID NO:7). FIG. 9 depicts the inhibition curves of representative variant BCMA polypeptides as described and controls as the percent maximum proliferation signal determined in the HEK293-huBCMA NFkappaB luciferase reporter cell assay in response to soluble huBAFF. FIG. 10 depicts the inhibition curves of representative variant BCMA polypeptides as described and controls as the percent maximum proliferation signal determined in the HEK293-huBCMA NFkappaB luciferase reporter cell assay in response to HEK-293 cells expressing membrane huBAFF.

The results indicate that the [S40G]huBCMA ECD variant-Ig, BCMA ECD-Ig, and Z-TACI-Ig polypeptides can block soluble as well as membrane-bound huBAFF from stimulating the full-length BCMA receptor, as measured by the decrease in BCMA-receptor-driven luciferase signal. Furthermore, the variant BCMA polypeptides appear to be more potent at inhibiting the huBAFF-BCMA receptor interaction than the [S40G]huBCMA-Ig and Z-TACI-Ig polypeptides.

### Example 13: Inhibition of B Cells and Immunological Responses by BCMA Variant Polypeptides In Vivo in Mice

A lupus-prone mouse study was performed to determine the in vivo effects of antagonists with distinct affinities for mouse BAFF (muBAFF) and mouse APRIL (muAPRIL). The effects of IgG fusion proteins, including a variant BCMA ECD-Ig fusion protein (BAv9-1-Ig, CRD SEQ ID NO:3) with high affinity for muBAFF and muAPRIL, TACI-Ig with relatively low affinity for muBAFF and muAPRIL, and mBR3-Ig, a specific antagonist of muBAFF containing the ECD of mouse BR3 produced as described above for huBCMA-Ig, were evaluated on various biomarkers of B-cell activity and kidney injury.

NZB/W-F1 mice, which spontaneously develop an autoimmune disease resembling human systemic lupus erythematosus, were purchased from Japan SLC, Inc. at 4 months of age. IgG fusion proteins BAv9-1-Ig (CRD SEQ ID NO:3), mBR3-IG, and TACI-Ig, each at a dose of 10 mg/kg, were administered to 5-month old mice. Fusion proteins were administered weekly, via the subcutaneous route, for a total of 8 weeks. Serum immunoglobulins, anti-dsDNA-antibody levels, splenic B cells, IgG, and anti-dsDNA secreting bone marrow plasma cells were analyzed at the end of 8 weekly treatments. A similar additional experiment was performed, using additional mice and observed for up to 16 weeks. Total IgA, CD19 positive B cells and proteinuria was measured for 16 weeks.

Anti-dsDNA antibodies were measured by ELISA assay. ELISA plates (Nunc, Cat. No. 436110) were coated overnight with 100 μg/ml dsDNA (Sigma, Cat. No. D7656), washed, blocked with 1% BSA for 1 h, followed by addition of serum sample (3000-fold diluted with 1% BSA). After incubation for 2 hrs, the plates were washed and incubated with Horse Radish Peroxidase (HRP)-conjugated anti-mouse IgM or IgG (Invitrogen, Cat. No. 61-6820 and 61-6520, respectively) for 1 h. After washing, the plates were developed by chemiluminescence ELISA substrate (Roche, Cat. No. 1582950) and measured using a luminometer. Results were expressed in arbitrary units in reference to a standard curve obtained with a serum pool from 10-month old NZB/W-F1 mice.

Total immunoglobulin levels were measured by Mouse IgM, IgG, and IgA Quantitation Set (Bethyl Laboratories, Cat. Nos. E90-101, E90-131 and E90-103, respectively) according to manufacturer's instructions. B cell levels were determined by FACS analyses of peripheral blood stained with Alexa Fluor 647 labeled anti-mouse CD19 antibody (eBioscienses, clone 1D3) and expressed as CD19 positive cell proportion in lymphocyte fraction. CD19 is associated with B cell activation and activity in systemic lupus erythematosus (Abu-Zahab et al., Egypt J Immunol. 2017 June; 24(2):141-149).

For measurement of total IgG-secreting and anti-dsDNA (nucleosome) secreting bone marrow plasma cells, femurs from NZB/W-F1 mice were harvested and flushed with RPMI 1640 to disperse cells. The number of anti-nucleosome-secreting and IgG-secreting bone marrow cells was determined by ELISPOT (Mabtech, Mariemont, OH, Mouse IgG ELISpot Plus kit) on plates coated with nucleosome (Sigma Chemicals, 100 μg/ml histone plus 100 μg/ml dsDNA) or anti-murine IgG antibodies (Mabtech), respectively.

For renal histology measurements, kidneys from NZB/W-F1 mice were harvested at sacrifice and stained with PAS (Periodic acid-Schiff) and the glomerular severity score determined by an independent observer using a 0-4 scale for glomerular damage. Scores 0-1 were defined as focal, mild, or early proliferative glomerulonephritis. Scores 1-2 were defined as moderate or definite proliferative glomerulonephritis with increased matrix. Scores 2-3 were defined as diffuse and focal or diffuse proliferative glomerulonephritis. Scores 3-4 were defined as severe diffuse proliferative glomerulonephritis with crescents/sclerosis. Changes to glomerular structure and proteinuria may be correlated with renal damage, such as autoimmune-mediated renal damage. Proteinuria (total protein in urine, in mg/dL) was measured over the course of 16 weeks.

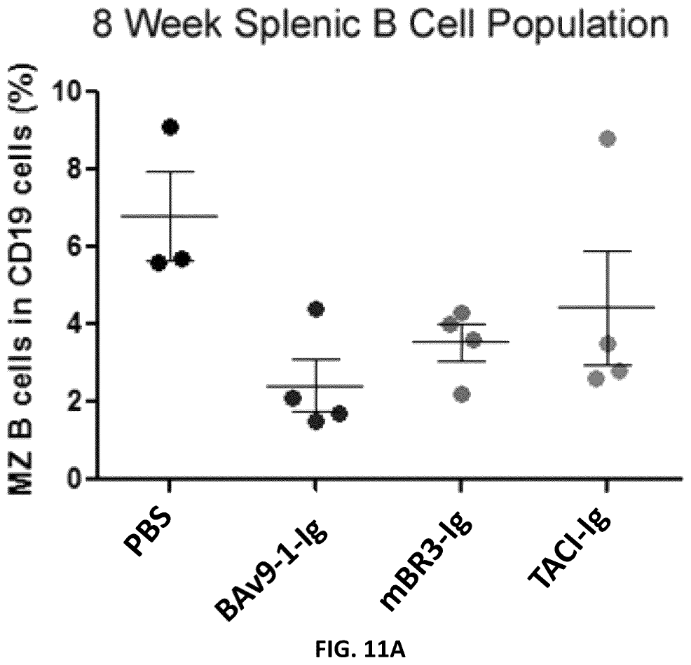

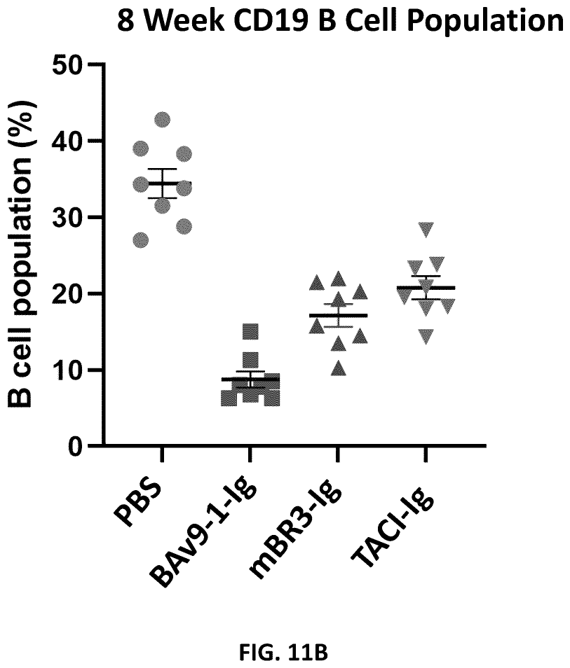

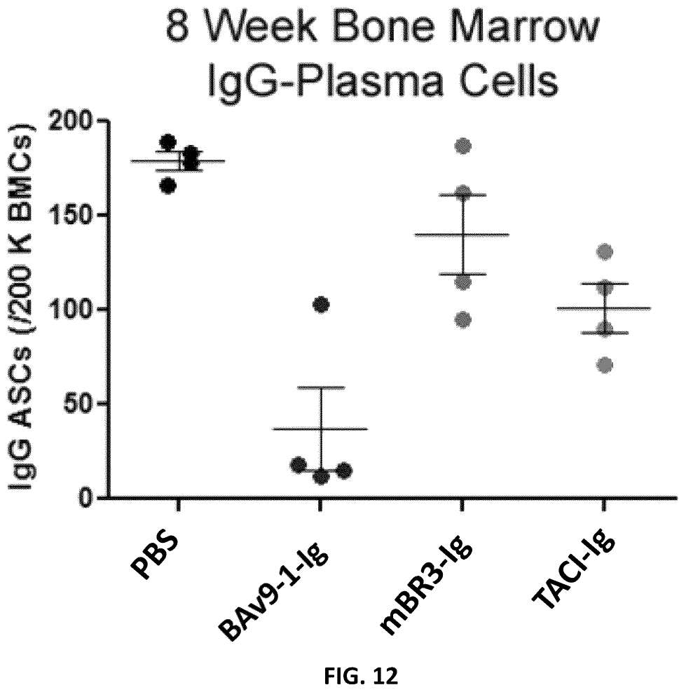

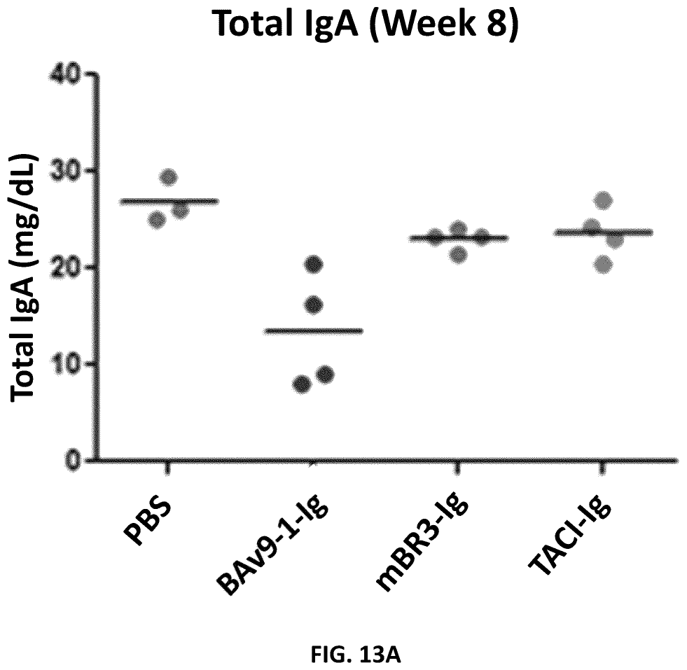

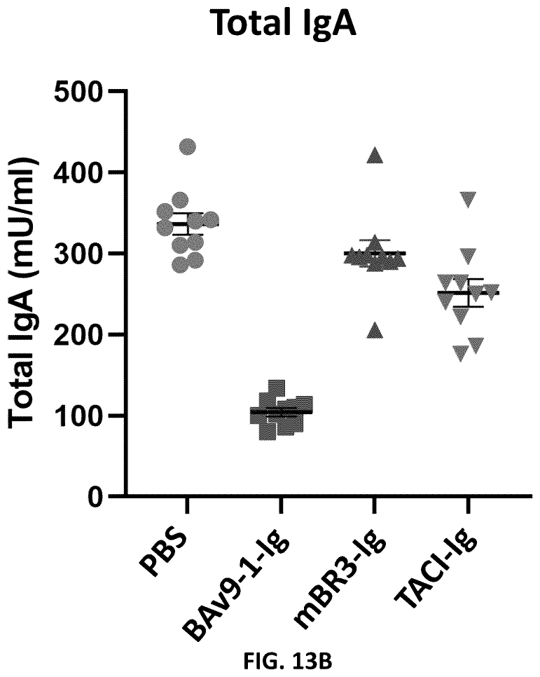

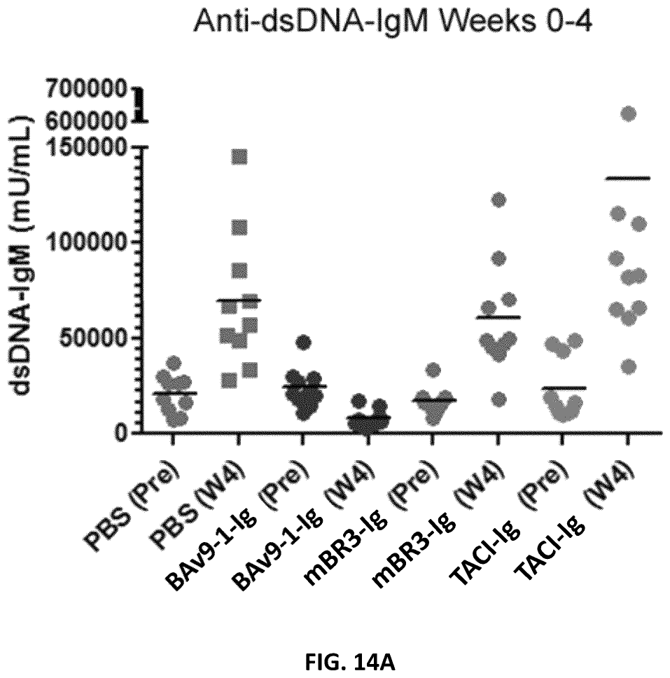

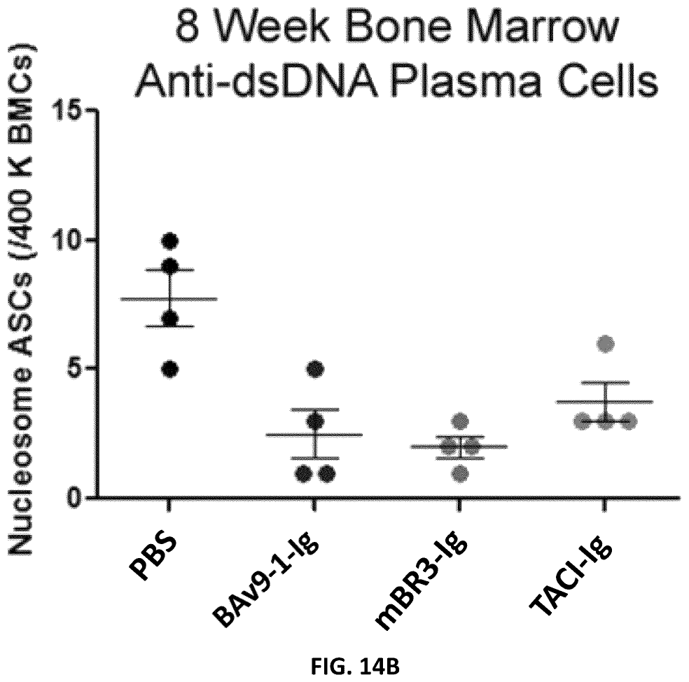

Inhibitory effects on various biomarkers of B cell activity and the lupus-prone disease model were determined for all groups. FIG. 11A shows the percentage of marginal zone B cells from individual mice after 8 weekly treatments with the IgG fusion proteins BAv9-1-Ig, TACI-Ig, mBR3-Ig, and a PBS control and a PBS control. FIG. 11B shows the percentage of CD19 positive B cells in response to treatment with IgG fusion proteins BAv9-1-Ig, TACI-Ig, mBR3-Ig, and a PBS control from the additional experiment, at 8 weeks. FIG. 12 shows the reduction in IgG-secreting bone marrow plasma cells from NZB/W-F1 mice after 8 weekly treatments with test compounds. FIG. 13A shows the reduction in total serum IgA levels from NZB/W-F1 mice after 8 weekly treatments with test compounds. FIG. 13B depicts total IgA serum levels in lupus-prone mice following treatment with BAv9-1-Ig, TACI-Ig, mBR3-Ig, or PBS control, from the additional experiment, at 16 weeks. FIG. 14A shows a comparison of total serum anti-dsDNA IgM levels from NZB/W-F1 mice after 4 weekly treatments (W4) with test compounds versus pretreatment levels (Pre). FIG. 14B shows the reduction in anti-dsDNA-secreting bone marrow plasma cells from NZB/W-F1 mice after 8 weekly treatments with test compounds.

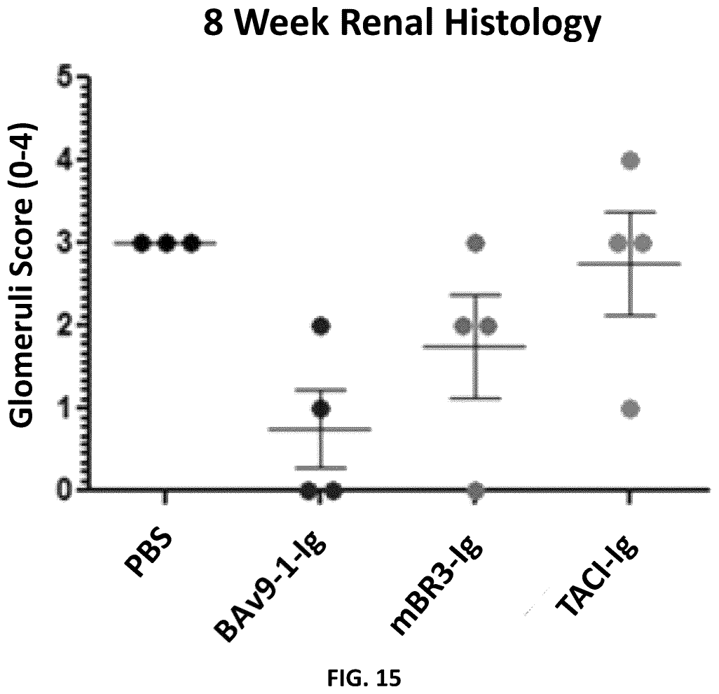

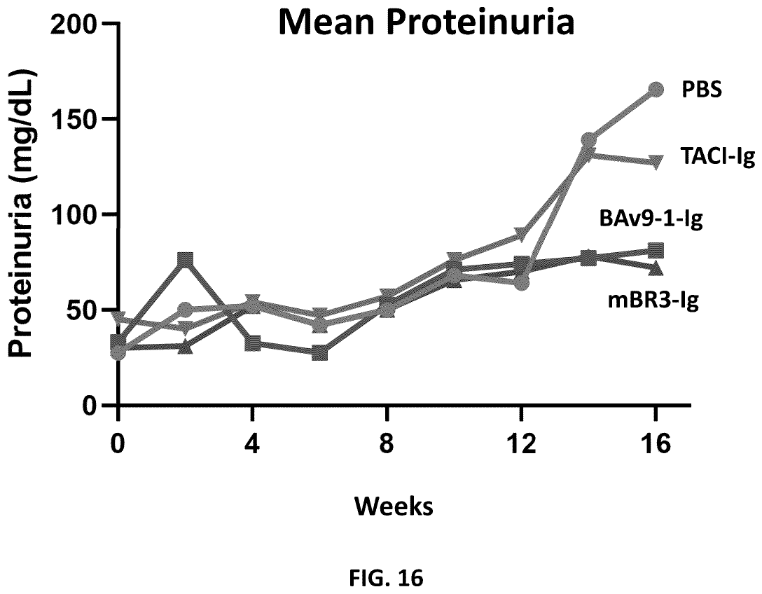

FIG. 15 shows the reduction in glomeruli scores from renal histology measurements from NZB/W-F1 mice after 8 weekly subcutaneous injections with the test IgG fusion proteins or PBS control. FIG. 16 shows mean proteinuria (mg/dL) over 16 weeks in response to treatment with IgG fusion proteins BAv9-1-Ig (CRD SEQ ID NO:3), TACI-Ig, mBR3-Ig, and a PBS control.

In comparison with mBR3-Ig and TACI-Ig, treatment with BCMA variant IgG fusion protein BAv9-1-Ig was associated with the most potent reduction of B cell biomarkers, such as the percentage of CD19-positive B cells and immunoglobulin levels Regarding markers of renal damage, the BAv9-1-Ig treated mice showed the lowest signs of glomerular damage and relatively low levels of proteinuria, similar to the treatment with mBR3-Ig. In comparison, TACI-Ig treated mice exhibited more severe renal damage, and higher levels of proteinuria were comparable to those of PBS-treated mice.

These data are consistent with the conclusion that an exemplary BCMA-Ig polypeptide is more potent than TACI-Ig and mBR3-Ig at inhibiting B cells and immunological responses in vivo.

### Example 14: Inhibition of B Cells and Immunological Responses by BCMA Variant Polypeptides In Vivo in Cynomolgus Monkey

For normal PK and PD (immune response) studies, cynomolgus monkeys (4-5 kg) were treated once with the IgG fusion proteins TACI-Ig and an exemplary variant BCMA IgG fusion polypeptide BAv9-1-Ig. For the tetanus toxoid (TTx) immunization study, cynomolgus monkeys (3-4 kg) were immunized with TTx 30 days prior to six weekly treatments with IgG fusion proteins, TACI-Ig and an exemplary variant BCMA IgG fusion polypeptide BAv9-1-Ig (CRD SEQ ID NO:3). Administration of IgG fusion proteins (at 0.1, 0.3, or 1.0 mg/kg) was through intravenous or subcutaneous routes. Serum immunoglobulins (IgA, IgM, and IgG), anti-TTx-antibody levels (IgM and IgG), and peripheral blood CD19′ B cells were analyzed weekly.

Serum IgA ELISA were measured using the human IgA Quantification Set (Bethyl, Cat. No. E80-102). Results were expressed in arbitrary units in reference to a standard curve obtained with serum from independent cynomolgus. Serum IgM and IgG were measured in ELISA plates (Nunc, Cat. No. 349454) coated with anti-monkey IgM rabbit polyclonal antibody (Convance, Cat/Mp/SIG-3414-1000) or anti-human IgG (Bethyl, Cat. No. A80-104A, 100 fold dilution in PBS) and detected with HRP-conjugated anti-monkey IgM or IgG, respectively (KPL, Cat. Nos. 074-1-031 and 074-1-031, respectively). Serum anti-TTx-IgM and IgG antibody levels were measured on ELISA plates (Nunc, Cat. No. 349454) coated with Tetanus-toxoid (Denka-Seiken, Japan) (100-fold diluted in PBS) and detected as above with HRP-conjugated anti-monkey IgM or IgG, respectively. Plates were developed by colorimetric POD substrate (Sumitomo Bakelite, Cat. No. ML-1120T) and measured by spectrometer. Results were expressed in arbitrary units in reference to a standard curve obtained with serum from independent cynomolgus.

Peripheral blood CD19⁺ B cells were measured in 100 μL of whole monkey blood after staining with APC-H7 conjugated anti-Human CD20 (BD Biosciences, Cat. No. 560734) and FITC-conjugated anti-Human CD3e (BD Biosciences, Cat. No. 556611), followed by fixation with FACS Lysing Solution (BD Biosciences, Cat. No. 349202). The cells were washed and subjected to FACS analysis. B cell population was expressed as a CD20 positive/CD3 negative cell population in the peripheral blood lymphocyte fraction.

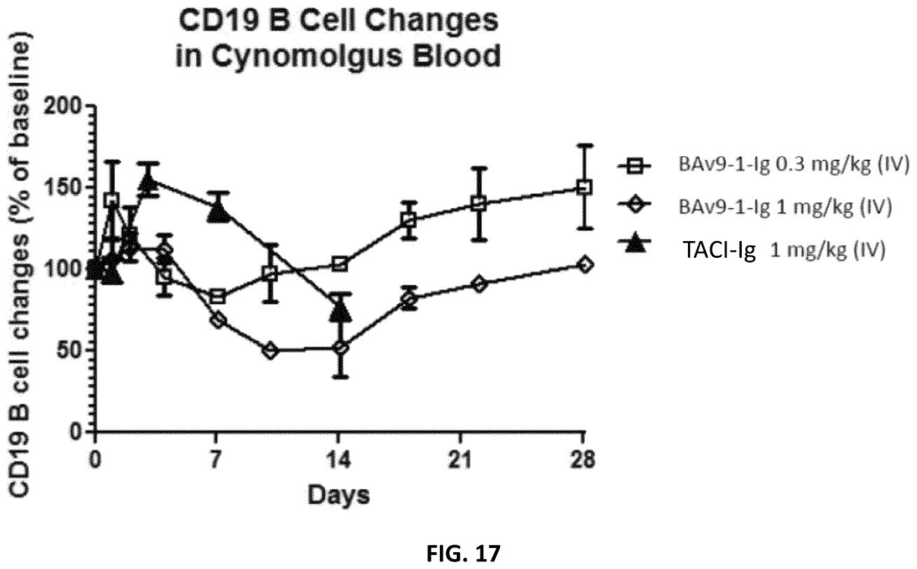

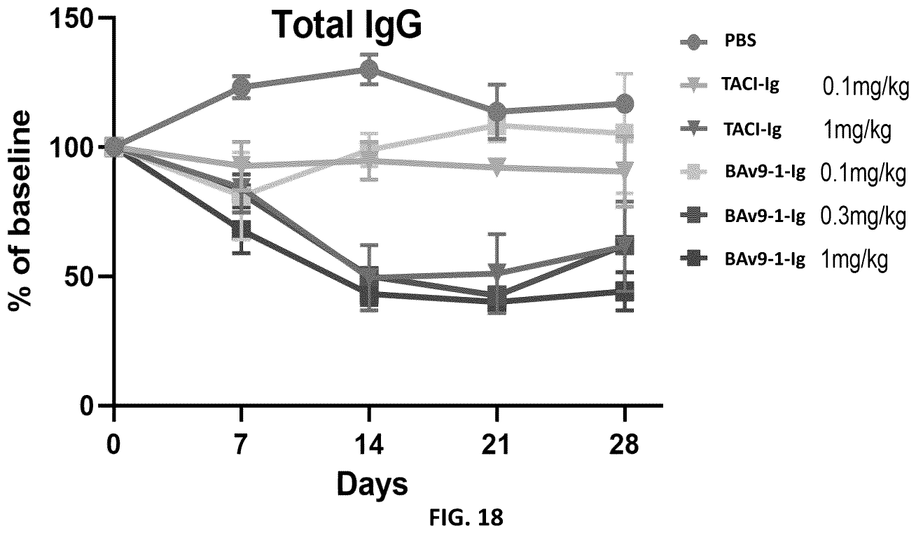

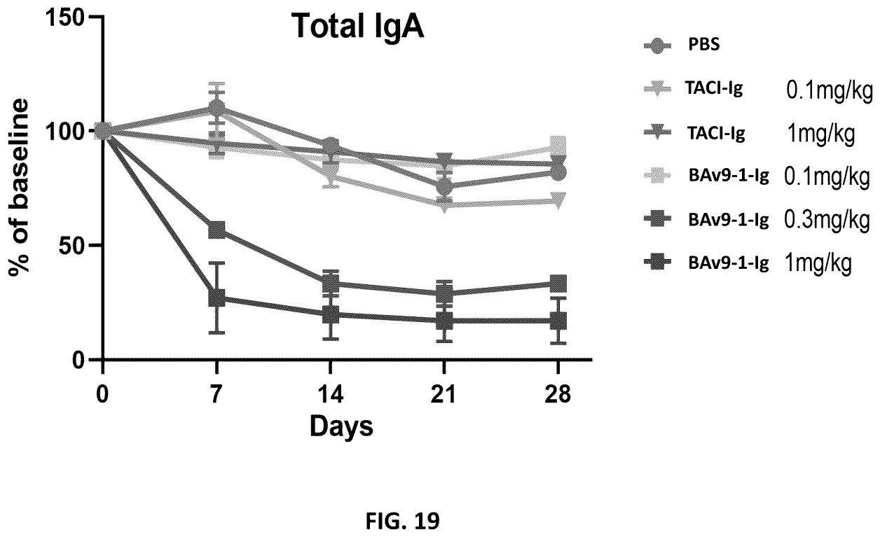

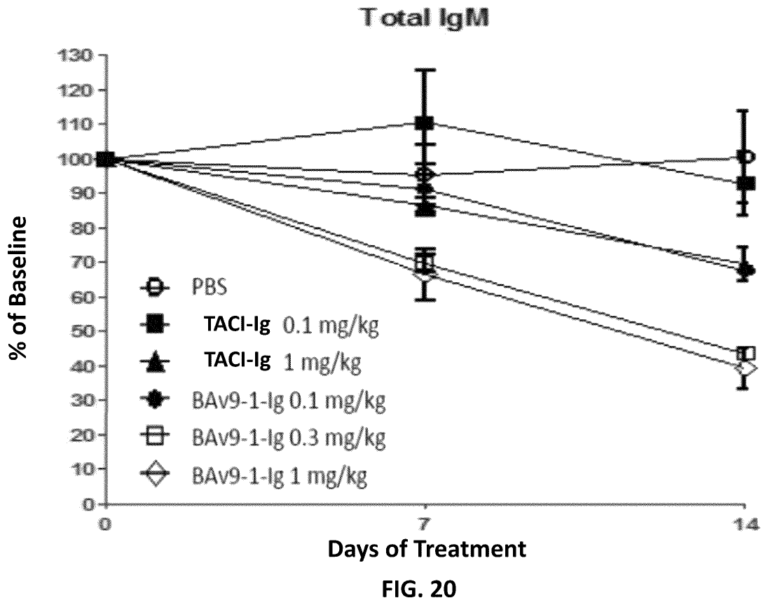

FIG. 17 shows that an exemplary variant BCMA IgG fusion polypeptide as described (BAv9-1-Ig at 1.0 mg/kg) more potently reduced CD19-positive peripheral blood B lymphocytes (%) from cynomolgus monkeys at over 28 days, after a single IV treatment in comparison to the same dose of TACI-Ig. FIG. 18 shows more potent reduction in total serum IgG from cynomolgus monkeys over the course of 28 days after intravenous treatments with BAv9-1-Ig at 0.3 mg/kg versus TACI-Ig at 1.0 mg/kg. FIG. 19 shows more potent reduction in total serum IgA from cynomolgus monkeys over the course of 28 days after intravenous treatments with BAv9-1-Ig at 0.3 and 1.0 mg/kg versus TACI-Ig control protein at 1.0 mg/kg. FIG. 20 shows more potent reduction in total serum IgM from cynomolgus monkeys over the course of 14 days after intravenous treatments with BAv9-1-Ig showing greater inhibition of IgM at equivalent doses (1.0 mg/kg) versus TACI-Ig (1.0 mg/kg) as well as showing that a 10-fold lower dose (0.1 mg/kg) of the representative variant BCMA polypeptide as described is equivalent to TACI-Ig at 1.0 mg/kg.

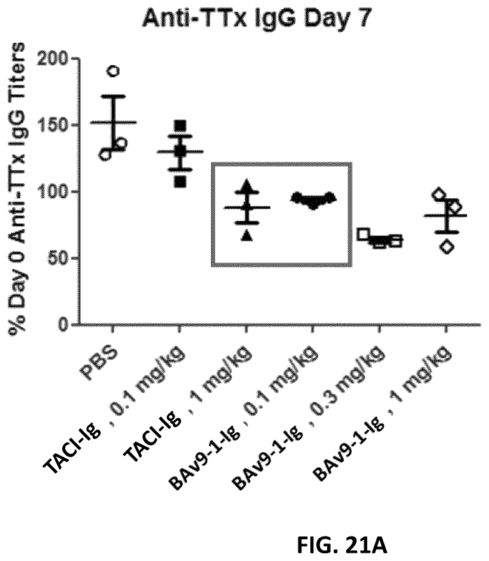

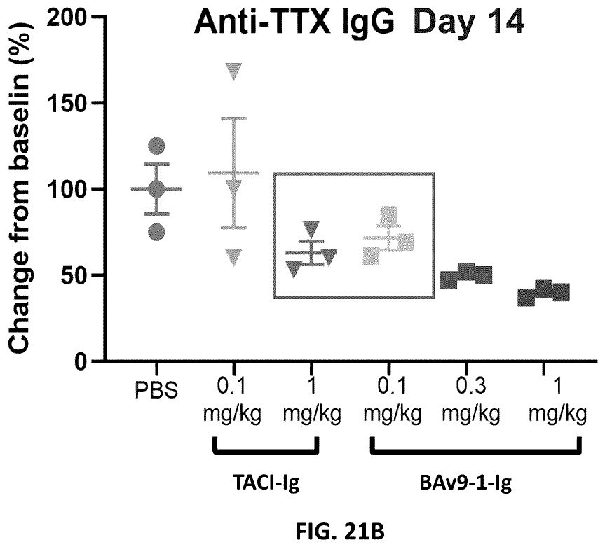

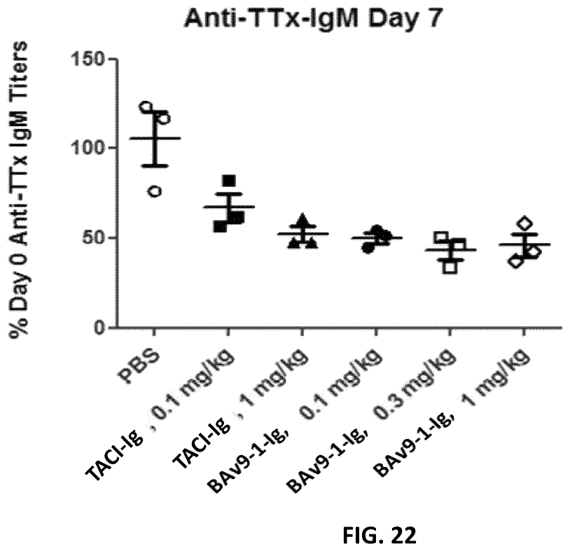

FIG. 21A shows comparable changes in anti-TTx IgG from baseline levels in cynomolgus monkeys at day 7 after intravenous treatment between BAv9-1-Ig (0.1 mg/kg) and a 10-fold higher dose of TACI-Ig (1.0 mg/kg), with the most potent inhibition resulting from treatment with BAv9-1-Ig at 0.3 mg/kg and 1.0 mg/kg. FIG. 21B shows a similar trend in anti-TTx IgG inhibition at day 14. FIG. 22 shows at least 10-fold greater potency of BAv9-1-Ig in inhibition of serum anti-tetanus toxoid (TTx) IgM antibodies from cynomolgus monkeys at day 7 after intravenous treatment: 0.1 mg/kg of BAv9-1-Ig showed equivalent effects as a 10-fold higher dose of TACI-Ig (1.0 mg/kg).

Treatment with an exemplary variant BCMA IgG fusion polypeptide resulted in more potent reduction of markers of B cell activation and antibody production. Together, these data are consistent with the in vitro results and mouse studies, demonstrating improved potency of the exemplary variant BCMA polypeptide BAv9-1-Ig, compared to TACI-Ig.

### Example 15: Inhibition of Peripheral B Cells and Bone Marrow Plasma Cells In Vivo Under Induction and Maintenance Dosing of BAFF- and BAFF/APRIL Blockade

The inhibitory effects of a BAFF-selective variant BCMA fusion polypeptide (238833-Ig) on B cells were investigated in this Example. B-cell survival and immunoglobulin levels were evaluated in mice subject to induction therapy with 238833-Ig and maintenance therapy with a negative control, 238833-Ig, or mouse BAFF antagonist mBR3-Ig.

Six to eight week old female Balb/c mice were given weekly subcutaneous injections of 238833-Ig (PIg18) (10 mg/kg) or vehicle (1% mouse serum in PBS) for three weeks. Then, mice were either maintained on vehicle, 238833-Ig, or switched from receiving 238833-Ig to receiving weekly subcutaneous injections of mBR3-Ig (50 mg/kg) for an additional three weeks.

At weekly intervals, three mice per treatment group were sacrificed to monitor the effects of induction and maintenance dosing on B cell populations and immunoglobulin levels. Single cell suspensions were prepared from spleens, and the percent of Follicular B cells (B220⁺, FSC^{lo}, CD21/35⁺, CD23⁺), Transitional B cells (B220^{mid}, FSC^{lo}, CD21/35⁻, CD23⁻), Marginal Zone B cells (B220⁻, FSC^{mid}, CD21/35⁺, CD23⁻), and Marginal Zone B cell Precursors (B220⁻, FSC^{hi}, CD21/35^{hi}, CD23⁺), was determined by flow cytometry. Bone marrow was flushed from femurs, red blood cells were lysed, and the number of IgG-secreting bone marrow cells was determined by ELISPOT (Mouse IgG ELISpot Plus kit, Mabtech, Mariemont OH). Serum was collected and total serum immunoglobulin levels (IgA, IgG1, IgG2a, IgG2b, IgG3, IgM) were determined by multiplexed ELISA (Mouse Isotyping Panel 1 Assay Kit, Meso Scale Delivery, Gaithersburg, MD).

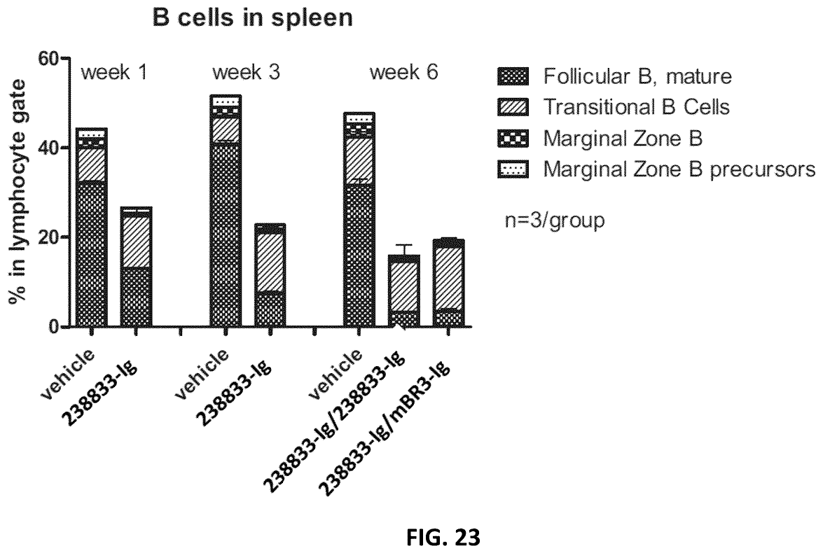

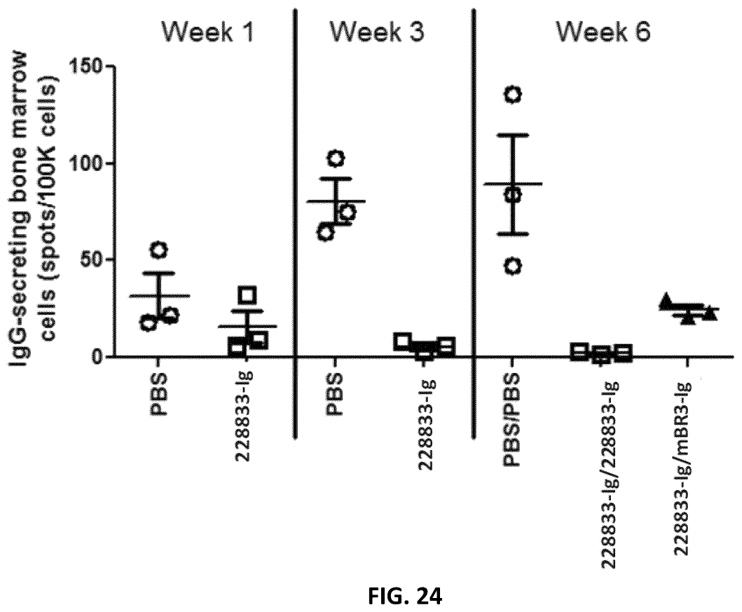

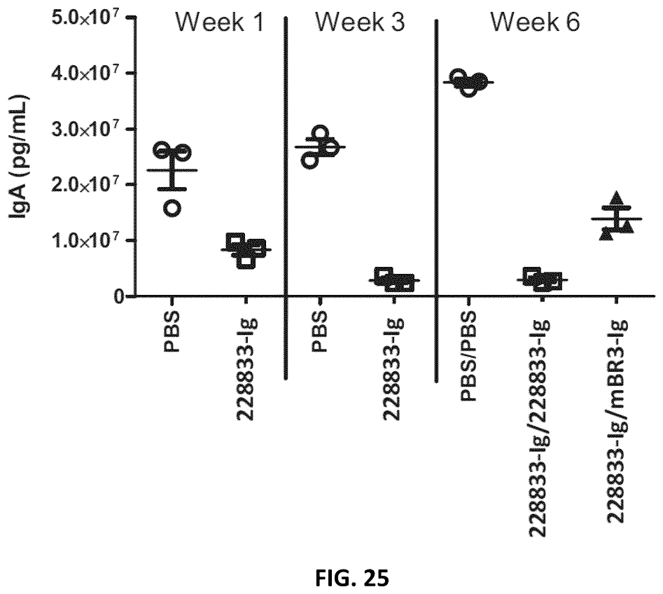

Four weekly “induction” treatments of normal mice with high doses (10 mg/kg) of a BAFF-selective variant BCMA 238833-Ig inhibited splenic, follicular B220⁺ B cells as well as marginal zone B cells (FIG. 23). This result was believed to be due to efficient blockade of BAFF. In addition, by week three “induction” treatments with 238833-Ig inhibited bone marrow plasma cells (FIG. 24) and serum IgA (FIG. 25), which are consistent with blockade of both BAFF and APRIL. Furthermore, when induction mice were treated for an additional three weeks with either high doses of 238833-Ig (10 mg/kg) to continually block both BAFF and APRIL or switched to a maintenance treatment with mBR3-Ig to block only BAFF, the suppressive effects on splenic, follicular B220⁺ B cells as well as marginal zone B cells stayed similar in both groups (FIG. 23). Conversely, induction mice switched to a maintenance treatment with mBR3-Ig recovered bone marrow plasma cells (FIG. 24) and serum IgA (FIG. 25) by week 6. This result is consistent with recovery of free APRIL. Induction mice that continued on high doses of 238833-Ig did not recover bone marrow plasma cells (FIG. 24) or serum IgA (FIG. 25) by week 6.

Switching to maintenance therapy with BAFF-selective mBR3-Ig resulted in bone marrow plasma cell recovery and increased serum IgA, indicating potential BAFF/APRIL binding activity of 238833-Ig. Together, these results are consistent with continued blockade of both BAFF and APRIL in mice that received 238833-Ig for induction and maintenance therapy.

### Example 16: Inhibition of Peripheral B Cells and Bone Marrow Plasma Cells In Vivo with BAFF-Selective BCMA Variant Polypeptides

The inhibitory effects of BAFF-selective variant BCMA fusion polypeptide BAv66-Ig on B cells were evaluated. The effects of two different doses of BAv66-Ig on B cell survival and immunoglobulin levels were compared in this Example.

Six to eight week old female Balb/c mice were given three weekly subcutaneous injections of vehicle, 500 μg/mouse BAv66-Ig (BAv66 ECD component corresponds to SEQ ID NO:124), or 150 μg/mouse BAv66-Ig. At week three, three mice per treatment group were sacrificed to monitor the effects of induction and maintenance dosing on B cell populations and immunoglobulin levels. Single cell suspensions were prepared from spleens, and the percent of Follicular B cells (B220⁺, FSC^{lo}, CD21/35⁺, CD23⁺), Transitional B cells (B220^{mid}, FSC^{lo}, CD21/35⁻, CD23⁻), Marginal Zone B cells (B220⁺, FSC^{mid}, CD21/35⁺, CD23⁻), and Marginal Zone B cell Precursors (B220⁺, FSC^{hi}, CD21/35^{hi}, CD23⁺), was determined by flow cytometry. Bone marrow was flushed from femurs, red blood cells were lysed, and the number of IgG-secreting bone marrow cells was determined by ELISPOT (Mabtech, Mariemont, OH, Mouse IgG ELISpot Plus kit). Serum was collected and total serum immunoglobulin levels (IgA, IgG1, IgG2a, IgG2b, IgG3, IgM) were determined by multiplexed ELISA (Meso Scale Delivery, Gaithersburg, MD, Mouse Isotyping Panel 1 Assay Kit).

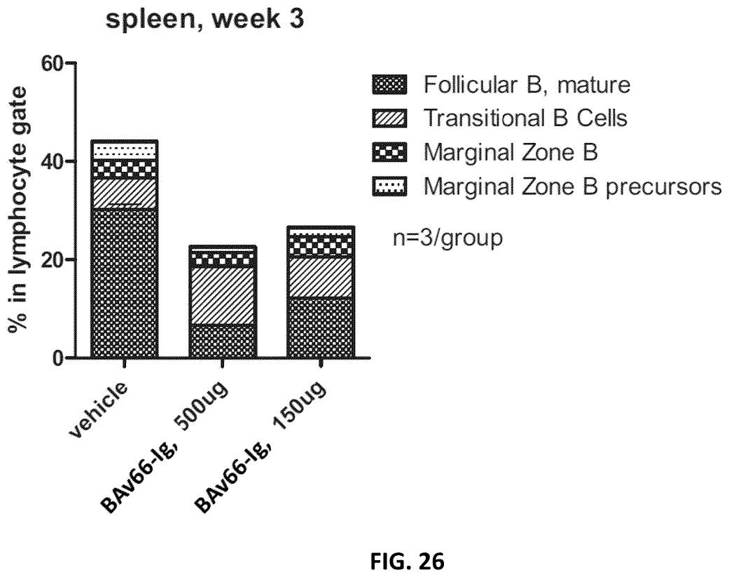

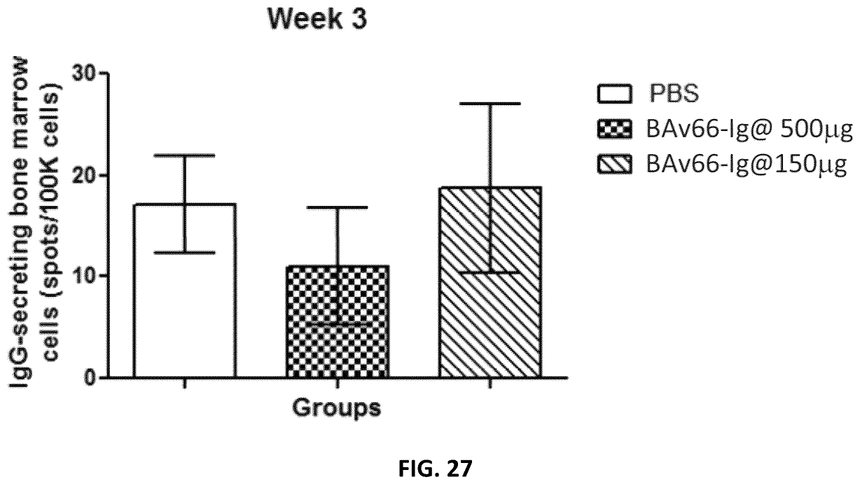

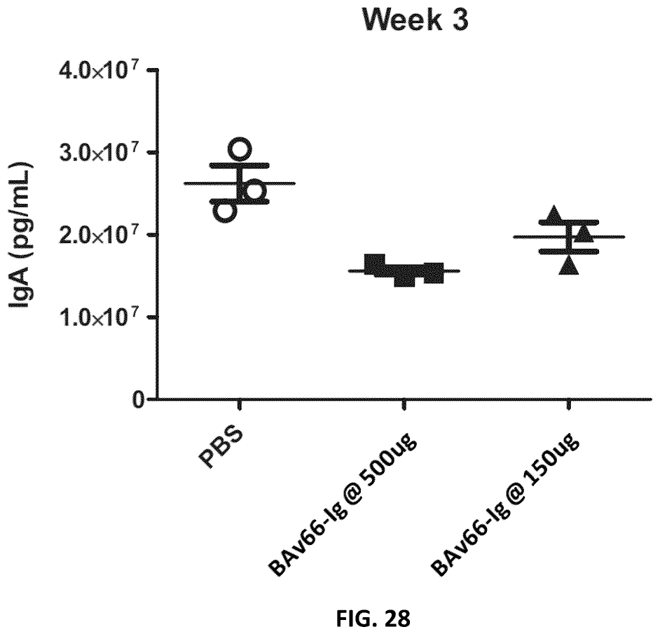

Four weekly treatments of normal mice with titrated doses (150 or 500 μg/mouse) of BAFF-selective variant BCMA polypeptide as described (BAv66-Ig) inhibited splenic, follicular B220⁺ B cells as well as marginal zone B cell precursors (FIG. 26) demonstrating efficient blockade of BAFF across a range of dose levels. The higher dose (500 g) was more potent than the lower (150 g) dose of BAv66-Ig at inhibiting bone marrow plasma cells (FIG. 27) and serum IgA (FIG. 28), in spite of the fact that both doses had similar effects on inhibiting splenic, follicular B220⁺ B cells.

Both tested doses of BAv66-Ig inhibited B-cell survival and reduced immunoglobulin levels, consistent with strong binding activity for BAFF. In comparison to the higher dose, these results indicate that the lower dose (150 g) of BAv66-Ig may have utility as a maintenance therapy, inhibiting pathogenic and malignant peripheral mature B cells, while sparing peripheral and bone marrow plasma cells and plasmablasts.

### Example 17: Fc Fusions with Low Post-Translational Modifications and Improved PK

This Example describes the construction of various plasmids encoding BAv9-1 variant fusion proteins and the post-translational processing of the generated polypeptides. BAFF and APRIL binding activity was also evaluated to determine whether varying the stalk region affected the binding of a variant fusion polypeptide to its target. Three Ig Fc variant BAv9-1 fusion polypeptides, containing different Fc regions, were then evaluated in vivo to assess half-life.

Fusion polypeptides representing different combinations of the BCMA-ECD stalk region (residues 46-50 with respect to SEQ ID NO:152) and Ig Fc were generated in the context of the BAv9-1-ECD (BAv9-1 CRD corresponds to SEQ ID NO:3) and compared with BAv9-1-PIg18. Polynucleotides encoding the above polypeptides were expressed and purified using the pcDNA3.1 (Life Technologies) expression system described in Example 6. The starting plasmid for these derivatives was the pcDNA-BAv9-1-PIg18 expression vector generated for expression of BAv9-1-PIg18 (CRD SEQ ID NO:3), as described in Example 10). This plasmid has a unique NheI site upstream of the first ATG codon of the CTLA4-signal sequence, a unique AgeI site toward the end of the DNA encoding the CTLA4-signal and just upstream of the BAv9-1 coding region, a unique KpnI site 12 bps upstream of the junction between the BAv9-1 and pIg18 coding regions, a unique PmlI site within the DNA encoding the CH2 domain of pIg18 and a unique PmeI site downstream of the stop codon following the PIg18 coding region. These unique restriction sites facilitated construction of the different constructs. Most of the constructs were generated in the pcDNA transient expression vector system and the proteins were expressed and purified as described in Examples 6-8. As described previously, the terminal lysine for these proteins may be cleaved off during post-translational processing, depending on the expression host used. A subset of the constructs were generated in the CET1019AS stable expression vector system and the proteins were expressed and purified as described in Example 2. In some cases, for example BAv9-1-PIg18, the encoding polynucleotide was excised from the pcDNA vector using the AgeI-PmeI sites and used to replace the AgeI-PmeI region in the CET1019-BCMA-PIg18 plasmid (Example 1) to obtain plasmid CET1019-BAv9-1-PIg18 which encodes the same protein as pcDNA-BAv9-1-PIg18. The construction of the various plasmids is described below:
a) BAv9-1-PI222 expression vector: PIg22 is a chimeric Ig-Fc consisting of the CH2 domain from PIg18 (IgG2 derivative, described in Example 1) and the CH3 domain from IgG1. DNA encoding the CTLA4-signal, BAv9-1 ECD, and the CH2 domain of the PIg18 was PCR amplified from plasmid pcDNA-BAv9-1-PIg18 and DNA encoding the CH3 domain of IgG1 was amplified from plasmid CET1019AS-z-TACI−. The two PCR fragments were joined using the splicing by overlap extension method (Horton et al., Gene 1989; 77:61-68) to obtain a single polynucleotide BAv9-1-PIg22 (CRD SEQ ID NO: 3) that encoded an in-frame fusion polypeptide as follows: CTLA4-signal, BAv9-1-ECD, and a chimeric Ig Fc consisting of the CH2 domain from PIg18 and the CH3 domain of IgG1. The PCR fragment was digested with AgeI and PmeI and used to replace the AgeI-PmeI region of plasmid pcDNA-BAv9-1-PIg18 to obtain plasmid pcDNA-BAv9-1-PIg22. As described previously, the terminal lysine of the encoded protein may be cleaved off during post-translational processing.
b) BAv9-1-zIg expression vector: DNA encoding the zIg (Ig Fc from z-TACI-Ig) was PCR amplified from z-TACI-Ig vector using a forward primer that added a unique KpnI site and the last 12 bps of the BAv9-1-ECD-coding region to the 5′ end of the zIg coding region and a reverse primer that introduced a PmeI site downstream of the stop codon. The resulting zIg-encoding PCR fragment was digested with KpnI and PmeI and used to replace the KpnI-PmeI fragment from plasmid pcDNA3.1-BAv9-1-PIg18. The resulting plasmid pcDNA-BAv9-1-zIg contains a polynucleotide that encodes an in-frame fusion polypeptide as follows: the CTLA4-signal, BAv9-1-ECD, and zIg Fc. As described previously, the terminal lysine of the encoded protein may be cleaved off during post-translational processing.
c) BAv9-1-K46G-PIg18 expression vector: The BAv9-1-ECD-coding region between AgeI and KpnI was PCR amplified from plasmid pcDNA-BAv9-1-PIg18 using a mutagenic reverse primer that introduced the K46G mutation into the BAv9-1-coding fragment. The resulting PCR fragment encoding the BAv9-1K46G was digested with AgeI and KpnI and used to replace the AgeI-KpnI fragment in pcDNA-BAv9-1-PIg18 to obtain plasmid pcDNA-BAv9-1K46G-PIg18, which contains a polynucleotide that encodes an in-frame fusion polypeptide as follows: the CTLA4-signal, BAv9-1-ECD with the K46G substitution, and PIg18 Fc. As described previously, the terminal lysine of the encoded protein may be cleaved off during post-translational processing.
d) BAv9-1-K46G-PIg22 expression vector: The BAv9-1-K46G PCR fragment obtained above in (c) was digested with AgeI and KpnI and used to replace the AgeI-KpnI fragment in pcDNA-BAv9-1-PIg22 (described in (a)) to obtain plasmid pcDNA-BAv9-1-K46G-PIg22, which contains a polynucleotide that encodes an in-frame fusion polypeptide as follows: the CTLA4-signal, BAv9-1-ECD with the K46G substitution, and PIg22 Fc. As described previously, the terminal lysine of the encoded protein may be cleaved off during post-translational processing.
e) BAv9-1-K46G-zIg expression vector: The BAv9-1-K46G PCR fragment obtained above in (c) was digested with AgeI and KpnI and used to replace the AgeI-KpnI fragment in pcDNA-BAv9-1-zIg (described in (b)) to obtain plasmid pcDNA-BAv9-1K46G-zIg, which contains a polynucleotide that encodes an in-frame fusion polypeptide as follows: the CTLA4-signal, BAv9-1-ECD with the K46G substitution, and zIg Fc. As described previously, the terminal lysine of the encoded protein may be cleaved off during post-translational processing.
f) BAv9-1-[K46-A50]del-zIg expression vector: DNA encoding a truncated BAv9-1 starting from the AgeI site on the 5′ end but ending 15 bps upstream of zIg junction was PCR amplified from plasmid pcDNA-BAv9-1-zIg. DNA encoding the entire zIg was PCR amplified from the same template. The two PCR fragments were joined using SOE technique (Horton et al., Gene 1989; 77:61-68), digested with AgeI and PmeI and used to replace the AgeI-PmeI region from pcDNA-BAv9-1-pIg18. The resulting plasmid pcDNA-BAv9-1 des[K46-A50]-zIg contains a polynucleotide that encodes a fusion polypeptide as follows: the CTLA4-signal, a BAv9-1 CRD (without K46-A50 residues; CRD SEQ ID NO:3), and the zIg Fc. As described previously, the terminal lysine of the encoded protein may be cleaved off during post-translational processing.
g) BAv9-1-[K46-A50]del-PI222 expression vector: DNA encoding the entire pIg22 was PCR amplified from the pcDNA-BAv9-1-pIg22 plasmid and was joined to the 3′ end of the truncated BAv9-1 PCR fragment generated above in (f) using the splicing by overlap extension method (Horton et al., Gene 1989; 77:61-68). The resulting PCR fragment was digested with AgeI and PmeI and used to replace the AgeI-PmeI region of plasmid pcDNA-BAv9-1-pIg18. The resulting plasmid pcDNA-BAv9-1-des[K46-A50]-PIg22 contains a polynucleotide that encodes a fusion polypeptide as follows: the CTLA4-signal, a BAv9-1-CRD (CRD SEQ ID NO:3), and the PIg22 Fc. As described previously, the terminal lysine of the encoded protein may be cleaved off during post-translational processing.
h) BAv9-1-5L-PIg18 expression vector: DNA encoding the BAv9-1-ECD was PCR amplified from plasmid pcDNA-BAv9-1-PIg18 using a forward primer that began upstream of the NheI site. A mutagenic reverse primer was used to convert the last 15 bps of the BAv9-1-ECD-coding region, encoding the five C-terminal residues of the variant ECD, to a sequence encoding GGGGS (SEQ ID NO:156). DNA encoding the entire PIg18 Fc region was PCR amplified from plasmid pcDNA-BAv9-1-PIg18. The two PCR fragments were joined using the splicing by overlap extension method (Horton et al., Gene 1989; 77:61-68) and the resulting PCR fragment was digested with AgeI and PmeI and used to replace the AgeI-PmeI region of pcDNA-BAv9-1-PIg18 to obtain plasmid pcDNA-BAv9-1-5L-PIg18, which contains a polynucleotide sequence that encodes an in-frame fusion polypeptide as follows: of the CTLA4-signal, BAv9-1 CRD (SEQ ID NO:3), a five amino-acid linker consisting of GGGGS (SEQ ID NO:156), and the PIg18 Fc. As described previously, the terminal lysine of the encoded protein may be cleaved off during post-translational processing.
i) BAv9-1-5L-zIg expression vector: The BAv9-1-5L DNA fragment obtained above in (h) was joined to the zIg DNA fragment (obtained in (b) above) using the splicing by overlap extension method (Horton et al., Gene 1989; 77:61-68) and cloned using AgeI-PmeI sites as above. The resulting plasmid pcDNA-BAv9-1-5L-zIg contains a polynucleotide sequence that encodes an in-frame fusion polypeptide as follows: the CTLA4-signal, BAv9-1-CRD (SEQ ID NO:3), a five amino-acid linker consisting of GGGGS (SEQ ID NO:156), and the zIg Fc. As described previously, the terminal lysine of the encoded protein may be cleaved off during post-translational processing.
j) BAv9-1-5L-PI222 expression vector: The BAv9-1-5L DNA fragment obtained above in (h) was joined to the PIg22 DNA fragment (obtained in (g)) using the splicing by overlap extension method (Horton et al., Gene 1989; 77:61-68) and cloned using AgeI-PmeI sites as above. The resulting plasmid pcDNA-BAv9-1-5L-PIg22 contains a polynucleotide sequence that encodes an in-frame fusion polypeptide as follows: the CTLA4-signal, BAv9-1-CRD (SEQ ID NO:3), a five amino-acid linker consisting of GGGGS (SEQ ID NO:156), and the pIg22 Fc. The BAv9-1-5L-PIg22 encoding polynucleotide was excised from the pcDNA-BAv9-1-5L-PIg22 plasmid using flanking NheI and PmeI sites and was transferred to the stable expression vector CET1019AS (described in Example 1) using the same restriction sites to obtain plasmid CET-BAv9-1-5L-PIg22. As described previously, the terminal lysine may be cleaved off during post-translational processing depending on the expression host used, i.e., the polypeptide produced therefrom corresponds to BAv9-1-5L-pIg22 [K278]del.
k) BAv49, 81, 83, 85-5L-PIg22 expression vectors: The BAv49-5L-PIg22 polynucleotide was generated using the same method described in (j) except that pcDNA-BAv49-PIg18 (obtained as described in Example 10) was used as the template to generate the BAv49-5L DNA fragment. The BAv49-5L-PIg22 fragment was cloned directly into the CET1019AS vector using NheI and PmeI cloning sites to obtain CET-BAv49-5L-PIg22 which contains a polynucleotide that encodes an in-frame fusion polypeptide as follows: CTLA4-signal, BAv49 ECD (CRD SEQ ID NO:107), a GGGGS linker (SEQ ID NO:156), and PIg22 Fc. CET1019AS-based expression vectors for BAv81-5L-PIg22 (CRD SEQ ID NO:139), BAv83-5L-PIg22 (CRD SEQ ID NO:141), BAv85-5L-PIg22 (CRD SEQ ID NO:143) were constructed in a similar manner. As described previously, the terminal lysine of the encoded proteins may be cleaved off during post-translational processing.
l) BAv9-1-5L-PIg23, BAv49-5L-PIg23, BAv81-5L-PIg23, BAv83-5L-PIg23, BAv85-5L-PIg23 expression vectors: PIg23 Fc was derived from PIg22 Fc by mutating the valine at 178 to methionine (M). The mutation was introduced into a PCR fragment encoding the PIg22-Fc using the splicing by overlap extension method (Horton et al., Gene 1989; 77:61-68) to obtain a DNA fragment encoding the PIg23 Fc. This fragment was joined to the BAv9-1-5L DNA fragment obtained as described in (h) above and the resulting polynucleotide was cloned into the CET1019AS vector using AgeI and PmeI cloning sites. The resulting plasmid CET-BAv9-1-5L-pIg23 contains a polynucleotide that encodes a fusion polypeptide as follows: the CTLA4-signal, BAv9-1 (CRD SEQ ID NO:3), a GGGGS linker (SEQ ID NO:156), and the PIg23 Fc. CET expression vectors for BAv49-5L-PIg23 (CRD SEQ ID NO:107), BAv81-5L-pIg23 (CRD SEQ ID NO:139), BAv83-5L-pIg23 (CRD SEQ ID NO:141), BAv85-5L-pIg23 (CRD SEQ ID NO:143) were generated in a similar manner. As described previously, the terminal lysine of the encoded protein may be cleaved off during post-translational processing.

The various fusion proteins described above were expressed and purified as described previously (Examples 2 and 3 for the CET1019AS based plasmids and Examples 7 and 8 for the pcDNA based plasmids). The signal peptide is typically cleaved during processing and thus the secreted BAv9-1-Ig Fc fusion protein does not ordinarily contain the signal peptide sequence. The BAv9-1-Ig Fc proteins typically exist in solution as a dimeric fusion protein. The purified fusion proteins were assayed for binding activity using BIACORE (Example 9) and KINEXA (Example 11) assays. The binding activity of the various BAv9-1-based fusion proteins with different stalk (residues 46-50 of the BCMA ECD SEQ ID NO:152) and Ig Fc combinations are shown in Table 6. The results showed that the various BAv9-1 fusion proteins have comparable binding activity for huBAFF (BIACORE and KINEXA), cyAPRIL (BIACORE), and huAPRIL (KINEXA) as compared to the BAv9-1-PIg88 fusion protein. In this study, changing the stalk and Ig Fc combination used in this Example, from PIg18 to 5L-PIg22 did not alter the BAFF and APRIL binding characteristics of BAv9-1 ECD. Similarly, Table 7 shows that changing the stalk and Ig Fc combination used in this Example, from PIg18 to 5L-PIg22 did not change the binding characteristics of BAv49, BAv81, BAv83, and BAv85 ECDs.

TABLE 6
Binding activities of variant BCMA polypeptides comprising substitutions in the sequence
corresponding to the BCMA stalk region and different combinations of Ig Fc

| | BIACORE* | | huBAFF KINEXA | | | huAPRIL KINEXA | | |
|---|---|---|---|---|---|---|---|---|
| | huBAFF | cyAPRIL | | High | Low | | High | Low |
| | KD | KD | KD | 95% | 95% | KD | 95% | 95% |
| Clone name | (pM) | (pM) | (pM) | CL | CL | (pM) | CL | CL |
| BAv9-1-pIg18 | | | 0.424 | 0.949 | 0.137 | 0.198 | 0.537 | 0.0279 |
| BAv9-1-pIg22 | 15.700 | 33.700 | 0.537 | 0.857 | 0.299 | 0.088 | 0.342 | 0 |
| BAv9-1K46G- | 18.700 | 46.800 | 0.495 | 1.100 | 0.127 | 0.056 | 0.734 | 0 |
| pIg18 | | | | | | | | |
| BAv9-1K46G- | 16.200 | 36.200 | 0.349 | 0.658 | 0.131 | 0.341 | 0.609 | 0.156 |
| zIg | | | | | | | | |
| BAv9-1K46G- | 16.500 | 45.600 | 0.484 | 0.803 | 0.244 | 0.32 | 0.632 | 0.112 |
| pIg22 | | | | | | | | |
| BAv9-1-5L- | 17.500 | 42.500 | 0.301 | 0.718 | 0.040 | 1.260 | 3.680 | 0.009 |
| pIg18 | | | | | | | | |
| BAv9-1-5L-zIg | 14.600 | 34.100 | 0.272 | 0.510 | 0.095 | 0.279 | 0.484 | 0.13 |
| BAv9-1-5L- | 16.500 | 41.800 | 0.864 | 1.82 | 0.284 | 0.611 | 1.65 | 0.113 |
| pIg22 | | | | | | | | |
| BAv9-1-[K46- | 16.900 | 37.800 | 0.228 | 0.954 | 0.001 | 0.461 | 0.910 | 0.164 |
| A50]del-zIg | | | | | | | | |
| BAv9-1- [K46- | 20.400 | 41.800 | 0.417 | 0.924 | 0.103 | 0.31 | 0.819 | 0.028 |
| A50]del-pIg22 | | | | | | | | |
| BAv9-1-5L- | | | 0.291 | 1.030 | 0.011 | | | |
| pIg23 | | | | | | | | |
| BCMA[S40G]- | 121 | 15.1 | 225.620 | 369.920 | 158.16 | 0.159 | 0.366 | 0.158 |
| pIg18 | | | | | | | | |

*BIACORE detection limit ~50 pM.
**A proportion of the purified polypeptide may be missing the terminal lysine residue.

TABLE 7
Binding activities of variant BCMA polypeptides comprising substitutions in the sequence
corresponding to the BCMA stalk region and different combinations of Ig Fc

| | BIACORE* | | huBAFF KINEXA | | | huAPRIL KINEXA | | |
|---|---|---|---|---|---|---|---|---|
| | huBAFF | cyAPRIL | | High | Low | | High | Low |
| | K_{D} | K_{D} | K_{D} | 95% | 95% | K_{D} | 95% | 95% |
| Clone name | (pM) | (pM) | (pM) | CL | CL | (pM) | CL | CL |
| BAv49-pIg18 | 16.2 | 1450.00 | 0.84234 | 4.2 | 0.0003 | 313.1 | 399.74 | 188.26 |
| BAv81-pIg18 | 8.31 | 2060.00 | 0.24603 | 1.02 | 0.00089 | 363.57 | 419.2 | 269.94 |
| BAv83-pIg18 | 8.09 | 707.00 | 0.254 | 1.03 | 0.001 | 113.730 | 136.580 | 84.400 |
| BAv85-pIg18 | 8.03 | 773.00 | 0.085 | 0.374 | 0.000 | 132.280 | 165.850 | 94.770 |
| BAv49-5L-pIg22 | 14.10 | 1760.00 | | | | | | |
| BAv81-5L-pIg22 | 6.75 | 2420.00 | | | | | | |
| BAv83-5L-pIg22 | 8.61 | 861.00 | | | | | | |
| BAv85-5L-pIg22 | 6.76 | 784.00 | | | | | | |
| BCMA[S40G]- | 121 | 15.1 | 225.620 | 369.920 | 158.160 | 0.159 | 0.366 | 0.158 |
| pIg18 | | | | | | | | |

*BIACORE detection limit ~50 pM.

### A. Quantitative Measurement of Lysine Hydroxylation and Methionine Oxidation

Variant polypeptides were analyzed for post-translational modifications (PTM). PTM is the chemical modification of a protein after its translation. PTM, such as lysine hydroxylation and methionine oxidation, were detected in the polypeptides from the variant BCMA molecules described herein. The PTM sites and the percentage of modified (PTM) variant BCMA molecules described herein were determined by peptide mapping. In this method, the protein was enzymatically digested into peptides that were measured by HPLC coupled with mass spectrometry. In carrying out this method, variant BCMA molecules described herein were first reduced by Dithiothreitol (DTT) to open the disulfide bonds and then the free thiols were alkylated with iodoacetamide in preparation for completion of enzymatic digestion in the next step. The thiol protected protein was incubated with Trypsin for 16 hours at 37° C. in a protein to enzyme ratio of 20. Under these conditions, digested protein peptides had no more than one PTM on each peptide. The peptide mixture was separated by a RP-HPLC column (Agilent, 1×50 mm, C18) and detected by mass spectrometry (Thermo Scientific, OrbiTrap) using selected ion monitoring (SIM). The SIM peak area of a PTM peptide over the total SIM peak area of the peptide (both PTM and non-PTM) yielded the percentage of a PTM modification (Table 8).

TABLE 8
Post-translational Modifications (PTM) of variant BCMA-Ig Fc fusion proteins

| | | Methione | Methione | Methione | Methione |
|---|---|---|---|---|---|
| | | sulfoxidation | sulfoxidation | sulfoxidation | sulfoxidation |
| | Hydroxylation | at position | at position | at position | at position |
| Clone Name | at Lysine 46 | 33* | 139* | 178* | 209* |
| BAv9-1-pIg18 | 59.1% | 3.5% | 35.8% | 6.3% | 7.4% |
| BCMA-zIg | 51.5% | 9.9% | — | — | 4.4% |
| BAV9-1-K46G-pIg18 | — | 4.4% | 31.2% | 9.0% | 9.1% |
| Bav9-1-zIg | 23.8% | 5.4% | — | — | 5.7% |
| Bav9-1-pIg22 | 25.4% | 5.7% | — | — | 7.0% |
| BAv9-1-K46G-zIg | — | 5.4% | — | — | 5.0% |
| BAv9-1-5L-pIg18 | — | 4.9% | 9.8% | 4.2% | 5.2% |
| BAv9-1-5L-zIg | — | 5.7% | — | — | 5.8% |
| BAv9-1-5L-pIg22 | — | 8.3% | — | — | 5.0% |
| BAv9-1-K46G-pIg22 | — | 5.8% | — | — | 7.8% |
| BAv9-1-5L-pIg22 | — | 7.6% | — | — | 1.0% |
| BAv9-1-5L-pIg23 | — | 7.0% | — | 1.4% | 1.1% |

*Position number with reference to human IgG2 Fc polypeptide.

Elimination of lysine at position 46 in the BCMA ECD of variant BCMAs by substitution with glycine (K46G) or replacement of residues 46-51 with GGGGS (SEQ ID NO:156) effectively eliminated hydroxylation for the resulting proteins (Table 8). Elimination of methionines at positions 2 and/or 3 in the IgG2 Fc region by replacement with valine or leucine effectively eliminated methionine sulfoxidation at these positions while maintaining very low levels of methionine sulfoxidation at the remaining methionine at positions 1 and 4 (Table 8).

### B. Half-Life Measurements of Fc Variants

To determine and compare serum concentration levels of three Fc variants, groups of three 6-8 week old female Balb/c mice were given single, subcutaneous injections of 100 g of one of the three variants. Blood was collected (100 l) from each mouse on days 2, 4, 7, 10, and 14. Serum was analyzed for free drug by muBAFF ELISA as follows. 96-well Maxisorp plates (Nunc) were coated overnight with recombinant mouse BAFF (R and D Systems) at 0.25 μg/ml. Plates were blocked with 2% BSA in TBST buffer. After incubation of mouse serum dilutions with the plates, the binding of free drug to immobilized muBAFF was detected with anti-human IgG(H+L) HRP antibody (Life Technologies) and BM chemiluminescence POD substrate (Roche). Plates were read on Analyst HT 96-384 (Molecular Devices) and analyzed with Criterion Host Software (Molecular Devices).

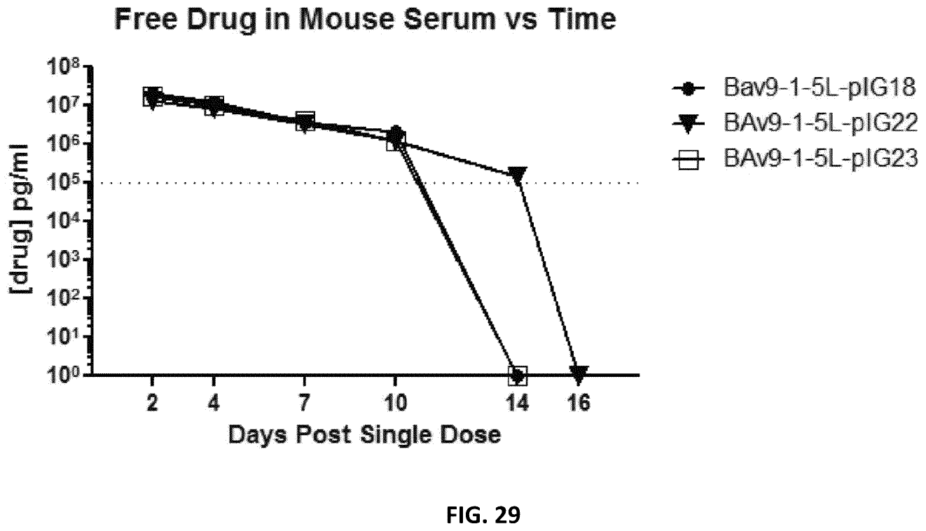

Comparison of the half-lives of the three Fc variants show that variant BCMA ECD polypeptides fused to pIg22 appear to have more sustained kinetics than variant BCMA ECD polypeptides fused to either pIg18 or pIg23 (FIG. 29). Mutation of methionines at positions 139 and 178 in the IgG2 Fc region by replacement with valine or leucine (such as pIg22) resulted in a protein with longer half-life than a variant with mutation of only methionine at position 139 (such as pIg23).

### Example 18: Removal of Glycosylation Site From CRD Region of BCMA ECD

This Example describes manipulation of plasmids encoding variant BCMA ECDs to prevent N-linked glycosylation. The mutants lacking glycosylation were then compared to the glycosylated polypeptides to assess whether the mutations impacted BAFF/APRIL binding activity.

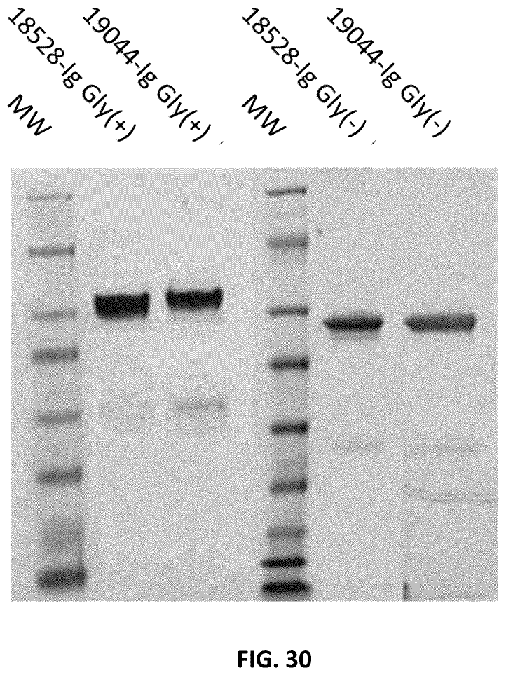

BCMA ECD contains a recognition sequence for N-linked glycosylation (NAS) in the stalk region of the natural receptor (residues 38-40). The NAS sequence is not glycosylated on the natural BCMA ECD of the cell surface (Pelletier et al., J Biol Chem 2003; 278(35):33127-33; Gras et al., Int Immunol. 1995; 7(7):1093-106; Thompson et al., J Exp Med. 2000; 192(1):129-3). When variant BCMAs, such as 18528-Gly(+) and 19044-Gly(+) (i.e., containing S40 as in the native huBCMA ECD (SEQ ID NO:152) were fused to a TPA N-terminal signal sequence and IgG2 FcSS domains, the resulting secreted fusion proteins were glycosylated at the NAS sequence in the ECD as evidenced by the broad band seen in SDS-PAGE gels (FIG. 30). The codon encoding serine at residue 40 (AGC) was mutated to glycine (GGC) on the plasmids encoding 18528-Gly(+)-IgG2 FcSS and 19044-Gly(+)-IgG2 FcSS to prevent N-linked glycosylation. The resulting plasmids 18528-Gly(−)-IgG2 FcSS and 19044-Gly(−)-IgG2 FcSS were transfected into CHO cells and the resulting purified proteins lacked glycosylation as evidenced by the tight band seen in SDS-PAGE gels (FIG. 30). The affinity of the S40G Gly(−) mutants 18528-Gly(−) and 19044-Gly(−) had binding constants to murine BAFF and APRIL that were not markedly different to 18528-Gly(+) and 19044-Gly(+) versions of the same variant BCMA (Table 9).

TABLE 9
Binding properties of G40 (Gly(+)) and S40
(Gly(−)) variant BCMA polypeptides*

| | muBAFF | muBAFF | muBAFF | muAPRIL | muAPRIL | muAPRIL |
|---|---|---|---|---|---|---|
| Clone Name | ka (1/Ms) | kd (1/s) | K_{D} (pM) | ka (1/Ms) | kd (1/s) | K_{D} (pM) |
| 18528-Gly(+) | 4.11E+06 | 2.20E−04 | 54 | 1.87E+06 | 7.05E−05 | 38 |
| 18528-Gly(−) | 4.62E+06 | 2.96E−04 | 64 | 2.39E+06 | 7.88E−05 | 33 |
| 19044-Gly(+) | 8.35E+05 | 1.34E−03 | 1600 | 1.50E+06 | 8.22E−05 | 55 |
| 19044-Gly(−) | 1.47E+06 | 1.34E−03 | 920 | 2.49E+06 | 9.33E−05 | 38 |

*Gly (+) and Gly (−) refer to an amino acid sequence having or not having the [S40G] substitution, respectively, relative to the native human BCMA ECD (SEQ ID NO: 152).

These results demonstrate that BAFF/APRIL binding activity is retained upon removal of an N-linked glycosylation site. Preventing glycosylation can yield a more homogeneous protein due to the absence of different glycoforms produced by a glycosylating host cell.

### Example 19: Improved Efficacy of BCMA-Ig Variants Versus Human BCMA Surrogate Control Protein in Lupus Mice

The effects of various antagonists, selective for BAFF, APRIL, or both BAFF and APRIL, were assessed in lupus-prone mice. The effects of human BCMA and variant BCMA fusion polypeptides were compared.

NZB/W-F1 mice were purchased from Japan SLC, Inc. at 4 months age. Administration of test proteins (at 1 or 10 mg/kg) was started at 7 months age and the proteins were administered weekly, via subcutaneous route, for a total of 10 weeks. Urine protein and urine creatinine were measured weekly during the dosing period. Splenic B cells were analyzed at the end of 10 weekly treatments.

The lupus-prone mouse study was performed and analyzed as described above during treatment with protein preparations of various antagonists of muBAFF (mouse BAFF) and muAPRIL (mouse APRIL), including a variant BCMA ECD-Ig fusion protein (BAv9-1-5L-PIg22[desK278]) with strong activity for muBAFF and muAPRIL, human BCMA surrogate control protein, [S40G]BCMA ECD-IgG2 Fc fusion protein (containing the extracellular domain of the S40G-substituted human BCMA ECD fused to a human IgG2 Fc domain, or control Fc (human IgG1 Fc protein from Bioxcel).

Urine samples were collected and the urine protein content was determined using the Bradford assay and the urine creatinine determined using the creatinine assay kit (Abcam, Cambridge, MA). Urine protein excretion was estimated as the urine protein/urine creatinine ratio (Upro/Ucre) and ratios above 3 were considered evidence of kidney pathology.

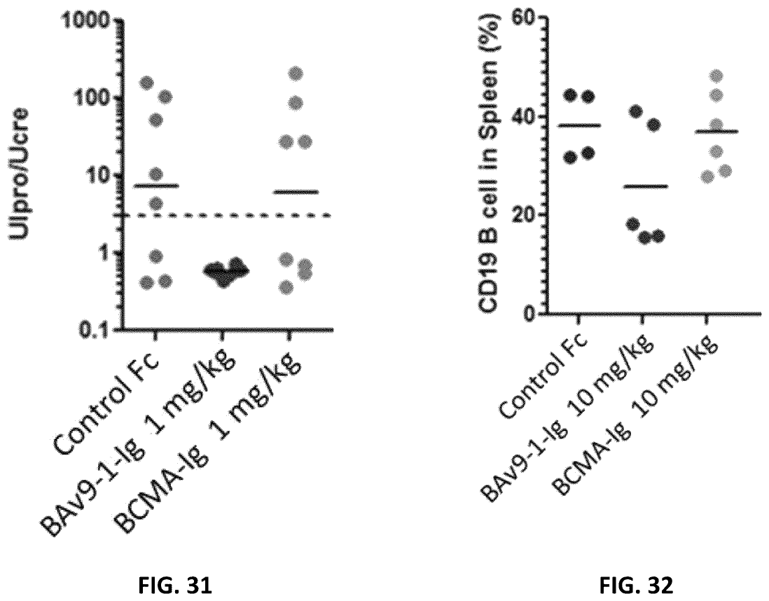

Inhibitory effects on various biomarkers of B cell biology and the lupus-prone disease model were determined for all groups. FIG. 31 shows urine protein/urine creatinine ratios (Upro/Ucre) from individual mice after 6 weekly treatments with test compounds: control Fc protein, variant BAv9-1-5L-pIg22[desK278]-(labeled “BAv-9-1-Ig”), or human BCMA surrogate control protein (labeled “BCMA-Ig”), [S40G]BCMA ECD-IgG2 Fc, respectively. FIG. 32 shows the reduction in splenic B-cells from NZB/W-F1 mice after 10 weekly treatments with the same test compounds. All animals had been given 10 weekly treatments treated with the listed compounds administered subcutaneously. More potent inhibition on all biomarkers described above was seen with the equivalent dose of variant BCMA BAv9-1-5L-pIg22[desK278] versus the human BCMA surrogate control protein, [S40G]BCMA ECD-IgG2 Fc.

The data are consistent with an observation that the variant BCMA-Ig polypeptides are more potent than wild type huBCMA-Ig (as demonstrated with respect to the human BCMA surrogate control protein in this experiment), at inhibiting B cells and preventing lupus disease manifestations in vivo.

### Example 20: Generation of Cell Lines to Produce BCMA CRD Variant CRD-IgG4 Fusion Protein

This example describes the generation of a cell line for producing a B-cell maturation antigen (BCMA) CRD-IgG4 fusion protein.

### A. Construction of the DNA Plasmid Vector

A plasmid construct was generated for the production of an exemplary variant BCMA CRD polypeptide, fused with a peptide linker and a human IgG4 Fc variant. As demonstrated herein, the exemplary variant BCMA CRD polypeptide-IgG4 Fc variant fusion protein exhibited dual binding to BAFF and APRIL to prevent differentiation and survival of long-lived plasma B cells, and enhanced BAFF binding. Further, the fusion protein comprising the human IgG4 variant led to a prevention of in vivo and in vitro IgG4 Fab-arm exchange and reduced effector functions.

The plasmid vector construct encoded an exemplary BCMA CRD-IgG4 Fc fusion protein as follows: a signal sequence (SEQ ID NO:153, encoding SEQ ID NO:154), BAv9-1 CRD (SEQ ID NO: 159, encoding SEQ ID NO:3), a GGGGSA linker (SEQ ID NO:158, encoded by SEQ ID NO:157), and a variant human IgG4 Fc comprising the S228P/F234A/L235A/L445P substitutions (substitutions annotated by EU numbering; SEQ ID NO:238, encoding SEQ ID NO:163).

An exemplary vector also included the components as set forth in Table 10 below, including a human EF1α promoter, nucleic acids encoding the fusion protein, an internal ribosome entry site (IRES)-mediated bicistronic expression cassette of membrane-anchored green fluorescent protein (GFP) flanked by LoxP sites, a puromycin resistant gene for mammalian selection, and an ampicillin gene for bacteria propagation, and sequences encoding a switchable membrane reporter (SwiMR) to facilitate the isolation of highly productive cells via fluorescence activated cell sorting (FACS).

TABLE 10
Plasmid Construct and Elements

| Element | Function |
|---|---|
| Xbal | Unique Xbal restriction enzyme site used for the |
| | insertion of the BCMA CRD variant-IgG4 |
| | Fc sequence |
| Signal peptide | Targets BCMA CRD variant-IgG4 Fc for |
| | secretion from CHO cells |
| BCMA CRD | Exemplary BCMA CRD variant (BAv9-1; S12H, |
| variant | L14I, H15R, S40G, encoding SEQ ID NO: 3) |
| Peptide linker | Encoding GGGGSA peptide linker (encoding |
| | SEQ ID NO: 158) |
| Human IgG4 Fc | Engineered IgG4 Fc region with mutations |
| region variant | (S228P, F234A, L235A, and L445P by EU |
| | numbering, encoding SEQ ID NO: 163) |
| | S228P for stabilized dimer |
| | F234A and L235A for reduced effector function |
| BamHI | Unique BamHI restriction enzyme site |
| | used for the insertion of the BCMA |
| | CRD variant-IgG4 Fc sequences |
| LoxP | LoxP flanks the IRES-MAR cassette used |
| | for clone selection |
| | Addition of cyclization recombinase |
| | (Cre) removes the IRES-MAR cassette |
| Sequencing | Sequencing primer |
| primer | |
| IRES | Internal ribosome entry site (IRES)-mediated |
| | bicistronic expression cassette of |
| | membrane-anchored GFP |
| Signal peptide | IL2 sequence peptide for membrane targeting |
| | of GFP |
| GPF | Green fluorescent protein that is used for cell |
| | selection via FACS The IRES-MAR |
| | cassette is removed post cell selection by |
| | using the flanked LoxP and Cre |
| LoxP | LoxP flanks the IRES-MAR cassette |
| | used for clone selection |
| | Addition of cyclization recombinase |
| | (Cre) removes the IRES-MAR |
| | cassette between the LoxP sites |
| bGH-Poly-A | Bovine growth hormone polyadenylation |
| | region Transcription termination and mRNA |
| | stability for the encoded BCMA CRD |
| | variant-IgG4 Fc transcript |
| NotI | Unique NotI restriction enzyme site |
| SV40 | Promoter to drive transcription of puromycin |
| | resistant gene |
| Puro | Puromycin resistant gene for CHO cell selection |
| SV40 Poly-A | Transcription termination and mRNA stability |
| | for puromycin resistance |
| pUC | Plasmid cloning vector for E coli |
| Amp | Puromycin resistant gene for microbial selection |
| FspI | Unique FspI restriction enzyme site |
| AscI | Unique AscI restriction enzyme site used for |
| | the linearization of vector |
| hEF1α | Promoter for strong constitutive mammalian |
| | cell expression |
| Sequencing | Sequencing primer |
| primer 2 | |

The polynucleotide sequence encoding the fusion protein (including the signal sequence) is set forth in SEQ ID NO:239. The amino acid sequence of the mature expressed exemplary BCMA CRD-IgG4 Fc fusion protein (BAv9-1 CRD-IgG4 Fc) is set forth in SEQ ID NO:167.

### B. Generation of Cell Line

For generation of a cell line to produce the exemplary BCMA CRD variant CRD-IgG4 fusion protein, Chinese hamster ovary (CHO) cells were transfected with the vector construct described above.

CHO-S cells were expanded in five passages of chemically defined medium and cryopreserved to create an intermediate bank of cells. Plasmid vectors encoding the BCMA CRD variant BAv9-1 CRD-IgG4 fusion protein as described above were linearized and transfected into CHO-S cells thawed from the intermediate bank and expanded for four passages chemically defined medium. The transfected cell population were subject to two rounds of FACS sorting. Candidate clones were expanded and selected based on growth and titer performance and cryopreserved. Monoclonality of the candidate clone was confirmed based on single cell printing.

### Example 21: Comparative Binding Affinity of a Dual BAFF/APRIL Antagonist Comprising a BCMA CRD and an IgG4 Fc

In this Example, the binding of a fusion protein that includes an engineered BCMA CRD and an Ig Fc domain was evaluated. In some aspects, an IgG4 Fc domain was included in the fusion protein. IgG4 Fc domains minimize effector function and are immunologically inert, which may be advantageous, in cases where widespread immune activation is more harmful, for example in adverse events. IgG4 Fc domains are associated with a reduced cytotoxic response, in some cases due to weak affinity to the Fcγ receptors, leading to reduced or minimal antibody-dependent cellular cytotoxicity (ADCC), antibody-dependent cellular phagocytosis (ADCP), and complement-dependent cytotoxicity (CDC) activity. As the treatment of autoimmune disease requires a strategy of targeted immunosuppression, the reduced effector function and cytotoxic response can provide an advantage.

The K_{D} of an exemplary variant BCMA polypeptide, BAv9-1 CRD-Ig Fc, generated generally as described in Example 20 above, and various other binding molecules, to huBAFF or huAPRIL were compared, as shown in Table 11. The binding affinity was compared to that of other BAFF or APRIL binding molecules, the BAFF/APRIL binding molecule Atacicept, the anti-BAFF antibody Benlysta (belilumab), and the anti-APRIL antibody BION-1301, to huBAFF or huAPRIL. Atacicept is a human recombinant fusion protein that includes the extracellular domain of TACI, fused to a modified IgG1 Fc, which could bind both BAFF and APRIL, with substantially equal affinity. As shown, the binding affinity of BAv9-1 CRD-Ig Fc to both human BAFF and human APRIL were substantially higher than the other molecules to each of its binding targets, with K_{D} for both human BAFF and human APRIL being in the sub-picomolar range. For example, the binding affinity of the exemplary BCMA CRD variant-Ig Fc was approximately 600- to 800-fold higher compared to the binding affinity Atacicept to BAFF and APRIL. The binding affinity of BAv9-1 CRD-Ig Fc was higher for APRIL than BAFF.

The results are consistent with the exemplary BCMA CRD variant exhibiting substantially improved binding affinity to both BAFF and APRIL compared to other therapeutic molecules that bind to either or both. The results support substantial advantages of the exemplary BCMA CRD variant described herein for use in therapy. For example, higher binding affinity can lead to improved therapeutic effect, improved safety profiles, potential to reduce dosing frequency or levels, and improved patient compliance.

TABLE 11
Affinity to Human Ligands BAFF and/or APRIL

| | BAFF K_{D} (pM) | APRIL K_{D} (pM) |
|---|---|---|
| BAv9-1 CRD-Ig Fc | 0.24 | 0.15 |
| Atacicept | 150 | 130 |
| Benlysta | 2.3 | N/A |
| BION-1301 | N/A | 400 |

### Example 22: Comparative Binding Affinity of a Dual BAFF/APRIL Antagonist Comprising a BCMA CRD and an IgG4 Fc Using Surface Plasmon Resonance

In this Example, the binding of a fusion protein that includes an engineered BCMA CRD and an Ig Fc domain was evaluated using a different assay, surface plasmon resonance (SPR). The K_{D} of the exemplary variant BCMA polypeptide, BAv9-1 CRD-Ig Fc, and various other binding molecules, to human or murine BAFF or human or murine APRIL were compared.

The K_{D} of a BAv9-1 CRD-IgG4 Fc fusion protein, and other BAFF or APRIL binding molecules, the BAFF/APRIL binding molecule Atacicept and a different recombinant TACI-Fc fusion protein RC18 (also known as Telitacicept), to human or murine BAFF or human or murine APRIL were determined by surface plasmon resonance (Carterra LSA, HC30M sensor chip).

The determined K_{D} are listed in Tables 12 and 13. As shown, the binding affinity of BAv9-1 CRD-IgG4 Fc to both human BAFF and APRIL (Table 12) were higher, compared to the binding affinity of Atacicept or Telitacicept to each. Also, the binding affinity of BAv9-1 CRD-Ig Fc to both murine BAFF and APRIL (Table 13) were substantially higher, compared to the binding affinity of Atacicept or Telitacicept to each.

TABLE 12
Affinity to Human Ligands BAFF and/or APRIL

| | BAFF K_{D} (pM) | APRIL K_{D} (pM) |
|---|---|---|
| BAv9-1 CRD-Ig Fc | 117 | 25 |
| Atacicept | 919 | 67 |
| RC18 (Telitacicept) | 616 | 82 |

TABLE 13
Affinity to Murine Ligands BAFF and/or APRIL

| | BAFF K_{D} (pM) | APRIL K_{D} (pM) |
|---|---|---|
| BAv9-1 CRD-Ig Fc | 31 | 0.15 |
| Atacicept | 532 | 130 |
| RC18 (Telitacicept) | 2.3 | N/A |

These affinity data demonstrate that exemplary BCMA CRD-Ig Fc fusion protein binds to human and murine BAFF and APRIL with high affinity, and show the highest binding affinity to both BAFF and APRIL compared to other molecules targeting BAFF and APRIL. In some aspects, this could be attributable to the engineered modifications to the BCMA binding pocket and CRD. The results support substantial advantages of the exemplary BCMA CRD variant described herein for use in therapy. For example, higher binding affinity can lead to improved therapeutic effect, improved safety profiles, potential to reduce dosing frequency or levels, and improved patient compliance.

### Example 23: Assessment of Binding of a Dual BAFF/APRIL Antagonist Comprising a BCMA CRD and an IgG4 Fc

As described above in Example 20, a fusion protein of an exemplary BCMA CRD and IgG4 Fc was generated. The polynucleotide sequence encoding the exemplary BCMA CRD-IgG4 Fc fusion protein contained nucleic acid sequences encoding the following: a signal sequence (SEQ ID NO:154), BAv9-1 CRD (SEQ ID NO:3), a GGGGSA linker (SEQ ID NO:158), and an IgG4 Fc (SEQ ID NO:163). The polynucleotide sequence encoding the fusion protein is set forth in SEQ ID NO:239. The amino acid sequence of the mature expressed exemplary BCMA CRD-IgG4 Fc fusion protein (BAv9-1 CRD-IgG4 Fc) is set forth in SEQ ID NO:167.

A colorimetric ELISA was used to assess the binding of BAFF/APRIL binding molecules to human and murine BAFF and APRIL. Inhibition curves based on binding to soluble human and murine BAFF and APRIL were used to determine the IC₅₀ values for binding of the exemplary BCMA CRD variant-IgG4 fusion protein BAv9-1 CRD-IgG4 Fc, Atacicept and a different recombinant TACI-Fc fusion protein RC18 (also known as Telitacicept).

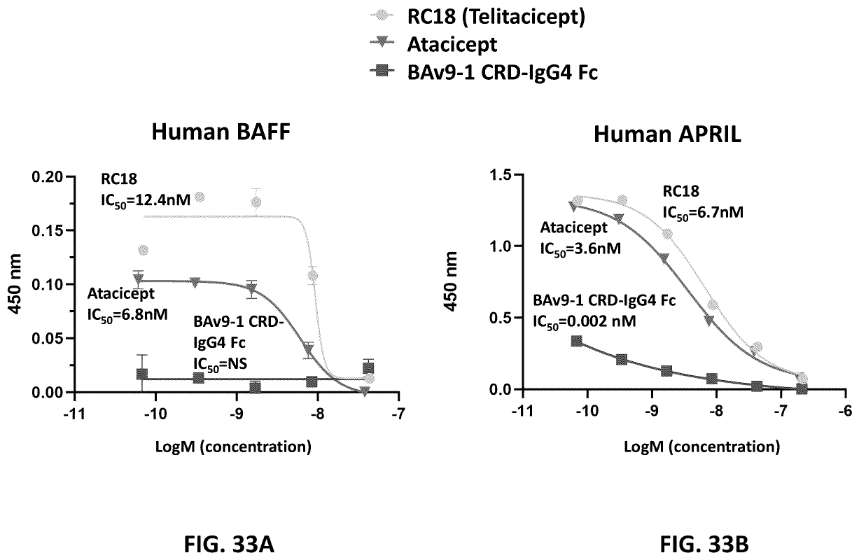

FIG. 33A and FIG. 33B show the inhibition curves of the three different BAFF/APRIL binding molecules to human BAFF (huBAFF; FIG. 33A) and human APRIL (huAPRIL; FIG. 33B). Compared to the IC₅₀ values determined for Atacicept and RC18 (Telitacicept), the IC₅₀ of the exemplary BCMA CRD variant-IgG4 fusion protein BAv9-1 CRD-IgG4 Fc was orders of magnitude lower for both huBAFF and huAPRIL. Total inhibition was observed for all tested levels of the BCMA CRD variant-IgG4 fusion protein for human BAFF, and the IC₅₀ value for huAPRIL in this assay was 0.002 nM.

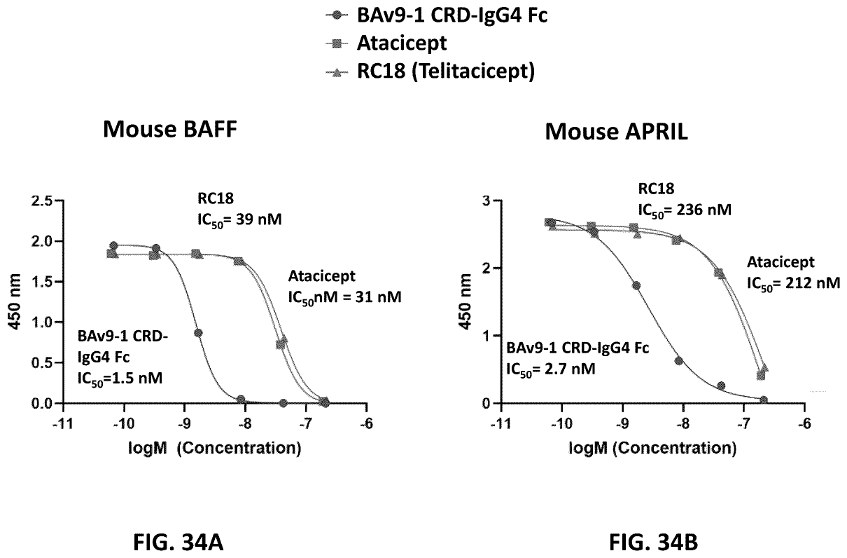

FIG. 34A and FIG. 34B show the inhibition curves of the three different BAFF/APRIL binding molecules to murine BAFF (FIG. 34A) and murine APRIL (FIG. 34B). Compared to the IC₅₀ values determined for Atacicept and RC18 (Telitacicept), the IC₅₀ of the exemplary BCMA CRD variant-IgG4 fusion protein BAv9-1 CRD-IgG4 Fc was orders of magnitude lower for both murine BAFF and murine APRIL. The IC₅₀ values for BAv9-1 CRD-IgG4 Fc for murine BAFF and murine APRIL were 1.5 nM and 2.7 nM respectively.

The results are consistent with the potent inhibition of human and murine BAFF and APRIL by the exemplary BCMA CRD variant-IgG4 fusion protein, at a much lower concentration compared to other BAFF/APRIL binding molecules, indicating substantially greater binding and potency of the exemplary BCMA CRD variant-IgG4 fusion protein.

### Example 24: Determination of Affinity of Dual BAFF/APRIL Binding Molecules for Heparin Sulfate Proteoglycans (HSPGs)

The binding of an exemplary BCMA variant CRD-IgG4 fusion protein, Atacicept, and RC18 (Telitacicept) to heparin sulfate proteoglycan (HSPG) was assessed using a colorimetric ELISA assay. HSPGs are required for normal B cell maturation, differentiation and function. Use of a molecule that binds to HSPGs may interfere with HSPG-mediated functions in immune responses, such as in regulating cell adhesion, cytokine and chemokine function, sensing tissue injury, and mediating inflammatory reactions, for example, in T cell-independent responses to bacterial infections.

The EC₅₀ for the binding of an exemplary BCMA variant CRD-IgG4 fusion protein BAv9-1 CRD-IgG4 Fc (as described in Example 20), Atacicept, and RC18 (Telitacicept) to cell surface transmembrane HSPGs syndecan-1 or syndecan-2, were determined based on binding curves.

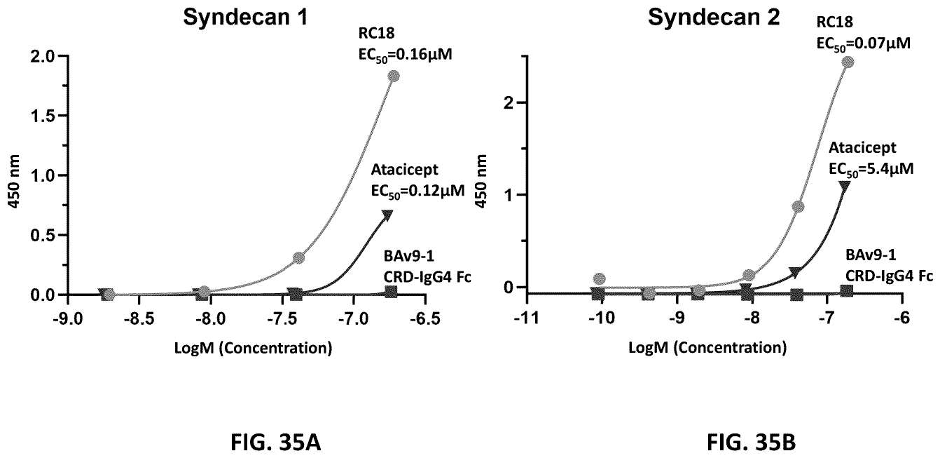

FIG. 35A and FIG. 35B show the binding curves and binding affinities of BAv9-1 CRD-IgG4 Fc, Atacicept, and RC18 (Telitacicept) for syndecan-1 (FIG. 35A) and syndecan-2 (FIG. 35B), respectively. In contrast to the EC₅₀ values for Atacicept and RC18 (Telitacicept), a complete absence of binding was observed for the exemplary variant BCMA CRD-IgG4 fusion protein. The results demonstrate that Atacicept and RC18 (Telitacicept) were capable of binding HSPGs at the tested concentrations, whereas the exemplary variant BCMA CRD-IgG4 Fc fusion protein was not. These results support that the exemplary variant BCMA CRD-IgG4 Fc fusion protein results support that BCMA-IgG4 does not substantially bind HSPGs, and accordingly, would not interfere with HSPG-mediated immune function and activities. Accordingly, the exemplary variant BCMA CRD-IgG4 fusion proteins would provide an advantages in therapy, for example, of preserving HSPG-mediated immune functions in the subjects.

### Example 25: Binding of a Dual BAFF/APRIL Antagonist Comprising a BCMA CRD and an IgG4 Fc to Human B Cells Stimulated with Human BAFF, Human APRIL, and Human BAFF-60 Mer in the Presence of an Anti-IgM Antibody and Inhibition

This example describes the binding of and inhibition by an exemplary BCMA variant CRD-IgG4 fusion protein, Atacicept, and RC18 (Telitacicept) on primary human B cells from a donor, stimulated with human BAFF, human APRIL, and human BAFF-60mer in the presence of anti-IgM antibody.

### A. B Cell Enrichment and Receptor Expression

Primary human B cells were obtained from three human donors (Donor 1, Donor 2, and Donor 3). Buffy coats were processed to obtain peripheral blood mononuclear cells (PBMCs). PBMCs were enriched to obtain an enriched population of B cells and resuspended in RPMI supplemented with 10%-HI-Low-IgG-FBS and Penicillin-Streptomycin-Glutamine (PSG) (Assay Medium). Enrichment efficiency was assessed by flow cytometry. Enriched B cells were stained with antibodies specific for CD19, BAFF-R, TACI and BCMA. Unstained cells were included as control.

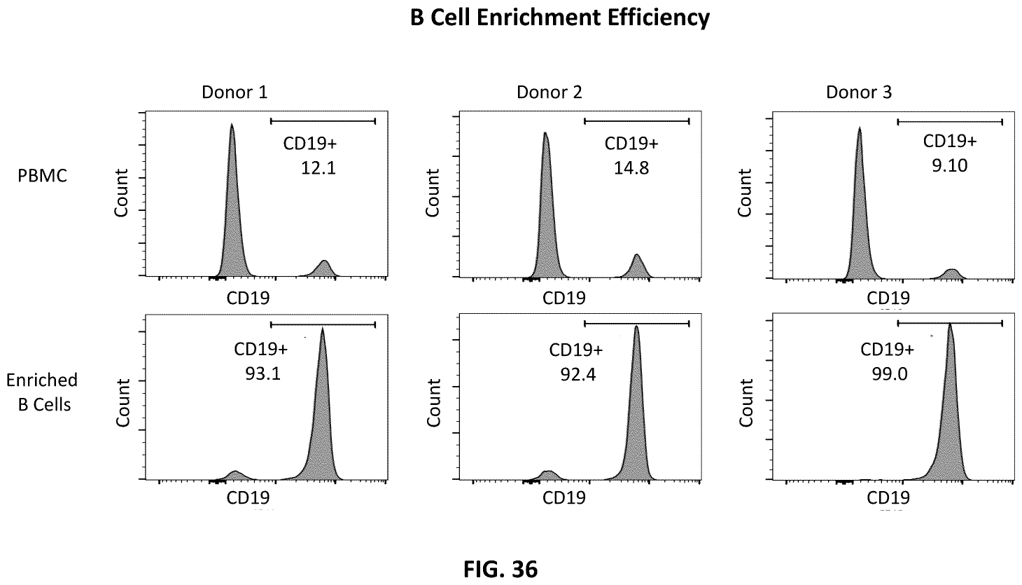

FIG. 36 depicts the B cell enrichment efficiency assessment, gated on lymphocytes. As shown, high efficiency of B cell enrichment, as indicated by more than 90% CD19+ cells in the enriched population, was achieved in all three donors.

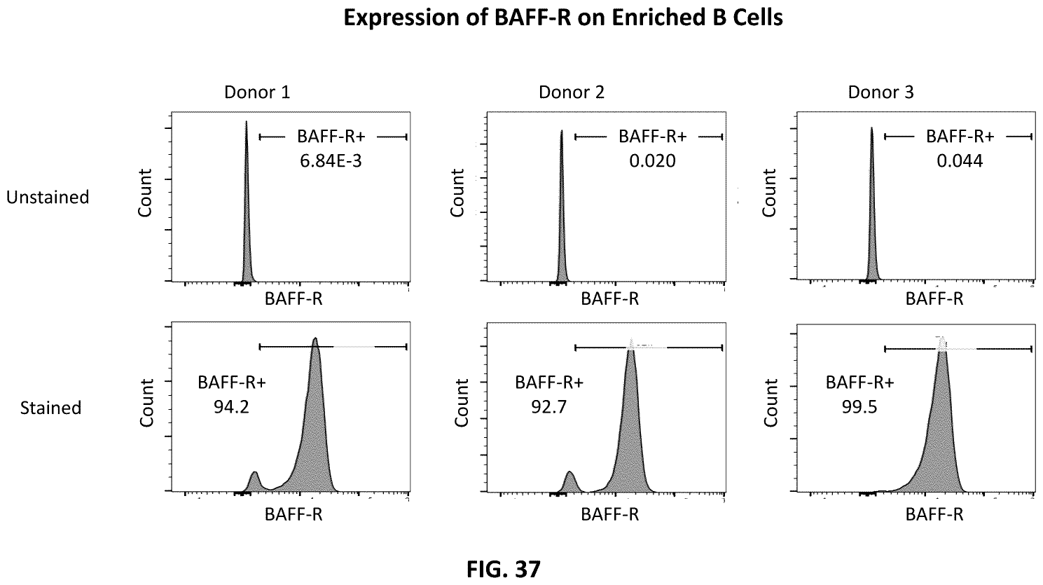

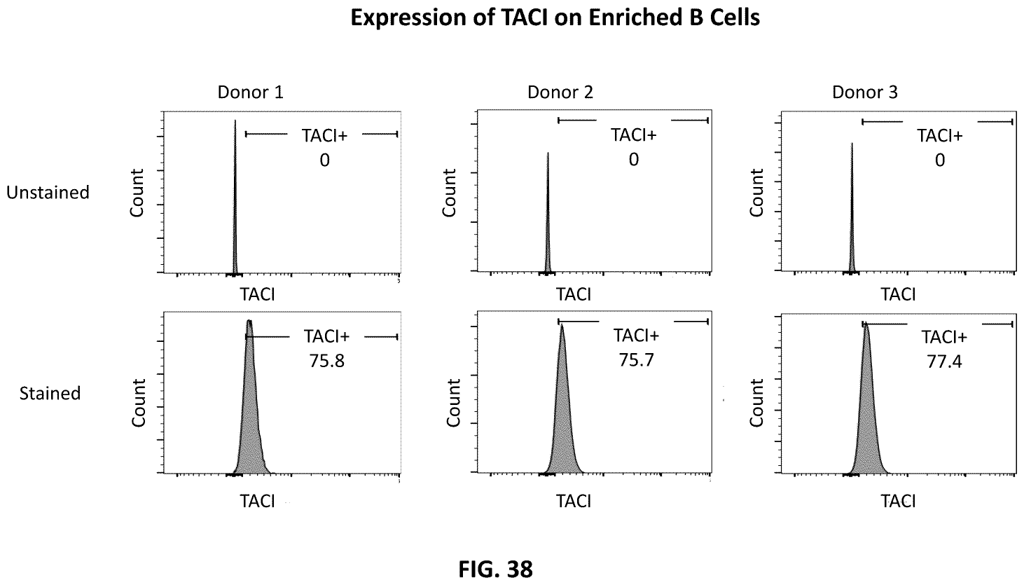

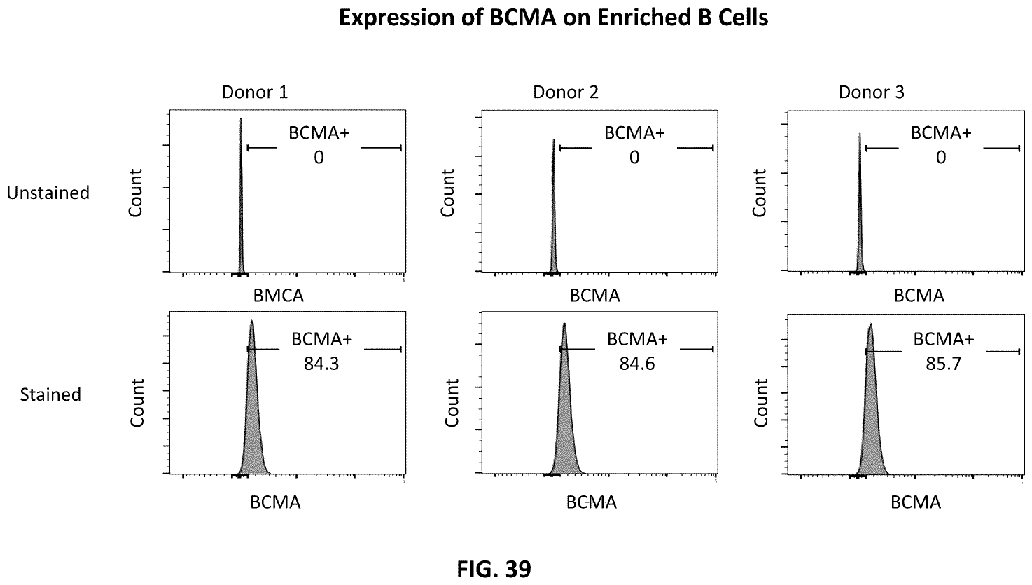

FIG. 37 depicts BAFF-R expression of the enriched B cell population. As shown, for all three donors, more than 90% of the cells in the enriched B cell population was BAFF-R+. FIG. 38 depicts the TACI expression of the enriched B cell population. As shown, for all three donors, more than 75% of the cells in the enriched B cell population was TACI+. FIG. 39 depicts BCMA expression of the enriched B cell population. As shown, for all three donors, more than 80% of the cells in the enriched B cell population was BCMA+.

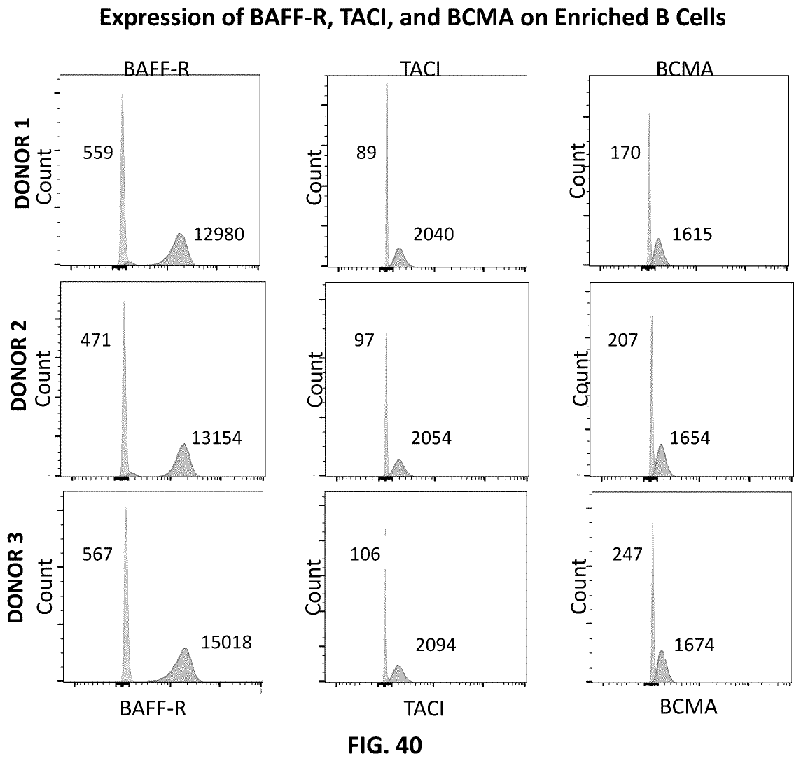

FIG. 40 depicts the expression of BAFF-R, TACI, and BCMA in the enriched B cell population for the individual donors. The compiled histogram overlays depict the signal for BAFF-R, TACI, and BCMA for Donors 1, 2 and 3, and the mean fluorescence intensity values (MFI values) are indicated next to the respective histograms, indicating high levels of BAFF-R, TACI and BCMA, substantially above the background level for unstained control cells. In general, the level of expression of TACI and BCMA in this study was observed to be lower than the expression of BAFF-R in all donors.

The results showed that the enrichment resulted in a primary human population highly enriched for B cells, which expressed high levels of BAFF-R, TACI and BCMA.

B. Stimulation of Enriched B Cells with BAFF, APRIL, and BAFF-60Mer and Anti-IgM Antibody and Inhibition by Exemplary BCMA Variant, Atacicept, and RC18

The inhibitory effect of an exemplary BCMA CRD variant BAv9-1 CRD-IgG4 Fc, Atacicept, and RC18 (Telitacicept) on enriched primary human B cells stimulated with ligands were assessed. Enriched B cells were adjusted to a concentration of 0.5 million cells/mL in Assay Medium. Before plating the cells, 96-well plates were coated with anti-IgM antibody at 37° C. and 5% CO₂ in a humidified standard cell culture incubator for approximately 2 hours, followed by wash with PBS. Uncoated wells (no anti-IgM antibody) were used as controls. Four-fold serial dilutions (starting from 120 nM) of BAv9-1 CRD-IgG4 Fc, Atacicept, or RC18 (Telitacicept) were added to enriched B cell populations that were stimulated with the plate-bound anti-IgM antibody and one of the three soluble ligands: human BAFF (0.4 nM, Acros Biosystems), human APRIL (0.4 nM, Acros Biosystems) or human BAFF-60mer (0.4 nM, Adipogen). Plates were incubated at 37° C. and 5% CO₂ in a humidified standard cell culture incubator for 96 hours. The extent of stimulation and proliferation of the B cells were determined by assessing the viable cell numbers using the CellTiter-Glo® Luminescent Cell Viability Assay (Promega). Luminescence was assessed using a plate reader (i3x-Molecular Devices), and the Relative Luminescence Units (RLU), and the IC₅₀ values were determined based on inhibition curves from a plot of RLU over concentration of the inhibitor.

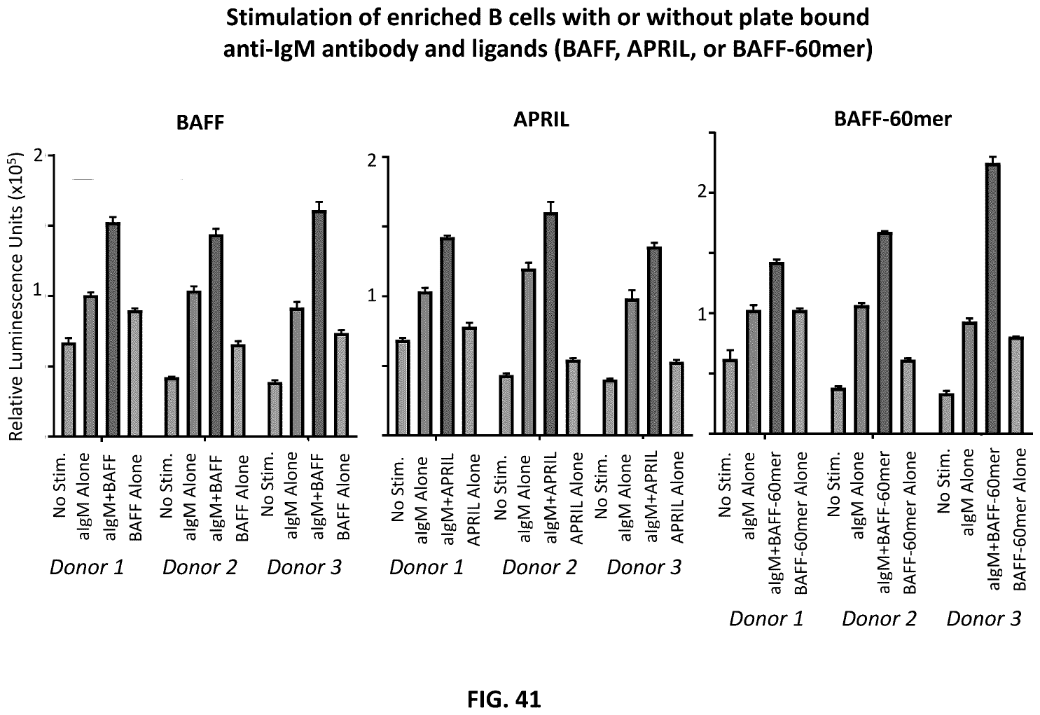

FIG. 41 depicts the stimulation and proliferation of the enriched B cell population in the presence of stimulatory ligands (BAFF, APRIL, or BAFF-60mer), with or without plate bound anti-IgM antibody for all three donors: no stimulation (“no stim”), anti-IgM antibody alone (“aIgM alone”), anti-IgM antibody with ligand (“aIgM+BAFF,” “aIgM+APRIL,” or “aIgM+BAFF-60mer”) and ligand alone (“BAFF,” “APRIL,” or “BAFF-60mer”), for all three donors. For all three donors and for all the ligands BAFF, APRIL or BAFF-60mer, the enriched B cell population showed highest stimulation and proliferation in the presence of plate bound anti-IgM antibodies. In general, B cells stimulated with each of the ligands alone without anti-IgM antibodies, did not result in substantial enhancement of proliferation compared to the no stimulation control, and cells stimulated with anti-IgM antibodies alone resulted in intermediate level of stimulation and proliferation.

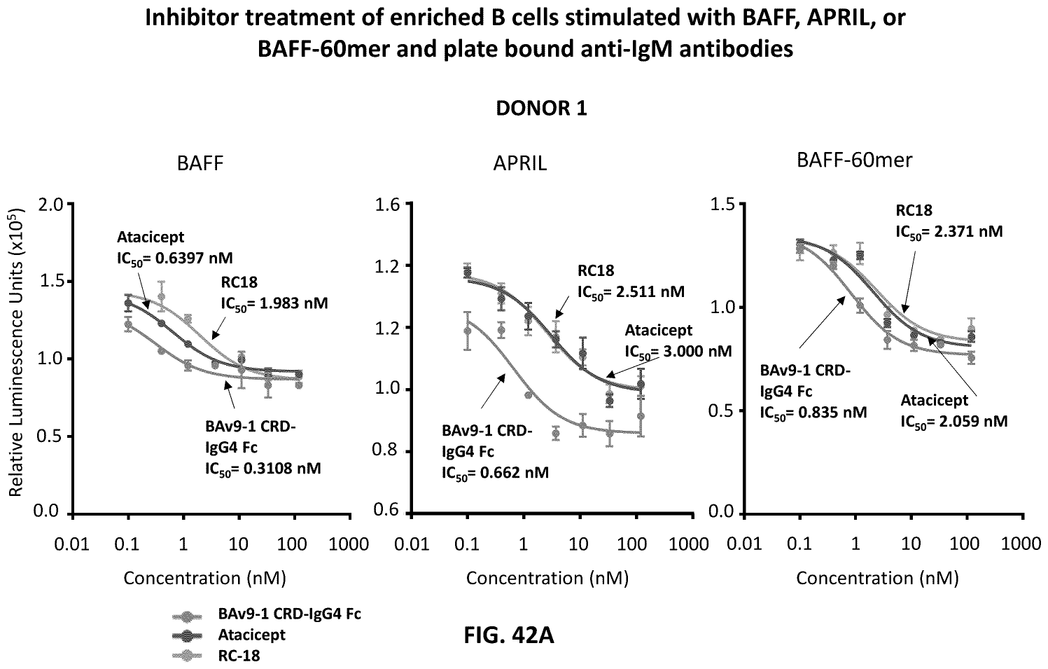

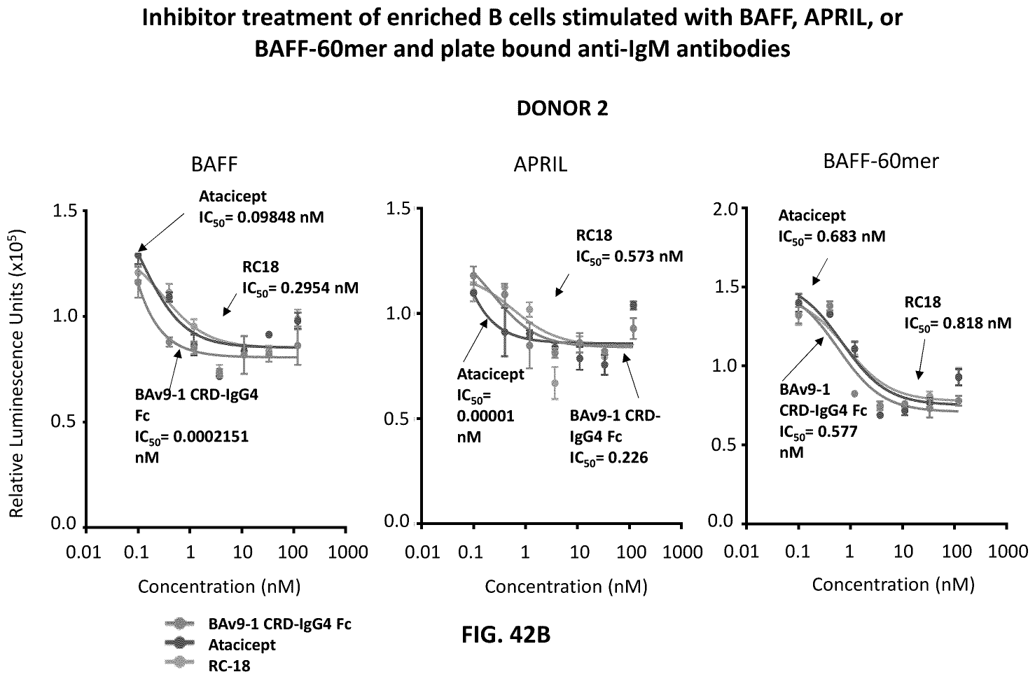

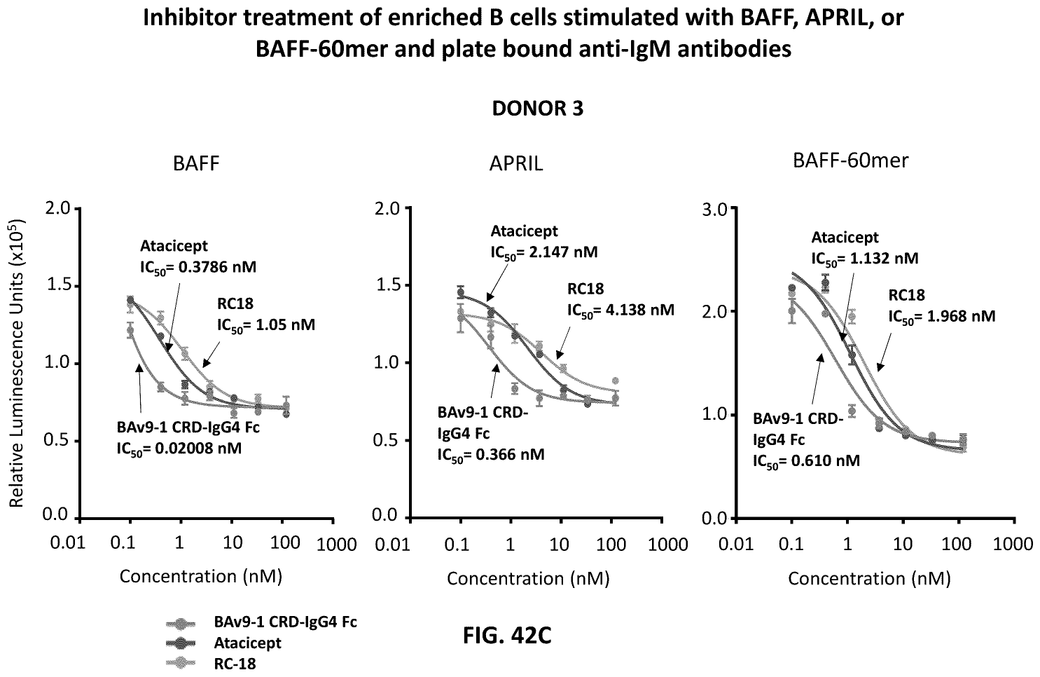

FIGS. 42A-42C depict the inhibition curves of the three inhibitors BAv9-1 CRD-IgG4 Fc, Atacicept, and RC18 (Telitacicept), for proliferation of the enriched B cell population stimulated with human BAFF, human APRIL, or human BAFF-60mer and anti-IgM antibodies, for Donor 1 (FIG. 42A), Donor 2 (FIG. 42B), and Donor 3 (FIG. 42C). The IC₅₀ values are summarized in Table 14.

TABLE 14
IC₅₀ for proliferation of stimulated B cell populations.

| | | |
|---|---|---|
| BAv9-1 | | RC18 |
| CRD-IgG4 Fc | Atacicept | (Telitacicept) |

Donor 1 (IC₅₀, nM)

| | | | |
|---|---|---|---|
| BAFF | 0.3108 | 0.6397 | 1.983 |
| APRIL | 0.662 | 3 | 2.511 |
| BAFF-60mer | 0.835 | 2.059 | 2.371 |

Donor 2 (IC₅₀, nM)

| | | | |
|---|---|---|---|
| BAFF | 0.000215 | 0.09848 | 0.2954 |
| APRIL | 0.226 | 0.00001 | 0.573 |
| BAFF-60mer | 0.577 | 0.683 | 0.818 |

Donor 3 (IC₅₀, nM)

| | | | |
|---|---|---|---|
| BAFF | 0.02008 | 0.3786 | 1.05 |
| APRIL | 0.366 | 2.147 | 4.138 |
| BAFF-60mer | 0.61 | 1.132 | 1.968 |

For all three donors and for all the ligands BAFF, APRIL or BAFF-60mer, the exemplary BCMA CRD variant BAv9-1 CRD-IgG4 Fc generally exhibited the lowest IC₅₀ for inhibiting the proliferation of stimulated B cells. In some of the cases (for example, BAFF for Donors 2 and 3) the IC₅₀ for BAv9-1 CRD-IgG4 Fc was more than an order of magnitude lower than the IC₅₀ of Atacicept and RC18 (Telitacicept). Accordingly, the results support substantially better inhibition of B cell proliferation after stimulation, by the exemplary BCMA CRD variant BAv9-1 CRD-IgG4 Fc, at a much lower concentration compared to the other tested inhibitors Atacicept and RC18 (Telitacicept). The results show that the exemplary protein is more potent that the TACI-Fc (Atacicept and Telitacicept) fusion proteins in reducing BAFF and APRIL-costimulated proliferation of primary human B cells and mouse splenocytes. The results demonstrate the improved potency of the exemplary BCMA CRD variant in inhibiting the function of both BAFF and APRIL, compared to the other tested inhibitors.

### Example 26: Inhibition of B Cells and Immunological Responses by Dual BAFF/APRIL Antagonist Comprising a BCMA CRD and an IgG4 Fc In Vivo in Mice

A lupus-prone mouse study was performed as therapeutic model of Systemic Lupus Erythematosus (SLE) to determine the in vivo effects of an exemplary BCMA CRD variant fusion protein.

NZB/W-F1 mice, randomized by body weight and proteinuria scores, were treated by weekly injections (1, 3, or 10 mg/kg, subcutaneously) of the exemplary BCMA CRD variant BAv9-1 CRD-IgG4 Fc fusion protein from 28 to 34 weeks of age. Proteinuria (total protein in urine, in mg/dL) was measured weekly using Albustix® strips. Serum anti-dsDNA antibodies and cytokines were measured by ELISA at week 34. For renal histology, kidneys from NZB/W-F1 mice were formalin-fixed and stained with hematoxylin and eosin, and scored by a board-certified veterinary pathologist.

The exemplary BCMA CRD variant BAv9-1 CRD-IgG4 Fc fusion protein, when administered in a lupus mouse model, significantly improved survival at all doses tested and reduced both proteinuria and histological markers of renal damage. A dose-dependent reduction of anti-dsDNA autoantibodies were observed. At all doses tested, the administered BAv9-1 CRD-IgG4 Fc fusion protein significantly decreased the secretion of T cell cytokines IFNγ and IL-17A.

The results demonstrate that the exemplary BCMA CRD variant CRD fusion protein therapeutically reduced several markers of disease activity and improved overall survival. Additionally, the fusion protein was well-tolerated in mice, with no adverse effects. The findings support the utility of the exemplary BCMA CRD variant CRD fusion protein in the treatment of autoimmune diseases, including SLE.

### Example 27: Assessment of Dual BAFF/APRIL Antagonist Comprising a BCMA CRD and an IgG4 Fc In Vivo in Cynomolgus Monkeys

The effect of an exemplary dual BAFF/APRIL antagonist fusion protein, comprising an exemplary engineered BCMA CRD and an IgG4 Fc, were assessed in a non-human primate model.

Non-human primates (cynomolgus monkeys) were administered either a single dose (1 mg/kg, subcutaneously) or weekly doses (5, 20, or 80 mg/kg, subcutaneously) of an exemplary BCMA CRD variant BAv9-1 CRD-IgG4 Fc fusion protein. Serum immunoglobulins and peripheral blood B cells were measured, generally as described in Example 14 above.

The results showed that a single subcutaneous dose of the exemplary BCMA CRD variant BAv9-1 CRD-IgG4 Fc fusion protein suppressed proliferation of peripheral B cells. Weekly dosing over 4 weeks resulted in sustained reductions in all serum immunoglobulins. Over the month of dosing, the exemplary BCMA CRD variant BAv9-1 CRD-IgG4 Fc fusion protein remained well-tolerated in the cynomolgus monkeys with no adverse findings at any dose level tested.

Together, these results demonstrate that treatment with an exemplary BCMA CRD variant BAv9-1 CRD-IgG4 Fc fusion protein resulted also in reductions of B cell activation and serum immunoglobulin production. Additionally, the exemplary BCMA CRD variant BAv9-1 CRD-IgG4 Fc fusion protein was also well-tolerated, with no adverse effects in non-human primates, such as cynomolgus monkeys. These findings highlight the potential utility for the described exemplary dual BAFF/APRIL antagonist fusion proteins in the treatment of autoimmune diseases.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| SEQ | | |
|---|---|---|
| ID | | |
| NO: | SEQUENCE | ANNOTATION |
| 1 | AGQCSQNEYFDSLLHACIPCQLRCSSNTPPLTCQRYCNASVTNSV | BCMA CRD (aa) |
| 2 | GCTGGGCAGTGCTCCCAAAATGAATATTTTGACAGTTTGTTGCATGCTTGCATACCTT | BCMA CRD (nt) |
| | GTCAACTTCGATGTTCTTCTAATACTCCTCCTCTAACATGTCAGCGTTATTGTAATGC | |
| | AAGTGTGACCAATTCAGTG | |
| 3 | AGQCSQNEYFDHLIRACIPCQLRCSSNTPPLTCQRYCNAGVTNSV | BAv9-1 |
| 4 | AGQCSQNEYFDRLIRACIPCQLRCSSNTPPLTCQRYCNAGVTNSV | BAv3-10 |
| 5 | AGQCSQNEYFDRLVRACIPCQLRCSSNTPPLTCQRYCNAGVTNSV | BAv9-5 |
| 6 | AGQCSQNEYFDHLLRACIPCQVRCSSNTPPLTCQRYCNAGVTNSV | BAv8-10 |
| 7 | AGQCSQNEYFDRLLRACIPCQVRCSSNTPPLTCQRYCNAGVTNSV | BAv11-6 |
| 8 | AGQCSQNEYFDHLLRACIPCQLRCSSNTPPLTCQRYCNAGVTNSV | BAv8-9 |
| 9 | AGQCSQNEYFDRLVNACIPCQLRCSSNTPPLTCQRYCNAGVTNSV | BAv1-12 |
| 10 | AQQCSQNEYFDRLVNSCVPCRLRCSSNNPPLTCQHYCNAGVTNSV | 50238779 |
| 11 | AQQCSQNEYFDRLVNSCIPCQLRCSSNNPPLTCQHYCNAGVTNSV | BAvl |
| 12 | AGQCSQNEYFDRLVNSCIPCQLRCSSNNPPLTCQHYCNAGVTNSV | BAv1-1 |
| 13 | AQQCSQNEYFDSLVNSCIPCOLRCSSNNPPLTCQHYCNAGVTNSV | BAv1-2 |
| 14 | AQQCSQNEYFDRLLNSCIPCQLRCSSNNPPLTCQHYCNAGVTNSV | BAv1-3 |
| 15 | AQQCSQNEYFDRLVHSCIPCQLRCSSNNPPLTCQHYCNAGVTNSV | BAv1-4 |
| 16 | AQQCSQNEYFDRLVNACIPCQLRCSSNNPPLTCQHYCNAGVTNSV | BAv1-5 |
| 17 | AQQCSQNEYFDRLVNSCIPCOLRCSSNTPPLTCQHYCNAGVTNSV | BAv1-6 |
| 18 | AQQCSQNEYFDRLVNSCIPCQLRCSSNNPPLTCQRYCNAGVTNSV | BAv1-7 |
| 19 | AGQCSQNEYFDRLVNSCIPCQLRCSSNTPPLTCQRYCNAGVTNSV | BAv1-11 |
| 20 | AGQCSQNEYFDSLVNACIPCQLRCSSNTPPLTCQRYCNAGVTNSV | BAv1-13 |
| 21 | AGQCSQNEYFDHLVNACIPCQLRCSSNTPPLTCQRYCNAGVTNSV | BAv1-14 |
| 22 | AGQCSQNEYFDHLVNSCIPCQLRCSSNTPPLTCQRYCNAGVTNSV | BAv1-15 |
| 23 | AGQCSQNEYFDSLVNSCIPCQLRCSSNTPPLTCQRYCNAGVTNSV | BAv1-16 |
| 24 | AGQCSQNEYFDRLVNSCIPCQVRCSSNTPPLTCQRYCNAGVTNSV | BAv1-17 |
| 25 | AGQCSQNEYFDRLVNACIPCQVRCSSNTPPLTCQRYCNAGVTNSV | BAv1-18 |
| 26 | AGQCSQNEYFDHLVNACIPCQVRCSSNTPPLTCQRYCNAGVTNSV | BAv1-19 |
| 27 | AGQCSQNEYFDSLVNACIPCQVRCSSNTPPLTCQRYCNAGVTNSV | BAv1-20 |
| 28 | AQQCSPNEYFDRLIRACIPCQLRCSSNTPPVTCQRYCNTGVTNSV | 50240054 |
| 29 | AQQCSPNEYFDRLIRACIPCQLRCSSNTPPLTCQRYCNTGVTNSV | BAv3 |
| 30 | AGQCSPNEYFDRLIRACIPCQLRCSSNTPPLTCQRYCNTGVTNSV | BAv3-1 |
| 31 | AQQCSQNEYFDRLIRACIPCQLRCSSNTPPLTCQRYCNTGVTNSV | BAv3-2 |
| 32 | AQQCSPNEYFDSLIRACIPCQLRCSSNTPPLTCQRYCNTGVTNSV | BAv3-3 |
| 33 | AQQCSPNEYFDRLLRACIPCQLRCSSNTPPLTCQRYCNTGVTNSV | BAv3-4 |
| 34 | AQQCSPNEYFDRLIHACIPCQLRCSSNTPPLTCQRYCNTGVTNSV | BAv3-5 |
| 35 | AQQCSPNEYFDRLIRACIPCQLRCSSNTPPLTCQRYCNAGVTNSV | BAv3-6 |
| 36 | AGQCSQNEYFDSLIRACIPCQLRCSSNTPPLTCQRYCNAGVTNSV | BAv3-13 |
| 37 | AGQCSQNEYFDRLIRACIPCQVRCSSNTPPLTCQRYCNAGVTNSV | BAv3-14 |
| 38 | AGQCSQNEYYDHLLRACVSCHVRCSSNNPPAICQRYCNAGVTNSV | 50237847 |
| 39 | AGQCSQNEYFDHLLRACISCQVRCSSNNPPLTCQRYCNAGVTNSV | BAv8 |
| 40 | AGQCSQNEYFDSLLRACISCQVRCSSNNPPLTCQRYCNAGVTNSV | BAv8-1 |
| 41 | AGQCSQNEYFDHLLHACISCOVRCSSNNPPLTCQRYCNAGVTNSV | BAv8-2 |
| 42 | AGQCSQNEYFDHLLRACIPCQVRCSSNNPPLTCQRYCNAGVTNSV | BAv8-3 |
| 43 | AGQCSQNEYFDHLLRACISCOLRCSSNNPPLTCQRYCNAGVTNSV | BAv8-4 |
| 44 | AGQCSQNEYFDHLLRACISCQVRCSSNTPPLTCQRYCNAGVTNSV | BAv8-5 |
| 45 | AGQCSQNEYFDHLIRACIPCDVRCSSNNPPVTCQRYCNAGVTNSV | 50237854 |
| 46 | AGQCSQNEYFDHLIRACIPCQVRCSSNNPPLTCQRYCNAGVTNSV | BAv9 |
| 47 | AGQCSQNEYFDHLIRACIPCQVRCSSNTPPLTCQRYCNAGVTNSV | BAv9-2 |
| 48 | AGQCSQNEYFDHLVRACIPCQVRCSSNTPPLTCQRYCNAGVTNSV | BAv9-3 |
| 49 | AGQCSQNEYFDRLVRACIPCQVRCSSNTPPLTCQRYCNAGVTNSV | BAv9-4 |
| 50 | ARQCSQNEYYDRLLRSCVPCHVRCSSNNPPVTCQRYCNAGVTNSV | 50239698 |
| 51 | ARQCSQNEYFDRLLRSCIPCQVRCSSNNPPLTCQRYCNAGVTNSV | BAv11 |
| 52 | AGQCSQNEYFDRLLRSCIPCQLRCSSNTPPLTCQRYCNAGVTNSV | BAv11-1 |
| 53 | AGQCSQNEYFDRLLRACIPCQLRCSSNTPPLTCQRYCNAGVTNSV | Bav11-2 |
| 54 | AGQCSQNEYFDSLLRSCIPCQLRCSSNTPPLTCQRYCNAGVTNSV | BAv11-3 |
| 55 | AGQCSQNEYFDRLLRSCIPCQVRCSSNTPPLTCQRYCNAGVTNSV | BAv11-4 |
| 56 | AGQCSQNEYFDSLLRACIPCQLRCSSNTPPLTCQRYCNAGVTNSV | BAv11-5 |
| 57 | AGQCSQNDYYDHLLRACIPCQLRCSSNNPPLTCQRYCNAGVTNSV | 50234224 |
| 58 | AGQCSQNEYFDHLLRACIPCQLRCSSNNPPLTCQRYCNAGVTNSV | BAv2 |
| 59 | AGQCSQSEYYDRLIRACIPCOLRCSSNNPPVICQRYCNAGVTNSV | 50237844 |
| 60 | AGQCSQSEYFDRLIRACIPCQLRCSSNNPPLTCQRYCNAGVTNSV | BAv4 |
| 61 | AGQCSQNEYYDRLIRACLPCHVRCSSNTPPATCQRYCNAGVTNSV | 50238504R |
| 62 | AGQCSQNEYFDRLIRACIPCQVRCSSNTPPLTCQRYCNAGVTNSV | BAv6 |
| 63 | AGQCSQNEYYDHLIRACISCOIRCSSNNPPPICQRYCNAGVTNSV | 50238475 |
| 64 | AGQCSQNEYFDHLIRACISCOIRCSSNNPPLTCQRYCNAGVTNSV | BAv12 |
| 65 | ARQCSPSEYYDHLIRSCVPCQLRCSSNTPPVTCQRYCDAGVTNSV | 50239040 |
| 66 | ARQCSPSEYEDHLIRSCIPCOLRCSSNTPPLTCQRYCDAGVTNSV | BAv5 |
| 67 | AGQCSQSEYYDRLIHACVPCOLRCSSNNPPATCQRYCNAGVTNSV | 50238494 |
| 68 | AGQCSQSEYFDRLIHACIPCQLRCSSNNPPLTCQRYCNAGVTNSV | BAv7 |
| 69 | ARQCSPNEYYDSLIRACVPCQLRCSSNNPPVTCQRYCNAGVTNSV | 50239978 |
| 70 | ARQCSPNEYFDSLIRACIPCQLRCSSNNPPLTCQRYCNAGVTNSV | BAv10 |
| 71 | AGQCSQNEYFDPLLRACIPCQVRCSSNTPPLTCQRYCNAGVTNSV | Bav13 |
| 72 | AGQCSQNEYFDTLLRACIPCQVRCSSNTPPLTCQRYCNAGVTNSV | Bav14 |
| 73 | AGQCSQNEYFDNLLRACIPCQVRCSSNTPPLTCQRYCNAGVTNSV | Bav15 |
| 74 | AGQCSQNEYFDQLLRACIPCQVRCSSNTPPLTCQRYCNAGVTNSV | BAv16 |
| 75 | AGQCSQNEYFDHLPRACIPCQVRCSSNTPPLTCQRYCNAGVTNSV | BAv17 |
| 76 | AGQCSQNEYFDHLTRACIPCQVRCSSNTPPLTCQRYCNAGVTNSV | Bav18 |
| 77 | AGQCSQNEYFDHLLAACIPCQVRCSSNTPPLTCQRYCNAGVTNSV | Bav19 |
| 78 | AGQCSQNEYFDHLLRTCIPCQVRCSSNTPPLTCQRYCNAGVTNSV | Bav20 |
| 79 | AGQCSQNEYFDHLLRNCIPCQVRCSSNTPPLTCQRYCNAGVTNSV | Bav21 |
| 80 | AGQCSQNEYFDHLLRQCIPCQVRCSSNTPPLTCQRYCNAGVTNSV | Bav22 |
| 81 | AGQCSQNEYFDHLLRVCIPCQVRCSSNTPPLTCQRYCNAGVTNSV | BAv23 |
| 82 | AGQCSQNEYFDHLLRYCIPCQVRCSSNTPPLTCQRYCNAGVTNSV | Bav24 |
| 83 | AGQCSQNEYFDHLLRICIPCQVRCSSNTPPLTCQRYCNAGVTNSV | BAv25 |
| 84 | AGQCSQNEYFDHLLRFCIPCQVRCSSNTPPLTCQRYCNAGVTNSV | BAv26 |
| 85 | AGQCSQNEYFDHLLRACNPCQVRCSSNTPPLTCQRYCNAGVTNSV | BAv27 |
| 86 | AGQCSQNEYFDHLLRACQPCQVRCSSNTPPLTCQRYCNAGVTNSV | Bav28 |
| 87 | AGQCSQNEYFDHLLRACSPCQVRCSSNTPPLTCQRYCNAGVTNSV | Bav29 |
| 88 | AGQCSQNEYFDHLLRACTPCQVRCSSNTPPLTCQRYCNAGVTNSV | BAv30 |
| 89 | AGQCSQNEYFDHLLRACAPCQVRCSSNTPPLTCQRYCNAGVTNSV | Bav31 |
| 90 | AGQCSQNEYFDHLLRACIACQVRCSSNTPPLTCQRYCNAGVTNSV | BAv32 |
| 91 | AGQCSQNEYFDHLLRACIGCQVRCSSNTPPLTCQRYCNAGVTNSV | BAv33 |
| 92 | AGQCSQNEYFDHLLRACITCQVRCSSNTPPLTCQRYCNAGVTNSV | Bav34 |
| 93 | AGQCSQNEYFDHLLRACINCQVRCSSNTPPLTCQRYCNAGVTNSV | Bav35 |
| 94 | AGQCSQNEYFDHLLRACIQCQVRCSSNTPPLTCQRYCNAGVTNSV | Bav36 |
| 95 | AGQCSQNEYFDHLLRACIPCIVRCSSNTPPLTCQRYCNAGVTNSV | Bav37 |
| 96 | AGQCSQNEYFDHLLRACIPCQARCSSNTPPLTCQRYCNAGVTNSV | Bav38 |
| 97 | AGQCSQNEYFDHLLRACIPCQYRCSSNTPPLTCQRYCNAGVTNSV | Bav39 |
| 98 | AGQCSQNEYFDHLLRACIPCQFRCSSNTPPLTCQRYCNAGVTNSV | BAv40 |
| 99 | AGQCSQNEYFDHLLRACIPCQVACSSNTPPLTCQRYCNAGVTNSV | Bav41 |
| 100 | AGQCSQNEYFDHLLRACIPCQVGCSSNTPPLTCQRYCNAGVTNSV | BAv42 |
| 101 | AGQCSQNEYFDHLLRACIPCQVSCSSNTPPLTCQRYCNAGVTNSV | Bav43 |
| 102 | AGQCSQNEYFDHLLRACIPCQVTCSSNTPPLTCQRYCNAGVTNSV | Bav44 |
| 103 | AGQCSQNEYFDHLLRACIPCQVNCSSNTPPLTCQRYCNAGVTNSV | Bav45 |
| 104 | AGQCSQNEYFDHLLRACIPCQVQCSSNTPPLTCQRYCNAGVTNSV | Bav46 |
| 105 | AGQCSQNEYFDHLLRACIPCQVWCSSNTPPLTCQRYCNAGVTNSV | Bav47 |
| 106 | AGQCSQNEYFDHLLRACIPCQVHCSSNTPPLTCQRYCNAGVTNSV | BAv48 |
| 107 | AGQCSQNEYFDPLIRACIPCQVRCSSNTPPLTCQRYCNAGVTNSV | BAv49 |
| 108 | AGQCSQNEYFDNLLRACIPCQYRCSSNTPPLTCQRYCNAGVTNSV | BAv50 |
| 109 | AGQCSQNEYFDHLIRACIPCQARCSSNTPPLTCQRYCNAGVTNSV | BAv51 |
| 110 | AGQCSQNEYFDHLLRYCIPCQYRCSSNTPPLTCQRYCNAGVTNSV | BAv52 |
| 111 | AGQCSQNEYFDSLIRHCIPCQVRCSSNTPPLTCQRYCNAGVTNSV | BAv53 |
| 112 | AGQCSQNEYFDHLSRACIPCQVRCSSNTPPLTCQRYCNAGVTNSV | BAv54 |
| 113 | AGQCSQNEYFDHLGRACIPCQVRCSSNTPPLTCQRYCNAGVTNSV | BAv55 |
| 114 | AGQCSQNEYFDHLFRACIPCQVRCSSNTPPLTCQRYCNAGVTNSV | BAv56 |
| 115 | AGQCSQNEYFDHLIRACIPCQSRCSSNTPPLTCQRYCNAGVTNSV | BAv57 |
| 116 | AGQCSQNEYFDSLVRHCIPCQIRCSSNTPPLTCQRYCNAGVTNSV | BAv58 |
| 117 | AGQCSQNEYFDHLLRTCIPCQYRCSSNTPPLTCQRYCNAGVTNSV | BAv59 |
| 118 | AGQCSQNEYFDSLVRHCIPCQVRCSSNTPPLTCQRYCNAGVTNSV | BAv60 |
| 119 | AGQCSQNEYFDSLIRHCIPCQIRCSSNTPPLTCQRYCNAGVTNSV | BAv61 |
| 120 | AGQCSQNEYFDSLVRTCIPCQIRCSSNTPPLTCQRYCNAGVTNSV | BAv62 |
| 121 | AGQCSQNEYEDHLARACIPCQVRCSSNTPPLTCQRYCNAGVTNSV | BAv63 |
| 122 | AGQCSQNEYFDHLYRACIPCQVRCSSNTPPLTCQRYCNAGVTNSV | BAv64 |
| 123 | AGQCSQNEYFDHLIRACIPCQTRCSSNTPPLTCQRYCNAGVTNSV | BAv65 |
| 124 | AGQCSQNEYFDHLIRACIPCQGRCSSNTPPLTCQRYCNAGVTNSV | BAv66 |
| 125 | AGQCSQNEYFDSLVRACQPCQVRCSSNTPPLTCQRYCNAGVTNSV | BAv67 |
| 126 | AGQCSQNEYFDSLIRACQPCQVRCSSNTPPLTCQRYCNAGVTNSV | BAv68 |
| 127 | AGQCSQNEYFDHLVRACQPCQLRCSSNTPPLTCQRYCNAGVTNSV | BAv69 |
| 128 | AGQCSQNEYFDHLIRACQPCOLRCSSNTPPLTCQRYCNAGVTNSV | BAv70 |
| 129 | AGQCSQNEYFDHLIRICIPCQVRCSSNTPPLTCQRYCNAGVTNSV | BAv71 |
| 130 | AGQCSQNEYFDHLVRICIPCQVRCSSNTPPLTCQRYCNAGVTNSV | BAv72 |
| 131 | AGQCSQNEYFDHLLRICIPCQYRCSSNTPPLTCQRYCNAGVTNSV | BAv73 |
| 132 | AGQCSQNEYFDHLIRACTPCQVRCSSNTPPLTCQRYCNAGVTNSV | BAv74 |
| 133 | AGQCSQNEYFDHLVRACTPCQVRCSSNTPPLTCQRYCNAGVTNSV | BAv75 |
| 134 | AGQCSQNEYFDHLIRACIPCQVTCSSNTPPLTCQRYCNAGVTNSV | BAv76 |
| 135 | AGQCSQNEYEDHLVRACIPCQVTCSSNTPPLTCQRYCNAGVTNSV | BAv77 |
| 136 | AGQCSQNEYFDHLLRACIPCQYTCSSNTPPLTCQRYCNAGVTNSV | BAv78 |
| 137 | AGQCSQNEYFDPLIRACIPCQARCSSNTPPLTCQRYCNAGVTNSV | BAv79 |
| 138 | AGQCSQNEYFDPLIRACIPCQIRCSSNTPPLTCQRYCNAGVTNSV | BAv80 |
| 139 | AGQCSQNEYFDPLVRACIPCQVRCSSNTPPLTCQRYCNAGVTNSV | BAv81 |
| 140 | AGQCSQNEYFDPLVRACIPCQARCSSNTPPLTCQRYCNAGVTNSV | BAv82 |
| 141 | AGQCSQNEYFDPLVRACIPCQIRCSSNTPPLTCQRYCNAGVTNSV | BAv83 |
| 142 | AGQCSQNEYFDHLIRACIPCQIRCSSNTPPLTCQRYCNAGVTNSV | BAv84 |
| 143 | AGQCSQNEYFDHLVRACIPCQARCSSNTPPLTCQRYCNAGVTNSV | BAv85 |
| 144 | AGQCSQNEYFDHLVRACIPCQIRCSSNTPPLTCQRYCNAGVTNSV | BAv86 |
| 145 | AGQCSQNEYFDSLVRHCIPCQARCSSNTPPLTCQRYCNAGVTNSV | BAv87 |
| 146 | AGQCSQNEYFDSLIRHCIPCQARCSSNTPPLTCQRYCNAGVTNSV | BAv88 |
| 147 | SLSCRKEQGKFYDHLLRDCISCASICGQHPKQCAYFCENKLR | TACI-CRD2 |
| 148 | ATTGTTCTCAACATTCTAGCTGCTCTTGCTGCATTTGCTCTGGAATTCTTGTAGAGAT | Full length BCMA |
| | ATTACTTGTCCTTCCAGGCTGTTCTTTCTGTAGCTCCCTTGTTTTCTTTTTGTGATCA | (nt) |
| | TGTTGCAGATGGCTGGGCAGTGCTCCCAAAATGAATATTTTGACAGTTTGTTGCATGC | |
| | TTGCATACCTTGTCAACTTCGATGTTCTTCTAATACTCCTCCTCTAACATGTCAGCGT | |
| | TATTGTAATGCAAGTGTGACCAATTCAGTGAAAGGAACGAATGCGATTCTCTGGACCT | |
| | GTTTGGGACTGAGCTTAATAATTTCTTTGGCAGTTTTCGTGCTAATGTTTTTGCTAAG | |
| | GAAGATAAACTCTGAACCATTAAAGGACGAGTTTAAAAACACAGGATCAGGTCTCCTG | |
| | GGCATGGCTAACATTGACCTGGAAAAGAGCAGGACTGGTGATGAAATTATTCTTCCGA | |
| | GAGGCCTCGAGTACACGGTGGAAGAATGCACCTGTGAAGACTGCATCAAGAGCAAACC | |
| | GAAGGTCGACTCTGACCATTGCTTTCCACTCCCAGCTATGGAGGAAGGCGCAACCATT | |
| | CTTGTCACCACGAAAACGAATGACTATTGCAAGAGCCTGCCAGCTGCTTTGAGTGCTA | |
| | CGGAGATAGAGAAATCAATTTCTGCTAGGTAATTAACCATTTCGACTCGAGCAGTGCC | |
| | ACTTTAAAAATCTTTTGTCAGAATAGATGATGTGTCAGATCTCTTTAGGATGACTGTA | |
| | TTTTTCAGTTGCCGATACAGCTTTTTGTCCTCTAACTGTGGAAACTCTTTATGTTAGA | |
| | TATATTTCTCTAGGTTACTGTTGGGAGCTTAATGGTAGAAACTTCCTTGGTTTCATGA | |
| | TTAAACTCTTTTTTTTCCTGA | |
| 149 | MLQMAGQCSQNEYFDSLLHACIPCQLRCSSNTPPLTCQRYCNASVTNSVKGTNAILWT | Full length human |
| | CLGLSLIISLAVFVLMFLLRKINSEPLKDEFKNTGSGLLGMANIDLEKSRTGDEIILP | BCMA (aa); |
| | RGLEYTVEECTCEDCIKSKPKVDSDHCFPLPAMEEGATILVTTKTNDYCKSLPAALSA | NP 001183.2 |
| | TEIEKSISAR | |
| 150 | MLQMAGQCSQNEYFDSLLHACIPCQLRCSSNTPPLTCQRYCNASVTNSVKGTNAILWT | Full length human |
| | CLGLSLIISLAVFVLMELLRKISSEPLEDEFKNTGSGLLGMANIDLEKSRTGDEIILP | BCMA (aa); |
| | RGLEYTVEECTCEDCIKSKPKVDSDHCFPLPAMEEGATILVTTKTNDYCKSLPAALSA | AAH58291.1 |
| | TEIEKSISAR | |
| 151 | GCTGGGCAGTGCTCCCAAAATGAATATTTTGACAGTTTGTTGCATGCTTGCATACCTT | BCMA ECD (nt) |
| | GTCAACTTCGATGTTCTTCTAATACTCCTCCTCTAACATGTCAGCGTTATTGTAATGC | |
| | AAGTGTGACCAATTCAGTGAAAGGAACGAATGCG | |
| 152 | AGQCSQNEYFDSLLHACIPCQLRCSSNTPPLTCQRYCNASVTNSVKGTNA | BCMA ECD (aa) |
| 153 | atggcctggatgatgcttctcctcggactccttgcttatggatcaggagtcgactct | signal sequence |
| | | (nt) |
| 154 | MAWMMLLLGLLAYGSGVDS | Signal sequence |
| | | (aa) |
| 155 | ggcggcggtggcagcgc | linker (nt) |
| 156 | GGGGS | linker (aa) |
| 157 | ggcggcggtggcagcgct | linker (nt) |
| 158 | GGGGSA | linker (aa) |
| 159 | gccggacagtgtagccagaacgagtacttcgatcacctgattcgcgcttgtataccgt | BAv9-1 (nt) |
| | gccaacttagatgctcgagtaatactccaccactgacatgccagcgttattgcaacgc | |
| | cggcgtcacaaacagcgtc | |
| 160 | tgagtccaaatatggtcccccatgcccaccatgcccagcaccagaggccgccggggga | IgG4 Fc (nt) |
| | ccatcagtcttcctgttccccccaaaacccaaggacactctcatgatctcccggaccc | |
| | ctgaggtcacgtgcgtggtggtggacgtgagccaggaagaccccgaggtccagttcaa | |
| | ctggtacgtggatggcgtggaggtgcataatgccaagacaaagccgcgggaggagcag | |
| | ttcaacagcacgtaccgtgtggtcagcgtcctcaccgtcctgcaccaggactggctga | |
| | acggcaaggagtacaagtgcaaggtctccaacaaaggcctcccgtcctccatcgagaa | |
| | aaccatctccaaagccaaagggcagccccgagagccacaggtgtacaccctgccccca | |
| | tcccaggaggagatgaccaagaaccaggtcagcctgacctgcctggtcaaaggcttct | |
| | accccagcgacatcgccgtggagtgggagagcaatgggcagccggagaacaactacaa | |
| | gaccacgcctcccgtgctggactccgacggctccttcttcctctacagcaggctcacc | |
| | gtggacaagagcaggtggcaggaggggaatgtcttctcatgctccgtgatgcatgagg | |
| | ctctgcacaaccactacacacagaagagcctctccctgtctccgggtaaatgag | |
| 161 | AESKYGPPCPPCPAPEAAGGPSVFLFPPKPKDTLMISRTPEVTCVVVDVSQEDPEVOF | IgG4 Fc (aa) |
| | NWYVDGVEVHNAKTKPREEQFNSTYRVVSVLTVLHQDWLNGKEYKCKVSNKGLPSSIE | |
| | KTISKAKGQPREPQVYTLPPSQEEMTKNQVSLTCLVKGFYPSDIAVEWESNGOPENNY | |
| | KTTPPVLDSDGSFFLYSRLTVDKSRWQEGNVFSCSVMHEALHNHYTQKSLSLSPGK | |
| 162 | gagtccaaatatggtcccccatgcccaccatgcccagcaccagaggccgccgggggac | IgG4 Fc (nt) |
| | catcagtcttcctgttccccccaaaacccaaggacactctcatgatctcccggacccc | |
| | tgaggtcacgtgcgtggtggtggacgtgagccaggaagaccccgaggtccagttcaac | |
| | tggtacgtggatggcgtggaggtgcataatgccaagacaaagccgcgggaggagcagt | |
| | tcaacagcacgtaccgtgtggtcagcgtcctcaccgtcctgcaccaggactggctgaa | |
| | cggcaaggagtacaagtgcaaggtctccaacaaaggcctcccgtcctccatcgagaaa | |
| | accatctccaaagccaaagggcagccccgagagccacaggtgtacaccctgcccccat | |
| | cccaggaggagatgaccaagaaccaggtcagcctgacctgcctggtcaaaggcttcta | |
| | ccccagcgacatcgccgtggagtgggagagcaatgggcagccggagaacaactacaag | |
| | accacgcctcccgtgctggactccgacggctccttcttcctctacagcaggctcaccg | |
| | tggacaagagcaggtggcaggaggggaatgtcttctcatgctccgtgatgcatgaggc | |
| | tctgcacaaccactacacacagaagagcctctccctgtctccgggtaaatgag | |
| 163 | ESKYGPPCPPCPAPEAAGGPSVFLFPPKPKDTLMISRTPEVTCVVVDVSQEDPEVQEN | IgG4 Fc (aa) |
| | WYVDGVEVHNAKTKPREEQFNSTYRVVSVLTVLHQDWLNGKEYKCKVSNKGLPSSIEK | |
| | TISKAKGQPREPQVYTLPPSQEEMTKNQVSLTCLVKGFYPSDIAVEWESNGQPENNYK | |
| | TTPPVLDSDGSFFLYSRLTVDKSRWQEGNVFSCSVMHEALHNHYTQKSLSLSPGK | |
| 164 | atggcctggatgatgcttctcctcggactccttgcttatggatcaggagtcgactctg | BAv9-1 - linker - |
| | ccggacagtgtagccagaacgagtacttcgatcacctgattcgcgcttgtataccgtg | IgG4 Fc w/ signal |
| | ccaacttagatgctcgagtaatactccaccactgacatgccagcgttattgcaacgcc | sequence (nt) |
| | ggcgtcacaaacagcgtcggcggcggtggcagcgctgagtccaaatatggtcccccat | |
| | gcccaccatgcccagcaccagaggccgccgggggaccatcagtcttcctgttcccccc | |
| | aaaacccaaggacactctcatgatctcccggacccctgaggtcacgtgcgtggtggtg | |
| | gacgtgagccaggaagaccccgaggtccagttcaactggtacgtggatggcgtggagg | |
| | tgcataatgccaagacaaagccgcgggaggagcagttcaacagcacgtaccgtgtggt | |
| | cagcgtcctcaccgtcctgcaccaggactggctgaacggcaaggagtacaagtgcaag | |
| | gtctccaacaaaggcctcccgtcctccatcgagaaaaccatctccaaagccaaagggc | |
| | agccccgagagccacaggtgtacaccctgcccccatcccaggaggagatgaccaagaa | |
| | ccaggtcagcctgacctgcctggtcaaaggcttctaccccagcgacatcgccgtggag | |
| | tgggagagcaatgggcagccggagaacaactacaagaccacgcctcccgtgctggact | |
| | ccgacggctccttcttcctctacagcaggctcaccgtggacaagagcaggtggcagga | |
| | ggggaatgtcttctcatgctccgtgatgcatgaggctctgcacaaccactacacacag | |
| | aagagcctctccctgtctccgggtaaatgag | |
| 165 | MAWMMLLLGLLAYGSGVDSAGQCSQNEYFDHLIRACIPCQLRCSSNTPPLTCQRYCNA | BAv9-1 - linker - |
| | GVTNSVGGGGSAESKYGPPCPPCPAPEAAGGPSVFLFPPKPKDTLMISRTPEVTCVVV | IgG4 Fc w/ signal |
| | DVSQEDPEVQFNWYVDGVEVHNAKTKPREEQFNSTYRVVSVLTVLHQDWLNGKEYKCK | sequence (aa) |
| | VSNKGLPSSIEKTISKAKGQPREPQVYTLPPSQEEMTKNQVSLTCLVKGFYPSDIAVE | |
| | WESNGQPENNYKTTPPVLDSDGSFFLYSRLTVDKSRWQEGNVFSCSVMHEALHNHYTQ | |
| | KSLSLSPGK | |
| 166 | gccggacagtgtagccagaacgagtacttcgatcacctgattcgcgcttgtataccgt | BAv9-1 - linker - |
| | gccaacttagatgctcgagtaatactccaccactgacatgccagcgttattgcaacgc | IgG4 Fc (nt) |
| | cggcgtcacaaacagcgtcggcggcggtggcagcgctgagtccaaatatggtccccca | |
| | tgcccaccatgcccagcaccagaggccgccgggggaccatcagtcttcctgttccccc | |
| | caaaacccaaggacactctcatgatctcccggacccctgaggtcacgtgcgtggtggt | |
| | ggacgtgagccaggaagaccccgaggtccagttcaactggtacgtggatggcgtggag | |
| | gtgcataatgccaagacaaagccgcgggaggagcagttcaacagcacgtaccgtgtgg | |
| | tcagcgtcctcaccgtcctgcaccaggactggctgaacggcaaggagtacaagtgcaa | |
| | ggtctccaacaaaggcctcccgtcctccatcgagaaaaccatctccaaagccaaaggg | |
| | cagccccgagagccacaggtgtacaccctgcccccatcccaggaggagatgaccaaga | |
| | accaggtcagcctgacctgcctggtcaaaggcttctaccccagcgacatcgccgtgga | |
| | gtgggagagcaatgggcagccggagaacaactacaagaccacgcctcccgtgctggac | |
| | tccgacggctccttcttcctctacagcaggctcaccgtggacaagagcaggtggcagg | |
| | aggggaatgtcttctcatgctccgtgatgcatgaggctctgcacaaccactacacaca | |
| | gaagagcctctccctgtctccgggtaaatgag | |
| 167 | AGQCSQNEYFDHLIRACIPCQLRCSSNTPPLTCQRYCNAGVTNSVGGGGSAESKYGPP | BAv9-1 - linker - |
| | CPPCPAPEAAGGPSVFLFPPKPKDTLMISRTPEVTCVVVDVSQEDPEVQFNWYVDGVE | IgG4 Fc (aa) |
| | VHNAKTKPREEQFNSTYRVVSVLTVLHQDWLNGKEYKCKVSNKGLPSSIEKTISKAKG | |
| | QPREPQVYTLPPSQEEMTKNQVSLTCLVKGFYPSDIAVEWESNGQPENNYKTTPPVLD | |
| | SDGSFFLYSRLTVDKSRWQEGNVFSCSVMHEALHNHYTQKSLSLSPGK | |
| 168 | DKTHTCPPCPAPELEGGPSVFLFPPKPKDTLMISRTPEVTCVVVDVSHEDPEVKENWY | human IgG1 Fc |
| | VDGVEVHNAKTKPREEQYNSTYRVVSVLTVLHQDWLNGKAYACAVSNKALPAPIEKTI | |
| | SKAKGQPREPQVYTLPPSRDELTKNQVSLTCLVKGFYPSDIAVEWESNGQPENNYKTT | |
| | PPVLDSDGSFFLYSKLTVDKSRWQQGNVFSCSVMHEALHNHYTQKSLSLSPGK | |
| 169 | EPKSCDKTHTCPPCPAPELLGGPSVFLFPPKPKDTLMISRTPEVTCVVVDVSHEDPEV | human IgG1 Fc |
| | KFNWYVDGVEVHNAKTKPREEQYNSTYRVVSVLTVLHQDWLNGKEYKCKVSNKALPAP | |
| | IEKTISKAKGQPREPQVYTLPPSREEMTKNQVSLTCLVKGFYPSDIAVEWESNGQPEN | |
| | NYKTTPPVLDSDGSFFLYSKLTVDKSRWQQGNVFSCSVMHEALHNHYTQKSLSLSPGK | |
| 170 | EPKSCDKTHTCPPCPAPELEGGPSVFLFPPKPKDTLMISRTPEVTCVVVDVSHEDPEV | human IgG1 Fc |
| | KFNWYVDGVEVHNAKTKPREEQYNSTYRVVSVLTVLHQDWLNGKAYACAVSNKALPAP | |
| | IEKTISKAKGQPREPQVYTLPPSRDELTKNQVSLTCLVKGFYPSDIAVEWESNGOPEN | |
| | NYKTTPPVLDSDGSFFLYSKLTVDKSRWQQGNVFSCSVMHEALHNHYTQKSLSLSPGK | |
| 171 | ERKCCVECPPCPAPPVAGPSVFLFPPKPKDTLMISRTPEVTCVVVDVSHEDPEVQFNW | human IgG2 Fc |
| | YVDGVEVHNAKTKPREEQFNSTFRVVSVLTVVHQDWLNGKEYKCKVSNKGLPAPIEKT | |
| | ISKTKGQPREPQVYTLPPSREEMTKNQVSLTCLVKGFYPSDISVEWESNGQPENNYKT | |
| | TPPMLDSDGSFFLYSKLTVDKSRWQQGNVFSCSVMHEALHNHYTQKSLSLSPGK | |
| 172 | ESKYGPPCPSCPAPEFLGGPSVFLFPPKPKDTLMISRTPEVTCVVVDVSQEDPEVQEN | human IgG4 Fc |
| | WYVDGVEVHNAKTKPREEQFNSTYRVVSVLTVLHQDWLNGKEYKCKVSNKGLPSSIEK | |
| | TISKAKGQPREPQVYTLPPSQEEMTKNQVSLTCLVKGFYPSDIAVEWESNGQPENNYK | |
| | TTPPVLDSDGSFFLYSRLTVDKSRWQEGNVFSCSVMHEALHNHYTQKSLSLSLGK | |
| 173 | GS | linker |
| 174 | GGS | linker |
| 175 | GGGS | linker |
| 176 | GGGGGS | linker |
| 177 | GGGGGGS | linker |
| 178 | GGGGGGGS | linker |
| 179 | GGGGGGGGS | linker |
| 180 | GGGGGGGGGS | linker |
| 181 | GGGGGGGGGGS | linker |
| 182 | GGGGSGGGGS | linker |
| 183 | GGGGSGGGGSGGGGS | linker |
| 184 | GGGGSGGGGSGGGGSGGGGS | linker |
| 185 | GGGGSGGGGSGGGGSGGGGSGGGGS | linker |
| 186 | GGGGSGGGGSGGGGSGGGGSGGGGGGGGS | linker |
| 187 | GSA | linker |
| 188 | GGSA | linker |
| 189 | GGGSA | linker |
| 190 | GGGGGSA | linker |
| 191 | GGGGGGSA | linker |
| 192 | GGGGGGGSA | linker |
| 193 | GGGGGGGGSA | linker |
| 194 | GGGGGGGGGSA | linker |
| 195 | GGGGGGGGGGSA | linker |
| 196 | GGGGSAGGGGSA | linker |
| 197 | GGGGSAGGGGSAGGGGSA | linker |
| 198 | GGGGSAGGGGSAGGGGSGGGGSA | linker |
| 199 | GGGGSAGGGGSAGGGGSAGGGGSAGGGGSA | linker |
| 200 | GGGGSAGGGGSAGGGGSAGGGGSAGGGGSAGGGGSA | linker |
| 201 | GGGA | linker |
| 202 | GGGGA | linker |
| 203 | GGGGGA | linker |
| 204 | GGGGGGA | linker |
| 205 | GGGGGGGA | linker |
| 206 | GGGGGGGGA | linker |
| 207 | GGGGGGGGGA | linker |
| 208 | GGGGGGGGGGA | linker |
| 209 | GGGGAGGGGA | linker |
| 210 | GGGGAGGGGAGGGGA | linker |
| 211 | GGGGAGGGGAGGGGAGGGGA | linker |
| 212 | GGGGAGGGGAGGGGAGGGGAGGGGA | linker |
| 213 | GGGGAGGGGAGGGGAGGGGAGGGGAGGGGA | linker |
| 214 | MDDSTEREQSRLTSCLKKREEMKLKECVSILPRKESPSVRSSKDGKLLAATLLLALLS | BAFF isoform 1 |
| | CCLTVVSFYQVAALQGDLASLRAELQGHHAEKLPAGAGAPKAGLEEAPAVTAGLKIFE | |
| | PPAPGEGNSSQNSRNKRAVQGPEETVTQDCLQLIADSETPTIQKGSYTFVPWLLSFKR | |
| | GSALEEKENKILVKETGYFFIYGQVLYTDKTYAMGHLIQRKKVHVFGDELSLVTLFRC | |
| | IQNMPETLPNNSCYSAGIAKLEEGDELQLAIPRENAQISLDGDVTFFGALKLL | |
| 215 | MPASSPFLLAPKGPPGNMGGPVREPALSVALWLSWGAALGAVACAMALLTQQTELQSL | APRIL alpha |
| | RREVSRLQGTGGPSQNGEGYPWQSLPEQSSDALEAWENGERSRKRRAVLTQKOKKQHS | |
| | VLHLVPINATSKDDSDVTEVMWQPALRRGRGLQAQGYGVRIQDAGVYLLYSQVLFQDV | |
| | TFTMGQVVSREGQGROETLFRCIRSMPSHPDRAYNSCYSAGVFHLHQGDILSVIIPRA | |
| | RAKLNLSPHGTFLGFVKL | |
| 216 | MPASSPFLLAPKGPPGNMGGPVREPALSVALWLSWGAALGAVACAMALLTQQTELQSL | APRIL beta |
| | RREVSRLQGTGGPSQNGEGYPWQSLPEQSSDALEAWENGERSRKRRAVLTQKQKNDSD | |
| | VTEVMWQPALRRGRGLQAQGYGVRIQDAGVYLLYSQVLFQDVTFTMGQVVSREGQGRQ | |
| | ETLFRCIRSMPSHPDRAYNSCYSAGVFHLHQGDILSVIIPRARAKLNLSPHGTFLGFV | |
| | KL | |
| 217 | MPASSPFFVAPKGPPGNMGGPVREPALSVALWLSWGAALGAVACAMALLTQQTELQSL | APRIL |
| | RREVSRLQGTGGPSQNGEGYPWQSLPEQSSDALEAWENGERSRKRRAVLTQKOKKQHS | |
| | VLHLVPINATSKDDSDVTEVMWQPALRRGRGLQAQGYGVRIQDAGVYLLYSQVLFQDV | |
| | TFTMGQVVSREGQGRQETLERCIRSMPSHPDRAYNSCYSAGVFHLHQGDILSVIIPRA | |
| | RAKLNLSPHGTFLGFVKL | |
| 218 | CCCTATTCTATAGTGTCACCTAAATGCTAGAGCTCGCTGATCAGCCTCGACTGTGCCT | BGH poly A |
| | TCTAGTTGCCAGCCATCTGTTGTTTGCCCCTCCCCCGTGCCTTCCTTGACCCTGGAAG | sequence: |
| | GTGCCACTCCCACTGTCCTTTCCTAATAAAATGAGGAAATTGCATCGCATTGTCTGAG | |
| | TAGGTGTCATTCTATTCTGGGGGGTGGGGTGGGGCAGGACAGCAAGGGGGAGGATTGG | |
| | GAAGACAATAGCAGGCATGCTGGGGATGCGGTGGGCTCTATGGCTTCTGAGGCGGAAA | |
| | GAACCAGCTGGGGCTCGAGC | |
| 219 | AAAGGATCTTCTTGAGATCCTTTTTTTCTGCGCGTAATCTGCTGCTTGCACAACAAAA | pUC vector |
| | AAACCACCGCTACCAGCGGTGGTTTGTTTGCCGGATCAAGAGCCTACCAACTCTTTTT | sequence |
| | CCGAAGGTAACTGGCTTCAGCAGAGCGCAGATACCACAATACTGTCCTTCTAGTGTAG | |
| | CCGTAGTTAGGCCACCACTTCAAGAACTCCTGTAGCACCGCCTACATACCTCGCTCTG | |
| | CTAATCCTGTTACCAGTGGCTGCCTGCCAGTGGCGATAAGTCGTGTCTTACCGGGTTG | |
| | GACTCAAGACGATAGCTTACCGGATAAGGCGCAGCGGTCGGGCTGAACGGGGGGTTCG | |
| | TGCACACACGCCCAGCTTGGAGCGAACGACCTACACCGAACTGAGATACCTACAGCGT | |
| | GCAGCTATGAGAAAGCGCCACGCTTCCCGAAGGGAGAAAGGCGGACAGGTATCCCGGT | |
| | AAGCGGCAGGGTCGGAACAGGAGAGCGCACGAGGGAGCTTCCAGGCGGGAAACGCCTG | |
| | GTATCTTTATAGTCCTGTCGGGTTTCGCCACCTCTGACCTTGAGCGTCGATTTTTGTG | |
| | ATGCTCGTCAGGGGGGCGGAGCCTATGGAAACAACGCCAGCAACGCG | |
| 220 | ATGACCGAGTACAAGCCCACGGTGCGCCTCGCCACCCGCGACGACGTCCCCCGGGCCG | Puromycin |
| | TACGCACCCTCGCCGCCGCGTTCGCCGACTACCCCGCCACGCGCCACACCGTCGACCC | resistance gene |
| | GGACCGCCACATCGAGCGGGTCACCGAGCTGCAAGAACTCTTCCTCACGCGCGTCGGG | (nt) |
| | CTCGACATCGGCAAGGTGTGGGTCGCGGACGACGGCGCCGCGGTGGCGGTCTGGACCA | |
| | CGCCGGAGAGCGTCGAAGCGGGGGCGGTGTTCGCCGAGATCGGCCCGCGCATGGCCGA | |
| | GTTGAGCGGTTCCCGGCTGGCCGCGCAGCAACAGATGGAAGGCCTCCTGGCGCCGCAC | |
| | CGGCCCAAGGAGCCCGCGTGGTTCCTGGCCACCGTCGGCGTCTCGCCCGACCACCAGG | |
| | GCAAGGGTCTGGGCAGCGCCGTCGTGCTCCCCGGAGTGGAGGCGGCCGAGCGCGCCGG | |
| | GGTGCCCGCCTTCCTGGAGACCTCCGCGCCCCGCAACCTCCCCTTCTACGAGCGGCTC | |
| | GGCTTCACCGTCACCGCCGACGTCGAGGTGCCCGAAGGACCGCGCACCTGGTGCATGA | |
| | CCCGCAAGCCCGGTGCCTGA | |
| 221 | MTEYKPTVRLATRDDVPRAVRTLAAAFADYPATRHTVDPDRHIERVTELQELELTRVG | Puromycin |
| | LDIGKVWVADDGAAVAVWTTPESVEAGAVFAEIGPRMAELSGSRLAAQQQMEGLLAPH | resistance gene |
| | RPKEPAWFLATVGVSPDHQGKGLGSAVVLPGVEAAERAGVPAFLETSAPRNLPFYERL | (aa) |
| | GFTVTADVEVPEGPRTWCMTRKPGA | |
| 222 | ATGAGTATTCAACATTTCCGTGTCGCCCTTATTCCCTTTTTTGCGGCATTTTGCCTTC | Ampicillin |
| | CTGTTTTTGCTCACCCAGAAACGCTGGTGAAAGTAAAAGATGCTGAAGATCAGTTGGG | resistance gene |
| | TGCACGAGTGGGTTACATCGAACTGGATCTCAACAGCGGTAAGATCCTTGAGAGTTTT | (nt) |
| | CGCCCCGAAGAACGTTTTCCAATGATGAGCACTTTTAAAGTTCTGCTATGTGGCGCGG | |
| | TATTATCCCGTATTGACGCCGGGCAAGAGCAACTCGGTCGCCGCATACACTATTCTCA | |
| | GAATGACTTGGTTGAGTACTCACCAGTCACAGAAAAGCATCTTACGGATGGCATGACA | |
| | GTAAGAGAATTATGCAGTGCTGCCATAACCATGAGTGATAACACTGCGGCCAACTTAC | |
| | TTCTGACAACGATCGGAGGACCGAAGGAGCTAACCGCTTTTTTGCACAACATGGGGGA | |
| | TCATGTAACTCGCCTTGATCGTTGGGAACCGGAGCTGAATGAAGCCATACCAAACGAC | |
| | GAGCGTGACACCACGATGCCTGTAGCAATGGCAACAACGTTGCGCAAACTATTAACTG | |
| | GCGAACTACTTACTCTAGCTTCCCGGCAACAATTAATAGACTGGATGGAGGCGGATAA | |
| | AGTTGCAGGACCACTTCTGCGCTCGGCCCTTCCGGCTGGCTGGTTTATTGCTGATAAA | |
| | TCTGGAGCCGGTGAGCGTGGGTCTCGCGGTATCATTGCAGCACTGGGGCCAGATGGTA | |
| | AGCCCTCCCGTATCGTAGTTATCTACACGACGGGGAGTCAGGCAACTATGGATGAACG | |
| | AAATAGACAGATCGCTGAGATAGGTGCCTCACTGATTAAGCATTGGTAA | |
| 223 | MSIQHFRVALIPFFAAFCLPVFAHPETLVKVKDAEDQLGARVGYIELDLNSGKILESE | Ampicillin |
| | RPEERFPMMSTFKVLLCGAVLSRIDAGQEQLGRRIHYSQNDLVEYSPVTEKHLTDGMT | resistance gene |
| | VRELCSAAITMSDNTAANLLLTTIGGPKELTAFLHNMGDHVTRLDRWEPELNEAIPND | (aa) |
| | ERDTTMPVAMATTLRKLLTGELLTLASRQQLIDWMEADKVAGPLLRSALPAGWFIADK | |
| | SGAGERGSRGIIAALGPDGKPSRIVVIYTTGSQATMDERNRQIAEIGASLIKHW | |
| 224 | AGQCSQNEYFDSLLHACIPCQLRCSSNTPPLTCQRYCNAGVTNSVKGTNA | serine 40 to |
| | | glycine 40 |
| | | huBCMA ECD |
| 225 | CTGGGCTGAGACCCGCAGAGGAAGACGCTCTAGGGATTTGTCCCGGACTAGCGAGATG | hEF 1 alpha |
| | GCAAGGCTGAGGACGGGAGGCTGATTGAGAGGCGAAGGTACACCCTAATCTCAATACA | promoter sequence |
| | ACCCTTGGAGCTAAGCCAGCAATGGTAGAGGGAAGATTCTGCACGTCCCTTCCAGGCG | |
| | GCCTCCCCGTCACCACCCACCCCAACCCGCCCCGACCGGAGCTGAGAGTAATTCATAC | |
| | AAAAGGACTCGCCCCTGCCTTGGGGAATCCCAGGGACCGTCGTTAAACTCCCACTAAC | |
| | GTAGAACCCAGAGATCGCTGCGTTCCCGCCCCCTCACCCGCCCGCTCTCGTCATCACT | |
| | GAGGTGGAGAAGAGCATGCGTGAGGCTCCGGTGCCCGTCAGTGGGCAGAGCGCACATC | |
| | GCCCACAGTCCCCGAGAAGTTGGGGGGAGGGGTCGGCAATTGAACCGGTGCCTAGAGA | |
| | AGGTGGCGCGGGGTAAACTGGGAAAGTGATGTCGTGTACTGGCTCCGCCTTTTTCCCG | |
| | AGGGTGGGGGAGAACCGTATATAAGTGCAGTAGTCGCCGTGAACGTTCTTTTTCGCAA | |
| | CGGGTTTGCCGCCAGAACACAGGTAAGTGCCGTGTGTGGTTCCCGCGGGCCTGGCCTC | |
| | TTTACGGGTTATGGCCCTTGCGTGCCTTGAATTACTTCCACGCCCCTGGCTGCAGTAC | |
| | GTGATTCTTGATCCCGAGCTTCGGGTTGAAAGTGGGTGGGAGAGTTCGAGGCCTTGCG | |
| | CTTAAGGAGCCCCTTCGCCTCGTGCTTGAGTTGAGGCCTGGCTTGGGCGCTGGGGCCG | |
| | CCGCGTGCGAATCTGGTGGCACCTTCGCGCCTATCTCGCTGCTTTCGATAAGTCTCTA | |
| | GCCATTTAAAATTTTTGATGACCTGCTGCGACGCTTTTTTTCTGGCAAGATAGTCTTG | |
| | TAAATGCGGGCCAAGATCTGCACACTGGTATTTCGGTTTTTGGGGCCGCGGGCGGCGA | |
| | CGGGGCCCGTGCGTCCCAGCGCACATGTTCGGCGAGGCGGGGCCTGCGAGCGCGGCCA | |
| | CCGAGAATCGGACGGGGGTAGTCTCAAGCTGGCCGGCCTGCTCTGGTGCCTGGCCTCG | |
| | CGCCGCCGTGTATCGCCCCGCCCTGGGCGGCAAGGCTGGCCCGGTCGGCACCAGTTGC | |
| | GTGAGCGGAAAGATGGCCGCTTCCCGGCCCTGCTGCAGGGAGCTCAAAATGGAGGACG | |
| | CGGCGCTCGGGAGAGCGGGCGGGTGAGTCACCCACACAAAGGAAAAGGGCCTTTCCGT | |
| | CCTCAGCCGTCGCTTCATGTGACTCCACGGAGTACCGGGCGCCGTCCAGGCACCTCGA | |
| | TTAGTTCTCGAGCTTTTGGAGTACGTCGTCTTTAGGTTGGGGGGAGGGGTTTTATGCG | |
| | ATGGAGTTTCCCCACACTGAGTGGGTGGAGACTGAAGTTAGGCCAGCTTGGCACTTGA | |
| | TGTAATTCTCCTTGGAATTTGCCCTTTTTGAGTTTGGATCTTGGTTCATTCTCAAGCC | |
| | TCAGACAGTGGTTCAAAGTTTTTTCCTTCCATTTCAGGTGTCGTGAAAACTACCCCTA | |
| | AAAGCCAAA | |
| 226 | PKSCDKTHTCPPCPAPEAEGAPSVELFPPKPKDTLMISRTPEVTCVVVDVSHEDPEVK | human IgG1 Fc |
| | FNWYVDGVEVHNAKTKPREEQYNSTYRVVSVLTVLHQDWLNGKAYACAVSNKALPAPI | |
| | EKTISKAKGQPREPQVYTLPPSRDELTKNQVSLTCLVKGFYPSDIAVEWESNGQPENN | |
| | YKATPPVLDSDGSFFLYSKLTVDKSRWQQGNVFSCSVMHEALHNHYTQKSLSLSPGK | |
| 227 | EPKSCDKTHTCPPCPAPELLGGPSVFLFPPKPKDTLMISRTPEVTCVVVDVSHEDPEV | human IgG1 Fc |
| | KFNWYVDGVEVHNAKTKPREEQYNSTYRVVSVLTVLHQDWLNGKEYKCAVSNKALPAP | |
| | IEKTISKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLSLSPGK | |
| 228 | EPKSCDKTHTCPPCPAPELLGGPSVFLFPPKPKDTLMISRTPEVTCVVVDVSHEDPEV | human IgG1 Fc |
| | KFNWYVDGVEVHNAKTKPREEQYNSTYRVVSVLTVLHQDWLNGKEYKCKVSNKALPAP | |
| | IEKTISKAKGQPREPQVYTLPPSRDELTKNQVSLTCLVKGFYPSDIAVEWESNGOPEN | |
| | NYKTTPPVLDSDGSFFLYSKLTVDKSRWQQGNVFSCSVMHEALHNHYTQKSLSLSPGK | |
| 229 | AEPKSCDKTHTCPPCPAPELLGGPSVFLFPPKPKDTLMISRTPEVTCVVVDVSHEDPE | human IgG1 Fc |
| | VKFNWYVDGVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVEWESNGQPE | |
| | NNYKTTPPVLDSDGSFFLYSKLTVDKSRWQQGNVFSCSVMHEALHNHYTQKSLSLSPG | |
| | K | |
| 230 | PKSCDKTHTCPPCPAPELKGGPSVFLFPPKPKDTLMISRTPEVTCVVVDVSHEDPEVK | human IgG1 Fc |
| | FNWYVDGVEVHNAKTKPREEQYNSTYRVVSVLTVLHQDWLNGKEYKCKVSNKALPAPI | |
| | EKTISKAKGQPREPQVYTLPPSRDELTKNQVSLTCLVKGFYPSDIAVEWESNGQPENN | |
| | YKTTPPVLDSDGSFFLYSKLTVDKSRWQQGNVFSCSVMHEALHNHYTQKSLSLSPGK | |
| 231 | AESKYGPPCPPCPAPEAAGGPSVFLFPPKPKDTLMISRTPEVTCVVVDVSQEDPEVQF | human IgG4 Fc |
| | NWYVDGVEVHNAKTKPREEQFNSTYRVVSVLTVLHQDWLNGKEYKCKVSNKGLPSSIE | |
| | KTISKAKGQPREPQVYTLPPSQEEMTKNQVSLTCLVKGFYPSDIAVEWESNGQPENNY | |
| | KTTPPVLDSDGSFFLYSRLTVDKSRWQEGNVFSCSVMHEALHNHYTQKSLSLSLGK | |
| 232 | ESKYGPPCPPCPAPEAAGGPSVFLFPPKPKDTLMISRTPEVTCVVVDVSQEDPEVQEN | human IgG4 Fc |
| | WYVDGVEVHNAKTKPREEQFNSTYRVVSVLTVLHQDWLNGKEYKCKVSNKGLPSSIEK | |
| | TISKAKGQPREPQVYTLPPSQEEMTKNQVSLTCLVKGFYPSDIAVEWESNGQPENNYK | |
| | TTPPVLDSDGSFFLYSRLTVDKSRWQEGNVFSCSVMHEALHNHYTQKSLSLSLGK | |
| 233 | ESKYGPPCPPCPAPEFAGGPSVFLFPPKPKDTLMISRTPEVTCVVVDVSQEDPEVQEN | human IgG4 Fc |
| | WYVDGVEVHNAKTKPREEQFNSTYRVVSVLTVLHQDWLNGKEYKCKVSNKGLPSSIEK | |
| | TISKAKGQPREPQVYTLPPSQEEMTKNQVSLTCLVKGFYPSDIAVEWESNGQPENNYK | |
| | TTPPVLDSDGSFFLYSRLTVDKSRWQEGNVFSCSVMHEALHNHYTQKSLSLSLGK | |
| 234 | ESKYGPPCPSCPAPEVAGGPSVFLFPPKPKDTLMISRTPEVTCVVVDVSQEDPEVQEN | human IgG4 Fc |
| | WYVDGVEVHNAKTKPREEQFNSTYRVVSVLTVLHQDWLNGKEYKCKVSNKGLPSSIEK | |
| | TISKAKGQPREPQVYTLPPSQEEMTKNQVSLTCLVKGFYPSDIAVEWESNGQPENNYK | |
| | TTPPVLDSDGSFFLYSRLTVDKSRWQEGNVFSCSVMHEALHNHYTQKSLSLSLGK | |
| 235 | ERKCCVECPPCPAPPVAGPSVFLFPPKPKDTLMISRTPEVTCVVVDVSHEDPEVQENW | human IgG2 Fc |
| | YVDGVEVHNAKTKPREEQFNSTFRVVSVLTVVHQDWLNGKEYKCKVSNKGLPAPIEKT | |
| | ISKTKGQPREPQVYTLPPSREEMTKNQVSLTCLVKGFYPSDIAVEWESNGQPENNYKT | |
| | TPPMLDSDGSFFLYSKLTVDKSRWQQGNVFSCSVMHEALHNHYTQKSLSLSPGK | |
| 236 | ERKCCVECPPCPAPPVAGPSVFLFPPKPKDTLMISRTPEVTCVVVDVKHEDPEVQENW | human IgG2 Fc |
| | YVDGVEVHNAKTKPREEQFNSTFRVVSVLTVVHQDWLNGKEYKCKVSNKGLPAPIEKT | |
| | ISKTKGQPREPQVYTLPPSREEMTKNQVSLTCLVKGFYPSDIAVEWESNGQPENNYKT | |
| | TPPMLDSDGSFFLYSKLTVDKSRWQQGNVFSCSVMHEALHNHYTQKSLSLSPGK | |
| 237 | AGQCSQNEYFDSLLHACIPCQLRCSSNTPPLTCQRYCNAGVTNSV | BCMA CRD |
| | | S40G (aa) |
| 238 | gagtccaaatatggtcccccatgcccaccatgcccagcaccagaggccgccgggggac | IgG4 Fc (nt) |
| | catcagtcttcctgttccccccaaaacccaaggacactctcatgatctcccggacccc | |
| | tgaggtcacgtgcgtggtggtggacgtgagccaggaagaccccgaggtccagttcaac | |
| | tggtacgtggatggcgtggaggtgcataatgccaagacaaagccgcgggaggagcagt | |
| | tcaacagcacgtaccgtgtggtcagcgtcctcaccgtcctgcaccaggactggctgaa | |
| | cggcaaggagtacaagtgcaaggtctccaacaaaggcctcccgtcctccatcgagaaa | |
| | accatctccaaagccaaagggcagccccgagagccacaggtgtacaccctgcccccat | |
| | cccaggaggagatgaccaagaaccaggtcagcctgacctgcctggtcaaaggcttcta | |
| | ccccagcgacatcgccgtggagtgggagagcaatgggcagccggagaacaactacaag | |
| | accacgcctcccgtgctggactccgacggctccttcttcctctacagcaggctcaccg | |
| | tggacaagagcaggtggcaggaggggaatgtcttctcatgctccgtgatgcatgaggc | |
| | tctgcacaaccactacacacagaagagcctctccctgtctccgggtaaatgag | |
| 239 | atggcctggatgatgcttctcctcggactccttgcttatggatcaggagtcgactctg | BAv9-1 - linker - |
| | ccggacagtgtagccagaacgagtacttcgatcacctgattcgcgcttgtataccgtg | IgG4 Fc w/ signal |
| | ccaacttagatgctcgagtaatactccaccactgacatgccagcgttattgcaacgcc | sequence (nt) |
| | ggcgtcacaaacagcgtcggcggcggtggcagcgctgagtccaaatatggtcccccat | |
| | gcccaccatgcccagcaccagaggccgccgggggaccatcagtcttcctgttcccccc | |
| | aaaacccaaggacactctcatgatctcccggacccctgaggtcacgtgcgtggtggtg | |
| | gacgtgagccaggaagaccccgaggtccagttcaactggtacgtggatggcgtggagg | |
| | tgcataatgccaagacaaagccgcgggaggagcagttcaacagcacgtaccgtgtggt | |
| | cagcgtcctcaccgtcctgcaccaggactggctgaacggcaaggagtacaagtgcaag | |
| | gtctccaacaaaggcctcccgtcctccatcgagaaaaccatctccaaagccaaagggc | |
| | agccccgagagccacaggtgtacaccctgcccccatcccaggaggagatgaccaagaa | |
| | ccaggtcagcctgacctgcctggtcaaaggcttctaccccagcgacatcgccgtggag | |
| | tgggagagcaatgggcagccggagaacaactacaagaccacgcctcccgtgctggact | |
| | ccgacggctccttcttcctctacagcaggctcaccgtggacaagagcaggtggcagga | |
| | ggggaatgtcttctcatgctccgtgatgcatgaggctctgcacaaccactacacacag | |
| | aagagcctctccctgtctccgggtaaatga | |
| 240 | ctattctatagtgtcacctaaatgctagagctcgctgatcagcctcgactgtgccttc | BGH poly A |
| | tagttgccagccatctgttgtttgcccctcccccgtgccttccttgaccctggaaggt | sequence |
| | gccactcccactgtcctttcctaataaaatgaggaaattgcatcgcattgtctgagta | |
| | ggtgtcattctattctggggggtggggtggggcaggacagcaagggggaggattggga | |
| | agacaatagcaggcatgctggggatgcggtgggctctatggcttctgaggcggaaaga | |
| | accagctggggctcgagc | |
| 241 | cgcgttgctggcgtttttccataggctccgcccccctgacgagcatcacaaaaatcga | pUC vector |
| | cgctcaagtcagaggtggcgaaacccgacaggactataaagataccaggcgtttcccc | sequence |
| | ctggaagctccctcgtgcgctctcctgttccgaccctgccgcttaccggatacctgtc | |
| | cgcctttctcccttcgggaagcgtggcgctttctcatagctcacgctgtaggtatctc | |
| | agttcggtgtaggtcgttcgctccaagctgggctgtgtgcacgaaccccccgttcagc | |
| | ccgaccgctgcgccttatccggtaactatcgtcttgagtccaacccggtaagacacga | |
| | cttatcgccactggcagcagccactggtaacaggattagcagagcgaggtatgtaggc | |
| | ggtgctacagagttcttgaagtggtggcctaactacggctacactagaaggacagtat | |
| | ttggtatctgcgctctgctgaagccagttaccttcggaaaaagagttggtagctcttg | |
| | atccggcaaacaaaccaccgctggtagcggtggtttttttgtttgcaagcagcagatt | |
| | acgcgcagaaaaaaaggatctcaagaagatccttt | |
| 242 | ttaccaatgcttaatcagtgaggcacctatctcagcgatctgtctatttcgttcatcc | Ampicillin |
| | atagttgcctgactccccgtcgtgtagataactacgatacgggagggcttaccatctg | resistance gene |
| | gccccagtgctgcaatgataccgcgagacccacgctcaccggctccagatttatcagc | (nt) |
| | aataaaccagccagccggaagggccgagcgcagaagtggtcctgcaactttatccgcc | |
| | tccatccagtctattaattgttgccgggaagctagagtaagtagttcgccagttaata | |
| | gtttgcgcaacgttgttgccattgctacaggcatcgtggtgtcacgctcgtcgtttgg | |
| | tatggcttcattcagctccggttcccaacgatcaaggcgagttacatgatcccccatg | |
| | ttgtgcaaaaaagcggttagctccttcggtcctccgatcgttgtcagaagtaagttgg | |
| | ccgcagtgttatcactcatggttatggcagcactgcataattctcttactgtcatgcc | |
| | atccgtaagatgcttttctgtgactggtgagtactcaaccaagtcattctgagaatag | |
| | tgtatgcggcgaccgagttgctcttgcccggcgtcaatacgggataataccgcgccac | |
| | atagcagaactttaaaagtgctcatcattggaaaacgttcttcggggcgaaaactctc | |
| | aaggatcttaccgctgttgagatccagttcgatgtaacccactcgtgcacccaactga | |
| | tcttcagcatcttttactttcaccagcgtttctgggtgagcaaaaacaggaaggcaaa | |
| | atgccgcaaaaaagggaataagggcgacacggaaatgttgaatactcat | |
| 243 | gccggacagtgtagccagaacgagtacttcgatcacctgattcgcgcttgtataccgt | BAv9-1 - linker - |
| | gccaacttagatgctcgagtaatactccaccactgacatgccagcgttattgcaacgc | IgG4 Fc (nt) |
| | cggcgtcacaaacagcgtcggcggcggtggcagcgctgagtccaaatatggtccccca | |
| | tgcccaccatgcccagcaccagaggccgccgggggaccatcagtcttcctgttccccc | |
| | caaaacccaaggacactctcatgatctcccggacccctgaggtcacgtgcgtggtggt | |
| | ggacgtgagccaggaagaccccgaggtccagttcaactggtacgtggatggcgtggag | |
| | gtgcataatgccaagacaaagccgcgggaggagcagttcaacagcacgtaccgtgtgg | |
| | tcagcgtcctcaccgtcctgcaccaggactggctgaacggcaaggagtacaagtgcaa | |
| | ggtctccaacaaaggcctcccgtcctccatcgagaaaaccatctccaaagccaaaggg | |
| | cagccccgagagccacaggtgtacaccctgcccccatcccaggaggagatgaccaaga | |
| | accaggtcagcctgacctgcctggtcaaaggcttctaccccagcgacatcgccgtgga | |
| | gtgggagagcaatgggcagccggagaacaactacaagaccacgcctcccgtgctggac | |
| | tccgacggctccttcttcctctacagcaggctcaccgtggacaagagcaggtggcagg | |
| | aggggaatgtcttctcatgctccgtgatgcatgaggctctgcacaaccactacacaca | |
| | gaagagcctctccctgtctccgggtaaatga | |
| 244 | tccctcccccccccctaacgttactggccgaagccgcttggaataaggccggtgtgcg | IRES sequence |
| | tttgtctatatgttattttccaccatattgccgtcttttggcaatgtgagggcccgga | |
| | aacctggccctgtcttcttgacgagcattcctaggggtctttcccctctcgccaaagg | |
| | aatgcaaggtctgttgaatgtcgtgaaggaagcagttcctctggaagcttcttgaaga | |
| | caaacaacgtctgtagcgaccctttgcaggcagcggaaccccccacctggcgacaggt | |
| | gcctctgcggccaaaagccacgtgtataagatacacctgcaaaggggcacaacccca | |
| | gtgccacgttgtgagttggatagttgtggaaagagtcaaatggctctcctcaagcgta | |
| | ttcaacaaggggctgaaggatgcccagaaggtaccccattgtatgggatctgatctgg | |
| | ggcctcggtacacatgctttacatgtgtttagtcgaggttaaaaaaacgtctaggccc | |
| | cccgaaccacggggacgtggttttcctttgaaaaacacgatgataatatggccaca | |
| 245 | atgtacaggatgcaactcctgtcttgcattgcactaagtcttgcacttgtcacgaatt | IL 2 signal peptide- |
| | cggtgagcaagggcgaggagctgttcaccggggtggtgcccatcctggtcgagctgga | GFP (nt) |
| | cggcgacgtaaacggccacaagttcagcgtgtccggcgagggcgagggcgatgccacc | |
| | tacggcaagctgaccctgaagttcatctgcaccaccggcaagctgcccgtgccctggc | |
| | ccaccctcgtgaccaccctgacctacggcgtgcagtgcttcagccgctaccccgacca | |
| | catgaagcagcacgacttcttcaagtccgccatgcccgaaggctacgtccaggagcgt | |
| | acgatcttcttcaaggacgacggcaactacaagacccgcgccgaggtgaagttcgagg | |
| | gcgacaccctggtgaaccgcatcgagctgaagggcatcgacttcaaggaggacggcaa | |
| | catcctggggcacaagctggagtacaactacaacagccacaacgtctatatcatggcc | |
| | gacaagcagaagaacggcatcaaggtgaacttcaagatccgccacaacatcgaggacg | |
| | gcagcgtgcagctcgccgaccactaccagcagaacacccccatcggcgacggccccgt | |
| | gctgctgcccgacaaccactacctgagcacccagtccgccctgagcaaagaccccaac | |
| | gagaagcgcgatcacatggtcctgctggagttcgtgaccgccgccgggatcactctcg | |
| | gcatggacgagctgtacaagtccggacatgaaacaaccccaaataaaggaagtggaac | |
| | cacttcaggtactacccgtcttctatctgggcacacgtgtttcacgttgacaggtttg | |
| | cttgggacgctagtaaccatgggcttgctgacttaa | |
| 246 | MYRMQLLSCIALSLALVTNSVSKGEELFTGVVPILVELDGDVNGHKFSVSGEGEGDAT | IL 2 signal peptide- |
| | YGKLTLKFICTTGKLPVPWPTLVTTLTYGVQCFSRYPDHMKQHDFFKSAMPEGYVQER | GFP (aa) |
| | TIFFKDDGNYKTRAEVKFEGDTLVNRIELKGIDFKEDGNILGHKLEYNYNSHNVYIMA | |
| | DKQKNGIKVNFKIRHNIEDGSVQLADHYQQNTPIGDGPVLLPDNHYLSTQSALSKDPN | |
| | EKRDHMVLLEFVTAAGITLGMDELYKSGHETTPNKGSGTTSGTTRLLSGHTCFTLTGL | |
| | LGTLVTMGLLT | |
| 247 | ggtgtggaaagtccccaggctccccagcaggcagaagtatgcaaagcatgcatctcaa | SV40 promoter |
| | ttagtcagcaaccaggtgtggaaagtccccaggctccccagcaggcagaagtatgcaa | |
| | agcatgcatctcaattagtcagcaaccatagtcccgcccctaactccgcccatcccgc | |
| | ccctaactccgcccagttccgcccattctccgccccatggctgactaattttttttat | |
| | ttatgcagaggccgaggccgcctcggcctctgagctattccagaagtagtgaggaggc | |
| | ttttttggaggcctaggcttttgcaaa | |
| 248 | cgacccgcagcgcccgaccgaaaggagcgcacgaccccatggctccgaccgaagccga | SV40 poly A |
| | cccgggcggccccgccgaccccgcacccgcccccgaggcccaccgactaggatcataa | region |
| | tcagccataccacatttgtagaggttttacttgctttaaaaaacctcccacacctccc | |
| | cctgaacctgaaacataaaatgaatgcaattgttgttgttaacttgtttattgcagct | |
| | tataatggttacaaataaagcaatagcatcacaaatttcacaaataaagcattttttt | |
| | cactgcattctagttgtggtttgtccaaactcatcaatgtatcttatcatgtctgaat | |
| | tcgatcgccagcttttgttccctttagtgagggttaatttcgagcttggcgtaatcat | |
| | ggtcatagctgtttcctgtgtgaaattgttatccgctcacaattccacacaacatacg | |
| | agccggaagcataaagtgtaaagcctggggtgcctaatgagtgagctaactcacatta | |
| | attgcgttgcgctcactgcccgctttccagtcgggaaacctgtcgtgccagctgcatt | |
| | aatgaatcggccaacgcgcggggagaggcggtttgcgtattgggcgctcttccgcttc | |
| | ctcgctcactgactcgctgcgctcggtcgttcggctgcggcgagcggtatcagctcac | |
| | tcaaaggcggtaatacggttatccacagaatcaggggataacgcaggaaagaacatgt | |
| | gagcaaaaggccagcaaaaggccaggaaccgtaaaaagg | |
| 249 | atgtacaggatgcaactcctgtcttgc | IL 2 signal peptide |
| | | (nt) |
| 250 | MYRMQLLSCIALSLALVTNS | IL 2 signal peptide |
| | | (aa) |
| 251 | acttgtcacgaattcggtgagcaagggcgaggagctgttcaccggggtggtgcccatc | GFP (nt) |
| | ctggtcgagctggacggcgacgtaaacggccacaagttcagcgtgtccggcgagggcg | |
| | agggcgatgccacctacggcaagctgaccctgaagttcatctgcaccaccggcaagct | |
| | gcccgtgccctggcccaccctcgtgaccaccctgacctacggcgtgcagtgcttcagc | |
| | cgctaccccgaccacatgaagcagcacgacttcttcaagtccgccatgcccgaaggct | |
| | acgtccaggagcgtacgatcttcttcaaggacgacggcaactacaagacccgcgccga | |
| | ggtgaagttcgagggcgacaccctggtgaaccgcatcgagctgaagggcatcgacttc | |
| | aaggaggacggcaacatcctggggcacaagctggagtacaactacaacagccacaacg | |
| | tctatatcatggccgacaagcagaagaacggcatcaaggtgaacttcaagatccgcca | |
| | caacatcgaggacggcagcgtgcagctcgccgaccactaccagcagaacacccccatc | |
| | ggcgacggccccgtgctgctgcccgacaaccactacctgagcacccagtccgccctga | |
| | gcaaagaccccaacgagaagcgcgatcacatggtcctgctggagttcgtgaccgccgc | |
| | cgggatcactctcggcatggacgagctgtacaagtccggacatgaaacaaccccaaat | |
| | aaaggaagtggaaccacttcaggtactacccgtcttctatctgggcacacgtgtttca | |
| | cgttgacaggtttgcttgggacgctagtaaccatgggcttgctgacttaa | |
| 252 | VSKGEELFTGVVPILVELDGDVNGHKESVSGEGEGDATYGKLTLKFICTTGKLPVPWP | GFP (aa) |
| | TLVTTLTYGVQCFSRYPDHMKQHDFFKSAMPEGYVQERTIFFKDDGNYKTRAEVKFEG | |
| | DTLVNRIELKGIDFKEDGNILGHKLEYNYNSHNVYIMADKQKNGIKVNFKIRHNIEDG | |
| | SVQLADHYQQNTPIGDGPVLLPDNHYLSTQSALSKDPNEKRDHMVLLEFVTAAGITLG | |
| | MDELYKSGHETTPNKGSGTTSGTTRLLSGHTCFTLTGLLGTLVTMGLLT | |

1. A variant B cell maturation antigen (BCMA) polypeptide, comprising a variant cysteine rich domain (CRD) comprising at least one amino acid substitution(s) selected from among: (1) a histidine, an arginine, a proline, or an asparagine at position 12 (S12H, S12R, S12P, or S12N); (2) an isoleucine or a valine at position 14 (L14I or L14V); (3) an arginine or an asparagine at position 15 (H15R or H15N); (4) a serine at position 16 (A16S); and (5) a valine, an isoleucine, or an alanine at position 22 (L22V, L22I, or L22A), with reference to the amino acid positions of SEQ ID NO:1.

2.-27. (canceled)

28. A fusion polypeptide, comprising:
a variant B cell maturation antigen (BCMA) polypeptide, comprising a variant cysteine rich domain (CRD) comprising at least one amino acid substitutions selected from among: (1) a histidine, an arginine, a proline, or an asparagine at position 12 (S12H, S12R, S12P, or S12N); (2) an isoleucine or a valine at position 14 (L14I or L14V); (3) an arginine or an asparagine at position 15 (H15R or H15N); (4) a serine at position 16 (A16S); and (5) a valine, an isoleucine, or an alanine at position 22 (L22V, L22I, or L22A); with reference to the amino acid positions of SEQ ID NO:1; and
an immunoglobulin (Ig) Fc polypeptide.

29.-180. (canceled)

181. A fusion polypeptide, comprising the variant BCMA polypeptide of claim 1, and an additional polypeptide.

182. A dimer, comprising a first monomer comprising the variant BCMA polypeptide of claim 1 or a fusion polypeptide comprising the variant BCMA polypeptide of claim 1 and an additional polypeptide; and
a second monomer comprising the variant BCMA polypeptide of claim 1 or the fusion polypeptide.

183. A conjugate, comprising
the variant BCMA polypeptide of claim 1;
a fusion polypeptide comprising the variant BCMA polypeptide of claim 1 and an additional polypeptide; or
a dimer, comprising a first monomer comprising the variant BCMA polypeptide of claim 1 or a fusion polypeptide comprising the variant BCMA polypeptide of claim 1 and an additional polypeptide; and
a second monomer comprising the variant BCMA polypeptide of claim 1 or the fusion polypeptide; and
an additional moiety that is covalently bound to the variant BCMA polypeptide of claim 1, the fusion polypeptide, or the dimer.

184. The variant BCMA polypeptide of claim 1;
a fusion polypeptide comprising the variant BCMA polypeptide of claim 1 and an additional polypeptide;
a dimer, comprising a first monomer comprising the variant BCMA polypeptide of claim 1 or a fusion polypeptide, comprising the variant BCMA polypeptide of claim 1 and an additional polypeptide; and
a second monomer comprising the variant BCMA polypeptide of claim 1 or the fusion polypeptide; or
the conjugate comprising the variant BCMA polypeptide of claim 1;
a fusion polypeptide comprising the variant BCMA polypeptide and an additional polypeptide; or
a dimer comprising a first monomer comprising the variant BCMA polypeptide of claim 1; or
a fusion polypeptide comprising the variant BCMA polypeptide of claim 1 and an additional polypeptide; and
a second monomer comprising the variant BCMA polypeptide of claim 1 or the fusion polypeptide; and
an additional moiety that is covalently bound to the variant BCMA polypeptide of claim 1, the fusion polypeptide, or the dimer,
wherein the variant BCMA polypeptide of claim 1, the fusion polypeptide, the dimer, the conjugate binds at least one selected from the group of B-cell Activating Factor of the TNF family (BAFF) and A Proliferation Inducing Ligand (APRIL) or variants thereof.

185. A polynucleotide comprising the nucleotide sequence encoding
the variant BCMA polypeptide of claim 1,
a fusion polypeptide comprising the variant BCMA polypeptide of claim 1 and an additional polypeptide; or
at least one selected from the group of a first monomer and a second monomer of the dimer, comprising a first monomer comprising the variant BCMA polypeptide of claim 1 or a fusion polypeptide, comprising the variant BCMA polypeptide of claim 1 and an additional polypeptide; and a second monomer comprising the variant BCMA polypeptide of claim 1 or the fusion polypeptide.

186. A vector comprising
the polynucleotide comprising a nucleotide sequence encoding the variant BCMA polypeptide of claim 1,
a fusion polypeptide, comprising the variant BCMA polypeptide of claim 1 and an additional polypeptide; or
at least one selected from the group of a first monomer and a second monomer of the dimer, comprising a first monomer comprising the variant BCMA polypeptide of claim 1 or a fusion polypeptide, comprising the variant BCMA polypeptide of claim 1 and an additional polypeptide; and a second monomer comprising the variant BCMA polypeptide of claim 1 or the fusion polypeptide.

187. A cell comprising the polynucleotide comprising a nucleotide sequence encoding the variant BCMA polypeptide of claim 1,
a fusion polypeptide, comprising the variant BCMA polypeptide of claim 1 and an additional polypeptide; or
at least one selected from the group of a first monomer and a second monomer of the dimer, comprising a first monomer comprising the variant BCMA polypeptide of claim 1 or a fusion polypeptide, comprising the variant BCMA polypeptide of claim 1 and an additional polypeptide; and a second monomer comprising the variant BCMA polypeptide of claim 1 or the fusion polypeptide.

188. A cell comprising a vector comprising the polynucleotide comprising a nucleotide sequence encoding the variant BCMA polypeptide of claim 1,
a fusion polypeptide, comprising the variant BCMA polypeptide of claim 1 and an additional polypeptide; or
at least one selected from the group of a first monomer and a second monomer of the dimer, comprising a first monomer comprising the variant BCMA polypeptide of claim 1 or a fusion polypeptide, comprising the variant BCMA polypeptide of claim 1 and an additional polypeptide; and a second monomer comprising the variant BCMA polypeptide of claim 1 or the fusion polypeptide.

189. A method of manufacturing a variant BCMA polypeptide, a fusion polypeptide, or a dimer, wherein the method comprises:
1) introducing the polynucleotide of claim 185 into a cell;
2) culturing the host cell under conditions suitable for expression of the polypeptide;
3) recovering or isolating the polypeptide; and optionally
4) purifying the polypeptide.

190. A method of manufacturing a variant BCMA polypeptide, a fusion polypeptide, or a dimer, wherein the method comprises:
1) introducing the vector of claim 186 into a cell;
2) culturing the host cell under conditions suitable for expression of the polypeptide;
3) recovering or isolating the polypeptide; and optionally
4) purifying the polypeptide.

191. A pharmaceutical composition comprising
the variant BCMA polypeptide of claim 1;
a fusion polypeptide comprising
the variant BCMA polypeptide of claim 1 and an additional polypeptide;
a dimer comprising
a first monomer comprising the variant BCMA polypeptide of claim 1 or a fusion polypeptide, comprising the variant BCMA polypeptide of claim 1 and an additional polypeptide; and a second monomer comprising the variant BCMA polypeptide of claim 1 or the fusion polypeptide;
a conjugate comprising
the variant BCMA polypeptide of claim 1; the fusion polypeptide; or the dimer; and an additional moiety that is covalently bound to the variant BCMA polypeptide of claim 1, the fusion polypeptide, or the dimer;
a polynucleotide comprising a nucleotide sequence encoding
the variant BCMA polypeptide of claim 1;
a fusion polypeptide comprising the variant BCMA polypeptide of claim 1 and an additional polypeptide; or
at least one selected from the group of a first monomer and a second monomer of the dimer, comprising a first monomer comprising the variant BCMA polypeptide of claim 1 or a fusion polypeptide, comprising the variant BCMA polypeptide of claim 1 and an additional polypeptide; and a second monomer comprising the variant BCMA polypeptide of claim 1 or the fusion polypeptide;
a vector comprising the polynucleotide;
a cell comprising the polynucleotide; or
a cell comprising the vector.

192. The variant BCMA polypeptide of claim 1 for treating a disease or disorder.

193. A method of treatment of a disease or disorder, the method comprising administering the variant BCMA polypeptide of claim 1 to a subject having the disease or disorder.

194. The method of claim 193, wherein the disease or disorder is a B-cell- or antibody-mediated disease or disorder.

195. Use of a variant BCMA polypeptide of claim 1 in the manufacture of a medicament for the treatment of a disease or disorder.


SEQUENCE LISTING (254 sequences)

SEQ ID NO: 1 - protein, 45 aa, synthetic construct
AGQCSQNEYFDSLLHACIPCQLRCSSNTPPLTCQRYCNASVTNSV

SEQ ID NO: 2 - DNA, 135 bp, synthetic construct
  source 1..135: mol_type = other DNA
gctgggcagtgctcccaaaatgaatattttgacagtttgttgcatgcttgcataccttgtcaacttcgatgttcttctaatactcctcctctaacatgtcagcgttattgtaatgcaagtgtgaccaattcagtg

SEQ ID NO: 3 - protein, 45 aa, synthetic construct
AGQCSQNEYFDHLIRACIPCQLRCSSNTPPLTCQRYCNAGVTNSV

SEQ ID NO: 4 - protein, 45 aa, synthetic construct
AGQCSQNEYFDRLIRACIPCQLRCSSNTPPLTCQRYCNAGVTNSV

SEQ ID NO: 5 - protein, 45 aa, synthetic construct
AGQCSQNEYFDRLVRACIPCQLRCSSNTPPLTCQRYCNAGVTNSV

SEQ ID NO: 6 - protein, 45 aa, synthetic construct
AGQCSQNEYFDHLLRACIPCQVRCSSNTPPLTCQRYCNAGVTNSV

SEQ ID NO: 7 - protein, 45 aa, synthetic construct
AGQCSQNEYFDRLLRACIPCQVRCSSNTPPLTCQRYCNAGVTNSV

SEQ ID NO: 8 - protein, 45 aa, synthetic construct
AGQCSQNEYFDHLLRACIPCQLRCSSNTPPLTCQRYCNAGVTNSV

SEQ ID NO: 9 - protein, 45 aa, synthetic construct
AGQCSQNEYFDRLVNACIPCQLRCSSNTPPLTCQRYCNAGVTNSV

SEQ ID NO: 10 - protein, 45 aa, synthetic construct
AQQCSQNEYFDRLVNSCVPCRLRCSSNNPPLTCQHYCNAGVTNSV

SEQ ID NO: 11 - protein, 45 aa, synthetic construct
AQQCSQNEYFDRLVNSCIPCQLRCSSNNPPLTCQHYCNAGVTNSV

SEQ ID NO: 12 - protein, 45 aa, synthetic construct
AGQCSQNEYFDRLVNSCIPCQLRCSSNNPPLTCQHYCNAGVTNSV

SEQ ID NO: 13 - protein, 45 aa, synthetic construct
AQQCSQNEYFDSLVNSCIPCQLRCSSNNPPLTCQHYCNAGVTNSV

SEQ ID NO: 14 - protein, 45 aa, synthetic construct
AQQCSQNEYFDRLLNSCIPCQLRCSSNNPPLTCQHYCNAGVTNSV

SEQ ID NO: 15 - protein, 45 aa, synthetic construct
AQQCSQNEYFDRLVHSCIPCQLRCSSNNPPLTCQHYCNAGVTNSV

SEQ ID NO: 16 - protein, 45 aa, synthetic construct
AQQCSQNEYFDRLVNACIPCQLRCSSNNPPLTCQHYCNAGVTNSV

SEQ ID NO: 17 - protein, 45 aa, synthetic construct
AQQCSQNEYFDRLVNSCIPCQLRCSSNTPPLTCQHYCNAGVTNSV

SEQ ID NO: 18 - protein, 45 aa, synthetic construct
AQQCSQNEYFDRLVNSCIPCQLRCSSNNPPLTCQRYCNAGVTNSV

SEQ ID NO: 19 - protein, 45 aa, synthetic construct
AGQCSQNEYFDRLVNSCIPCQLRCSSNTPPLTCQRYCNAGVTNSV

SEQ ID NO: 20 - protein, 45 aa, synthetic construct
AGQCSQNEYFDSLVNACIPCQLRCSSNTPPLTCQRYCNAGVTNSV

SEQ ID NO: 21 - protein, 45 aa, synthetic construct
AGQCSQNEYFDHLVNACIPCQLRCSSNTPPLTCQRYCNAGVTNSV

SEQ ID NO: 22 - protein, 45 aa, synthetic construct
AGQCSQNEYFDHLVNSCIPCQLRCSSNTPPLTCQRYCNAGVTNSV

SEQ ID NO: 23 - protein, 45 aa, synthetic construct
AGQCSQNEYFDSLVNSCIPCQLRCSSNTPPLTCQRYCNAGVTNSV

SEQ ID NO: 24 - protein, 45 aa, synthetic construct
AGQCSQNEYFDRLVNSCIPCQVRCSSNTPPLTCQRYCNAGVTNSV

SEQ ID NO: 25 - protein, 45 aa, synthetic construct
AGQCSQNEYFDRLVNACIPCQVRCSSNTPPLTCQRYCNAGVTNSV

SEQ ID NO: 26 - protein, 45 aa, synthetic construct
AGQCSQNEYFDHLVNACIPCQVRCSSNTPPLTCQRYCNAGVTNSV

SEQ ID NO: 27 - protein, 45 aa, synthetic construct
AGQCSQNEYFDSLVNACIPCQVRCSSNTPPLTCQRYCNAGVTNSV

SEQ ID NO: 28 - protein, 45 aa, synthetic construct
AQQCSPNEYFDRLIRACIPCQLRCSSNTPPVTCQRYCNTGVTNSV

SEQ ID NO: 29 - protein, 45 aa, synthetic construct
AQQCSPNEYFDRLIRACIPCQLRCSSNTPPLTCQRYCNTGVTNSV

SEQ ID NO: 30 - protein, 45 aa, synthetic construct
AGQCSPNEYFDRLIRACIPCQLRCSSNTPPLTCQRYCNTGVTNSV

SEQ ID NO: 31 - protein, 45 aa, synthetic construct
AQQCSQNEYFDRLIRACIPCQLRCSSNTPPLTCQRYCNTGVTNSV

SEQ ID NO: 32 - protein, 45 aa, synthetic construct
AQQCSPNEYFDSLIRACIPCQLRCSSNTPPLTCQRYCNTGVTNSV

SEQ ID NO: 33 - protein, 45 aa, synthetic construct
AQQCSPNEYFDRLLRACIPCQLRCSSNTPPLTCQRYCNTGVTNSV

SEQ ID NO: 34 - protein, 45 aa, synthetic construct
AQQCSPNEYFDRLIHACIPCQLRCSSNTPPLTCQRYCNTGVTNSV

SEQ ID NO: 35 - protein, 45 aa, synthetic construct
AQQCSPNEYFDRLIRACIPCQLRCSSNTPPLTCQRYCNAGVTNSV

SEQ ID NO: 36 - protein, 45 aa, synthetic construct
AGQCSQNEYFDSLIRACIPCQLRCSSNTPPLTCQRYCNAGVTNSV

SEQ ID NO: 37 - protein, 45 aa, synthetic construct
AGQCSQNEYFDRLIRACIPCQVRCSSNTPPLTCQRYCNAGVTNSV

SEQ ID NO: 38 - protein, 45 aa, synthetic construct
AGQCSQNEYYDHLLRACVSCHVRCSSNNPPAICQRYCNAGVTNSV

SEQ ID NO: 39 - protein, 45 aa, synthetic construct
AGQCSQNEYFDHLLRACISCQVRCSSNNPPLTCQRYCNAGVTNSV

SEQ ID NO: 40 - protein, 45 aa, synthetic construct
AGQCSQNEYFDSLLRACISCQVRCSSNNPPLTCQRYCNAGVTNSV

SEQ ID NO: 41 - protein, 45 aa, synthetic construct
AGQCSQNEYFDHLLHACISCQVRCSSNNPPLTCQRYCNAGVTNSV

SEQ ID NO: 42 - protein, 45 aa, synthetic construct
AGQCSQNEYFDHLLRACIPCQVRCSSNNPPLTCQRYCNAGVTNSV

SEQ ID NO: 43 - protein, 45 aa, synthetic construct
AGQCSQNEYFDHLLRACISCQLRCSSNNPPLTCQRYCNAGVTNSV

SEQ ID NO: 44 - protein, 45 aa, synthetic construct
AGQCSQNEYFDHLLRACISCQVRCSSNTPPLTCQRYCNAGVTNSV

SEQ ID NO: 45 - protein, 45 aa, synthetic construct
AGQCSQNEYFDHLIRACIPCDVRCSSNNPPVTCQRYCNAGVTNSV

SEQ ID NO: 46 - protein, 45 aa, synthetic construct
AGQCSQNEYFDHLIRACIPCQVRCSSNNPPLTCQRYCNAGVTNSV

SEQ ID NO: 47 - protein, 45 aa, synthetic construct
AGQCSQNEYFDHLIRACIPCQVRCSSNTPPLTCQRYCNAGVTNSV

SEQ ID NO: 48 - protein, 45 aa, synthetic construct
AGQCSQNEYFDHLVRACIPCQVRCSSNTPPLTCQRYCNAGVTNSV

SEQ ID NO: 49 - protein, 45 aa, synthetic construct
AGQCSQNEYFDRLVRACIPCQVRCSSNTPPLTCQRYCNAGVTNSV

SEQ ID NO: 50 - protein, 45 aa, synthetic construct
ARQCSQNEYYDRLLRSCVPCHVRCSSNNPPVTCQRYCNAGVTNSV

SEQ ID NO: 51 - protein, 45 aa, synthetic construct
ARQCSQNEYFDRLLRSCIPCQVRCSSNNPPLTCQRYCNAGVTNSV

SEQ ID NO: 52 - protein, 45 aa, synthetic construct
AGQCSQNEYFDRLLRSCIPCQLRCSSNTPPLTCQRYCNAGVTNSV

SEQ ID NO: 53 - protein, 45 aa, synthetic construct
AGQCSQNEYFDRLLRACIPCQLRCSSNTPPLTCQRYCNAGVTNSV

SEQ ID NO: 54 - protein, 45 aa, synthetic construct
AGQCSQNEYFDSLLRSCIPCQLRCSSNTPPLTCQRYCNAGVTNSV

SEQ ID NO: 55 - protein, 45 aa, synthetic construct
AGQCSQNEYFDRLLRSCIPCQVRCSSNTPPLTCQRYCNAGVTNSV

SEQ ID NO: 56 - protein, 45 aa, synthetic construct
AGQCSQNEYFDSLLRACIPCQLRCSSNTPPLTCQRYCNAGVTNSV

SEQ ID NO: 57 - protein, 45 aa, synthetic construct
AGQCSQNDYYDHLLRACIPCQLRCSSNNPPLTCQRYCNAGVTNSV

SEQ ID NO: 58 - protein, 45 aa, synthetic construct
AGQCSQNEYFDHLLRACIPCQLRCSSNNPPLTCQRYCNAGVTNSV

SEQ ID NO: 59 - protein, 45 aa, synthetic construct
AGQCSQSEYYDRLIRACIPCQLRCSSNNPPVICQRYCNAGVTNSV

SEQ ID NO: 60 - protein, 45 aa, synthetic construct
AGQCSQSEYFDRLIRACIPCQLRCSSNNPPLTCQRYCNAGVTNSV

SEQ ID NO: 61 - protein, 45 aa, synthetic construct
AGQCSQNEYYDRLIRACLPCHVRCSSNTPPATCQRYCNAGVTNSV

SEQ ID NO: 62 - protein, 45 aa, synthetic construct; identical to SEQ ID NO: 37

SEQ ID NO: 63 - protein, 45 aa, synthetic construct
AGQCSQNEYYDHLIRACISCQIRCSSNNPPPICQRYCNAGVTNSV

SEQ ID NO: 64 - protein, 45 aa, synthetic construct
AGQCSQNEYFDHLIRACISCQIRCSSNNPPLTCQRYCNAGVTNSV

SEQ ID NO: 65 - protein, 45 aa, synthetic construct
ARQCSPSEYYDHLIRSCVPCQLRCSSNTPPVTCQRYCDAGVTNSV

SEQ ID NO: 66 - protein, 45 aa, synthetic construct
ARQCSPSEYFDHLIRSCIPCQLRCSSNTPPLTCQRYCDAGVTNSV

SEQ ID NO: 67 - protein, 45 aa, synthetic construct
AGQCSQSEYYDRLIHACVPCQLRCSSNNPPATCQRYCNAGVTNSV

SEQ ID NO: 68 - protein, 45 aa, synthetic construct
AGQCSQSEYFDRLIHACIPCQLRCSSNNPPLTCQRYCNAGVTNSV

SEQ ID NO: 69 - protein, 45 aa, synthetic construct
ARQCSPNEYYDSLIRACVPCQLRCSSNNPPVTCQRYCNAGVTNSV

SEQ ID NO: 70 - protein, 45 aa, synthetic construct
ARQCSPNEYFDSLIRACIPCQLRCSSNNPPLTCQRYCNAGVTNSV

SEQ ID NO: 71 - protein, 45 aa, synthetic construct
AGQCSQNEYFDPLLRACIPCQVRCSSNTPPLTCQRYCNAGVTNSV

SEQ ID NO: 72 - protein, 45 aa, synthetic construct
AGQCSQNEYFDTLLRACIPCQVRCSSNTPPLTCQRYCNAGVTNSV

SEQ ID NO: 73 - protein, 45 aa, synthetic construct
AGQCSQNEYFDNLLRACIPCQVRCSSNTPPLTCQRYCNAGVTNSV

SEQ ID NO: 74 - protein, 45 aa, synthetic construct
AGQCSQNEYFDQLLRACIPCQVRCSSNTPPLTCQRYCNAGVTNSV

SEQ ID NO: 75 - protein, 45 aa, synthetic construct
AGQCSQNEYFDHLPRACIPCQVRCSSNTPPLTCQRYCNAGVTNSV

SEQ ID NO: 76 - protein, 45 aa, synthetic construct
AGQCSQNEYFDHLTRACIPCQVRCSSNTPPLTCQRYCNAGVTNSV

SEQ ID NO: 77 - protein, 45 aa, synthetic construct
AGQCSQNEYFDHLLAACIPCQVRCSSNTPPLTCQRYCNAGVTNSV

SEQ ID NO: 78 - protein, 45 aa, synthetic construct
AGQCSQNEYFDHLLRTCIPCQVRCSSNTPPLTCQRYCNAGVTNSV

SEQ ID NO: 79 - protein, 45 aa, synthetic construct
AGQCSQNEYFDHLLRNCIPCQVRCSSNTPPLTCQRYCNAGVTNSV

SEQ ID NO: 80 - protein, 45 aa, synthetic construct
AGQCSQNEYFDHLLRQCIPCQVRCSSNTPPLTCQRYCNAGVTNSV

SEQ ID NO: 81 - protein, 45 aa, synthetic construct
AGQCSQNEYFDHLLRVCIPCQVRCSSNTPPLTCQRYCNAGVTNSV

SEQ ID NO: 82 - protein, 45 aa, synthetic construct
AGQCSQNEYFDHLLRYCIPCQVRCSSNTPPLTCQRYCNAGVTNSV

SEQ ID NO: 83 - protein, 45 aa, synthetic construct
AGQCSQNEYFDHLLRICIPCQVRCSSNTPPLTCQRYCNAGVTNSV

SEQ ID NO: 84 - protein, 45 aa, synthetic construct
AGQCSQNEYFDHLLRFCIPCQVRCSSNTPPLTCQRYCNAGVTNSV

SEQ ID NO: 85 - protein, 45 aa, synthetic construct
AGQCSQNEYFDHLLRACNPCQVRCSSNTPPLTCQRYCNAGVTNSV

SEQ ID NO: 86 - protein, 45 aa, synthetic construct
AGQCSQNEYFDHLLRACQPCQVRCSSNTPPLTCQRYCNAGVTNSV

SEQ ID NO: 87 - protein, 45 aa, synthetic construct
AGQCSQNEYFDHLLRACSPCQVRCSSNTPPLTCQRYCNAGVTNSV

SEQ ID NO: 88 - protein, 45 aa, synthetic construct
AGQCSQNEYFDHLLRACTPCQVRCSSNTPPLTCQRYCNAGVTNSV

SEQ ID NO: 89 - protein, 45 aa, synthetic construct
AGQCSQNEYFDHLLRACAPCQVRCSSNTPPLTCQRYCNAGVTNSV

SEQ ID NO: 90 - protein, 45 aa, synthetic construct
AGQCSQNEYFDHLLRACIACQVRCSSNTPPLTCQRYCNAGVTNSV

SEQ ID NO: 91 - protein, 45 aa, synthetic construct
AGQCSQNEYFDHLLRACIGCQVRCSSNTPPLTCQRYCNAGVTNSV

SEQ ID NO: 92 - protein, 45 aa, synthetic construct
AGQCSQNEYFDHLLRACITCQVRCSSNTPPLTCQRYCNAGVTNSV

SEQ ID NO: 93 - protein, 45 aa, synthetic construct
AGQCSQNEYFDHLLRACINCQVRCSSNTPPLTCQRYCNAGVTNSV

SEQ ID NO: 94 - protein, 45 aa, synthetic construct
AGQCSQNEYFDHLLRACIQCQVRCSSNTPPLTCQRYCNAGVTNSV

SEQ ID NO: 95 - protein, 45 aa, synthetic construct
AGQCSQNEYFDHLLRACIPCIVRCSSNTPPLTCQRYCNAGVTNSV

SEQ ID NO: 96 - protein, 45 aa, synthetic construct
AGQCSQNEYFDHLLRACIPCQARCSSNTPPLTCQRYCNAGVTNSV

SEQ ID NO: 97 - protein, 45 aa, synthetic construct
AGQCSQNEYFDHLLRACIPCQYRCSSNTPPLTCQRYCNAGVTNSV

SEQ ID NO: 98 - protein, 45 aa, synthetic construct
AGQCSQNEYFDHLLRACIPCQFRCSSNTPPLTCQRYCNAGVTNSV

SEQ ID NO: 99 - protein, 45 aa, synthetic construct
AGQCSQNEYFDHLLRACIPCQVACSSNTPPLTCQRYCNAGVTNSV

SEQ ID NO: 100 - protein, 45 aa, synthetic construct
AGQCSQNEYFDHLLRACIPCQVGCSSNTPPLTCQRYCNAGVTNSV

SEQ ID NO: 101 - protein, 45 aa, synthetic construct
AGQCSQNEYFDHLLRACIPCQVSCSSNTPPLTCQRYCNAGVTNSV

SEQ ID NO: 102 - protein, 45 aa, synthetic construct
AGQCSQNEYFDHLLRACIPCQVTCSSNTPPLTCQRYCNAGVTNSV

SEQ ID NO: 103 - protein, 45 aa, synthetic construct
AGQCSQNEYFDHLLRACIPCQVNCSSNTPPLTCQRYCNAGVTNSV

SEQ ID NO: 104 - protein, 45 aa, synthetic construct
AGQCSQNEYFDHLLRACIPCQVQCSSNTPPLTCQRYCNAGVTNSV

SEQ ID NO: 105 - protein, 45 aa, synthetic construct
AGQCSQNEYFDHLLRACIPCQVWCSSNTPPLTCQRYCNAGVTNSV

SEQ ID NO: 106 - protein, 45 aa, synthetic construct
AGQCSQNEYFDHLLRACIPCQVHCSSNTPPLTCQRYCNAGVTNSV

SEQ ID NO: 107 - protein, 45 aa, synthetic construct
AGQCSQNEYFDPLIRACIPCQVRCSSNTPPLTCQRYCNAGVTNSV

SEQ ID NO: 108 - protein, 45 aa, synthetic construct
AGQCSQNEYFDNLLRACIPCQYRCSSNTPPLTCQRYCNAGVTNSV

SEQ ID NO: 109 - protein, 45 aa, synthetic construct
AGQCSQNEYFDHLIRACIPCQARCSSNTPPLTCQRYCNAGVTNSV

SEQ ID NO: 110 - protein, 45 aa, synthetic construct
AGQCSQNEYFDHLLRYCIPCQYRCSSNTPPLTCQRYCNAGVTNSV

SEQ ID NO: 111 - protein, 45 aa, synthetic construct
AGQCSQNEYFDSLIRHCIPCQVRCSSNTPPLTCQRYCNAGVTNSV

SEQ ID NO: 112 - protein, 45 aa, synthetic construct
AGQCSQNEYFDHLSRACIPCQVRCSSNTPPLTCQRYCNAGVTNSV

SEQ ID NO: 113 - protein, 45 aa, synthetic construct
AGQCSQNEYFDHLGRACIPCQVRCSSNTPPLTCQRYCNAGVTNSV

SEQ ID NO: 114 - protein, 45 aa, synthetic construct
AGQCSQNEYFDHLFRACIPCQVRCSSNTPPLTCQRYCNAGVTNSV

SEQ ID NO: 115 - protein, 45 aa, synthetic construct
AGQCSQNEYFDHLIRACIPCQSRCSSNTPPLTCQRYCNAGVTNSV

SEQ ID NO: 116 - protein, 45 aa, synthetic construct
AGQCSQNEYFDSLVRHCIPCQIRCSSNTPPLTCQRYCNAGVTNSV

SEQ ID NO: 117 - protein, 45 aa, synthetic construct
AGQCSQNEYFDHLLRTCIPCQYRCSSNTPPLTCQRYCNAGVTNSV

SEQ ID NO: 118 - protein, 45 aa, synthetic construct
AGQCSQNEYFDSLVRHCIPCQVRCSSNTPPLTCQRYCNAGVTNSV

SEQ ID NO: 119 - protein, 45 aa, synthetic construct
AGQCSQNEYFDSLIRHCIPCQIRCSSNTPPLTCQRYCNAGVTNSV

SEQ ID NO: 120 - protein, 45 aa, synthetic construct
AGQCSQNEYFDSLVRTCIPCQIRCSSNTPPLTCQRYCNAGVTNSV

SEQ ID NO: 121 - protein, 45 aa, synthetic construct
AGQCSQNEYFDHLARACIPCQVRCSSNTPPLTCQRYCNAGVTNSV

SEQ ID NO: 122 - protein, 45 aa, synthetic construct
AGQCSQNEYFDHLYRACIPCQVRCSSNTPPLTCQRYCNAGVTNSV

SEQ ID NO: 123 - protein, 45 aa, synthetic construct
AGQCSQNEYFDHLIRACIPCQTRCSSNTPPLTCQRYCNAGVTNSV

SEQ ID NO: 124 - protein, 45 aa, synthetic construct
AGQCSQNEYFDHLIRACIPCQGRCSSNTPPLTCQRYCNAGVTNSV

SEQ ID NO: 125 - protein, 45 aa, synthetic construct
AGQCSQNEYFDSLVRACQPCQVRCSSNTPPLTCQRYCNAGVTNSV

SEQ ID NO: 126 - protein, 45 aa, synthetic construct
AGQCSQNEYFDSLIRACQPCQVRCSSNTPPLTCQRYCNAGVTNSV

SEQ ID NO: 127 - protein, 45 aa, synthetic construct
AGQCSQNEYFDHLVRACQPCQLRCSSNTPPLTCQRYCNAGVTNSV

SEQ ID NO: 128 - protein, 45 aa, synthetic construct
AGQCSQNEYFDHLIRACQPCQLRCSSNTPPLTCQRYCNAGVTNSV

SEQ ID NO: 129 - protein, 45 aa, synthetic construct
AGQCSQNEYFDHLIRICIPCQVRCSSNTPPLTCQRYCNAGVTNSV

SEQ ID NO: 130 - protein, 45 aa, synthetic construct
AGQCSQNEYFDHLVRICIPCQVRCSSNTPPLTCQRYCNAGVTNSV

SEQ ID NO: 131 - protein, 45 aa, synthetic construct
AGQCSQNEYFDHLLRICIPCQYRCSSNTPPLTCQRYCNAGVTNSV

SEQ ID NO: 132 - protein, 45 aa, synthetic construct
AGQCSQNEYFDHLIRACTPCQVRCSSNTPPLTCQRYCNAGVTNSV

SEQ ID NO: 133 - protein, 45 aa, synthetic construct
AGQCSQNEYFDHLVRACTPCQVRCSSNTPPLTCQRYCNAGVTNSV

SEQ ID NO: 134 - protein, 45 aa, synthetic construct
AGQCSQNEYFDHLIRACIPCQVTCSSNTPPLTCQRYCNAGVTNSV

SEQ ID NO: 135 - protein, 45 aa, synthetic construct
AGQCSQNEYFDHLVRACIPCQVTCSSNTPPLTCQRYCNAGVTNSV

SEQ ID NO: 136 - protein, 45 aa, synthetic construct
AGQCSQNEYFDHLLRACIPCQYTCSSNTPPLTCQRYCNAGVTNSV

SEQ ID NO: 137 - protein, 45 aa, synthetic construct
AGQCSQNEYFDPLIRACIPCQARCSSNTPPLTCQRYCNAGVTNSV

SEQ ID NO: 138 - protein, 45 aa, synthetic construct
AGQCSQNEYFDPLIRACIPCQIRCSSNTPPLTCQRYCNAGVTNSV

SEQ ID NO: 139 - protein, 45 aa, synthetic construct
AGQCSQNEYFDPLVRACIPCQVRCSSNTPPLTCQRYCNAGVTNSV

SEQ ID NO: 140 - protein, 45 aa, synthetic construct
AGQCSQNEYFDPLVRACIPCQARCSSNTPPLTCQRYCNAGVTNSV

SEQ ID NO: 141 - protein, 45 aa, synthetic construct
AGQCSQNEYFDPLVRACIPCQIRCSSNTPPLTCQRYCNAGVTNSV

SEQ ID NO: 142 - protein, 45 aa, synthetic construct
AGQCSQNEYFDHLIRACIPCQIRCSSNTPPLTCQRYCNAGVTNSV

SEQ ID NO: 143 - protein, 45 aa, synthetic construct
AGQCSQNEYFDHLVRACIPCQARCSSNTPPLTCQRYCNAGVTNSV

SEQ ID NO: 144 - protein, 45 aa, synthetic construct
AGQCSQNEYFDHLVRACIPCQIRCSSNTPPLTCQRYCNAGVTNSV

SEQ ID NO: 145 - protein, 45 aa, synthetic construct
AGQCSQNEYFDSLVRHCIPCQARCSSNTPPLTCQRYCNAGVTNSV

SEQ ID NO: 146 - protein, 45 aa, synthetic construct
AGQCSQNEYFDSLIRHCIPCQARCSSNTPPLTCQRYCNAGVTNSV

SEQ ID NO: 147 - protein, 42 aa, synthetic construct
SLSCRKEQGKFYDHLLRDCISCASICGQHPKQCAYFCENKLR

SEQ ID NO: 148 - DNA, 891 bp, Homo sapiens
  source 1..891: mol_type = genomic DNA
attgttctcaacattctagctgctcttgctgcatttgctctggaattcttgtagagatattacttgtccttccaggctgttctttctgtagctcccttgttttctttttgtgatcatgttgcagatggctgggcagtgctcccaaaatgaatattttgacagtttgttgcatgcttgcataccttgtcaacttcgatgttcttctaatactcctcctctaacatgtcagcgttattgtaatgcaagtgtgaccaattcagtgaaaggaacgaatgcgattctctggacctgtttgggactgagcttaataatttctttggcagttttcgtgctaatgtttttgctaaggaagataaactctgaaccattaaaggacgagtttaaaaacacaggatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctaggtaattaaccatttcgactcgagcagtgccactttaaaaatcttttgtcagaatagatgatgtgtcagatctctttaggatgactgtatttttcagttgccgatacagctttttgtcctctaactgtggaaactctttatgttagatatatttctctaggttactgttgggagcttaatggtagaaacttccttggtttcatgattaaactcttttttttcctga

SEQ ID NO: 149 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 150 - protein, 184 aa, Homo sapiens [sequence omitted]

SEQ ID NO: 151 - protein, 150 aa, Homo sapiens
GCTGGGCAGTGCTCCCAAAATGAATATTTTGACAGTTTGTTGCATGCTTGCATACCTTGTCAACTTCGATGTTCTTCTAATACTCCTCCTCTAACATGTCAGCGTTATTGTAATGCAAGTGTGACCAATTCAGTGAAAGGAACGAATGCG

SEQ ID NO: 152 - protein, 50 aa, Homo sapiens
AGQCSQNEYFDSLLHACIPCQLRCSSNTPPLTCQRYCNASVTNSVKGTNA

SEQ ID NO: 153 - DNA, 57 bp, synthetic construct
  source 1..57: mol_type = other DNA
atggcctggatgatgcttctcctcggactccttgcttatggatcaggagtcgactct

SEQ ID NO: 154 - protein, 19 aa, synthetic construct
MAWMMLLLGLLAYGSGVDS

SEQ ID NO: 155 - DNA, 17 bp, synthetic construct
  source 1..17: mol_type = other DNA
ggcggcggtggcagcgc

SEQ ID NO: 156 - protein, 5 aa, synthetic construct
GGGGS

SEQ ID NO: 157 - DNA, 18 bp, synthetic construct
  source 1..18: mol_type = other DNA
ggcggcggtggcagcgct

SEQ ID NO: 158 - protein, 6 aa, synthetic construct
GGGGSA

SEQ ID NO: 159 - DNA, 135 bp, synthetic construct
  source 1..135: mol_type = other DNA
gccggacagtgtagccagaacgagtacttcgatcacctgattcgcgcttgtataccgtgccaacttagatgctcgagtaatactccaccactgacatgccagcgttattgcaacgccggcgtcacaaacagcgtc

SEQ ID NO: 160 - DNA, 692 bp, synthetic construct
  source 1..692: mol_type = other DNA
tgagtccaaatatggtcccccatgcccaccatgcccagcaccagaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaatgag

SEQ ID NO: 161 - protein, 230 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 162 - DNA, 691 bp, synthetic construct
  source 1..691: mol_type = other DNA
gagtccaaatatggtcccccatgcccaccatgcccagcaccagaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaatgag

SEQ ID NO: 163 - protein, 229 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 164 - DNA, 901 bp, synthetic construct
  source 1..901: mol_type = other DNA
atggcctggatgatgcttctcctcggactccttgcttatggatcaggagtcgactctgccggacagtgtagccagaacgagtacttcgatcacctgattcgcgcttgtataccgtgccaacttagatgctcgagtaatactccaccactgacatgccagcgttattgcaacgccggcgtcacaaacagcgtcggcggcggtggcagcgctgagtccaaatatggtcccccatgcccaccatgcccagcaccagaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaatgag

SEQ ID NO: 165 - protein, 299 aa, synthetic construct
MAWMMLLLGLLAYGSGVDSAGQCSQNEYFDHLIRACIPCQLRCSSNTPPLTCQRYCNAGVTNSVGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 166 - DNA, 844 bp, synthetic construct
  source 1..844: mol_type = other DNA
gccggacagtgtagccagaacgagtacttcgatcacctgattcgcgcttgtataccgtgccaacttagatgctcgagtaatactccaccactgacatgccagcgttattgcaacgccggcgtcacaaacagcgtcggcggcggtggcagcgctgagtccaaatatggtcccccatgcccaccatgcccagcaccagaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaatgag

SEQ ID NO: 167 - protein, 280 aa, synthetic construct
AGQCSQNEYFDHLIRACIPCQLRCSSNTPPLTCQRYCNAGVTNSVGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 168 - protein, 227 aa, Homo sapiens
DKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 232 aa, Homo sapiens
EPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 172 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 173-174 - skipped (2 entries)

SEQ ID NO: 175 - protein, 4 aa, synthetic construct
GGGS

SEQ ID NO: 176 - protein, 6 aa, synthetic construct
GGGGGS

SEQ ID NO: 177 - protein, 7 aa, synthetic construct
GGGGGGS

SEQ ID NO: 178 - protein, 8 aa, synthetic construct
GGGGGGGS

SEQ ID NO: 179 - protein, 9 aa, synthetic construct
GGGGGGGGS

SEQ ID NO: 180 - protein, 10 aa, synthetic construct
GGGGGGGGGS

SEQ ID NO: 181 - protein, 11 aa, synthetic construct
GGGGGGGGGGS

SEQ ID NO: 182 - protein, 10 aa, synthetic construct
GGGGSGGGGS

SEQ ID NO: 183 - protein, 15 aa, synthetic construct
GGGGSGGGGSGGGGS

SEQ ID NO: 184 - protein, 20 aa, synthetic construct
GGGGSGGGGSGGGGSGGGGS

SEQ ID NO: 185 - protein, 25 aa, synthetic construct
GGGGSGGGGSGGGGSGGGGSGGGGS

SEQ ID NO: 186 - protein, 30 aa, synthetic construct
GGGGSGGGGSGGGGSGGGGSGGGGSGGGGS

SEQ ID NO: 187 - skipped

SEQ ID NO: 188 - protein, 4 aa, synthetic construct
GGSA

SEQ ID NO: 189 - protein, 5 aa, synthetic construct
GGGSA

SEQ ID NO: 190 - protein, 7 aa, synthetic construct
GGGGGSA

SEQ ID NO: 191 - protein, 8 aa, synthetic construct
GGGGGGSA

SEQ ID NO: 192 - protein, 9 aa, synthetic construct
GGGGGGGSA

SEQ ID NO: 193 - protein, 10 aa, synthetic construct
GGGGGGGGSA

SEQ ID NO: 194 - protein, 11 aa, synthetic construct
GGGGGGGGGSA

SEQ ID NO: 195 - protein, 12 aa, synthetic construct
GGGGGGGGGGSA

SEQ ID NO: 196 - protein, 12 aa, synthetic construct
GGGGSAGGGGSA

SEQ ID NO: 197 - protein, 18 aa, synthetic construct
GGGGSAGGGGSAGGGGSA

SEQ ID NO: 198 - protein, 23 aa, synthetic construct
GGGGSAGGGGSAGGGGSGGGGSA

SEQ ID NO: 199 - protein, 30 aa, synthetic construct
GGGGSAGGGGSAGGGGSAGGGGSAGGGGSA

SEQ ID NO: 200 - protein, 36 aa, synthetic construct
GGGGSAGGGGSAGGGGSAGGGGSAGGGGSAGGGGSA

SEQ ID NO: 201 - protein, 4 aa, synthetic construct
GGGA

SEQ ID NO: 202 - protein, 5 aa, synthetic construct
GGGGA

SEQ ID NO: 203 - protein, 6 aa, synthetic construct
GGGGGA

SEQ ID NO: 204 - protein, 7 aa, synthetic construct
GGGGGGA

SEQ ID NO: 205 - protein, 8 aa, synthetic construct
GGGGGGGA

SEQ ID NO: 206 - protein, 9 aa, synthetic construct
GGGGGGGGA

SEQ ID NO: 207 - protein, 10 aa, synthetic construct
GGGGGGGGGA

SEQ ID NO: 208 - protein, 11 aa, synthetic construct
GGGGGGGGGGA

SEQ ID NO: 209 - protein, 10 aa, synthetic construct
GGGGAGGGGA

SEQ ID NO: 210 - protein, 15 aa, synthetic construct
GGGGAGGGGAGGGGA

SEQ ID NO: 211 - protein, 20 aa, synthetic construct
GGGGAGGGGAGGGGAGGGGA

SEQ ID NO: 212 - protein, 25 aa, synthetic construct
GGGGAGGGGAGGGGAGGGGAGGGGA

SEQ ID NO: 213 - protein, 30 aa, synthetic construct
GGGGAGGGGAGGGGAGGGGAGGGGAGGGGA

SEQ ID NO: 214 - protein, 285 aa, synthetic construct
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 215 - protein, 250 aa, synthetic construct [sequence omitted]

SEQ ID NO: 216 - protein, 234 aa, synthetic construct
MPASSPFLLAPKGPPGNMGGPVREPALSVALWLSWGAALGAVACAMALLTQQTELQSLRREVSRLQGTGGPSQNGEGYPWQSLPEQSSDALEAWENGERSRKRRAVLTQKQKNDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 217 - protein, 250 aa, synthetic construct [sequence omitted]

SEQ ID NO: 218 - DNA, 310 bp, synthetic construct
  source 1..310: mol_type = other DNA
ccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagc

SEQ ID NO: 219 - DNA, 627 bp, synthetic construct
  source 1..627: mol_type = other DNA
aaaggatcttcttgagatcctttttttctgcgcgtaatctgctgcttgcacaacaaaaaaaccaccgctaccagcggtggtttgtttgccggatcaagagcctaccaactctttttccgaaggtaactggcttcagcagagcgcagataccacaatactgtccttctagtgtagccgtagttaggccaccacttcaagaactcctgtagcaccgcctacatacctcgctctgctaatcctgttaccagtggctgcctgccagtggcgataagtcgtgtcttaccgggttggactcaagacgatagcttaccggataaggcgcagcggtcgggctgaacggggggttcgtgcacacacgcccagcttggagcgaacgacctacaccgaactgagatacctacagcgtgcagctatgagaaagcgccacgcttcccgaagggagaaaggcggacaggtatcccggtaagcggcagggtcggaacaggagagcgcacgagggagcttccaggcgggaaacgcctggtatctttatagtcctgtcgggtttcgccacctctgaccttgagcgtcgatttttgtgatgctcgtcaggggggcggagcctatggaaacaacgccagcaacgcg

SEQ ID NO: 220 - DNA, 600 bp, synthetic construct
  source 1..600: mol_type = other DNA
atgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 221 - protein, 199 aa, synthetic construct
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 222 - DNA, 861 bp, synthetic construct
  source 1..861: mol_type = other DNA
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 223 - protein, 286 aa, synthetic construct
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 224 - protein, 50 aa, synthetic construct
AGQCSQNEYFDSLLHACIPCQLRCSSNTPPLTCQRYCNAGVTNSVKGTNA

SEQ ID NO: 225 - DNA, 1575 bp, synthetic construct
  source 1..1575: mol_type = other DNA
ctgggctgagacccgcagaggaagacgctctagggatttgtcccggactagcgagatggcaaggctgaggacgggaggctgattgagaggcgaaggtacaccctaatctcaatacaacccttggagctaagccagcaatggtagagggaagattctgcacgtcccttccaggcggcctccccgtcaccacccaccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttgaaagtgggtgggagagttcgaggccttgcgcttaaggagccccttcgcctcgtgcttgagttgaggcctggcttgggcgctggggccgccgcgtgcgaatctggtggcaccttcgcgcctatctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagttttttccttccatttcaggtgtcgtgaaaactacccctaaaagccaaa

SEQ ID NO: 226 - protein, 231 aa, Homo sapiens
PKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 227 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 233 aa, Homo sapiens
AEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - protein, 231 aa, Homo sapiens
PKSCDKTHTCPPCPAPELKGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 230 aa, Homo sapiens
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 232 - protein, 229 aa, Homo sapiens
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 233 - protein, 229 aa, Homo sapiens
ESKYGPPCPPCPAPEFAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 234 - protein, 229 aa, Homo sapiens
ESKYGPPCPSCPAPEVAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 235 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 236 - protein, 228 aa, Homo sapiens
ERKCCVECPPCPAPPVAGPSVFLFPPKPKDTLMISRTPEVTCVVVDVKHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 237 - protein, 45 aa, synthetic construct
AGQCSQNEYFDSLLHACIPCQLRCSSNTPPLTCQRYCNAGVTNSV

SEQ ID NO: 238 - DNA, 691 bp, synthetic construct; identical to SEQ ID NO: 162
  source 1..691: mol_type = other DNA

SEQ ID NO: 239 - DNA, 900 bp, synthetic construct
  source 1..900: mol_type = other DNA
atggcctggatgatgcttctcctcggactccttgcttatggatcaggagtcgactctgccggacagtgtagccagaacgagtacttcgatcacctgattcgcgcttgtataccgtgccaacttagatgctcgagtaatactccaccactgacatgccagcgttattgcaacgccggcgtcacaaacagcgtcggcggcggtggcagcgctgagtccaaatatggtcccccatgcccaccatgcccagcaccagaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaatga

SEQ ID NO: 240 - DNA, 308 bp, synthetic construct
  source 1..308: mol_type = other DNA
ctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagc

SEQ ID NO: 241 - DNA, 615 bp, synthetic construct
  source 1..615: mol_type = other DNA
cgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatccttt

SEQ ID NO: 242 - DNA, 861 bp, synthetic construct
  source 1..861: mol_type = other DNA
ttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 243 - DNA, 843 bp, synthetic construct
  source 1..843: mol_type = other DNA
gccggacagtgtagccagaacgagtacttcgatcacctgattcgcgcttgtataccgtgccaacttagatgctcgagtaatactccaccactgacatgccagcgttattgcaacgccggcgtcacaaacagcgtcggcggcggtggcagcgctgagtccaaatatggtcccccatgcccaccatgcccagcaccagaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaatga

SEQ ID NO: 244 - DNA, 578 bp, synthetic construct
  source 1..578: mol_type = other DNA
tccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccaca

SEQ ID NO: 245 - DNA, 906 bp, synthetic construct
  source 1..906: mol_type = other DNA
atgtacaggatgcaactcctgtcttgcattgcactaagtcttgcacttgtcacgaattcggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgtacgatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggacatgaaacaaccccaaataaaggaagtggaaccacttcaggtactacccgtcttctatctgggcacacgtgtttcacgttgacaggtttgcttgggacgctagtaaccatgggcttgctgacttaa

SEQ ID NO: 246 - protein, 301 aa, synthetic construct
MYRMQLLSCIALSLALVTNSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGHETTPNKGSGTTSGTTRLLSGHTCFTLTGLLGTLVTMGLLT

SEQ ID NO: 247 - DNA, 317 bp, synthetic construct
  source 1..317: mol_type = other DNA
ggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 248 - DNA, 793 bp, synthetic construct
  source 1..793: mol_type = other DNA
cgacccgcagcgcccgaccgaaaggagcgcacgaccccatggctccgaccgaagccgacccgggcggccccgccgaccccgcacccgcccccgaggcccaccgactaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgaattcgatcgccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaagg

SEQ ID NO: 249 - DNA, 27 bp, synthetic construct
  source 1..27: mol_type = other DNA
atgtacaggatgcaactcctgtcttgc

SEQ ID NO: 250 - protein, 20 aa, synthetic construct
MYRMQLLSCIALSLALVTNS

SEQ ID NO: 251 - DNA, 862 bp, synthetic construct
  source 1..862: mol_type = other DNA
acttgtcacgaattcggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgtacgatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggacatgaaacaaccccaaataaaggaagtggaaccacttcaggtactacccgtcttctatctgggcacacgtgtttcacgttgacaggtttgcttgggacgctagtaaccatgggcttgctgacttaa

SEQ ID NO: 252 - protein, 281 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGHETTPNKGSGTTSGTTRLLSGHTCFTLTGLLGTLVTMGLLT

SEQ ID NO: 253 - protein, 6 aa, synthetic construct
HHHHHH

SEQ ID NO: 254 - protein, 38 aa, synthetic construct
CRKEQGKFYDHLLRDCISCASICGQHPKQCAYFCENKL